(19) United States
(12) Patent Application Publication
(10) Pub. No.: US 2023/0219962 A1
(43) Pub. Date: Jul. 13, 2023

(54) PYRAZOLO[4,3-d]PYRIMIDINE DERIVATIVES AND METHODS OF USE THEREOF FOR THE TREATMENT OF CELLULAR PROLIFERATIVE DISORDERS

(71) Applicants: Jianming BAO, Princeton, NJ (US); Natalija CERNAKA, South San Francisco, CA (US); Scott D. HOLLINGSWORTH, South San Francisco, CA (US); Madeleine Eileen KIEFFER, South San Francisco, CA (US); Simon B. LANG, South San Francisco, CA (US); Akash M. PATEL, South San Francisco, CA (US); W. Michael SEGANISH, South San Francisco, CA (US); Merck Sharp & Dohme LLC, Rahway, NJ (US)

(72) Inventors: Jianming Bao, Princeton, NJ (US); Natalija Cernaka, Redwood City, CA (US); Scott A. Hollingsworth, Redwood City, CA (US); Madeleine Eileen Kieffer, Burlingame, CA (US); Simon B. Lang, San Francisco, CA (US); Akash M. Patel, Millbrae, CA (US); W. Michael Seganish, Castro Valley, CA (US)

(73) Assignee: Merck Sharp & Dohme LLC, Rahway, NJ (US)

(21) Appl. No.: 17/927,222

(22) PCT Filed: Jun. 3, 2021
(86) PCT No.: PCT/US2021/035622

§ 371 Date: Nov. 22, 2022

Related U.S. Application Data
(60) Provisional application No. 63/034,687, filed on Jun. 4, 2020.

(51) Int. Cl. C07D 487/04; C07D 519/00; A61K 39/395; A61K 31/519

(52) U.S. Cl. CPC C07D 487/04; C07D 519/00; A61K 39/3955; A61K 31/519

(57) ABSTRACT

The present invention relates to novel Pyrazolo[4,3 -d]Pyrimidine Derivatives of Formula (I): (I) and pharmaceutically acceptable salts thereof, wherein R¹, R² and R³ are as defined herein. The present invention also relates to compositions comprising at least one Pyrazolo[4,3-d]Pyrimidine Derivative, and methods of using the Pyrazolo[4,3-d]Pyrimidine Derivatives for treating or preventing a cellular proliferative disorder in a patient.

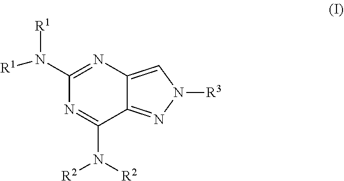

### FIELD OF THE INVENTION

The present invention relates to novel Pyrazolo[4,3-d]Pyrimidine Derivatives, compositions comprising at least one Pyrazolo[4,3-d]Pyrimidine Derivative, and methods of using the Pyrazolo[4,3-d]Pyrimidine Derivatives for treating or preventing a cellular proliferative disorder in a patient.

### BACKGROUND OF THE INVENTION

In 2020, an estimated 1.9 million new cancer cases will be diagnosed in the United States, with, approximately 630,000 cancer-related deaths. Cancer places a tremendous burden on individuals, and society as a whole. The Agency for Healthcare Research and Quality estimates that the direct medical costs for cancer in the U.S. in 2015 were an overwhelming $80.2 billion.

Over the last decade, significant advances in research, education, early detection methods, and treatment have boosted cancer survival rates while new therapies continue to be developed. The recent introduction of cancer immunotherapies, in particular those based on immune checkpoint inhibitors, has created a paradigm shift in clinical oncology. These drugs work by unleashing the body's own immune responses to promote elimination of cancer cells.

Small-molecule agonists at Toll-like receptor 7 (TLR7), and Toll-like receptor 8 (TLR8) have sparked a vivid interest in cancer research owing to their demonstrated antitumor activity. The scientific and clinical interest in TLR7 and TLR8 for cancer biology has originated from the antitumoral activity of some small-molecule compounds, which have later been shown to act as agonists at one or both receptors. The imidazoquinoline compound imiquimod, for example, is marketed as a topical formulation, and is efficacious against many primary skin tumors and cutaneous metastases. The predominant antitumoral mode of action of these small-molecule agonists is TLR7/8-mediated activation of the central transcription factor nuclear factor-κB, which leads to induction of proinflammatory cytokines and other mediators. Cutaneous dendritic cells are the primary responsive cell type and initiate a strong Th1-weighted antitumoral cellular immune response. In addition, it has been shown that the anti-tumor effects of a TLR7/8 agonist can be enhanced through combination with checkpoint inhibitors and co-stimulatory agonists. Mullins et al., J. Immunotherapy Cancer, 7, 244 (2019).

There exists a need for novel compounds useful for the treatment of cellular proliferative disorders, such as cancer, alone or in combination with other therapeutic agent(s). The present invention helps address that need.

### SUMMARY OF THE INVENTION

In one aspect, the present invention provides Compounds of Formula (I):

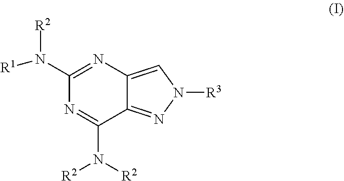

or a pharmaceutically acceptable salt thereof,
wherein:

each occurrence of R¹ is independently selected from H and C₁-C₆ alkyl;

each occurrence of R² is independently selected from H, C₁-C₈ alkyl, —(C₁-C₆ alkylene)-O—(C₁-C₆ alkyl), C₁-C₈ hydroxyalkyl, C₃-C₇ cycloalkyl, wherein said C₃-C₇ cycloalkyl group can be optionally substituted with one or more R⁴ groups, which can be the same or different; or two R² groups, together with the nitrogen atom to which they are attached, can join to form a 5- to 7-membered monocyclic heterocycloalkyl group, wherein said 5- to 7-membered monocyclic heterocycloalkyl group can be optionally substituted with one or more R⁴ groups, which can be the same or different;

R³ is selected from C₁-C₈ alkyl, —C₁-C₈ aminoalkyl, —(CH₂)ₙ-phenyl, —(CH₂)ₙ—(C₃-C₇ cycloalkyl), —(CH₂)ₙ-(4 to 7-membered monocyclic heterocycloalkyl), —(CH₂)ₙ-(5- or 6-membered monocyclic heteroaryl), and —CH₂-(7- to 10-membered bicyclic heteroaryl), wherein the phenyl moiety of said benzyl group and the phenyl moiety of said —(CH₂)ₙ-phenyl group, the 4 to 7-membered monocyclic heterocycloalkyl moiety of said —(CH₂)ₙ-(4 to 7-membered monocyclic heterocycloalkyl); and the 5- or 6-membered monocyclic heteroaryl moiety of said —(CH₂)ₙ-(5- or 6-membered monocyclic heteroaryl) group, can be optionally substituted with one or more R⁵ groups, which can be the same or different; the 7- to 10-membered bicyclic heteroaryl moiety of said —(CH₂)ₙ-(7- to 10-membered bicyclic heteroaryl) group can be optionally substituted with one or more R⁶ groups, which can be the same or different; and the C₃-C₇ cycloalkyl moiety of said —(CH₂)ₙ—(C₃-C₇ cycloalkyl) group can be optionally substituted with one or more R⁷ groups, which can be the same or different;

each occurrence of R⁴ is independently selected from C₁-C₈ alkyl, C₁-C₈ hydroxyalkyl, halo, and —OH;

each occurrence of R⁵ is independently selected from C₁-C₆ alkyl, C₃-C₇ cycloalkyl, C₁-C₆ aminoalkyl, —O—(C₁-C₆ alkyl), C₁-C₆ hydroxyalkyl, —O—(C₁-C₆ hydroxyalkyl), —O—(C₁-C₆ alkylene)-C(O)OR⁸, —O—(C₁-C₆ haloalkyl), —S—CH₂CH(NH)—C(O)OR⁸, NHC(O)—(C₁-C₆ alkyl), C₁-C₆ haloalkyl, halo, —(CH₂)ₙ-(4 to 7-membered monocyclic heterocycloalkyl), -6- to 11-membered spirocyclic bicyclic heterocycloalkyl, —N(R⁸)₂, —(C₁-C₃ alkylene)ₙ-N(R⁸)₂, —C(O)—(C₁-C₃ alkylene)-R^{C}, —(C₁-C₃ alkylene)ₙ-N(R⁸)—(C₁-C₃ alkylene)ₙ-R^{C}, —(C₁-C₃ alkylene)ₙ-NHC(O)—(C₁-C₃ alkylene)-R^{C}, —(C₁-C₃ alkylene)-(C₁-C₆ aminoalkyl), —CH(N(R⁸)₂)(C₁-C₆ aminoalkyl), —(C₁-C₃ alkylene)ₙ-N(R⁸)—(C₁-C₆ aminoalkyl), —(C₁-C₃ alkylene)ₙ-N(R⁸)—(C₁-C₃ alkylene)-NHC(O)-(5- or 6-membered monocyclic heteroaryl), R^{A}, R^{B}, R^{C},and R^{D}, wherein said 6- to 11-membered bicyclic heterocycloalkyl group can be optionally substituted with —(C₁-C₃ alkylene)-(5- to 7-membered monocyclic heterocycloalkyl) or —(C₁-C₃ alkylene)-R^{C}, and the 4 to 7-membered monocyclic heterocycloalkyl moiety of said —(CH₂)ₙ-(4 to 7-membered monocyclic heterocycloalkyl) group can be optionally substituted with —(C₁-C₃ alkylene)ₙ-N(R⁸)₂ or —(C₁-C₃ alkylene)-R^{C};

each occurrence of R⁶ is independently selected from C₁-C₆ alkyl, C₃-C₇ cycloalkyl, C₁-C₆ aminoalkyl, —O—(C₁-C₆ alkyl), —O—(C₁-C₆ hydroxyalkyl), —O—(C₁-C₆ alkylene)-C(O)OR⁸, —O—(C₁-C₆ haloalkyl), C₁-C₆ hydroxyalkyl, halo, 6- to 11-membered spirocyclic bicyclic heterocycloalkyl, —N(R⁸)₂, —(C₁-C₃ alkylene)-N(R⁸)₂, —(C₁-C₃ alkylene)-(C₁-C₆ aminoalkyl), —(C₁-C₃ alkylene)-N(CH₃)—(C₁-C₆ aminoalkyl), —NH—(C₁-C₆ aminoalkyl), R^{A}, R^{B}, and R^{C}, wherein said 6- to 11-membered spirocyclic bicyclic heterocycloalkyl group can be optionally substituted with —(C₁-C₃ alkylene)-(5- to 7-membered monocyclic heterocycloalkyl);

each occurrence of R⁷ is independently selected from C₁-C₆ alkyl, C₃-C₇ cycloalkyl, C₁-C₆ aminoalkyl, —O—(C₁-C₆ alkyl), —O—(C₁-C₆ hydroxyalkyl), —O—(C₁-C₆ alkylene)-C(O)OR⁸, —O—(C₁-C₆ haloalkyl), C₁-C₆ hydroxyalkyl, halo, 6- to 11-membered spirocyclic bicyclic heterocycloalkyl, —N(R⁸)₂, —(C₁-C₃ alkylene)-N(R⁸)₂, —(C₁-C₃ alkylene)-(C₁-C₆ aminoalkyl), —(C₁-C₃ alkylene)-N(CH₃)—(C₁-C₆ aminoalkyl), —NH—(C₁-C₆ aminoalkyl), R^{A}, R^{B}, and R^{C}, wherein said 6- to 11-membered spirocyclic bicyclic heterocycloalkyl group can be optionally substituted with —(C₁-C₃ alkylene)-(5- to 7-membered monocyclic heterocycloalkyl);

each occurrence of R⁸ is independently selected from H and C₁-C₆ alkyl;

R^{A} is:

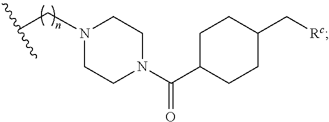

R^{B} is:

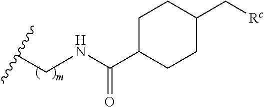

R^{C} is selected from C₁-C₆ aminoalkyl, —NHC(O)—(C₁-C₆) alkenyl,

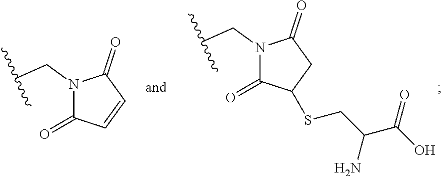 [SMILES: *CN1C(=O)C=CC1=O ; *CN1C(=O)CC(SCC(N)C(=O)O)C1=O]

R^{D} is:

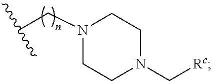

each occurrence of m is independently 1 or 2; and

each occurrence of n is independently 0 or 1.

The Compounds of Formula (I) (also referred to herein as the “Pyrazolo[4,3-d]Pyrimidine Derivatives”), and pharmaceutically acceptable salts thereof, can be useful for treating or preventing a cellular proliferative disorder in a patient. Without being bound by any specific theory, it is believed that the Pyrazolo[4,3-d]Pyrimidine Derivatives act as dual agonists of TLR7/8.

Accordingly, the present invention provides methods for treating or preventing a cellular proliferative disorder in a patient, comprising administering to the patient an effective amount of at least one Pyrazolo[4,3-d]Pyrimidine Derivative.

The details of the invention are set forth in the accompanying detailed description below.

Although any methods and materials similar to those described herein can be used in the practice or testing of the present invention, illustrative methods and materials are now described. Other embodiments, aspects and features of the present invention are either further described in or will be apparent from the ensuing description, examples and appended claims.

### DETAILED DESCRIPTION OF THE INVENTION

The present invention relates to novel Pyrazolo[4,3-d]Pyrimidine Derivatives, compositions comprising at least one Pyrazolo[4,3-d]Pyrimidine Derivative, and methods of using the Pyrazolo[4,3-d]Pyrimidine Derivatives for treating or preventing a cellular proliferative disorder in a patient.

### Definitions and Abbreviations

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name and an ambiguity exists between the structure and the name, it is to be understood that the structure predominates. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “—O-alkyl,” etc.

As used herein, and throughout this disclosure, the following terms, unless otherwise indicated, shall be understood to have the following meanings:

A “patient” is a human or non-human mammal. In one embodiment, a patient is a human.

The term “effective amount” as used herein, refers to an amount of Pyrazolo[4,3-d]Pyrimidine Derivative, and/or an additional therapeutic agent, or a composition thereof that is effective in producing the desired therapeutic, ameliorative, inhibitory or preventative effect when administered to a patient suffering from a cellular proliferative disorder. In the combination therapies of the present invention, an effective amount can refer to each individual agent or to the combination as a whole, wherein the amounts of all agents administered are together effective, but wherein the component agent of the combination may not be present individually in an effective amount.

The term “preventing,” as used herein with respect to a cellular proliferative disorder, refers to reducing the likelihood of a cellular proliferative disorder.

The term “alkyl,” as used herein, refers to an aliphatic hydrocarbon group having one of its hydrogen atoms replaced with a bond. An alkyl group may be straight or branched and contain from about 1 to about 20 carbon atoms. In one embodiment, an alkyl group contains from about 1 to about 12 carbon atoms. In different embodiments, an alkyl group contains from 1 to 8 carbon atoms (C₁-C₈ alkyl), from 1 to 6 carbon atoms (C₁-C₆ alkyl), or from about 1 to about 4 carbon atoms (C₁-C₄ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. An alkyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, NH(cycloalkyl), —O—C(O)-alkyl, —O—C(O)-aryl, —O—C(O)-cycloalkyl, —C(O)OH and —C(O)O-alkyl. Unless otherwise indicated, an alkyl group is unsubstituted. In one embodiment, an alkyl group is linear. In another embodiment, an alkyl group is branched.

The term “alkenyl,” as used herein, refers to an aliphatic hydrocarbon group containing at least one carbon-carbon double bond and having one of its hydrogen atoms replaced with a bond. An alkenyl group may be straight or branched and contain from about 2 to about 15 carbon atoms. In one embodiment, an alkenyl group contains from about 2 to about 12 carbon atoms. In another embodiment, an alkenyl group contains from about 2 to about 6 carbon atoms. Non-limiting examples of alkenyl groups include ethenyl, propenyl, n-butenyl, 3-methylbut-2-enyl, n-pentenyl, octenyl and decenyl. An alkenyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. Unless otherwise indicated, an alkenyl group is unsubstituted. The term “C₂-C₆ alkenyl” refers to an alkenyl group having from 2 to 6 carbon atoms.

The term “alkynyl,” as used herein, refers to an aliphatic hydrocarbon group containing at least one carbon-carbon triple bond and having one of its hydrogen atoms replaced with a bond. An alkynyl group may be straight or branched and contain from about 2 to about 15 carbon atoms. In one embodiment, an alkynyl group contains from about 2 to about 12 carbon atoms. In another embodiment, an alkynyl group contains from about 2 to about 6 carbon atoms. Non-limiting examples of alkynyl groups include ethynyl, propynyl, 2-butynyl and 3-methylbutynyl. An alkynyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. Unless otherwise indicated, an alkynyl group is unsubstituted. The term “C₂-C₆ alkynyl” refers to an alkynyl group having from 2 to 6 carbon atoms.

The term “alkylene,” as used herein, refers to an alkyl group, as defined above, wherein one of the alkyl group's hydrogen atoms has been replaced with a bond. An alkylene group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, NH(cycloalkyl), —O—C(O)-alkyl, —O—C(O)-aryl, —O—C(O)-cycloalkyl, —C(O)OH and —C(O)O-alkyl. Unless otherwise indicated, an alkylene group is unsubstituted. Non-limiting examples of alkylene groups include —CH₂—, —CH₂CH₂—, —CH₂CH₂CH₂—, —CH₂CH₂CH₂CH₂—, —CH(CH₃)CH₂CH₂—, —CH(CH₃)— and —CH₂CH(CH₃)CH₂—. In one embodiment, an alkylene group has from 1 to about 6 carbon atoms. In another embodiment, an alkylene group is branched. In another embodiment, an alkylene group is linear. In one embodiment, an alkylene group is —CH₂—. The term “C₁-C₆ alkylene” refers to an alkylene group having from 1 to 6 carbon atoms.

The term “aryl,” as used herein, refers to an aromatic monocyclic or multicyclic ring system comprising from about 6 to about 14 carbon atoms. In one embodiment, an aryl group contains from about 6 to about 10 carbon atoms. An aryl group can be optionally substituted with one or more “ring system substituents” which may be the same or different, and are as defined herein below. Unless otherwise indicated, an aryl group is unsubstituted. In one embodiment, an aryl group can be optionally fused to a cycloalkyl or cycloalkanoyl group. Non-limiting examples of aryl groups include phenyl and naphthyl. In one embodiment, an aryl group is phenyl. In another embodiment, an aryl group is napthalenyl.

The term “cycloalkyl,” as used herein, refers to a non-aromatic mono- or multicyclic ring system comprising from about 3 to about 10 ring carbon atoms. In one embodiment, a cycloalkyl contains from about 5 to about 10 ring carbon atoms. In another embodiment, a cycloalkyl contains from about 3 to about 7 ring atoms. In another embodiment, a cycloalkyl contains from about 5 to about 6 ring atoms. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbomyl and adamantyl. A cycloalkyl group can be optionally substituted with one or more “ring system substituents” which may be the same or different, and are as defined herein below. Unless otherwise indicated, a cycloalkyl group is unsubstituted. In one embodiment, a cycloalkyl group is unsubstituted. The term “3 to 7-membered cycloalkyl” refers to a cycloalkyl group having from 3 to 7 ring carbon atoms. A ring carbon atom of a cycloalkyl group may be functionalized as a carbonyl group. An illustrative example of such a cycloalkyl group (also referred to herein as a “cycloalkanoyl” group) includes, but is not limited to, cyclobutanoyl:

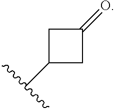 [SMILES: *C1CC(=O)C1]

The term “cycloalkenyl,” as used herein, refers to a non-aromatic mono- or multicyclic ring system comprising from about 4 to about 10 ring carbon atoms and containing at least one endocyclic double bond. In one embodiment, a cycloalkenyl contains from about 4 to about 7 ring carbon atoms. In another embodiment, a cycloalkenyl contains 5 or 6 ring atoms. Non-limiting examples of monocyclic cycloalkenyls include cyclopentenyl, cyclohexenyl, cyclohepta-1,3-dienyl, and the like. A cycloalkenyl group can be optionally substituted with one or more “ring system substituents” which may be the same or different, and are as defined herein below. A ring carbon atom of a cycloalkyl group may be functionalized as a carbonyl group. Unless otherwise indicated, a cycloalkyl group is unsubstituted. In one embodiment, a cycloalkenyl group is cyclopentenyl. In another embodiment, a cycloalkenyl group is cyclohexenyl. The term “4 to 6-membered cycloalkenyl” refers to a cycloalkenyl group having from 4 to 6 ring carbon atoms.

The term “halo,” as used herein, means —F, —Cl, —Br or —I.

The term “haloalkyl,” as used herein, refers to an alkyl group as defined above, wherein one or more of the alkyl group's hydrogen atoms has been replaced with a halogen. In one embodiment, a haloalkyl group has from 1 to 6 carbon atoms. In another embodiment, a haloalkyl group is substituted with from 1 to 3 F atoms. Non-limiting examples of haloalkyl groups include —CH₂F, —CHF₂, —CF₃, —CH₂Cl and —CCl₃. The term “C₁-C₆ haloalkyl” refers to a haloalkyl group having from 1 to 6 carbon atoms. The term “C₁-C₈ haloalkyl” refers to a haloalkyl group having from 1 to 8 carbon atoms.

The term “aminoalkyl,” as used herein, refers to an alkyl group as defined above, wherein one or more of the alkyl group's hydrogen atoms has been replaced with an —NH₂ group. In one embodiment, an aminoalkyl group has from 1 to 6 carbon atoms (C₁-C₆ aminoalkyl). In another embodiment, an aminoalkyl group has from 1 to 8 carbon atoms (C₁-C₈ aminoalkyl). Non-limiting examples of aminoalkyl groups include —CH₂NH₂, —CH₂CH₂ NH₂, —CH₂CH₂CH₂ NH₂ and —CH₂CH(NH₂)CH₃.

The term “hydroxyalkyl,” as used herein, refers to an alkyl group as defined above, wherein one or more of the alkyl group's hydrogen atoms has been replaced with an —OH group. In one embodiment, a hydroxyalkyl group has from 1 to 6 carbon atoms (C₁-C₆ hydroxyalkyl). In another embodiment, a hydroxyalkyl group has from 1 to 8 carbon atoms (C₁-C₈ hydroxyalkyl). Non-limiting examples of hydroxyalkyl groups include —CH₂OH, —CH₂CH₂OH, —CH₂CH₂CH₂OH and —CH₂CH(OH)CH₃.

The term “heteroaryl,” as used herein, refers to an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. In another embodiment, a heteroaryl group is bicyclic and had 9 or 10 ring atoms. A heteroaryl group can be optionally substituted by one or more “ring system substituents” which may be the same or different, and are as defined herein below. Unless otherwise indicated, a heteroaryl group is unsubstituted. A heteroaryl group is joined via a ring carbon atom, and any nitrogen atom of a heteroaryl can be optionally oxidized to the corresponding N-oxide. The term “heteroaryl” also encompasses a heteroaryl group, as defined above, which is fused to a benzene ring. Non-limiting examples of heteroaryls include pyridyl, pyrazinyl, furanyl, thienyl, pyrimidinyl, pyridone (including N-substituted pyridones), isoxazolyl, isothiazolyl, oxazolyl, oxadiazolyl, thiazolyl, pyrazolyl, furazanyl, pyrrolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, benzimidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like, and all isomeric forms thereof. The term “heteroaryl” also refers to partially saturated heteroaryl moieties such as, for example, tetrahydroisoquinolyl, tetrahydroquinolyl and the like. In one embodiment, a heteroaryl group is a 5-membered heteroaryl. In another embodiment, a heteroaryl group is a 6-membered heteroaryl. In another embodiment, a “9- or 10-membered bicyclic heteroaryl” group comprises a 5- to 6-membered heterocycloalkyl group fused to a benzene ring, such as:

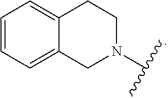 [SMILES: *N1CCc2ccccc2C1]

In still another embodiment, a “9- or 10-membered bicyclic heteroaryl” group comprises a 5- to 6-membered heteroaryl group fused to a cycloalkyl ring or a heterocycloalkyl ring, such as:

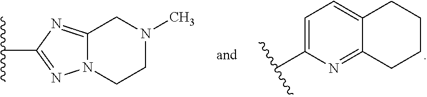 [SMILES: *c1nc2n(n1)CCN(C)C2 ; *c1ccc2c(n1)CCCC2]

The term “heteroarylene,” as used herein, refers to a bivalent group derived from an heteroaryl group, as defined above, by removal of a hydrogen atom from a ring carbon or ring heteroatom of a heteroaryl group. A heteroarylene group can be derived from a monocyclic or multicyclic ring system comprising about 5 to about 14 ring atoms, wherein from 1 to 4 of the ring atoms are each independently O, N or S and the remaining ring atoms are carbon atoms. A heteroarylene group can be optionally substituted by one or more “ring system substituents” which may be the same or different, and are as defined herein below. Unless otherwise indicated, a heteroarylene group is unsubstituted. A heteroarylene group is joined via a ring carbon atom or by a nitrogen atom with an open valence, and any nitrogen atom of a heteroarylene can be optionally oxidized to the corresponding N-oxide. The term “heteroarylene” also encompasses a heteroarylene group, as defined above, which is fused to a benzene ring. Non-limiting examples of heteroarylenes include pyridylene, pyrazinylene, furanylene, thienylene, pyrimidinylene, pyridonylene (including those derived from N-substituted pyridonyls), isoxazolylene, isothiazolylene, oxazolylene, oxadiazolylene, thiazolylene, pyrazolylene, thiophenylene, furazanylene, pyrrolylene, triazolylene, 1,2,4-thiadiazolylene, pyrazinylene, pyridazinylene, quinoxalinylene, phthalazinylene, oxindolylene, imidazo[1,2-a]pyridinylene, imidazo[2,1-b]thiazolylene, benzofurazanylene, indolylene, azaindolylene, benzimidazolylene, benzothienylene, quinolinylene, imidazolylene, benzimidazolylene, thienopyridylene, quinazolinylene, thienopyrimidylene, pyrrolopyridylene, imidazopyridylene, isoquinolinylene, benzoazaindolylene, 1,2,4-triazinylene, benzothiazolylene and the like, and all isomeric forms thereof. The term “heteroarylene” also refers to partially saturated heteroarylene moieties such as, for example, tetrahydroisoquinolylene, tetrahydroquinolylene, and the like. A heteroarylene group is divalent and unless specified otherwise, either available bond on a heteroarylene ring can connect to either group flanking the heteroarylene group. For example, the group “A-heteroarylene-B,” wherein the heteroarylene group is:

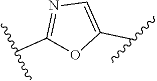 [SMILES: *c1cnc(*)o1]

is understood to represent both:

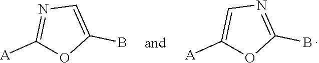

In one embodiment, a heteroarylene group is a monocyclic heteroarylene group or a bicyclic heteroarylene group. In another embodiment, a heteroarylene group is a monocyclic heteroarylene group. In another embodiment, a heteroarylene group is a bicyclic heteroarylene group. In still another embodiment, a heteroarylene group has from about 5 to about 10 ring atoms. In another embodiment, a heteroarylene group is monocyclic and has 5 or 6 ring atoms. In another embodiment, a heteroarylene group is bicyclic and has 9 or 10 ring atoms. In another embodiment, a heteroarylene group is a 5-membered monocyclic heteroarylene. In another embodiment, a heteroarylene group is a 6-membered monocyclic heteroarylene. In another embodiment, a bicyclic heteroarylene group comprises a 5- or 6-membered monocyclic heteroarylene group fused to a benzene ring. In still another embodiment, a heteroaryl group comprises a 5- to 6-membered monocyclic heteroarylene group fused to a cycloalkyl ring or a heterocycloalkyl ring.

The term “heterocycloalkyl,” as used herein, refers to a non-aromatic saturated monocyclic or multicyclic ring system comprising 3 to about 11 ring atoms, wherein from 1 to 4 of the ring atoms are independently O, S, N or Si, and the remainder of the ring atoms are carbon atoms. A heterocycloalkyl group can be joined via a ring carbon, ring silicon atom or ring nitrogen atom. In one embodiment, a heterocycloalkyl group is monocyclic and has from about 3 to about 7 ring atoms. In another embodiment, a heterocycloalkyl group is monocyclic has from about 4 to about 7 ring atoms. In another embodiment, a heterocycloalkyl group is bicyclic and has from about 7 to about 11 ring atoms. In still another embodiment, a heterocycloalkyl group is monocyclic and has 5 or 6 ring atoms. In one embodiment, a heterocycloalkyl group is monocyclic. In another embodiment, a heterocycloalkyl group is bicyclic. There are no adjacent oxygen and/or sulfur atoms present in the ring system. Any —NH group in a heterocycloalkyl ring may exist protected such as, for example, as an —N(BOC), —N(CBz), —N(Tos) group and the like; such protected heterocycloalkyl groups are considered part of this invention. A heterocycloalkyl group can be optionally substituted by one or more “ring system substituents” which may be the same or different, and are as defined herein below. The nitrogen or sulfur atom of the heterocycloalkyl can be optionally oxidized to the corresponding N-oxide, S-oxide or S,S-dioxide. Unless otherwise indicated, a heterocycloalkyl group is unsubstituted. Non-limiting examples of monocyclic heterocycloalkyl rings include oxetanyl, piperidyl, pyrrolidinyl, piperazinyl, morpholinyl, thiomorpholinyl, thiazolidinyl, 1,4-dioxanyl, tetrahydrofuranyl, tetrahydrothiophenyl, delta-lactam, delta-lactone, silacyclopentane, silapyrrolidine and the like, and all isomers thereof. Non-limiting illustrative examples of a silyl-containing heterocycloalkyl group include:

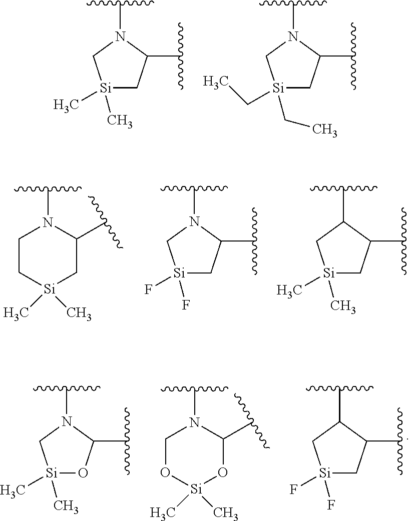

A ring carbon atom of a heterocycloalkyl group may be functionalized as a carbonyl group. Illustrative examples of such a heterocycloalkyl group include, but are not limited to:

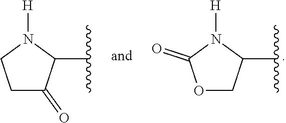 [SMILES: *C1NCCC1=O ; *C1COC(=O)N1]

A ring sulfur atom of a heterocycloalkyl group may also be functionalized as a sulfonyl group. An example of such a heterocycloalkyl group is:

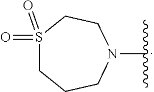 [SMILES: *N1CCCS(=O)(=O)CC1]

A bicyclic heterocycloalkyl group may be in the form of a fused ring system or a spirocyclic system. Examples of fused bicyclic heterocycloalkyl groups include, but are not limited to:

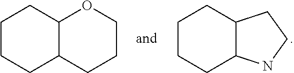 [SMILES: C1CCC2OCCCC2C1 ; C1CCC2NCCC2C1]

Examples of spirocyclic bicyclic heterocycloalkyl groups include, but are not limited to:

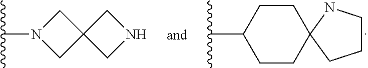 [SMILES: *N1CC2(CNC2)C1 ; "and" ; *C1CCC2(CCCN2)CC1 ; "."]

In one embodiment, a heterocycloalkyl group is a 5-membered monocyclic heterocycloalkyl. In another embodiment, a heterocycloalkyl group is a 6-membered monocyclic heterocycloalkyl. The term “5- to 7-membered monocyclic cycloalkyl” refers to a monocyclic heterocycloalkyl group having from 5 to 7 ring atoms. The term “4 to 6-membered monocyclic cycloalkyl” refers to a monocyclic heterocycloalkyl group having from 4 to 6 ring atoms. The term “9 to 10-membered bicyclic heterocycloalkyl” refers to a bicyclic heterocycloalkyl group having from 9 to 10 ring atoms.

The term “heterocycloalkenyl,” as used herein, refers to a heterocycloalkyl group, as defined above, wherein the heterocycloalkyl group contains from 4 to 10 ring atoms, and at least one endocyclic carbon-carbon or carbon-nitrogen double bond. A heterocycloalkenyl group can be joined via a ring carbon or ring nitrogen atom. In one embodiment, a heterocycloalkenyl group has from 4 to 6 ring atoms. In another embodiment, a heterocycloalkenyl group is monocyclic and has 5 or 6 ring atoms. In another embodiment, a heterocycloalkenyl group is bicyclic. A heterocycloalkenyl group can optionally substituted by one or more ring system substituents, wherein “ring system substituent” is as defined above. The nitrogen or sulfur atom of the heterocycloalkenyl can be optionally oxidized to the corresponding N-oxide, S-oxide or S,S-dioxide. A ring carbon atom of a heterocycloalkenyl group may be functionalized as a carbonyl group. Unless otherwise indicated, a heterocycloalkenyl group is unsubstituted. Non-limiting examples of heterocycloalkenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluoro-substituted dihydrofuranyl, 7-oxabicyclo[2.2.1]heptenyl, dihydrothiophenyl, dihydrothiopyranyl, and the like and the like. In one embodiment, a heterocycloalkenyl group is a 5-membered heterocycloalkenyl. In another embodiment, a heterocycloalkenyl group is a 6-membered heterocycloalkenyl. The term “4 to 6-membered heterocycloalkenyl” refers to a heterocycloalkenyl group having from 4 to 6 ring atoms.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “in substantially purified form,” as used herein, refers to the physical state of a compound after the compound is isolated from a synthetic process (e.g., from a reaction mixture), a natural source, or a combination thereof. The term “in substantially purified form,” also refers to the physical state of a compound after the compound is obtained from a purification process or processes described herein or well-known to the skilled artisan (e.g., chromatography, recrystallization and the like), in sufficient purity to be characterizable by standard analytical techniques described herein or well-known to the skilled artisan.

It should also be noted that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

Examples of “ring system substituents” include, but are not limited to, alkyl, alkenyl, alkynyl, aryl, heteroaryl, -alkylene-aryl, -arylene-alkyl, -alkylene-heteroaryl,-alkenylene-heteroaryl, -alkynylene-heteroaryl, —OH, hydroxyalkyl, haloalkyl, —O-alkyl, —O-haloalkyl, -alkylene-O-alkyl, —O-aryl, —O-alkylene-aryl, acyl, —C(O)-aryl, halo, —NO₂, —CN, —SF₅, —C(O)OH, —C(O)O-alkyl, —C(O)O-aryl, —C(O)O-alkylene-aryl, —S(O)-alkyl, —S(O)₂-alkyl, —S(O)-aryl, —S(O)₂-aryl, —S(O)-heteroaryl, —S(O)z-heteroaryl, —S-alkyl, —S-aryl, —S-heteroaryl, —S-alkylene-aryl, —S-alkyleneheteroaryl, —S(O)₂-alkylene-aryl, —S(O)₂-alkylene-heteroaryl, —Si(alkyl)₂, —Si(aryl)₂, Si(heteroaryl)₂—Si(alkyl)(aryl), —Si(alkyl)(cycloalkyl), —Si(alkyl)(heteroaryl), cycloalkyl, heterocycloalkyl, —O—C(O)-alkyl, —O—C(O)-aryl, —O—C(O)-cycloalkyl, —C(═N—CN)—NH₂, —C(═NH)—NH₂, —C(═NH)—NH(alkyl), —N(Y¹)(Y²), -alkylene-N(Y¹)(Y²), —C(O)N(Y¹)(Y²), and —S(O)₂N(Y¹)(Y²), wherein Y¹ and Y² can be the same or different and are independently selected from the group consisting of hydrogen, alkyl, aryl, cycloalkyl, and -alkylene-aryl. “Ring system substituent” may also mean a single moiety which simultaneously replaces two available hydrogens on two adjacent carbon atoms (one H on each carbon) on a ring system. Examples of such moiety are methylenedioxy, ethylenedioxy, —C(CH₃)₂— and the like which form moieties such as, for example:

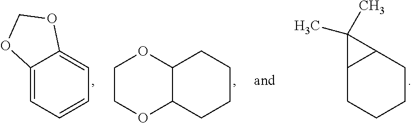

When any substituent or variable (e.g., R¹, m, etc.) occurs more than one time in any constituent or in Formula (I), its definition on each occurrence is independent of its definition at every other occurrence, unless otherwise indicated.

As used herein, the term “composition” is intended to encompass a product comprising the specified ingredients in the specified amounts, as well as any product which results from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to provide a Pyrazolo[4,3-d]Pyrimidine Derivative or a pharmaceutically acceptable salt or solvate of the compound. The transformation may occur by various mechanisms (e.g., by metabolic or chemical processes), such as, for example, through hydrolysis in blood.

For example, if a Pyrazolo[4,3-d]Pyrimidine Derivative contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkyl, α-amino(C₁-C₄)alkylene-aryl, arylacyl and α-aminoacyl, or α-aminoacyl-α-aminoacyl, where each α-aminoacyl group is independently selected from the naturally occurring L-amino acids, —P(O)(OH)₂, —P(O)(O(C₁-C₆)alkyl)₂ or glycosyl (the radical resulting from the removal of a hydroxyl group of the hemiacetal form of a carbohydrate), and the like.

Similarly, if a Pyrazolo[4,3-d]Pyrimidine Derivative contains an amine functional group, a prodrug can be formed by the replacement of a hydrogen atom in the amine group with a group such as, for example, R-carbonyl-, RO-carbonyl-, NRR′-carbonyl- wherein R and R′ are each independently (C₁-C₁₀)alkyl, (C₃-C₇) cycloalkyl, benzyl, a natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl; carboxy (C₁-C₆)alkyl; amino(C₁-C₄)alkyl or mono-N— or di-N,N—(C₁-C₆)alkylaminoalkyl; —C(Y⁴)Y⁵ wherein Y⁴ is H or methyl and Y⁵ is mono-N— or di-N,N—(C₁-C₆)alkylamino morpholino; piperidin-1-yl or pyrrolidin-1-yl, and the like.

Pharmaceutically acceptable esters of the present compounds include the following groups: (1) carboxylic acid esters obtained by esterification of the hydroxy group of a hydroxyl compound, in which the non-carbonyl moiety of the carboxylic acid portion of the ester grouping is selected from straight or branched chain alkyl (e.g., methyl, ethyl, n-propyl, isopropyl, t-butyl, sec-butyl or n-butyl), alkoxyalkyl (e.g., methoxymethyl), aralkyl (e.g., benzyl), aryloxyalkyl (for example, phenoxymethyl), aryl (e.g., phenyl optionally substituted with, for example, halogen, C₁₋₄alkyl, —O—(C₁₋₄alkyl) or amino); (2) sulfonate esters, such as alkyl- or aralkylsulfonyl (for example, methanesulfonyl); (3) amino acid esters (e.g., L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. A “hydrate” is a solvate wherein the solvent molecule is water.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvates, hydrates and the like are described by E. C. van Tonder et al, AAPS PharmSciTechours., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than room temperature, and cooling the solution at a rate sufficient to form crystals which are then isolated by standard methods. Analytical techniques such as, for example IR spectroscopy, show the presence of the solvent (or water) in the crystals as a solvate (or hydrate).

The Pyrazolo[4,3-d]Pyrimidine Derivatives can form salts which are also within the scope of this invention. The term “salt(s)”, as employed herein, denotes acidic salts formed with inorganic and/or organic acids, as well as basic salts formed with inorganic and/or organic bases. In addition, when a Pyrazolo[4,3-d]Pyrimidine Derivative contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the Compounds of Formula (I) may be formed, for example, by reacting a Pyrazolo[4,3-d]Pyrimidine Derivative with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, formates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates), trifluroacetates, and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Diastereomeric mixtures can be separated into their individual diastereomers on the basis of their physical chemical differences by methods well-known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Sterochemically pure compounds may also be prepared by using chiral starting materials or by employing salt resolution techniques. Also, some of the Pyrazolo[4,3-d]Pyrimidine Derivatives may be atropisomers (e.g., substituted biaryls), and are considered as part of this invention. Enantiomers can also be directly separated using chiral chromatographic techniques.

It is also possible that the Pyrazolo[4,3-d]Pyrimidine Derivatives may exist in different tautomeric forms, and all such forms are embraced within the scope of the invention. For example, all keto-enol and imine-enamine forms of the compounds are included in the invention.

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. If a Pyrazolo[4,3-d]Pyrimidine Derivative incorporates a double bond or a fused ring, both the cis- and trans-forms, as well as mixtures, are embraced within the scope of the invention.

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to apply equally to the salt, solvate, ester and prodrug of enantiomers, stereoisomers, rotamers, tautomers, positional isomers, racemates or prodrugs of the inventive compounds.

In the Compounds of Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (¹H), and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched Compounds of Formula (I) can be prepared without undue experimentation by conventional techniques well known to those skilled in the art or by processes analogous to those described in the Schemes and Examples herein using appropriate isotopically-enriched reagents and/or intermediates. In one embodiment, a Compound of Formula (I) has one or more of its hydrogen atoms replaced with deuterium.

Polymorphic forms of the Pyrazolo[4,3-d]Pyrimidine Derivatives, and of the salts, solvates, hydrates, esters and prodrugs of the Pyrazolo[4,3-d]Pyrimidine Derivatives, are intended to be included in the present invention.

The following abbreviations are used below and have the following meanings: Ac is acyl; AmPhos Pd G₃ is palladium G3-(4-(N,N-dimethylamino)phenyl)di-tert-butylphosphine, [4-(di-tert-butylphosphino)-N,N-dimethylaniline-2-(2′-aminobiphenyl)]palladium(II) methanesulfonate; BH₃·DMS is borane dimethylsulfide complex; Boc or boc is tert-butyloxycarbonyl; BOC-DL-ALA-OH is 2-(Boc-amino)propionic acid; Celite is diatomaceous earth; CMBP is cyanomethylene tributylphosphorane; DABCO is diazabicyclo[2.2.2]octane; DBU is 1,8-diazabicyclo[5.4.0]undec-7-ene; DCE is dichloroethane; DCM is dichloromethane; Dess-Martin Periodinane is 3-Oxo-1λ⁵,2-benziodoxole-1,1,1(3H)-triyltriacetate; DIEA is diisopropylethylamine; DME is dimethoxyethane; DMF is N,N-dimethylformamide; DMSO is dimethylsulfoxide; Et is ethyl, Et₃N is triethylamine; EtOAc is ethyl acetate; EtOH is ethanol; HATU is (1-[bis(dimethylamino)methylene]-1H-1,2,3-triazolo[4,5-b]pyridinium 3-oxide hexafluorophosphate; HPLC is high performance liquid chromatography; (Ir[dF(CF₃)ppy]₂(dtbpy))PF₆ is [4,4′-Bis(1,1-dimethylethyl)-2,2′-bipyridine-N1,N1′]bis[3,5-difluoro-2-[5-(trifluoromethyl)-2-pyridinyl-N]phenyl-C]Iridium(III) hexafluorophosphate; LCMS is liquid chromatography/mass spectrometry; LED is light-emitting diode; LHMDS is lithium hexamethyldisilazane; mCPBA is meta-chloroperoxybenzoic acid; Me is methyl; MeOH is methanol; MS is mass spectrometry; NBS is N-bromosuccinimide; NCS is N-chlorosuccinimide; Pd(Ph₃P)₄ is tetrakis triphenylphosphine palladium(0); PyBOP is (benzotriazol-1-yloxy) tripyrrolidinophosphonium hexafluorophosphate; RP-flash is reverse-phase flash column chromatography; TFA is trifluoroacetic acid; THF is tetrahydrofuran; TLC is thin-layer chromatography; and TMSCl is trimethylsilyl chloride.

### The Compounds of Formula (I)

The present invention provides Pyrazolo[4,3-d]Pyrimidine Derivatives of Formula (I):

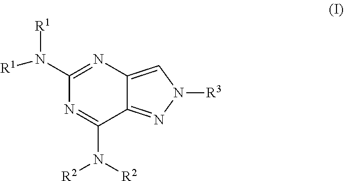

and pharmaceutically acceptable salts thereof, wherein R¹, R², and R³ are defined above for the Compounds of Formula (I).

In one embodiment, the present invention provides a compound of formula (I), having the formula (Ia):

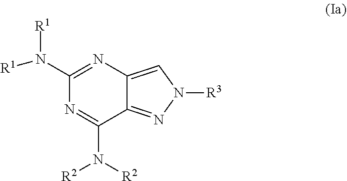

or a pharmaceutically acceptable salt thereof,
wherein:

each occurrence of R¹ is independently selected from H and C₁-C₆ alkyl;

each occurrence of R² is independently selected from H, C₁-C₈ alkyl, —(C₁-C₆ alkylene)-O—(C₁-C₆ alkyl), C₁-C₈ hydroxyalkyl, C₃-C₇ cycloalkyl, wherein said C₃-C₇ cycloalkyl group can be optionally substituted with one or more R⁴ groups, which can be the same or different; or two R² groups, together with the nitrogen atom to which they are attached, can join to form a 5- to 7-membered monocyclic heterocycloalkyl group, wherein said 5- to 7-membered monocyclic heterocycloalkyl group can be optionally substituted with one or more R⁴ groups, which can be the same or different;

R³ is selected from C₁-C₈ alkyl, C₁-C₈ aminoalkyl, benzyl, —(CH₂)₂-phenyl, —CH₂—(C₃-C₇ cycloalkyl), and —CH₂-(5- or 6-membered monocyclic heteroaryl), wherein the phenyl moiety of said benzyl group can be optionally substituted with one or more R⁵ groups, which can be the same or different; the phenyl moiety of said —(CH₂)₂-phenyl group can be optionally substituted with one or more R⁶ groups, which can be the same or different; the C₃-C₇ cycloalkyl moiety of said —CH₂—(C₃-C₇ cycloalkyl) group can be optionally substituted with one or more R⁷ groups, which can be the same or different; and the 5- or 6-membered monocyclic heteroaryl moiety of said —CH₂-(5- or 6-membered monocyclic heteroaryl) group can be optionally substituted with one or more R⁸ groups, which can be the same or different;

each occurrence of R⁴ is independently selected from C₁-C₈ alkyl, C₁-C₈ hydroxyalkyl, halo, and —OH;

each occurrence of R⁵ is independently selected from C₁-C₆ alkyl, —O—(C₁-C₆ alkyl), halo, —NH₂, C₁-C₆ aminoalkyl, —(CH₂)ₙ-(5- to 7-membered monocyclic heterocycloalkyl), R^{A}, and R^{B};

each occurrence of R⁶ is independently selected from C₁-C₆ alkyl, —O—(C₁-C₆ alkyl), halo, —NH₂, and C₁-C₆ aminoalkyl;

each occurrence of R⁷ is independently selected from C₁-C₆ alkyl, —O—(C₁-C₆ alkyl), halo, —NH₂, and C₁-C₆ aminoalkyl;

each occurrence of R⁸ is independently selected from C₁-C₆ alkyl, —O—(C₁-C₆ alkyl), halo, —NH₂, and C₁-C₆ aminoalkyl;

R^{A} is:

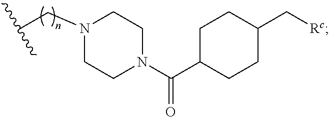

R^{B} is:

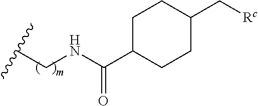

each R^{C} is independently selected from:

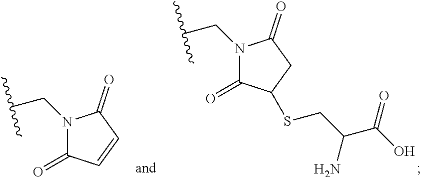 [SMILES: *CN1C(=O)C=CC1=O ; *CN1C(=O)CC(SCC(N)C(=O)O)C1=O]

each occurrence of m is independently 1 or 2; and

each occurrence of n is independently 0 or 1.

In one embodiment, for the compounds of formula (I) or (Ia), each occurrence of R¹ is H.

In one embodiment, for the compounds of formula (I) or (Ia), one occurrence of R² is H, and the other occurrence of R² is other than H.

In another embodiment, for the compounds of formula (I) or (Ia), one occurrence of R² is H, and the other occurrence of R² is selected from methyl, ethyl, isopropyl, isobutyl, n-butyl, n-pentyl, cyclopentyl, cyclohexyl, —(CH₂)₃—CH(CH₃)₂, —(CH₂)₂—CH(CH₃)₂, —(CH₂)₂OH, —(CH₂)₃OH, —(CH₂)₂OCH₃, —CH₂CH(OH)CH₃, —(CH₂)₂CH(OH)CH₃, —CH(CH₂CH₂CH₃)CH₂OH, —CH(CH₂CH₂CH₂CH₃)(CH₂CH₂OH), CH(CH₃)CH₂CH₂CH₃, —CH₂CH(CH₃)CH₂CH₃, and —CH(CH₂CH₂CH₃)CH₂CH₂CH₂CH₃, wherein said cyclopentyl group, and said cyclohexyl group, can be optionally substituted with one group selected from —OH and —CH₂OH; or two R² groups, together with the nitrogen atom to which they are attached, can join to form a pyrrolidinyl group.

In one embodiment, for the compounds of formula (I) and (Ia), R³ is benzyl, which can be optionally substituted with one or more R⁵ groups, which can be the same or different.

In another embodiment, for the compounds of formula (I) and (Ia), R³ is benzyl, which can be optionally substituted with up to three groups, which can be the same or different, and are selected from methyl, methoxy, ethoxy, isopropoxy, Cl, F, —NH₂, —CH₂NH₂, —CH₂CH₂NH₂, pyrrolidinyl, —CH₂-pyrroldinyl,

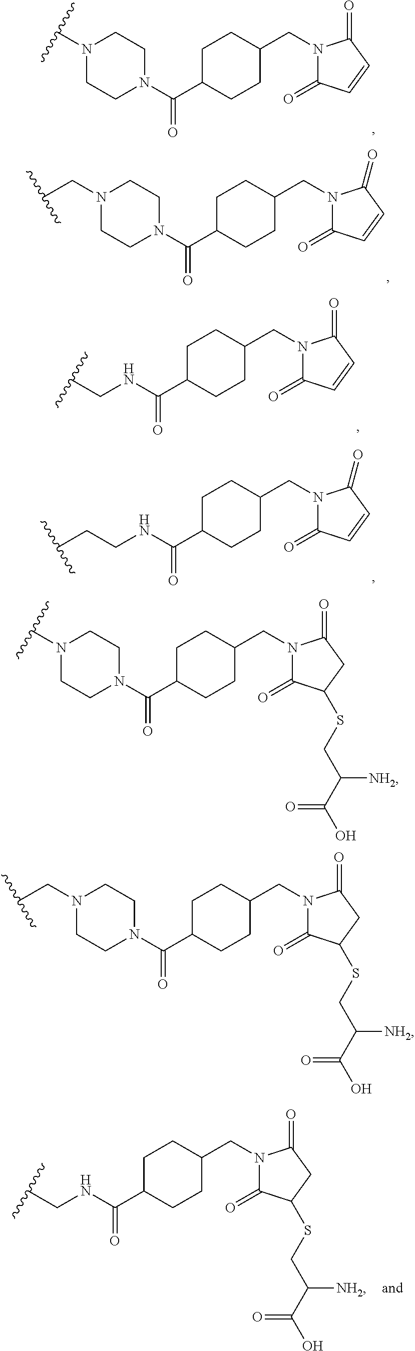

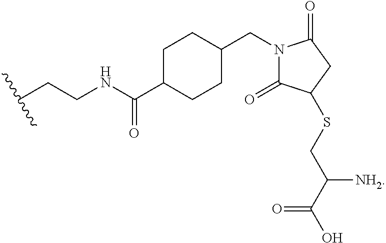 [SMILES: *CCNC(=O)C1CCC(CN2C(=O)CC(SCC(N)C(=O)O)C2=O)CC1]

In another embodiment, for the compounds of formula (I), (Ia), and (Ib), R³ selected from C₁-C₈ alkyl, C₁-C₈ aminoalkyl, —(CH₂)₂-phenyl, —CH₂—(C₃-C₇ cycloalkyl), and —CH₂-(5- or 6-membered monocyclic heteroaryl), wherein the phenyl moiety of said —(CH₂)₂-phenyl group, and the 5- or 6-membered monocyclic heteroaryl moiety of said —CH₂-(5- or 6-membered monocyclic heteroaryl) group can each be optionally substituted with an —O—(C₁-C₆ alkyl) group; and the C₃-C₇ cycloalkyl moiety of said —CH₂—(C₃-C₇ cycloalkyl) group can be optionally substituted with a C₁-C₆ aminoalkyl group.

In still another embodiment, for the compounds of formula (I) and (Ia), R³ is selected from —(CH₂)₄NH₂, —(CH₂)₈NH₂,

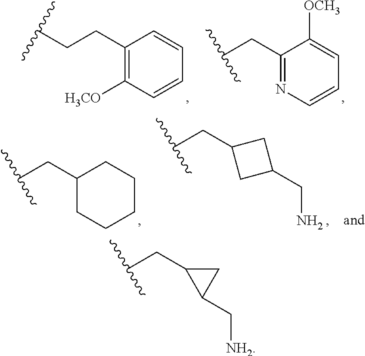

In another embodiment, the present invention provides a compound of formula (I), having the formula (Ib):

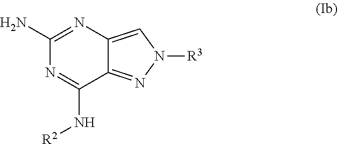

or a pharmaceutically acceptable salt thereof,
wherein:

R² is C₁-C₆ alkyl; and

R³ is selected from benzyl, and —CH₂-(5- or 6-membered monocyclic heteroaryl), wherein the phenyl moiety of said benzyl group and the 5- or 6-membered monocyclic heteroaryl moiety of said —CH₂-(5- or 6-membered monocyclic heteroaryl) group can be optionally substituted with one or more groups, which can be the same or different and are each independently selected from —O—(C₁-C₆ alkyl), halo, —NH₂, C₁-C₆ aminoalkyl, and 5- to 7-membered monocyclic heterocycloalkyl).

In one embodiment, for the compounds of formula (I), (Ia) and (Ib), R² is selected from methyl, ethyl, isopropyl, isobutyl, n-butyl, n-pentyl, cyclopentyl, cyclohexyl, —(CH₂)₃—CH(CH₃)₂, —(CH₂)₂OH, —(CH₂)₃OH, —(CH₂)₂OCH₃, —CH₂CH(OH)CH₃, —(CH₂)₂CH(OH)CH₃, —CH(CH₂CH₂CH₃)CH₂OH, —CH(CH₂CH₂CH₃)(CH₂)₂OH, —CH(CH₂CH₂CH₂CH₃)(CH₂)₃OH, —CH(CH₃)CH₂CH₂CH₃, —CH₂CH(CH₃)CH₂CH₃, and —CH(CH₂CH₂CH₃)CH₂CH₂CH₂CH₃, wherein said cyclopentyl group, and said cyclohexyl group, can be optionally substituted with one group selected from —OH and —CH₂OH.

In one embodiment, for the compound of formula (I), (Ia), and (Ib), R³ is selected from:

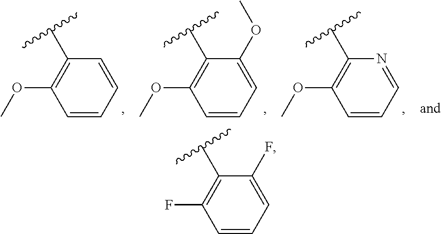

each of which groups can be optionally substituted with one group selected from —NH₂, —CH₂NH₂,

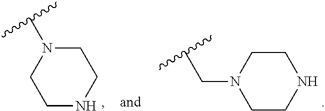 [SMILES: *N1CCNCC1 ; *CN1CCNCC1]

In another embodiment, the present invention provides a compound of formula (I), having the formula (Ic):

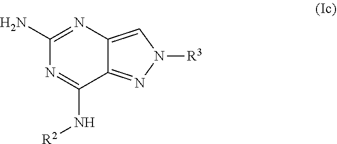

or a pharmaceutically acceptable salt thereof,
wherein:

R² is n-butyl or —CH(CH₂OH)CH₂CH₂CH₃; and

R³ is selected from:

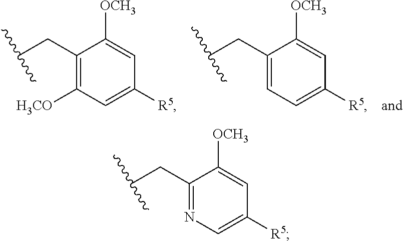

and

R⁵ is selected from —CH₂NH₂, —CH₂NHCH₃, —CH(CH₃)NHCH₃, —CH(—NHCH₃)CH₂CH₂CH₃, —CH(CH₃)NHCH₂CH₂CH₃, —CH(—NHCH₃)CH₂CH₂NHC(O)-pyridyl, piperazinyl, and —SCH₂CH(NH₂)C(O)OH.

In one embodiment, for the compounds of formula (I), (Ia), (Ib), and (Ic), R² is n-butyl.

In another embodiment, for the compounds of formula (Ic), R² is —CH(CH₂OH)CH₂CH₂CH₃.

In one embodiment, for the compounds of formula (Ic), R³ is:

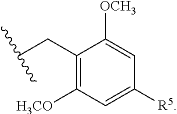

In another embodiment, for the compounds of formula (Ic), R³ is:

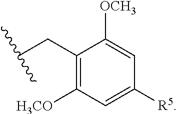

wherein R⁵ is selected from: —CH₂NH₂, —CH₂NHCH₃, —CH(CH₃)NHCH₃, —CH(—NHCH₃)CH₂CH₂CH₃, —CH(—NHCH₃)CH₂CH₂NHC(O)-pyridyl, and —SCH₂CH(NH₂)C(O)OH.

In one embodiment, for the compounds of formula (Ic), R³ is:

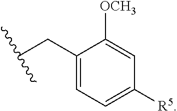

In another embodiment, for the compounds of formula (Ic), R³ is:

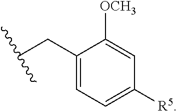

wherein R⁵ is selected from: —CH(CH₃)NHCH₂CH₂CH₃, and piperizinyl.

In one embodiment, for the compounds of formula (Ic), R³ is:

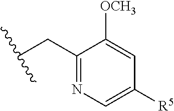

In another embodiment, for the compounds of formula (Ic), R³ is:

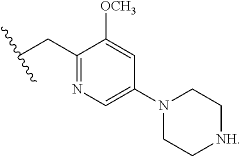 [SMILES: *Cc1ncc(N2CCNCC2)cc1OC]

In one embodiment, the compound of formula (I), (Ia), (Ib), or (Ic) is a pharmaceutically acceptable salt, which is a trifluoroacetate salt.

In another embodiment, the compound of formula (I), (Ia), (Ib), or (Ic) is a pharmaceutically acceptable salt, which is a formate salt.

In one embodiment, the compound of formula (I), (Ia), (Ib), or (Ic) is in substantially purified form.

Other embodiments of the present invention include the following:

(a) A pharmaceutical composition comprising an effective amount of a Pyrazolo[4,3-d]Pyrimidine Derivative, and a pharmaceutically acceptable carrier.

(b) The pharmaceutical composition of (a), further comprising a second therapeutic agent selected from the group consisting of anticancer agents.

(c) The pharmaceutical composition of (b), wherein the anticancer agent is an anti-human PD-1 antibody (or antigen-binding fragment thereof).

(d) A pharmaceutical combination that comprises: (i) a Pyrazolo[4,3-d]Pyrimidine Derivative, and (ii) a second therapeutic agent selected from the group consisting of anticancer agents, wherein the Pyrazolo[4,3-d]Pyrimidine Derivative, and the second therapeutic agent are each employed in an amount that renders the combination effective for inhibiting replication of cancer cells, or for treating cancer and/or reducing the likelihood or severity of symptoms of cancer.

(e) The combination of (d), wherein the second therapeutic agent is an anti-human PD-1 antibody (or antigen-binding fragment thereof).

(f) A method of inhibiting cancer cell replication in a subject in need thereof which comprises administering to the subject an effective amount of a Pyrazolo[4,3-d]Pyrimidine Derivative.

(g) A method of treating cancer and/or reducing the likelihood or severity of symptoms of cancer in a subject in need thereof which comprises administering to the subject an effective amount of a Pyrazolo[4,3-d]Pyrimidine Derivative.

(h) The method of (g), wherein the Pyrazolo[4,3-d]Pyrimidine Derivative is administered in combination with an effective amount of at least one second therapeutic agent selected from the group consisting of anticancer agents.

(i) The method of (h), wherein the second therapeutic agent is an anti-human PD-1 antibody (or antigen-binding fragment thereof).

(j) A method of inhibiting cancer cell replication in a subject in need thereof which comprises administering to the subject the pharmaceutical composition of (a), (b) or (c) or the combination of (d) or (e).

(k) A method of treating cancer and/or reducing the likelihood or severity of symptoms of cancer in a subject in need thereof which comprises administering to the subject the pharmaceutical composition of (a), (b) or (c) or the combination of (d) or (e).

The present invention also includes Pyrazolo[4,3-d]Pyrimidine Derivative for use (i) in, (ii) as a medicament for, or (iii) in the preparation of a medicament for: (a) medicine; (b) inhibiting cancer cell replication, or (c) treating cancer and/or reducing the likelihood or severity of symptoms of cancer. In these uses, the Pyrazolo[4,3-d]Pyrimidine Derivative can optionally be employed in combination with one or more additional therapeutic agents selected from anticancer agents.

It is further to be understood that the embodiments of compositions and methods provided as (a) through (k) above are understood to include all embodiments of the compounds, including such embodiments as result from combinations of embodiments.

Non-limiting examples of the Compounds of Formula (I) include compounds 1-241, as set forth in the Examples below, and pharmaceutically acceptable salts thereof.

### Methods for Making the Compounds of Formula (I)

The Compounds of Formula (I) may be prepared from known or readily prepared starting materials, following methods known to one skilled in the art of organic synthesis. Methods useful for making the Compounds of Formula (I) are set forth in the Examples below Alternative synthetic pathways and analogous structures will be apparent to those skilled in the art of organic synthesis.

One skilled in the art of organic synthesis will recognize that the synthesis of the bicyclic heterocycle cores contained in Compounds of Formula (I) may require protection of certain functional groups (i.e., derivatization for the purpose of chemical compatibility with a particular reaction condition). Suitable protecting groups for the various functional groups of these Compounds and methods for their installation and removal are well known in the art of organic chemistry. A summary of many of these methods can be found in Greene et al., Protective Groups in Organic Synthesis, Wiley-Interscience, New York, (1999).

One skilled in the art of organic synthesis will also recognize that one route for the synthesis of the bicyclic heterocycle cores of the Compounds of Formula (I) may be more desirable depending on the choice of appendage substituents.

Additionally, one skilled in the art will recognize that in some cases the order of reactions may differ from that presented herein to avoid functional group incompatibilities and thus adjust the synthetic route accordingly.

The preparation of multicyclic intermediates useful for making the bicyclic heterocycle cores of the Compounds of Formula (I) have been described in the literature and in compendia such as “Comprehensive Heterocyclic Chemistry” editions I, II and III, published by Elsevier and edited by A. R. Katritzky & R. J K Taylor. Manipulation of the required substitution patterns have also been described in the available chemical literature as summarized in compendia such as “Comprehensive Organic Chemistry” published by Elsevier and edited by D H R. Barton and W. D. Ollis; “Comprehensive Organic Functional Group Transformations” edited by edited by A. R. Katritzky & R. J K Taylor and “Comprehensive Organic Transformation” published by Wily-CVH and edited by R. C. Larock.

The starting materials used, and the intermediates prepared using the methods set forth in the Examples below may be isolated and purified if desired using conventional techniques, including but not limited to filtration, distillation, crystallization, chromatography and alike. Such materials can be characterized using conventional means, including physical constants and spectral data.

One skilled in the art will be aware of standard formulation techniques as set forth in the open literature as well as in textbooks such as Zheng, “Formulation and Analytical Development for Low-dose Oral Drug Products,” Wiley, 2009, ISBN.

### EXAMPLES

### General Methods

Solvents, reagents, and intermediates that are commercially available were used as received. Reagents and intermediates that are not commercially available were prepared in the manner as described below. ¹H NMR spectra are reported as ppm downfield from Me₄Si with number of protons, multiplicities, and coupling constants in Hertz indicated parenthetically. Where LC/MS data are presented, the retention time and observed parent ion are given. Flash column chromatography was performed using pre-packed normal phase silica or bulk silica, and using a gradient elution of hexanes/ethyl acetate, from 100% hexanes to 100% ethyl acetate. Alternatively, a gradient elution of DCM/MeOH from 100% DCM to 10% MeOH can be used for more polar compounds. Some compounds were purified using reverse phase HPLC using a gradient of acetonitrile/water containing either 0.01% trifluoracetic acid, 0.01% formic acid or 0.01% ammonium hydroxide.

### Example 1

### Preparation of Compound 1

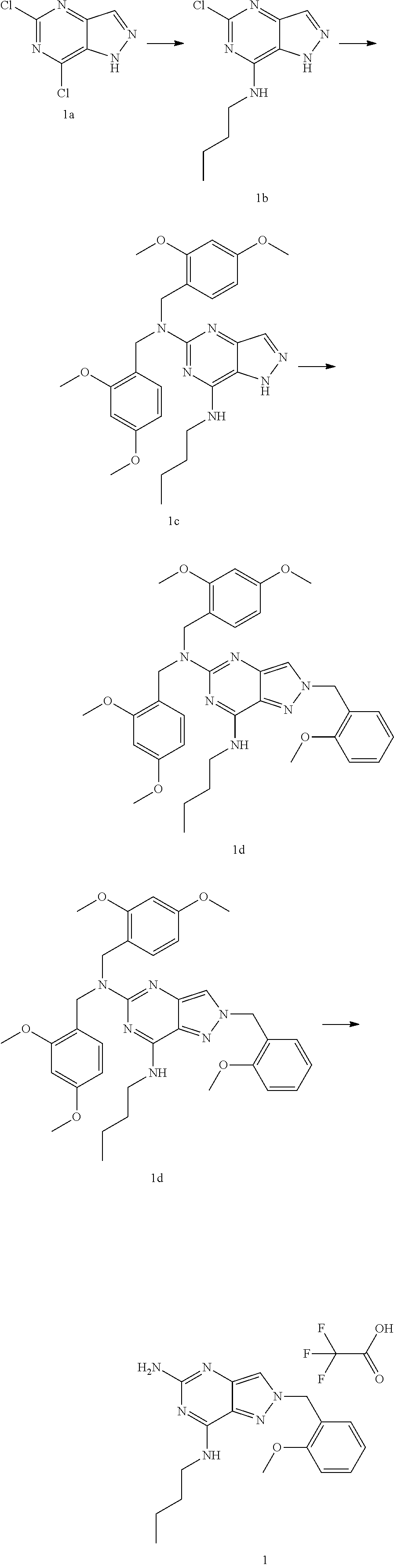

### Step A—Synthesis of Compound 1b

To a stirred solution of 5,7-dichloro-1H-pyrazolo[4,3-d]pyrimidine (6.6 g, 34.9 mmol) in THF (116 mL), at room temperature, was added triethylamine (7.30 ml, 52.4 mmol). The resulting reaction was cooled to 0° C., and butylamine (5.18 ml, 52.4 mmol) was added dropwise. The resulting mixture was warmed to room temperature, and allowed to stir overnight. The reaction mixture was concentrated in vacuo, and the resulting residue was purified using silica gel chromatography (24 gram ISCO Redisep Gold column, eluent 0-100% EtOAc/Hexanes). The purified compound was recrystallized from a mixture of EtOAc (40 mL), MeOH (30 mL), and hexanes (20 mL) to provide compound 1b. MS: m/z=226.0 [M+H].

### Step B—Synthesis of Compound 1c

To a solution of compound 1b (2 g, 8.86 mmol) in ethanol (15 mL), at room temperature, was added bis(2,4-dimethoxybenzyl)amine (4.22 g, 13.3 mmol). The resulting reaction was sealed, and allowed to stir in a microwave reactor at 120° C. for 16 hours. The reaction mixture was then concentrated in vacuo, and the resulting residue was purified using silica gel chromatography (40 gram ISCO Redisep Gold column, eluent 0-100% EtOAc/Hexanes) to provide compound 1c. MS: m/z=508.2 [M+H].

### Step C—Synthesis of Compound 1d

To a stirred solution of compound 1c (100 mg, 0.197 mmol) in DMF (750 μL) at 0° C., was added sodium hydride (60% dispersion in mineral oil, 8.68 mg, 0.217 mmol). The resulting reaction was warmed to room temperature, and allowed to stir for an additional 10 minutes. The reaction mixture was then cooled to 0° C., 1-(chloromethyl)-2-methoxybenzene (37.1 mg, 0.237 mmol) was added, and the resulting reaction was again warmed to room temperature, and allowed to stir at room temperature for 10 minutes. The reaction was then quenched with water (1 mL), and concentrated in vacuo, and the resulting residue was purified using silica gel chromatography (12 gram ISCO Redisep Gold column, eluent 0-100% EtOAc/Hexanes) to provide compound 1d. MS: m/z=627.2 [M+H].

### Step D—Synthesis of Compound 1

To a solution of compound 1d (72 mg, 0.115 mmol) in DCE (1150 μl), at room temperature, was added TFA (531 μl, 6.89 mmol), and the resulting reaction was allowed to stir overnight. The reaction mixture was concentrated in vacuo, and the resulting residue was purified using silica gel chromatography (14 gram ISCO Redisep Gold column, eluent 0-10% MeOH/DCM) to provide compound 1, as the TFA salt. MS: m/z=327.0 [M+H]. ¹H NMR (500 MHz, Methanol-d₄) δ 7.79 (s, 1H), 7.39-7.32 (m, 1H), 7.24 (dd, J=7.5, 1.3 Hz, 1H), 7.01 (d, J=8.3 Hz, 1H), 6.95 (t, J=7.4 Hz, 1H), 5.48 (s, 2H), 3.86 (s, 3H), 3.62 (t, J=7.2 Hz, 2H), 1.80-1.56 (m, 2H), 1.51-1.35 (m, 2H), 0.98 (t, J=7.4 Hz, 3H).

The following compounds of the present invention were made using the methods described in Example 1 above, and substituting the appropriate reactants and/or reagents:

| | | MS | |
|---|---|---|---|
| Compound | Structure | [M + H] | ¹H NMR |
| 2 | 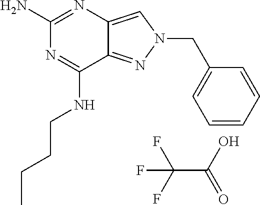 | 297.0 | (500 MHz, Methanol- d₄) δ 7.88 (s, 1H), 7.47- 7.18 (m, 5H), 5.52 (s, 2H), 3.64 (t, J = 7.2 Hz, 2H), 1.69 (dd, J = 8.4, 6.3 Hz, 2H), 1.49- 1.39 (m, 2H), 0.99 (t, J = 7.4 Hz, 3H). |
| 3 | 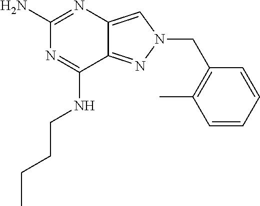 [SMILES: CCCCNc1nc(N)nc2cn(Cc3ccccc3C)nc12] | 311.5 | (500 MHz, Methanol- d₄) δ 7.54 (s, 1H), 7.28- 7.17 (m, 3H), 7.12 (d, J = 7.4 Hz, 1H), 5.50 (s, 2H), 3.51 (t, J = 7.2 Hz, 2H), 2.27 (s, 3H), 1.64 (q, J = 7.4 Hz, 2H), 1.43 (q, J = 7.5 Hz, 2H), 0.97 (t, J = 7.4 Hz, 3H). |
| 4 | 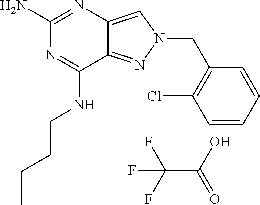 | 331.0 | (500 MHz, Methanol- d₄) δ 7.89 (s, 1H), 7.46 (dd, J = 7.7, 1.3 Hz, 1H), 7.34 (ddd, J = 15.8, 7.4, 1.6 Hz, 2H), 7.29 (dd, J = 7.4, 1.8 Hz, 1H), 5.66 (s, 2H), 3.63 (t, J = 7.3 Hz, 2H), 1.79-1.62 (m, 2H), 1.51-1.37 (m, 2H), 0.98 (t, J = 7.4 Hz, 3H). |

### Example 2

### Preparation of Compound 5

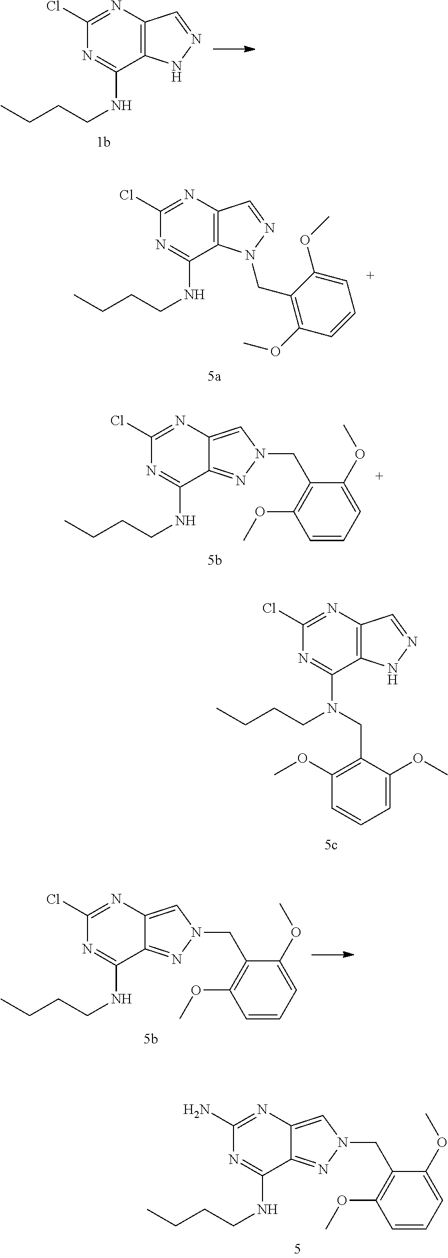

### Step A—Synthesis of Compound 5a, 5b and 5e

To a mixture of compound 1b (1 g, 4.43 mmol), and (2,6-dimethoxyphenyl)methanol (1.12 g, 6.65 mmol) in toluene (20 mL) was added CMBP (4.28 g, 17.7 mmol). The resulting reaction was heated to 110° C., and allowed to stir at this temperature for 16 hours. The reaction mixture was concentrated in vacuo, and the residue obtained was dissolved in DCM (300 mL). The resulting solution was washed sequentially with water (3×30 mL), and brine (sat., 3×10 mL), dried over anhydrous sodium sulfate, and filtered. The filtrate was concentrated in vacuo, and the resulting residue was purified using RP-flash chromatography (eluted with 0-50% acetonitrile in 5 mM aq. ammonium bicarbonate) to provide a crude product. The crude product was then further purified using Prep-Chiral-HPLC with the following conditions: Column: GreenSep Basic, Mobile Phase A: carbon dioxide, Mobile Phase B: MeOH (8 mM NH₃ in MeOH); Flow rate: 50 mL/minute; Gradient: 20% B; Detector: UV 254 nm; RT1: 3.58 minutes, RT2: 4.73 minutes and RT3: 6.11 minute.

The fractions of the first peak (RT1: 3.58 minutes) were collected and concentrated in vacuo to provide compound 5a. MS: m/z=376.1 [M+H].

The fractions of the second peak (RT2: 4.73 minutes) were collected and concentrated in vacuo to provide compound 5b. MS: m/z=376.1 [M+H].

The fractions of the third peak (RT3: 6.11 minute) were collected and concentrated in vacuo to provide compound 5c. MS: m/z=376.1 [M+H].

### Step B—Synthesis of Compound 5

A mixture of compound 5b (200 mg, 0.532 mmol) in ammonia (60 mL, 70% in 2-propanol, v/v) was sealed, heated to 80° C., and allowed to stir at this temperature for 20 hours. The reaction mixture was concentrated in vacuo, and the residue obtained was purified using Prep-HPLC with the following conditions: Column: XBridge C18 OBD Prep Column, 100 A, 10 μm, 19 mm×250 mm; Mobile Phase A: 10 mM aq. ammonium bicarbonate, Mobile Phase B: acetonitrile; Flow rate: 20 mL/minute; Gradient: 35% B to 70% B in 5.8 minutes; Detector: UV 210/254 nm; room temperature: 5.45 minutes. The fractions containing desired product were combined, and concentrated in vacuo to provide compound 5. MS: m/z=357.2 [M+H]. ¹H NMR (400 MHz, d₆-DMSO) δ 7.58 (s, 1H), 7.38-7.34 (m, 1H), 7.29 (s, 1H), 6.73 (d, J=9.2 Hz, 2H), 5.47 (s, 2H), 5.39 (s, 2H), 3.80 (s, 6H), 3.50-3.35 (m, 2H), 1.58-1.54 (m, 2H), 1.35-1.29 (m, 2H), 0.95-0.88 (m, 3H).

The following compounds of the present invention were made using the methods described in Example 2 above, and substituting the appropriate reactants and/or reagents:

| | | MS | |
|---|---|---|---|
| Compound | Structure | [M + H] | ¹H NMR |
| 6 | 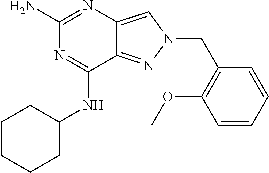 [SMILES: COc1ccccc1Cn1cc2nc(N)nc(NC3CCCCC3)c2n1] | 353.2 | (300 MHz, d₆- DMSO) δ 7.68 (s, 1H), 7.47-7.44 (m, 1H), 7.34-7.28 (m, 1H), 7.06-7.03 (m, 1H), 6.93-6.88 (m, 2H), 5.67 (s, 2H), 5.42 (s, 2H), 4.07- 4.01 (m, 1H), 3.83 (s, 3H), 1.85-1.59 (m, 5H), 1.45-1.15 |
| | | | (m, 5H). |
| 7 | 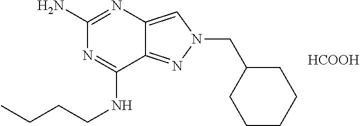 [SMILES: CCCCNc1nc(N)nc2cn(CC3CCCCC3)nc12 ; "HCOOH"] | 303.2 | (300 MHz, CDCl₃) δ 8.64 (s, 1H), 7.78 (s, 1H), 6.29 (s, 1H), 4.06-4.03 (m, 2H), 3.63-3.59 (m, 2H), 1.92-1.89 (m, 1H), 1.88-1.42 (m, 9 H), 1.21-1.10 (m, 3H), 1.01-0.92 (m, |
| | | | 5H). |
| 8 | 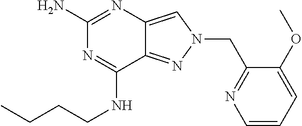 [SMILES: CCCCNc1nc(N)nc2cn(Cc3ncccc3OC)nc12] | 328.1 | (300 MHz, CDCl₃) δ 8.17-8.15 (m, 1H), 7.57 (s, 1H), 7.24-7.14 (m, 2H), 5.94 (s, 1H), 5.56 (s, 2H), 4.71 (s, 2H), 3.83 (s, 3H), 3.53- 3.49 (m, 2H), 1.63- 1.52 (m, 2H), 1.48- |
| | | | 1.32 (m, 2H), 0.93- |
| | | | 0.88 (m, 3H). |
| 9 | 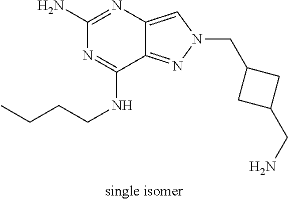 [SMILES: CCCCNc1nc(N)nc2cn(CC3CC(CN)C3)nc12] | 304.2 | (300 MHz, CD₃OD) δ 7.82 (s, 1H), 4.37- 4.34 (m, 2H), 3.68- 3.63 (m, 2H), 2.95- 2.80 (m, 3H), 2.56- 2.45 (m, 1H), 2.32- 2.23 (m, 2H), 1.75- 1.62 (m, 4H), 1.51- 1.38 (m, 2H), 1.02- 0.97 (m, 3H). |
| 10 | 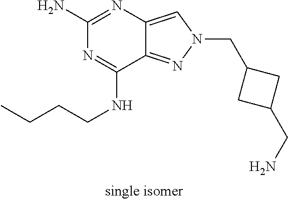 [SMILES: CCCCNc1nc(N)nc2cn(CC3CC(CN)C3)nc12] | 304.2 | (300 MHz, CD₃OD) δ 7.87 (s, 1H), 4.50- 4.44 (m, 2H), 3.68- 3.63 (m, 2H), 3.07- 2.92 (m, 3H), 2.71- 2.57 (m, 1H), 2.24- 1.94 (m, 4H), 1.75- 1.65 (m, 2H), 1.50- 1.30 (m, 2H), 1.02- 0.96 (m, 3H). |
| 11 | 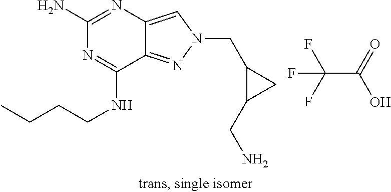 | 290.2 | (300 MHz, d₆- DMSO) δ 12.41 (s, 1H), 9.43-9.39 (m, 1H), 8.10-7.97 (m, 5H), 4.48-4.33 (m, 2H), 3.57-3.51 (m, 2H), 3.13-2.91 (m, 2H), 1.64-1.46 (m, 3H), 1.42-1.23 (m, 3H), 0.94-0.83 (m, 4H), 0.57-0.52 (m, 1H) |
| 12 | 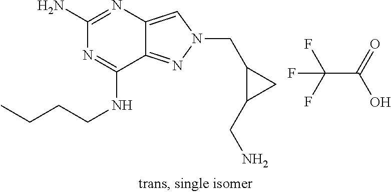 | 290.2 | (300 MHz, d₆- DMSO) δ 12.35 (s, 1H), 9.43-9.40 (m, 1H), 8.10-7.96 (m, 5H), 4.49-4.34 (m, 2H), 3.58-3.51 (m, 2H), 3.13-2.93 (m, 2H), 1.65-1.50 (m, 3H), 1.39-1.24 (m, 3H), 0.95-0.83 (m, 4H), 0.58-0.52 (m, 1H). |
| 13 | 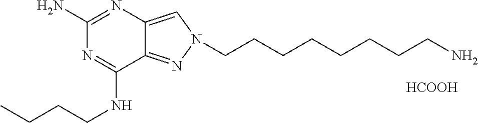 [SMILES: CCCCNc1nc(N)nc2cn(CCCCCCCCN)nc12 ; "HCOOH"] | 334.3 | (300 MHz, CD₃OD) δ 8.56 (s, 2H), 7.78 (s, 1H), 4.35-4.29 (m, 2H), 3.63-3.58 (m, 2H), 2.91-2.86 (m, 2H), 1.98-1.91 (m, 2H), 1.73-1.61 (m, 4H), 1.52-1.21 |
| | | | (m, 10H), 0.97- |
| | | | 0.86 (m, 3H). |
| 117 | 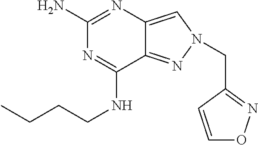 [SMILES: CCCCNc1nc(N)nc2cn(Cc3ccon3)nc12] | 288.1 | (300 MHz, CDCl₃) δ 8.38 (s, 1H), 7.55 (s, 1H), 6.24 (s, 1H), 5.87-5.80 (m, 1H), 5.52 (s, 2H), 4.86 (s, 2H), 3.58-3.56 (m, 2H), 1.70-1.68 (m, 2H), 1.47-1.45 (m, 2H), 1.38-1.36 (m, |
| | | | 3H). |

### Example 3

### Preparation of Compounds 14 and 15

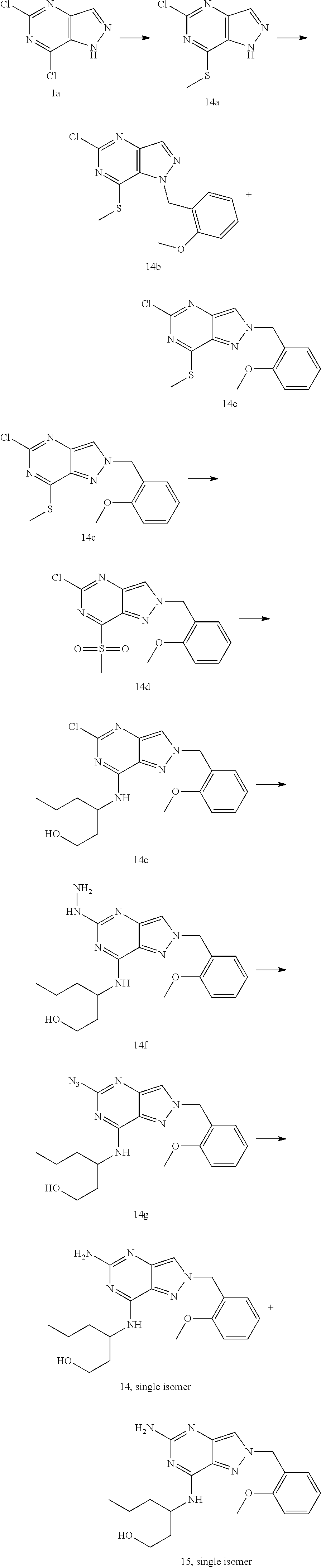

### Step A—Synthesis of Compound 14a

To a solution of compound 1a (200 mg, 1.058 mmol) in DMF (5 mL) at 0° C. under argon atmosphere was added sodium thiomethoxide (148 mg, 2.116 mmol). The resulting reaction was allowed to stir at 0° C. for 2 hours, and the reaction was quenched with saturated aqueous ammonium chloride (20 mL), and extracted with ethyl acetate (50 mL). The organic layer was washed with brine (20 mL), dried over anhydrous sodium sulfate, and concentrated in vacuo. The residue obtained was purified using silica gel column chromatography (eluted with 30-70% ethyl acetate in petroleum ether) to provide compound 14a. MS: m/z=201.1 [M+H].

### Step B—Synthesis of Compound 14b and 14c

Compounds 14b and 14c were made from compound 14a, using the method described in Example 2, step A, and substituting the appropriate reactants and/or reagents. The product mixture was then separated according to the methods described in Example 2, step A, and compound 14c was used in the next step. MS: m/z=321.1 [M+H].

### Step C—Synthesis of Compound 14d

To a solution of compound 14c (60 mg, 0.187 mmol) in DCM (1.5 mL) at 0° C., were added m-CPBA (97 mg, 0.561 mmol), and 4 Å molecular sieves (200 mg), and the resulting reaction was heated to 25° C., and allowed to stir at this temperature for 3 hours. The reaction was quenched with aqueous sodium hydrogen carbonate (10 mL), and extracted with DCM (2×15 mL). The combined organic extracts were washed with brine (20 mL), dried over magnesium sulfate, filtered, and concentrated in vacuo to provide compound 14d, which was used without further purification. MS: m/z=353.1 [M+H].

### Step D—Synthesis of Compound 14e

A stirred solution of 3-aminohexan-1-ol hydrochloride (130 mg, 0.852 mmol) in THF (3 mL) was cooled to 0° C., and put under nitrogen atmosphere. TEA (0.127 mL, 1.704 mmol) was added dropwise and the resulting reaction was allowed to stir for 10 minutes at 0° C. A solution of compound 14d (300 mg, 0.852 mmol) in THF (3 mL) was then added, and the resulting reaction was warmed to 25° C., and allowed to stir at this temperature for 1 hour. The reaction mixture was concentrated in vacuo, and the residue obtained was purified using silica gel column chromatography (eluted with 0˜80% ethyl acetate in petroleum ether) to provide compound 14e. MS: m/z=392.1 [M+H].

### Step E—Synthesis of Compound 14f

To a solution of compound 14e (210 mg, 0.539 mmol) in ethanol (0.5 mL) was added hydrazine hydrate (1.5 mL, 0.256 mmol), and the resulting reaction was heated to 80° C., and allowed to stir at this temperature for 16 hours. The reaction mixture was concentrated in vacuo, and the residue obtained was purified using silica gel column chromatography (eluted 0˜100% ethyl acetate in petroleum ether) to provide compound 14f MS: m/z=386.1 [M+H].

### Step F—Synthesis of Compound 14g

To a mixture of compound 14f (140 mg, 0.363 mmol) in acetic acid (1 mL) at 0° C. was added a solution of sodium nitrite (49.5 mg, 0.727 mmol) in water (0.1 mL), and the resulting reaction was allowed to stir for 3 hours at 0° C. The reaction mixture was then concentrated in vacuo, and the resulting residue was taken up in DCM (10 mL), and washed with brine (2×5 mL). The organic phase was collected, dried over sodium sulfate, filtered, and concentrated in vacuo. The resulting residue was purified using silica gel column chromatography (eluted with 0˜80% ethyl acetate in petroleum ether) to provide compound 14g. MS: m/z=397.2 [M+H].

### Step G—Synthesis of Compounds 14 and 15

To a stirred mixture of compound 14g (100 mg, 0.303 mmol) in THF (1 mL), and water (0.3 mL) was added trimethylphosphine (0.5 M in THF, 0.908 mL, 0.454 mmol), and the resulting reaction was allowed to stir for 3 hours at room temperature under nitrogen atmosphere. The reaction was quenched with MeOH (1 mL), and the resulting solution was concentrated in vacuo. The residue obtained was purified using silica gel column chromatography (eluted with 1˜5% MeOH in DCM) to provide a racemic mixture which was separated using Prep- CHIRAL-HPLC with the following conditions: Column: Chiralpak ID-2, 2×25 cm, 5 um; Mobile Phase A: Hex (0.5% 2M NH₃-MeOH), Mobile Phase B: EtOH; Flow rate: 16 mL/minute; Gradient: 5% B to 50% B in 22 minutes; Detector: UV 220/254 nm; RT1: 8.847 minutes, RT2: 13.905 minutes. The first eluting peak (RT1: 8.847 minutes) was collected, and concentrated in vacuo, and then lyophilized overnight to provide compound 14. MS: m/z=371.3 [M+H]. ¹H NMR (400 MHz, d₆-DMSO) δ 7.67 (s, 1H), 7.39-7.28 (m, 2H), 7.06-7.04 (m, 1H), 6.93-6.89 (m, 2H), 5.54 (s, 2H), 5.42 (s, 2H), 4.44-4.34 (m, 2H), 3.83 (s, 3H), 3.41-3.34 (m, 2H), 1.74-1.56 (m, 3H), 1.55-1.43 (m, 1H),1.35-1.23 (m, 2H) 0.92-0.78 (m, 3H).

The second eluting peak (RT2: 13.905 minutes) was collected, and concentrated in vacuo, and then lyophilized overnight to provide compound 15. MS: m/z=371.3 [M+H]. ¹H NMR (400 MHz, d₆-DMSO) δ 7.67 (s, 1H), 7.36-7.29 (m, 2H), 7.06-7.04 (m, 1H), 6.93-6.88 (m, 2H), 5.50 (s, 2H), 5.42 (s, 2H), 4.46-4.33 (m, 2H), 3.83 (s, 3H), 3.41-3.40 (m, 2H), 1.70-1.58 (m, 3H), 1.50-1.46 (m, 1H), 1.31-1.26 (m, 2H), 0.90-0.82 (m, 3H).

The following compounds of the present invention were made using the methods described in Example 3 above, and substituting the appropriate reactants and/or reagents:

| | | MS | |
|---|---|---|---|
| Compound | Structure | [M + H] | ¹H NMR |
| 16 | 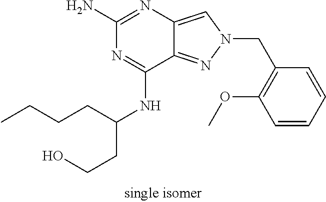 [SMILES: CCCCC(CCO)Nc1nc(N)nc2cn(Cc3ccccc3OC)nc12] | 385.2 | (400 MHz, CD₃OD) δ 7.61 (s, 1H), 7.35- 7.31 (m, 1H), 7.17- 7.15 (m, 1H), 7.03- 7.01 (m, 1H), 6.95- 6.92 (m, 1H), 5.46 (s, 2H), 4.86-4.39 (m, 1H), 3.85 (s, 3H), 3.60-3.56 (m, 2H), 1.90-1.83 (m, 1H), 1.69-1.61 (m, 3H), 1.43-1.33 (m, 4H), 0.91-0.88 (m, 3H). |
| 17 | 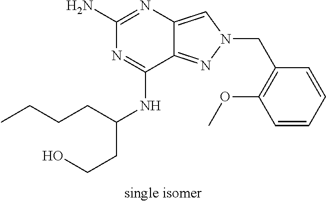 [SMILES: CCCCC(CCO)Nc1nc(N)nc2cn(Cc3ccccc3OC)nc12] | 385.2 | (400 MHz, CD₃OD) δ 7.61 (s, 1H), 7.35- 7.31 (m, 1H), 7.17- 7.15 (m, 1H), 7.03- 7.01 (m, 1H), 6.95- 6.92 (m, 1H), 5.46 (s, 2H), 4.86-4.39 (m, 1H), 3.85 (s, 3H), 3.60-3.56 (m, 2H), 1.90-1.83 (m, 1H), 1.69-1.61 (m, 3H), 1.40-1.28 (m, 4H), 0.94-0.88 (m, 3H). |
| 18 | 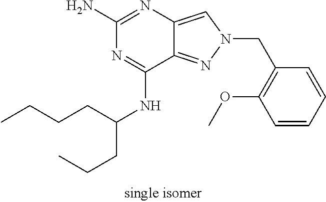 [SMILES: CCCCC(CCC)Nc1nc(N)nc2cn(Cc3ccccc3OC)nc12] | 383.2 | (400 MHz, CD₃OD) δ 7.60 (s, 1H), 7.34- 7.30 (m, 1H), 7.16- 7.14 (m, 1H), 7.01- 6.99 (m, 1H), 6.94- 6.91 (m, 1H), 5.46 (s, 2H), 4.34-4.30 (m, 1H), 3.84 (s, 3H), 1.62-1.48 (m, 4H), 1.43-1.38 (m, 6H), 0.87-0.85 (m, 6H). |
| 19 | 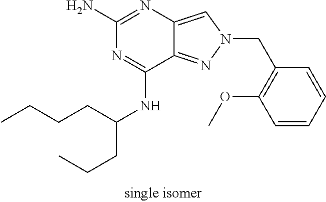 [SMILES: CCCCC(CCC)Nc1nc(N)nc2cn(Cc3ccccc3OC)nc12] | 383.2 | (400 MHz, CD₃OD) δ 7.62 (s, 1H), 7.37- 7.33 (m, 1H), 7.19- 7.17 (m, 1H), 7.05- 7.02 (m, 1H), 6.98- 6.94 (m, 1H), 5.47 (s, 2H), 4.36-4.32 (m, 1H), 3.87 (s, 3H), 1.64-1.36 (m, 10H), 0.96-0.88 (m, 6H). |
| 20 | 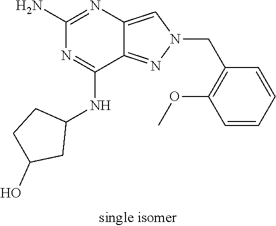 [SMILES: COc1ccccc1Cn1cc2nc(N)nc(NC3CCC(O)C3)c2n1] | 355.3 | (400 MHz, d₆- DMSO) δ 8.71-8.70 (m, 1H), 7.89 (s, 1H), 7.36-7.32 (m, 1H), 7.14 (s, 2H), 7.07- 7.05 (m, 1H), 6.97- 6.91 (m, 2H), 5.47 (s, 2H), 4.90-4.70 (m, 1H), 4.58-4.53 (m, 1H), 4.14-4.12 (m, 1H), 3.82 (s, 3H), 2.17-2.12 (m, 1H), 1.96-1.89 (m, 1H), 1.83-1.60 (m, 4H). |
| 21 | 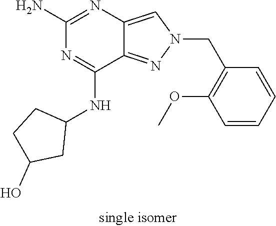 [SMILES: COc1ccccc1Cn1cc2nc(N)nc(NC3CCC(O)C3)c2n1] | 355.3 | (400 MHz, d₆- DMSO) δ 8.65-8.63 (m, 1H), 7.89 (s, 1H), 7.36-7.32 (m, 1H), 7.20 (s, 2H), 7.07- 7.05 (m, 1H), 6.97- 6.91 (m, 2H), 5.47 (s, 2H), 4.90-4.70 (m, 1H), 4.59-4.54 (m, 1H), 4.15-4.13 (m, 1H), 3.83 (s, 3H), 2.17-2.11 (m, 1H), 1.96-1.90 (m, 1H), 1.83-1.60 (m, 4H). |
| 22 | 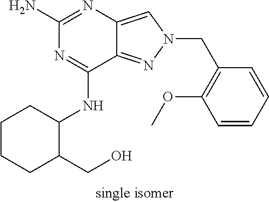 [SMILES: COc1ccccc1Cn1cc2nc(N)nc(NC3CCCCC3CO)c2n1] | 383.2 | (400 MHz, CDCl₃) δ 7.54 (s, 1H), 7.35- 7.33 (m, 1H), 7.12- 7.10 (m, 1H), 6.95- 6.91 (m, 2H), 6.10- 6.08 (m, 1H), 5.43 (s, 2H), 4.80-4.77 (m, 2H), 4.64-4.63 (m, 1H), 3.86 (s, 3H), 3.40-3.36 (m, 1H), 3.24-3.18 (m, 1H), 1.95-1.59 (m, 5H), 1.59-1.51 (m, 1H), |
| | | | 1.41-1.37 (m, 2H), |
| | | | 1.18-1.07 (m, 2H). |
| 23 | 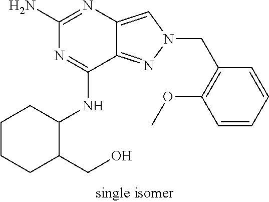 [SMILES: COc1ccccc1Cn1cc2nc(N)nc(NC3CCCCC3CO)c2n1] | 383.2 | (400 MHz, CDCl₃) δ 7.55 (s, 1H), 7.37- 7.33 (m, 1H), 7.15- 7.13 (m, 1H), 6.99- 6.92 (m, 2H), 5.43 (s, 2H), 4.70-4.63 (m, 1H), 3.87 (s, 3H), 3.44-3.40 (m, 1H), 3.30-3.25 (m, 1H), 1.96-1.93 (m, 2H), 1.81-1.74 (m, 4H), 1.64-1.40 (m, 3H), 1.18-1.07 (m, 1H). |
| 24 | 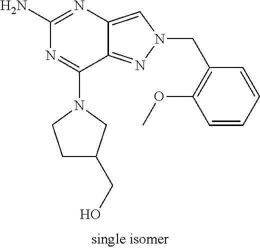 [SMILES: COc1ccccc1Cn1cc2nc(N)nc(N3CCC(CO)C3)c2n1] | 355.3 | (400 MHz, CD₃OD) δ 7.57 (s, 1H), 7.36- 7.32 (m, 1H), 7.09- 7.03 (m, 2H), 6.95- 6.91 (m, 1H), 5.49 (s, 2H), 4.42-4.34 (m, 1H), 4.09-3.99 (m, 1H), 3.96-3.89 (m, 4H), 3.72-3.50 (m, 3H), 2.61-2.49 (m, 1H), 2.10-2.09 (m, 1H), 1.85-1.80 (m, 1H). |
| 25 | 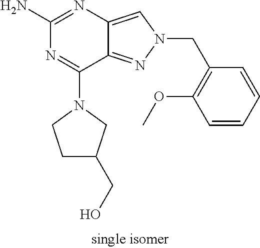 [SMILES: COc1ccccc1Cn1cc2nc(N)nc(N3CCC(CO)C3)c2n1] | 355.3 | (400 MHz, CD₃OD) δ 7.57 (s, 1H), 7.36- 7.32 (m, 1H), 7.05- 7.03 (m, 2H), 6.95- 6.91 (m, 1H), 5.49 (s, 2H), 4.39-4.33 (m, 1H), 4.09-3.99 (m, 1H), 3.96-3.89 (m, 4H), 3.72-3.50 (m, 3H), 2.61-2.48 (m, 1H), 2.21-2.10 (m, 1H), 1.91-1.81 (m, 1H). |
| 26 | 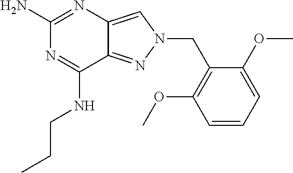 [SMILES: CCCNc1nc(N)nc2cn(Cc3c(OC)cccc3OC)nc12] | 343.1 | (300 MHz, d₆- DMSO) δ 7.64 (s, 1H), 7.39-7.30 (m, 2H), 6.75-6.72 (m, 2H), 5.54 (s, 2H), 5.39 (s, 2H), 3.80 (s, 6H), 3.35-3.30 (m, 2H) 1.63-1.55 (m, 2H), 0.91-0.86 (m, 3H). |
| 27 | 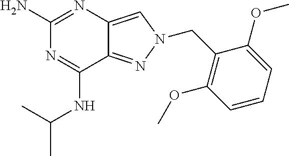 [SMILES: COc1cccc(OC)c1Cn1cc2nc(N)nc(NC(C)C)c2n1] | 343.2 | (300 MHz, CDCl₃) δ 8.70 (s, 1H) 7.43- 7.29 (m, 2H), 6.64- 6.62 (m, 2H), 6.24- 6.20 (m, 2H), 5.50 (s, 2H), 4.50-4.45 (m, 1H), 3.86 (s, 6H), 1.37-1.28 (m, 6H). |
| 28 | 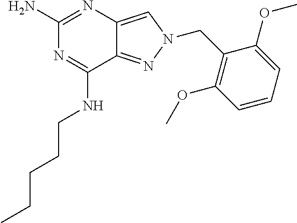 [SMILES: CCCCCNc1nc(N)nc2cn(Cc3c(OC)cccc3OC)nc12] | 371.1 | (300 MHz, CDCl₃) δ 8.68 (s, 1H), 7.41 (s, 1H), 7.36-7.26 (m, 1H), 6.61-6.58 (m, 2H), 6.24-6.21 (m, 2H), 5.47 (s, 2H), 3.83 (s, 6H), 3.60- 3.55 (m, 2H), 1.72- 1.66 (m, 2H), 1.58- 1.56 (m, 4H), 0.95- 0.90 (m, 3H). |

### Example 4

### Preparation of Compound 29

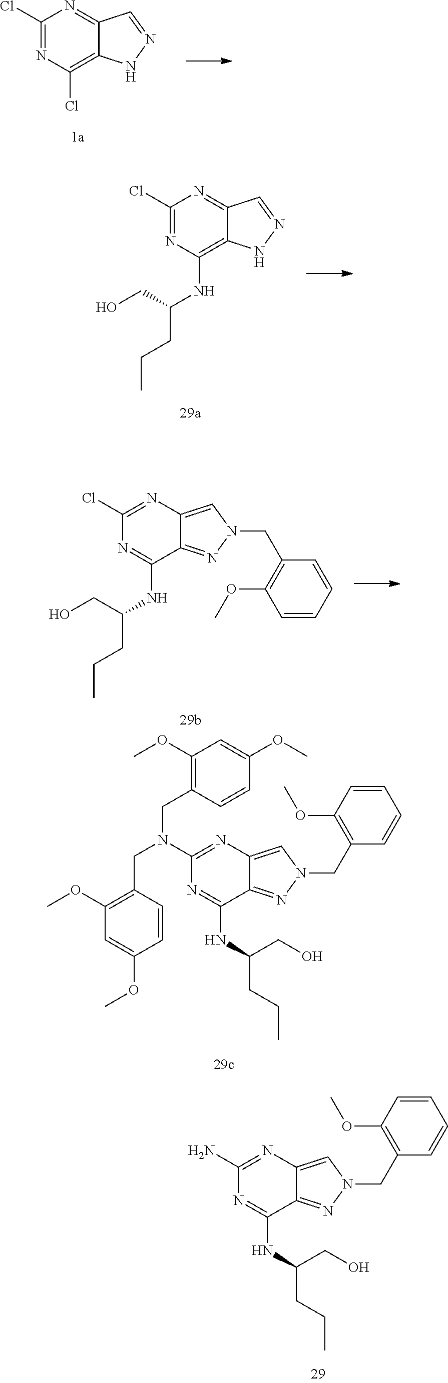 [SMILES: Clc1nc(Cl)c2[nH]ncc2n1 ; CCC[C@H](CO)Nc1nc(Cl)nc2cn[nH]c12 ; CCC[C@H](CO)Nc1nc(Cl)nc2cn(Cc3ccccc3OC)nc12 ; CCC[C@H](CO)Nc1nc(N(Cc2ccc(OC)cc2OC)Cc2ccc(OC)cc2OC)nc2cn(Cc3ccccc3OC)nc12 ; CCC[C@H](CO)Nc1nc(N)nc2cn(Cc3ccccc3OC)nc12]

### Step A—Synthesis of Compound 29a

To a stirred solution of (R)-2-aminopentan-1-ol (546 mg, 5.29 mmol) in DCM (3.5 mL) at 0° C. was added compound 1a (200 mg, 1.058 mmol) portion wise. The resulting reaction was warmed to room temperature and allowed to stir overnight. The reaction mixture was concentrated in vacuo, and the residue obtained was purified using silica gel chromatography (24 gram ISCO Redisep Gold column (eluent 0-10% MeOH/DCM) to provide compound 29a. MS: m/z=255.2 [M+H].

### Step B—Synthesis of Compound 29b

Compound 29b was made from compound 29a, using the method described in Example 1, step C, and substituting the appropriate reactants and/or reagents. MS: m/z=375.2 [M+H].

### Step C—Synthesis of Compound 29c

Compound 29c was made from compound 29b, using the method described in Example 1, step B, and substituting the appropriate reactants and/or reagents. MS: m/z=656.6 [M+H].

### Step C—Synthesis of Compound 29

Compound 29 was made from compound 29c, using the method described in Example 1, step D, and substituting the appropriate reactants and/or reagents. MS: m/z=357.4 [M+H]. ¹H NMR (500 MHz, Methanol-d₄) δ 7.62 (s, 1H), 7.39-7.28 (m, 1H), 7.16 (d, J=7.0 Hz, 1H), 7.02 (d, J=8.2 Hz, 1H), 6.94 (t, J=7.5 Hz, 1H), 5.46 (d, J=1.7 Hz, 2H), 4.35 (dd, J=9.0, 4.9 Hz, 1H), 3.85 (s, 3H), 3.64 (dd, J=5.1, 3.2 Hz, 2H), 1.69 (s, 1H), 1.64-1.54 (m, 1H), 1.44 (dd, J=14.6, 7.3 Hz, 2H), 0.96 (t, J=7.3 Hz, 3H).

### Example 5

### Preparation of Compounds 30 and 31

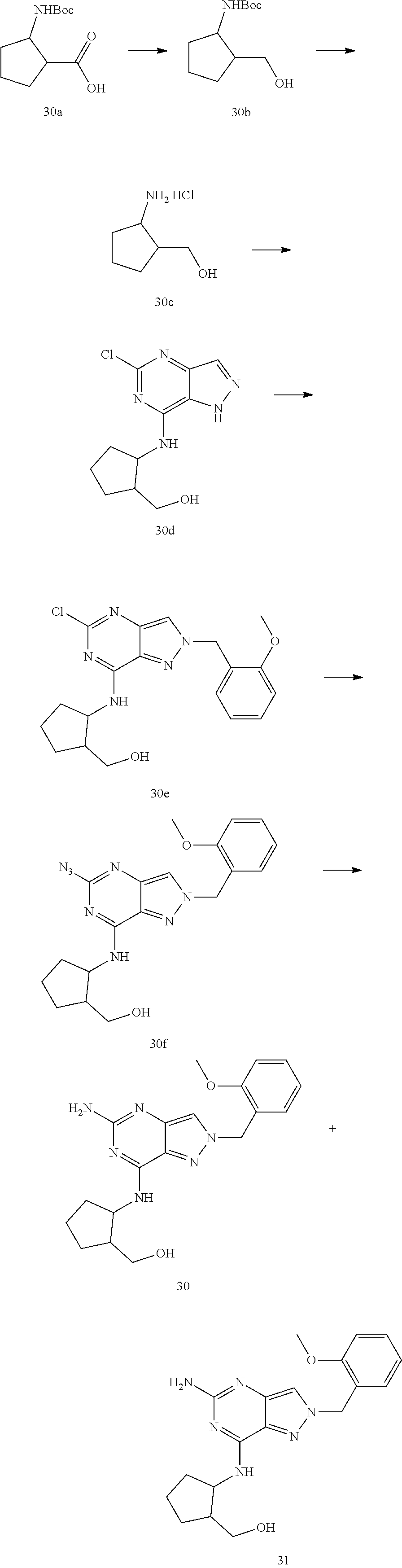

### Step A—Synthesis of Compound 30b

To a mixture of compound 30a (150 mg, 0.654 mmol) in THF (3 mL), under argon atmosphere at 0° C., was added BH₃·DMS (2M in THF, 0.654 mL, 1.308 mmol) dropwise. The resulting reaction was allowed to stir for 3 hours at room temperature, then the reaction was quenched with MeOH (2 mL), and concentrated in vacuo to provide compound 30b, which was used without further purification. MS: m/z=216.3 [M+H].

### Step B—Synthesis of Compound 30c

To a solution of compound 30b (100 mg, 0.464 mmol) in DCM (1 mL) was added HCl (4 M in dioxane, 1 mL, 4.00 mmol). The resulting reaction was allowed to stir for 1 hour at 20° C., and the reaction mixture was concentrated in vacuo to provide compound 30c, which was used without further purification. MS: m/z=116.1 [M+H].

### Step C—Synthesis of Compound 30d

Compound 30d was made from compound 30c, using the method described in Example 2, step B, and substituting the appropriate reactants and/or reagents. MS: m/z=268.1 [M+H].

### Step D—Synthesis of Compound 30e

To a mixture of compound 30d (150 mg, 0.560 mmol), and potassium carbonate (232 mg, 1.681 mmol) in acetonitrile (5 mL) at 20° C., was added 1-(chloromethyl)-2-methoxybenzene (132 mg, 0.840 mmol). The resulting reaction was heated to 80° C., and allowed to stir at this temperature for 2 hours. The reaction mixture was then filtered, the filtrate was concentrated in vacuo, and the residue obtained was purified using Prep-TLC, and eluted with (3/2 ethyl acetate/petroleum ether) to provide compound 30e. MS: m/z=388.2 [M+H].

### Step E—Synthesis of Compound 30f

To a solution of compound 30e (150 mg, 0.387 mmol) in acetic acid (0.8 mL), and ethanol (2.5 mL) was added sodium azide (32.7 mg, 0.503 mmol). The resulting reaction was heated to 100° C., and allowed to stir for 2 hours at this temperature. The reaction mixture was then concentrated in vacuo, and the resulting residue was diluted with ethyl acetate (80 mL). The resulting solution was washed sequentially with aqueous sodium hydrogen carbonate (sat., 20 mL), and brine (20 mL), dried over anhydrous sodium sulfate, and concentrated in vacuo to provide compound 30f, which was used without further purification. MS: m/z=395.2 [M+H].

### Step F—Synthesis of Compound 30 and 31

To a mixture of compound 30f (150 mg, 0.380 mmol) in THF (2.5 mL), and water (0.5 mL) was added trimethylphosphine (1 M in THF, 0.380 mL, 0.380 mmol). The resulting reaction was heated to 50° C., and allowed to stir at this temperature for 4 hours. The reaction was quenched with MeOH (2 mL), and the reaction mixture was concentrated in vacuo. The residue obtained was purified using Prep-TLC (DCM/MeOH=10/1) to provide the product as a racemic mixture. The racemic mixture was separated using chiral-HPLC with the following conditions: Column: CHIRALPAK IG, 2×25 cm, 5 um; Mobile Phase A: dimethoxy-ethane (0.5% 2M amine-MeOH), Mobile Phase B: EtOH; Flow rate: 15 mL/minute; Gradient: 50% B to 50% B in 12 minutes; Detector: UV 220/254 nm; RT1: 8.381 minutes, RT2: 9.669 minutes The fractions corresponding to the first peak (RT1: 8.381 min) were concentrated in vacuo to provide compound 30. MS: m/z=369.2 [M+H]. ¹H NMR (300 MHz, CDCl₃) δ 7.55 (s, 1H), 7.36-7.34 (m, 1H), 7.30-7.09 (m, 1H), 6.96-6.91 (m, 2H), 5.80 (s, 1H), 5.44 (s, 2H), 4.81 (s, 2H), 4.62-4.59 (m, 1H), 3.86 (s, 3H), 3.63-3.59 (m, 1H), 3.40-3.33 (m, 1H), 2.34-2.25 (m, 2H), 1.99-1.85 (m, 4H), 1.38-1.18 (m, 2H).

The fractions corresponding to the second peak (RT2: 9.669 min) were concentrated in vacuo to provide compound 31. MS: m/z=369.2 [M+H]. ¹H NMR (300 MHz, CDCl₃) δ 7.55 (s, 1H), 7.36-7.27 (m, 1H), 7.09-7.07 (m, 1H), 6.96-6.91 (m, 2H), 5.68 (s, 1H), 5.51 (s, 2H), 4.68-4.59 (m, 3H), 3.87 (s, 3H), 3.63-3.60 (m, 1H), 3.39-3.32 (m, 1H), 2.30-2.13 (m, 2H), 1.96-1.73 (m, 4H), 1.31-1.18 (m, 2H).

The following compounds of the present invention were made using the methods described in Example 5 above, and substituting the appropriate reactants and/or reagents:

| | | MS | |
|---|---|---|---|
| Compound | Structure | [M + H] | ¹H NMR |
| 32 | 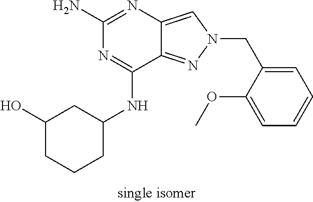 [SMILES: COc1ccccc1Cn1cc2nc(N)nc(NC3CCCC(O)C3)c2n1] | 369.2 | (300 MHz, CDCl₃) δ 7.54 (s, 1H), 7.35-7.30 (m, 1H), 7.08-7.05 (m, 1H), 6.95-6.90 (m, 2H), 5.73 (s, 1H), 5.43 (s, 2H), 5.00 (s, 2H), 4.58-4.57 (m, 1H), 4.20 (s, 1H), 3.86 (s, 3H), 2.08-1.89 (m, 4H), 1.67-1.63 (m, 4H). |
| 33 | 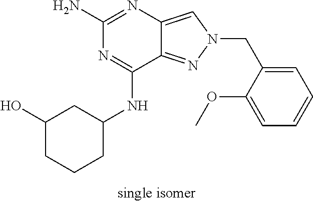 [SMILES: COc1ccccc1Cn1cc2nc(N)nc(NC3CCCC(O)C3)c2n1] | 369.2 | (300 MHz, CDCl₃) δ 7.53 (s, 1H), 7.35-7.29 (m, 1H), 7.07-7.05 (m, 1H), 6.95-6.90 (m, 2H), 6.01 (s, 1H), 5.44 (s, 2H), 4.74 (s, 2H), 4.25 (s, 1H), 3.86-3.75 (m, 4H), 2.38-2.34 (m, 1H), 2.05-1.85 (m, 3H), 1.49-1.24 (m, 4H). |
| 34 | 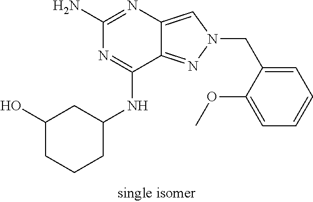 [SMILES: COc1ccccc1Cn1cc2nc(N)nc(NC3CCCC(O)C3)c2n1] | 369.2 | (300 MHz, CD₃OD) δ 7.63 (s, 1H), 7.37-7.32 (m, 1H), 7.18-7.15 (m, 1H), 7.04-7.01 (m, 1H), 6.97-6.92 (m, 1H), 5.46 (s, 2H), 4.58- 4.52 (m, 1H), 4.10 (s, 1H), 3.85 (s, 3H), 2.04- 1.91 (m, 2H), 1.86- 1.62 (m, 4H), 1.52- 1.29 (m, 2H). |
| 35 | 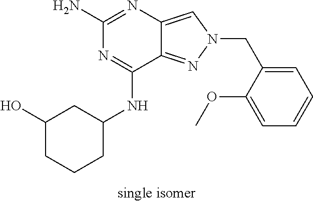 [SMILES: COc1ccccc1Cn1cc2nc(N)nc(NC3CCCC(O)C3)c2n1] | 369.2 | (300 MHz, CD₃OD) δ 7.60 (s, 1H), 7.35-7.30 (m, 1H), 7.16-7.12 (m, 1H), 7.03-6.99 (m, 1H), 6.95-6.90 (m, 1H), 5.45 (s, 2H), 4.19- 4.12 (m, 1H), 3.85 (s, 3H), 3.70-3.61 (m, 1H), 2.31-2.27 (m, 1H), 1.99-1.80 (m, 3H), 1.42-1.21 (m, 4H). |
| 36 | 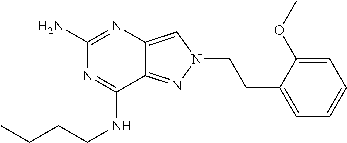 [SMILES: CCCCNc1nc(N)nc2cn(CCc3ccccc3OC)nc12] | 341.1 | (300 MHz, CDCl₃) δ 7.29-7.19 (m, 2H), 6.98-6.95 (m, 1H), 6.88-6.80 (m, 2H), 5.77 (s, 1H), 4.65 (s, 2H), 4.48-4.43 (m, 2H), 3.83 (s, 3H), 3.59- 3.58 (m, 2H), 3.23- 3.18 (m, 2H), 1.72- |
| | | | 1.62 (m, 2H), 1.52- |
| | | | 1.40 (m, 2H), 1.00- |
| | | | 0.95 (m, 3H). |
| 37 | 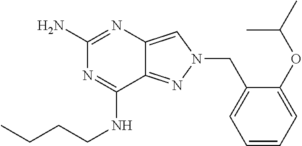 [SMILES: CCCCNc1nc(N)nc2cn(Cc3ccccc3OC(C)C)nc12] | 355.1 | (300 MHz, CDCl₃) δ 7.54 (s, 1H), 7.29-7.23 (m, 1H), 7.06-7.03 (m, 1H), 6.90-6.84 (m, 2H), 5.76 (s, 1H), 5.41 (s, 2H), 4.65-4.57 (m, 3H), 3.57-3.55 (m, 2H), 1.69-1.60 (m, 2H), 1.50-1.32 (m, 8H), 0.98-0.93 (m, |
| | | | 3H). |
| 38 | 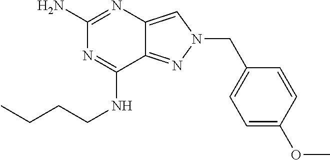 [SMILES: CCCCNc1nc(N)nc2cn(Cc3ccc(OC)cc3)nc12] | 327.1 | (300 MHz, CDCl₃) δ 7.69 (s, 1H), 7.07-7.04 (m, 2H), 6.93-6.90 (m, 2H), 5.58 (s, 2H), 4.71- 4.65 (m, 3H), 3.81 (s, 3H), 3.33-3.27 (m, 2H), 1.34-1.24 (m, 2H), 1.16-1.04 (m, 2H), 0.87-0.82 (m, 3H). |
| 39 | 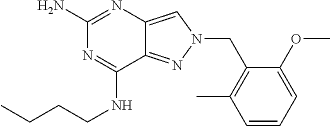 [SMILES: CCCCNc1nc(N)nc2cn(Cc3c(C)cccc3OC)nc12] | 341.2 | (300 MHz, CDCl₃) δ 7.63 (s, 1H), 7.27-7.21 (m, 1H), 6.87-6.78 (m, 2H), 5.69 (s, 1H), 5.49 (s, 2H), 4.59-4.53 (m, 2H), 3.87 (s, 3H), 3.58- 3.46 (m, 2H), 2.41 (s, 3H), 1.71-1.61 (m, |
| | | | 2H), 1.57-1.49 (m, |
| | | | 2H), 0.99-0.89 (m, |
| | | | 3H). |
| 40 | 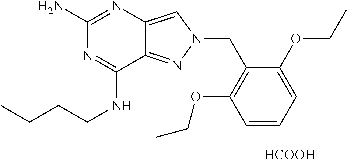 [SMILES: CCCCNc1nc(N)nc2cn(Cc3c(OCC)cccc3OCC)nc12 ; "HCOOH"] | 385.1 | (300 MHz, CDCl₃) δ 8.70 (s, 1H), 7.39 (s, 1H), 7.32-7.26 (m, 1H), 6.58-6.55 (m, 2H), 6.46 (s, 1H), 5.51 (s, 2H), 4.02-4.00 (m, 4H), 3.66-3.59 (m, 2H), 1.73-1.64 (m, 2H), 1.52-1.42 (m, 2H), 1.37-1.33 (m, |
| | | | 6H), 1.01-0.97 (m, |
| | | | 3H). |
| 41 | 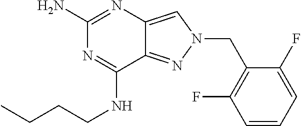 [SMILES: CCCCNc1nc(N)nc2cn(Cc3c(F)cccc3F)nc12] | 333.1 | (300 MHz, CDCl₃) δ 7.53 (s, 1H), 7.38-7.28 (m, 1H), 6.97-6.92 (m, 2H), 5.80 (s, 1H), 5.49 (s, 2H), 4.69 (s, 2H), 3.55-3.53 (m, 2H), 1.69-1.59 (m, 2H), 1.55-1.51 (m, 2H), |
| | | | 0.97-0.89 (m, 3H). |
| 42 | 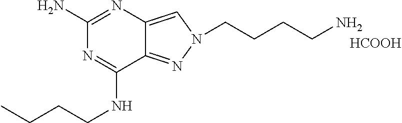 [SMILES: CCCCNc1nc(N)nc2cn(CCCCN)nc12 ; "HCOOH"] | 278.2 | (400 MHz, CD₃OD) δ 8.78 (s, 1H), 7.93 (s, 1H), 4.44-4.40 (m, 2H), 3.67-3.64 (m, 2H), 2.98-2.94 (m, 2H), 2.07-2.04 (m, 2H), 1.74-1.64 (m, 4H), 1.49-1.40 (m, |
| | | | 2H), 1.02-0.95 (m, |
| | | | 3H). |

### Example 6

### Preparation of Compound 43

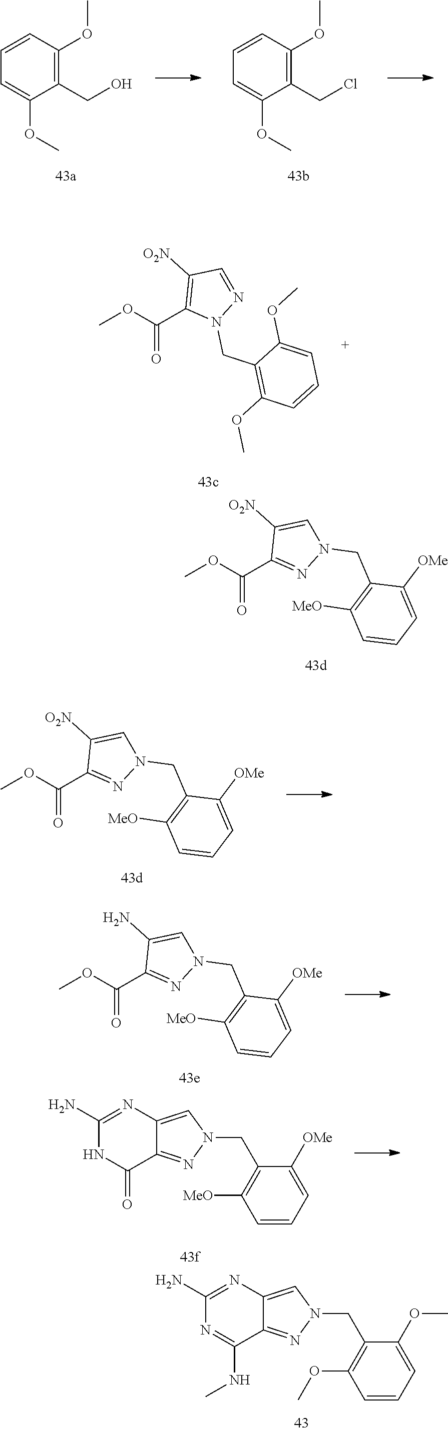

### Step A—Synthesis of Compound 43b

To a solution of NCS (437 mg, 3.27 mmol) in DCM (5 mL) at 0° C. under a nitrogen atmosphere was added dimethylsulfide (222 mg, 3.57 mmol) dropwise. The resulting reaction was allowed to stir at 0° C. for 5 minutes, then cooled to −20° C., and a solution of compound 43a (500 mg, 2.97 mmol) in DCM (1 mL) was added. The resulting reaction was warmed to 0° C. slowly, and allowed to stir for an additional 2 hours at 0° C. The reaction mixture was then poured into cold brine (10 mL), and extracted with diethyl ether (3×15 mL). The combined organic extracts were dried over Na₂SO₄, filtered, and concentrated in vacuo to provide compound 43b, which was used without further purification. MS: m/z=151.2 [M−Cl].

### Step B—Synthesis of Compound 43c and 43d

Compound 43c and 43d (in a ratio of 1:8) were made using the method described in Example 5, step D, and substituting the appropriate reactants and/or reagents. MS: m/z=322.2 [M+H].

### Step C—Synthesis of Compound 43e

To a solution of compound 43d (3 g, 9.34 mmol) in EtOAc (60 mL) was added Pd/C (10% wt %, 600 mg). The reaction mixture was degassed with nitrogen 3 times and allowed to stir under hydrogen (1 atm) for 3 hours at room temperature. The reaction mixture was filtered through Celite, and the filtrate was concentrated in vacuo to provide compound 43e, which was used without further purification. MS: m/z=292.2 [M+H].

### Step D—Synthesis of Compound 43f

A mixture of compound 43e (1 g, 3.43 mmol), TEA (0.868 g, 8.58 mmol), carbamimidic chloride hydrochloride (0.987 g, 8.58 mmol), and tetrahydrothiophene 1,1-dioxide (2.06 g, 17.2 mmol) in a sealed tube was heated to 120° C., and allowed to stir for 30 minutes at this temperature. The reaction mixture was quenched with water (16 mL), and the resulting solution was cooled in an ice bath, and the pH was adjusted to 8-9 with dropwise addition of NH₃·H₂O. The resulting suspension was left at room temperature for 15 hours, and then filtered. The collected solid was washed with EtOH/Et₂O (1/10), and dried in vacuo to provide compound 43f MS: m/z=302.2 [M+H].

### Step E—Synthesis of Compound 43

To a mixture of compound 43f (20 mg, 0.066 mmol), and PyBOP (44.9 mg, 0.086 mmol) in DMF (0.5 mL), at 0° C., was added DBU (0.015 ml, 0.100 mmol). The resulting reaction was allowed to stir for 10 minutes at 0° C. Then, a solution of methanamine (6.18 mg, 0.199 mmol) in DMF (0.1 mL) was added, and the resulting reaction was warmed to 20° C., and allowed to stir at this temperature for 3 hours. The reaction mixture was then directly purified using RP-flash chromatography (eluted with 0-40% acetonitrile in 10 mM aq. NH₄HCO₃) to provide compound 43. MS: m/z=315.2 [M+H]. ¹H NMR (300 MHz, d₆-DMSO) δ 7.69 (s, 1H), 7.38-7.32 (m, 2H), 6.74-6.71 (m, 2H), 5.70 (s, 2H), 5.39 (s, 2H), 3.79 (s, 6H), 2.88-2.87 (m, 3H).

The following compounds of the present invention were made using the methods described in Example 6 above, and substituting the appropriate reactants and/or reagents:

| | | MS | |
|---|---|---|---|
| Compound | Structure | [M + H] | ¹H NMR |
| 44 | 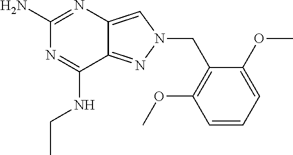 [SMILES: CCNc1nc(N)nc2cn(Cc3c(OC)cccc3OC)nc12] | 329.1 | (300 MHz, d₆- DMSO) δ 7.60 (s, 1H), 7.39-7.32 (m, 2H), 6.75- 6.72 (m, 2H), 5.51 (s, 2H), 5.39 (s, 2H), 3.80 (s, 6H), 3.46-3.42 (m, 2H), 1.18-1.13 (m, 3H). |
| 45 | 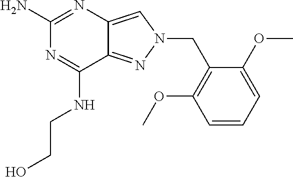 [SMILES: COc1cccc(OC)c1Cn1cc2nc(N)nc(NCCO)c2n1] | 345.2 | (300 MHz, d₆- DMSO) δ 7.84 (s, 1H), 7.40-7.33 (m, 2H), 6.74- 6.71 (m, 2H), 6.06 (s, 2H), 5.40 (s, 2H), 4.75 (s, 1H), 3.79 (s, 6H), 3.57- 3.50 (m, 4H). |
| 46 | 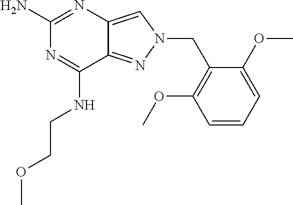 [SMILES: COCCNc1nc(N)nc2cn(Cc3c(OC)cccc3OC)nc12] | 359.2 | (300 MHz, d₆- DMSO) δ 7.52 (s, 1H), 7.39-7.33 (m, 2H), 6.75- 6.72 (m, 2H), 5.59 (s, 2H), 5.40 (s, 2H), 3.81 (s, 6H), 3.59-3.51 (m, 4H), 3.27 (s, 3H). |
| 47 | 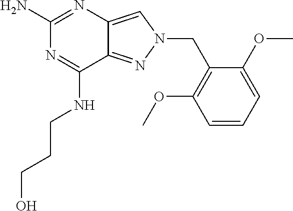 [SMILES: COc1cccc(OC)c1Cn1cc2nc(N)nc(NCCCO)c2n1] | 359.2 | (300 MHz, d₆- DMSO) δ 7.95 (s, 1H), 7.38-7.33 (m, 2H), 6.74- 6.71 (m, 2H), 6.01 (s, 2H), 5.39 (s, 2H), 4.52 (s, 1H), 3.79 (s, 6H), 3.55- 3.50 (m, 4H), 1.77- 1.68 (m, 2H). |
| 48 | 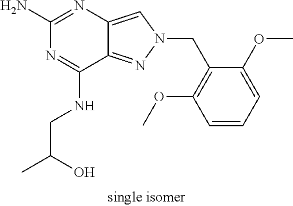 [SMILES: COc1cccc(OC)c1Cn1cc2nc(N)nc(NCC(C)O)c2n1] | 359.1 | (300 MHz, d₆- DMSO) δ 7.37- 7.31 (m, 2H), 7.24 (s, 1H), 6.73-6.71 (m, 2H), 5.45 (s, 2H), 5.38 (s, 2H), 4.88 (s, 1H), 3.87- 3.79 (m, 7H), 3.44- 3.40 (m, 1H), 3.31-3.25 (m, 1H), 1.07-1.05 (m, 3H). |
| 49 | 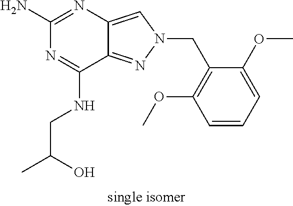 [SMILES: COc1cccc(OC)c1Cn1cc2nc(N)nc(NCC(C)O)c2n1] | 359.1 | (300 MHz, d₆- DMSO) δ 7.37- 7.32 (m, 2H), 7.26 (s, 1H), 6.73-6.71 (m, 2H), 5.47 (s, 2H), 5.38 (s, 2H), 4.88 (s, 1H), 3.88- 3.79 (m, 7H), 3.45- 3.38 (m, 1H), 3.31-3.25 (m, 1H), 1.07-1.05 (m, 3H). |
| 50 | 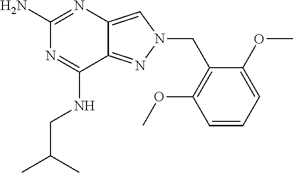 [SMILES: COc1cccc(OC)c1Cn1cc2nc(N)nc(NCC(C)C)c2n1] | 357.1 | (300 MHz, d₆- DMSO) δ 7.55 (s, 1H), 7.38-7.32 (m, 2H), 6.74- 6.71 (m, 2H), 5.42 (s, 2H), 5.38 (s, 2H), 3.79 (s, 6H), 3.23-3.19 (m, 2H), 2.01-1.96 (m, 1H), 0.88- 0.86 (m, 6H). |
| 51 | 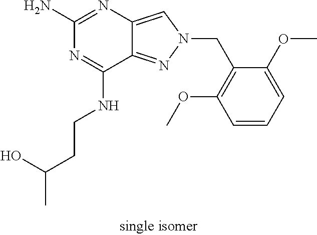 [SMILES: COc1cccc(OC)c1Cn1cc2nc(N)nc(NCCC(C)O)c2n1] | 373.1 | (300 MHz, d₆- DMSO) δ 7.53 (s, 1H), 7.37-7.29 (m, 2H), 6.73- 6.71 (m, 2H), 5.50 (s, 2H), 5.37 (s, 2H), 4.64 (s, 1H), 3.79 (s, 6H), 3.70- 3.66 (m, 1H), 3.54- 3.41 (m, 2H), 1.69-1.52 (m, 2H), 1.07-1.05 (m, 3H). |
| 52 | 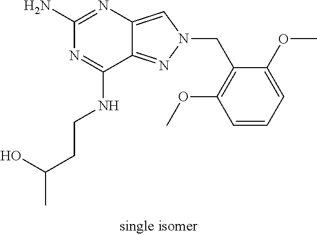 [SMILES: COc1cccc(OC)c1Cn1cc2nc(N)nc(NCCC(C)O)c2n1] | 373.1 | (300 MHz, d₆- DMSO) δ 7.52 (s, 1H), 7.34-7.29 (m, 2H), 6.73- 6.70 (m, 2H), 5.42 (s, 2H), 5.37 (s, 2H), 4.64 (s, 1H), 3.79 (s, 6H), 3.70- 3.66 (m, 1H), 3.54- 3.35 (m, 2H), 1.69-1.52 (m, 2H), 1.08-1.06 (m, 3H). |
| 53 | 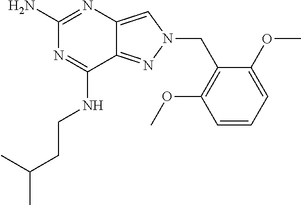 [SMILES: COc1cccc(OC)c1Cn1cc2nc(N)nc(NCCC(C)C)c2n1] | 371.3 | (300 MHz, d₆- DMSO) δ 7.48 (s, 1H), 7.37-7.32 (m, 2H), 6.74- 6.71 (m, 2H), 5.39- 5.38 (m, 4H), 3.79 (s, 6H), 3.42- 3.32 (m, 2H), 1.65-1.56 (m, 1H), 1.50-1.43 (m, 2H), 0.88- 0.86 (m, 6H). |
| 54 | 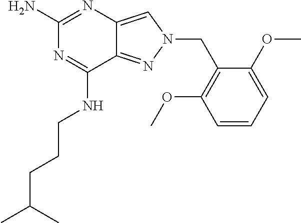 [SMILES: COc1cccc(OC)c1Cn1cc2nc(N)nc(NCCCC(C)C)c2n1] | 385.2 | (300 MHz, d₆- DMSO) δ 7.53 (s, 1H), 7.40-7.30 (m, 2H), 6.76- 6.73 (m, 2H), 5.39 (s, 4H), 3.81 (s, 6H), 3.42-3.32 (m, 2H), 1.58- 1.51 (m, 3H), 1.24- 1.16 (m, 2H), 0.88-0.86 (m, 6H). |
| 55 | 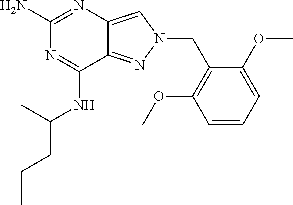 [SMILES: CCCC(C)Nc1nc(N)nc2cn(Cc3c(OC)cccc3OC)nc12] | 371.1 | (300 MHz, d₆- DMSO) δ 7.38- 7.26 (m, 3H), 6.74- 6.71 (m, 2H), 5.47-5.38 (m, 4H), 4.35-4.33 (m, 1H), 3.79 (s, 6H), 1.64-1.23 (m, 4H), 1.15- 1.13 (m, 3H), 0.88- 0.86 (m, 3H). |
| 56 | 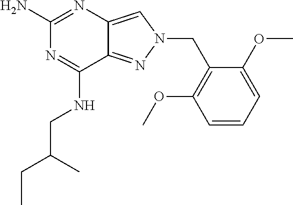 [SMILES: CCC(C)CNc1nc(N)nc2cn(Cc3c(OC)cccc3OC)nc12] | 371.3 | (300 MHz, d₆- DMSO) δ 7.56 (s, 1H), 7.38-7.27 (m, 2H), 6.74- 6.71 (m, 2H), 5.47- 5.38 (m, 4H), 3.79 (s, 6H), 3.24- 3.16 (m, 2H), 1.89-1.77 (m, 1H), 1.44-1.37 (m, 1H), 1.26- 1.23 (m, 1H), 0.88- 0.86 (m, 6H). |
| 57 | 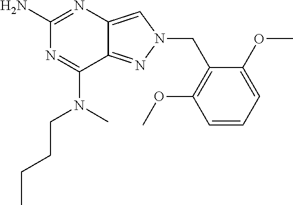 [SMILES: CCCCN(C)c1nc(N)nc2cn(Cc3c(OC)cccc3OC)nc12] | 371.2 | (300 MHz, CD₃OD) δ 7.41 (s, 1H), 7.36-7.30 (m, 1H), 6.70- 6.67 (m, 2H), 5.49 (s, 2H), 4.12-4.11 (m, 1H), 3.85- 3.84 (m, 7H), 3.24 (s, 3H), 1.69-1.59 (m, 2H), 1.38- 1.31 (m, 2H), 0.98- 0.86 (m, 3H). |
| 91 | 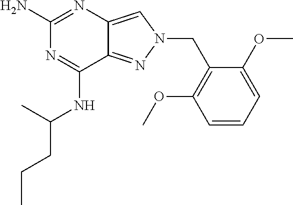 [SMILES: CCCC(C)Nc1nc(N)nc2cn(Cc3c(OC)cccc3OC)nc12] | 371.3 | (300 MHz, d₆- DMSO) δ 7.41- 7.23 (m, 3H), 6.77- 6.70 (m, 2H), 5.48 (s, 2H), 5.38 (s, 2H), 4.40- 4.26 (m, 1H), 3.79 (s, 6H), 1.69- 1.22 (m, 4H), 1.19- 1.10 (m, 3H), 0.91-0.82 (m, 3H). |
| 107 | 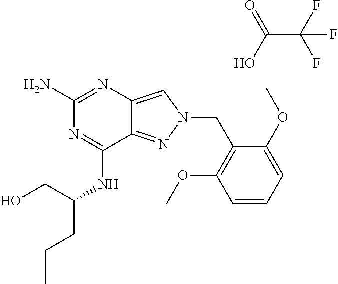 | 387.3 | (300 MHz, CDCl₃) δ 7.48 (s, 1H), 7.38- 7.32 (m, 1H), 6.90-6.87 (m, 1H), 6.63-6.60 (m, 2H), 5.52 (s, 2H), 4.41 (s, 1H), 3.85-3.72 (m, 8H), 1.70-1.61 (m, 2H), 1.48- 1.38 (m, 2H), 0.98- 0.93 (m, 3H). |
| 108 | 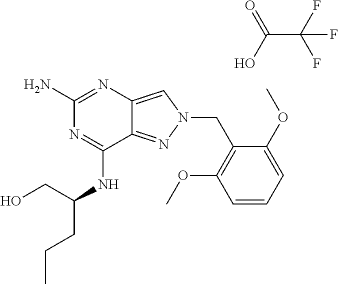 | 387.3 | (300 MHz, CDCl₃) δ 7.49 (s, 1H), 7.38- 7.32 (m, 1H), 6.99-6.96 (m, 1H), 6.63-6.60 (m, 2H), 5.52 (s, 2H), 4.39 (s, 1H), 3.87-3.70 (m, 8H), 1.68-1.59 (m, 2H), 1.45- 1.38 (m, 2H), 0.98- 0.93 (m, 3H). |
| 113 | 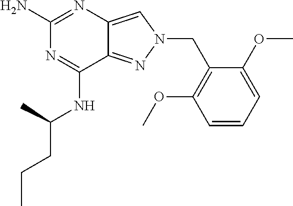 [SMILES: CCC[C@@H](C)Nc1nc(N)nc2cn(Cc3c(OC)cccc3OC)nc12] | 371.3 | (300 MHz, CDCl₃) δ 7.43 (s, 1H), 7.36- 7.30 (m, 1H), 6.61-6.58 (m, 2H), 5.96-5.94 (m, 1H), 5.48 (s, 2H), 4.42-4.39 (m, 1H), 3.84 (s, 6H), 1.68-1.65 (m, 2H), 1.33- 1.26 (m, 2H), 1.33- 1.26 (m, 3H), 0.97-0.92 (m, 3H). |
| 124 | 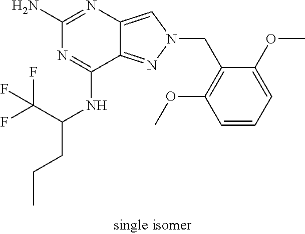 [SMILES: CCCC(Nc1nc(N)nc2cn(Cc3c(OC)cccc3OC)nc12)C(F)(F)F] | 425.3 | (400 MHz, CDCl₃) δ 7.47 (s, 1H), 7.34- 7.26 (m, 1H), 6.61-6.58 (m, 2H), 5.85 (brs, 1H), 5.51 (s, 2H), 5.13-5.10 (m, 1H), 4.93 (brs, 2H), 3.84 (s, 6H), 1.87-1.75 (m, 2H), 1.71-1.66 (m, 2H), 0.99- 0.97 (m, 3H). |
| 126 | 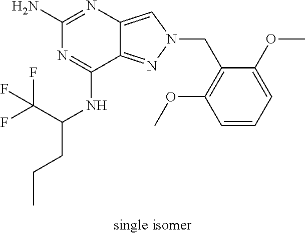 [SMILES: CCCC(Nc1nc(N)nc2cn(Cc3c(OC)cccc3OC)nc12)C(F)(F)F] | 425.3 | (400 MHz, CDCl₃) δ 7.47 (s, 1H), 7.33- 7.29 (m, 1H), 6.60-6.58 (m, 2H), 5.84 (brs, 1H), 5.51 (s, 2H), 5.15-5.11 (m, 1H), 4.83 (brs, 2H), 3.84 (s, 6H), 1.92-1.83 (m, 2H), 1.69-1.64 (m, 2H), 0.99- 0.97 (m, 3H) |
| 130 | 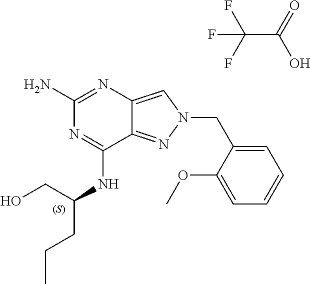 | 357.3 | (300 MHz, CD₃OD) δ 7.81 (s, 1H), 7.39-7.33 (m, 1H), 7.26- 7.24 (m, 1H), 7.04- 6.93 (m, 2H), 5.51 (s, 2H), 4.56- 4.51 (m, 1H), 3.85 (s, 3H), 3.72-3.61 (m, 2H), 1.70- 1.60 (m, 2H), 1.48- 1.36 (m, 2H), 0.98-0.89 (m, 3H) |
| 133 | 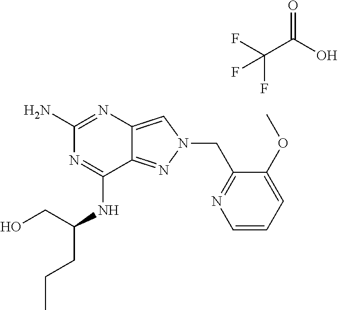 | 358.1 | (300 MHz, CD₃OD) δ 8.15- 8.13 (m, 1H), 7.94- 7.92 (m, 1H), 7.60-7.59 (m, 1H), 7.48-7.45 (m, 1H), 5.70- 5.67 (m, 2H), 4.56- 4.52 (m, 1H), 3.95 (s, 3H), 3.69- 3.61 (m, 2H), 1.69- 1.62 (m, 2H), 1.47-1.37 (m, 2H), 0.97-0.94 (m, 3H) |
| 135 | 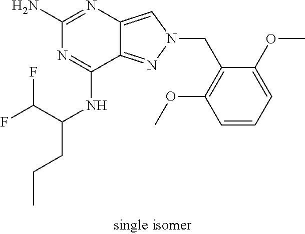 [SMILES: CCCC(Nc1nc(N)nc2cn(Cc3c(OC)cccc3OC)nc12)C(F)F] | 407.3 | (300 MHz, CDCl₃) δ 7.49 (s, 1H), 7.35- 7.31 (m, 1H), 6.63-6.60 (m, 2H), 6.11-5.82 (m, 2H), 5.53 (s, 2H), 4.79-4.76 (m, 3H), 3.86 (s, 6H), 1.67-1.23 (m, 4H), 1.01- 0.98 (m, 3H) |
| 136 | 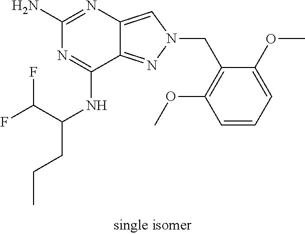 [SMILES: CCCC(Nc1nc(N)nc2cn(Cc3c(OC)cccc3OC)nc12)C(F)F] | 407.3 | (300 MHz, CDCl₃) δ 7.49 (s, 1H), 7.35- 7.31 (m, 1H), 6.63-6.60 (m, 2H), 6.11-5.82 (m, 2H), 5.53 (s, 2H), 4.79-4.76 (m, 3H), 3.86 (s, 6H), 1.67-1.24 (m, 4H), 1.01- 0.98 (m, 3H). |
| 184 | 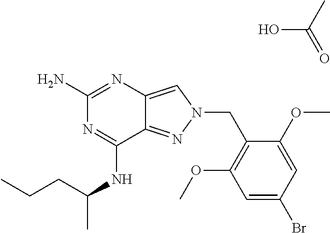 | 449.1 | (400 MHz, CD₃OD) δ 7.62 (s, 1H), 6.93 (s, 2H), 5.79 (s, 2H), 4.46- 4.41 (m, 1H), 3.88 (s, 6H), 1.68-1.60 (m, 2H), 1.45- 1.40 (m, 2H), 1.28- 1.26 (m, 3H), 0.99-0.95 (m, 3H). |

### Example 7

### Preparation of Compound 58

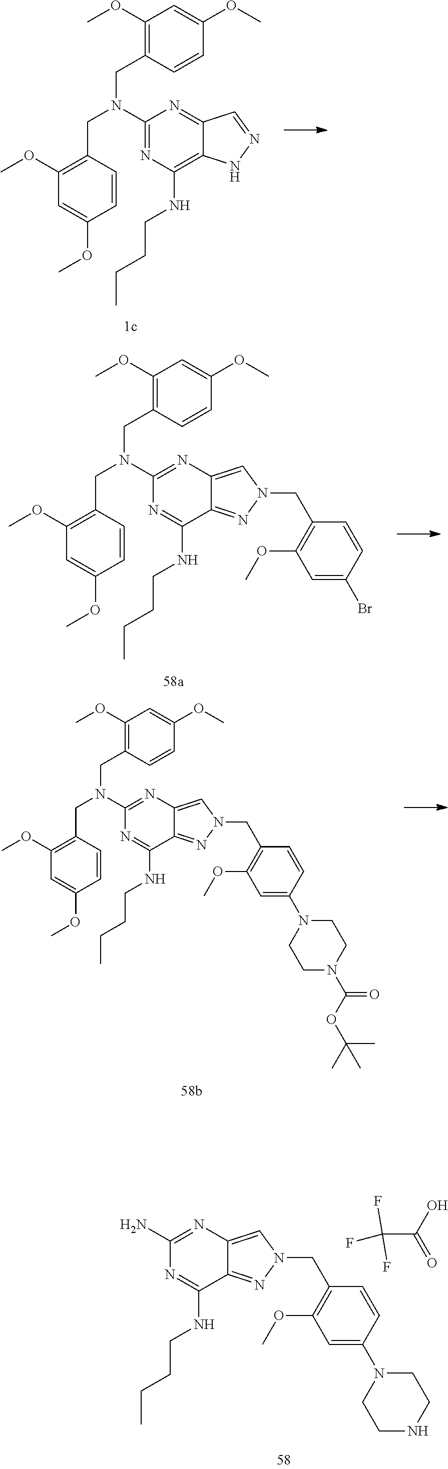

### Step A—Synthesis of Compound 58a

To a stirred solution of compound 1c (400 mg, 0.790 mmol) in acetonitrile (3 mL) was added 4-bromo-1-(bromomethyl)-2-methoxybenzene (530 mg, 1.89 mmol), followed by potassium carbonate (218 mg, 1.579 mmol). The resulting reaction was heated to 70° C., allowed to stir at this temperature for 10 minutes, and then cooled to room temperature, and allowed to stir overnight. The reaction mixture was then concentrated in vacuo, and the resulting residue was purified using silica gel chromatography (40 gram ISCO Redisep Gold column, eluent 0-100% EtOAc/Hexanes) to provide compound 58a. MS: m/z=707.2 [M+H].

### Step B—Synthesis of Compound 58b

A vial was charged with compound 58a (100 mg, 0.142 mmol), 1-boc-piperazine (31.7 mg, 0.170 mmol), and methanesulfonato(2-dicyclohexylphosphino-2′,6′-di-i-propoxy-1,1′-biphenyl)(2′-amino-1,1′-biphenyl-2-yl)palladium(II) (11.9 mg, 0.014 mmol). The vial was capped and put under nitrogen atmosphere. 1 M LHMDS in THF (2126 μl, 0.213 mmol) was then added, and the resulting reaction was heated to 80° C., and allowed to stir at this temperature for 10 minutes. The reaction mixture was then cooled to room temperature, filtered, and the collected solid was dried in vacuo, and the resulting residue was purified using silica gel chromatography (24 gram ISCO Redisep Gold column, eluent 0-100% EtOAc/Hexanes) to provide compound 58b. MS: m/z=811.4 [M+H].

### Step C—Synthesis of Compound 58

To a stirred solution of compound 58b (56 mg, 0.069 mmol) in DCE (691 μl) was added TFA (160 μl, 2.072 mmol). The resulting reaction was allowed to stir at room temperature overnight and was then concentrated in vacuo. The residue obtained was purified using reverse phase HPLC (Waters XBridge C18 10 μm 19 mm×250 mm column (eluent 10-70% acetonitrile/water with 0.05% TFA) to provide compound 58. MS: m/z=411.2 [M+H]. ¹H NMR (500 MHz, Methanol-d₄) δ 7.74 (s, 1H), 7.23 (d, J=8.3 Hz, 1H), 6.68 (d, J=2.1 Hz, 1H), 6.61 (dd, J=8.3, 2.2 Hz, 1H), 5.42 (s, 2H), 3.87 (s, 3H), 3.63 (t, J=7.2 Hz, 2H), 3.51-3.46 (m, 4H), 3.38 (d, J=5.1 Hz, 4H), 1.73-1.65 (m, 2H), 1.51-1.39 (m, 2H), 0.99 (t, J=7.4 Hz, 3H).

The following compounds of the present invention were made using the methods described in Example 7 above, and substituting the appropriate reactants and/or reagents:

| | | MS | |
|---|---|---|---|
| Compound | Structure | [M + H] | ¹H NMR |
| 59 | 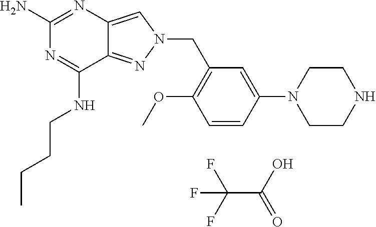 | 411.2 | (500 MHz, Methanol- d₄) δ 7.81 (s, 1H), 7.07 (dd, J = 8.8, 2.9 Hz, 1H), 7.05 − 6.99 (m, 2H), 5.49 (s, 2H), 3.84 (s, 3H), 3.64 (t, J = 7.2 Hz, 2H), 3.37 (dd, J = 6.3, 3.5 Hz, 4H), 3.30 (dd, J = 6.5, 3.4 Hz, 4H), 1.69 (q, J = 7.5 Hz, 2H), 1.50 − 1.37 (m, 2H), 0.99 (t, J = 7.4 Hz, 3H). |
| 116 | 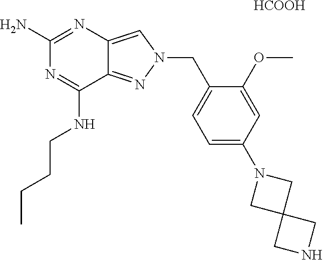 [SMILES: "HCOOH" ; CCCCNc1nc(N)nc2cn(Cc3ccc(N4CC5(CNC5)C4)cc3OC)nc12] | 423.1 | (300 MHz, CD₃OD) δ 8.51 (s, 1H), 7.71 − 7.65 (m, 1H), 7.17 − 7.11 (m, 1H), 6.12 − 6.05 (m, 2H), 5.44 (s, 2H), 4.92 − 4.90 (m, 1H), 4.27 (s, 3H), 4.13 − 4.09 (m, 1H), 4.09 − 3.81 (m, 3H), 3.81 − 3.60 (m, 3H), 3.60 − 3.53 (m, 2H), 1.72 − 1.62 (m, 2H), 1.48 − 1.36 (m, 2H), 0.97 − 0.91 (m, 3H). |
| 131 | 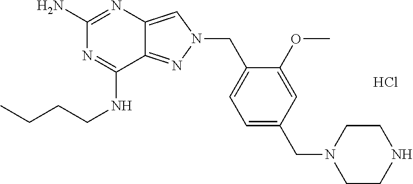 [SMILES: CCCCNc1nc(N)nc2cn(Cc3ccc(CN4CCNCC4)cc3OC)nc12 ; Cl] | 425.2 | (300 MHz, DMSO) δ 7.95 (s, 1H), 7.44 (s, 1H), 7.35 − 7.33 (m, 1H), 7.20 − 7.18 (m, lH), 5.58 (s, 2H), 4.44 (s, 2H), 3.98 (m, 3H), 3.71 − 3.62 (m, 7H), 3.62 − 3.50 (m, 3H), 1.73 − 1.65 (m, 2H), 1.56 − 1.46 (m, 2H), 1.01 − 0.92 (m, 3H). |
| 132 | 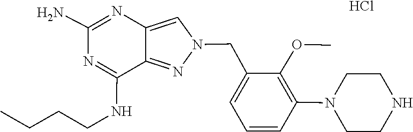 [SMILES: Cl ; CCCCNc1nc(N)nc2cn(Cc3cccc(N4CCNCC4)c3OC)nc12] | 411.2 | (300 MHz, CD₃OD) δ 7.78 (s, 1H), 7.05 − 7.00 (m, 2 H), 6.98 − 6.87 (m, 1H), 5.44 (s, 2H), 3.75 (s, 3H), 3.55 − 3.50 (m, 2H), 3.32 − 3.20 (m, 8H), 1.63 − 1.57 (m, 2H), 1.35 − 1.25 (m, 2H), 0.90 − |
| | | | 0.87 (m, 3H). |
| 137 | 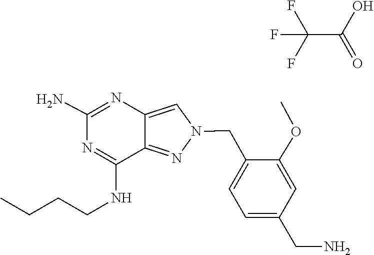 | 356.2 | (300 MHz, CD₃OD) δ 7.84 (s, 1H), 7.32 − 7.29 (m, 1H), 7.16 (s, 1H), 7.07 − 7.01 (m, 1H), 5.54 (s, 2H), 4.13 (s, 2H), 3.90 (s, 3H), 3.66 − 3.61 (m, 2H), 1.71 − 1.66 (m, 2H), 1.47 − 1.42 (m, 2H), 1.01 − 0.99 (m, 3H). |
| 146 | 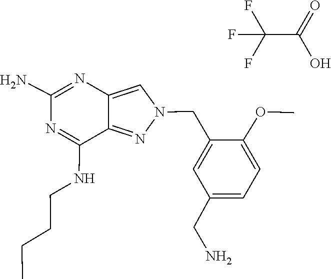 [SMILES: O=C(O)C(F)(F)F ; CCCCNc1nc(N)nc2cn(Cc3cc(CN)ccc3OC)nc12] | 356.2 | (300 MHz, CD₃OD) δ 7.85 (s, 1H), 7.50 − 7.47 (m, 1H), 7.33 (s, 1H), 7.15 − 7.12 (m, 1H), 5.57 (s, 2H), 4.05 (s, 2H), 3.91 (s, 3H), 3.67 − 3.55 (m, 2H), 1.74 − 1.69 (m, 2H), 1.50 − 1.37 (m, 2H), 1.01 − 0.99 (m, 3H). |
| 150 | 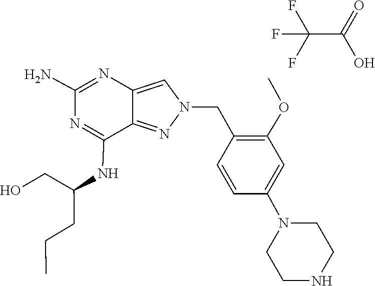 | 441.2 | (300 MHz, CD₃OD) δ 7.78 (s, 1H), 7.26 − 7.23 (m, 1H), 6.68 − 6.63 (m, 1H), 6.61 − 6.60 (m, 1H), 5.52 (s, 2H), 4.87 − 4.53 (m, 1H), 3.87 (s, 3H), 3.74 − 3.70 (m, 2H), 3.47 − 3.32 (m, 8H), 1.72 − 1.65 (m, 2H), 1.48 − 1.39 (m, 2H), 1.01 − 0.99 (m, 3H). |
| 191 | 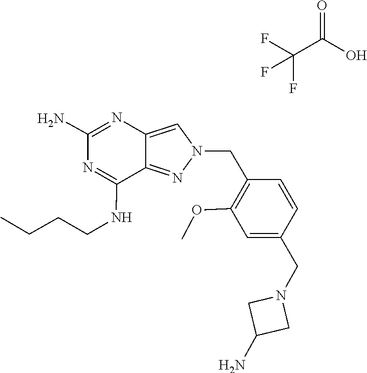 [SMILES: O=C(O)C(F)(F)F ; CCCCNc1nc(N)nc2cn(Cc3ccc(CN4CC(N)C4)cc3OC)nc12] | 411.3 | (300 MHz, CD₃OD) δ 7.86 (s, 1H), 7.30 − 7.26 (m, 1H), 7.18 (s, 1H), 7.08 − 7.05 (m, 1H), 5.52 (s, 2H), 4.47 − 4.32 (m, 7H), 3.96 (s, 3H), 3.64 − 3.59 (m, 2H), 1.71 − 1.61 (m, 2H), 1.47 − 1.44 (m, 2H), 1.02 − 0.97 (m, 3H). |
| 192 | 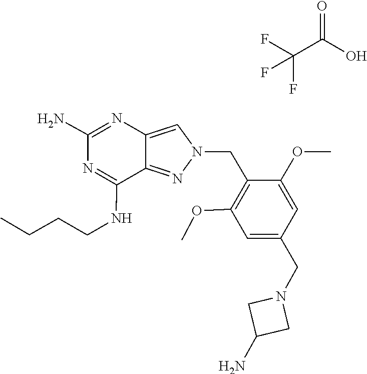 [SMILES: O=C(O)C(F)(F)F ; CCCCNc1nc(N)nc2cn(Cc3c(OC)cc(CN4CC(N)C4)cc3OC)nc12] | 441.2 | (300 MHz, CD₃OD) δ 7.74 (s, 1H), 6.87 (s, 2H), 5.56 (s, 2H), 4.87 − 4.38 (m, 7H), 3.92 (s, 6H), 3.65 − 3.60 (m, 2H), 1.73 − 1.63 (m, 2H), 1.48 − 1.44 (m, 2H), 1.01 − 0.97 (m, 3H). |
| 199 | 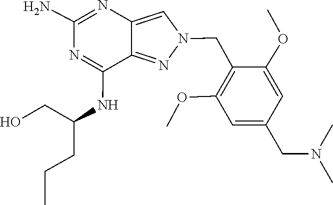 [SMILES: CCC[C@@H](CO)Nc1nc(N)nc2cn(Cc3c(OC)cc(CN(C)C)cc3OC)nc12] | 444.2 | (300 MHz, CD₃OD) δ 7.65 (s, 1H), 6.74 (s, 2H), 5.53 (s, 2H), 4.47 − 4.45 (m, 1H), 3.89 (s, 6H), 3.72 − 3.71 (m, 2H), 3.64 − 3.62 (m, 2H), 2.35 (s, 6H), 1.73 − 1.70 (m, 2H), 1.68 − 1.67 (m, 2H), 0.99 − 0.98 (m, 3H). |
| 213 | 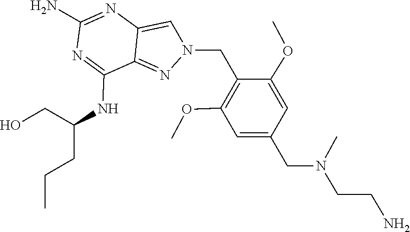 [SMILES: CCC[C@@H](CO)Nc1nc(N)nc2cn(Cc3c(OC)cc(CN(C)CCN)cc3OC)nc12] | 473.3 | (300 MHz, CD₃OD) δ 7.79 (s, 1H), 7.08 (s, 2H), 5.56 (s, 2H), 4.52 − 4.46 (m, 3H), 3.94 (s, 6H), 3.68 (m, 2H), 3.52 − 3.44 (m, 4H), 2.88 (s, 3H), 1.72 − 1.70 (m, 2H), 1.67 − 1.64 (m, 2H), 1.00 − 0.95 (m, 3H). |

### Example 8

### Preparation of Compound 60

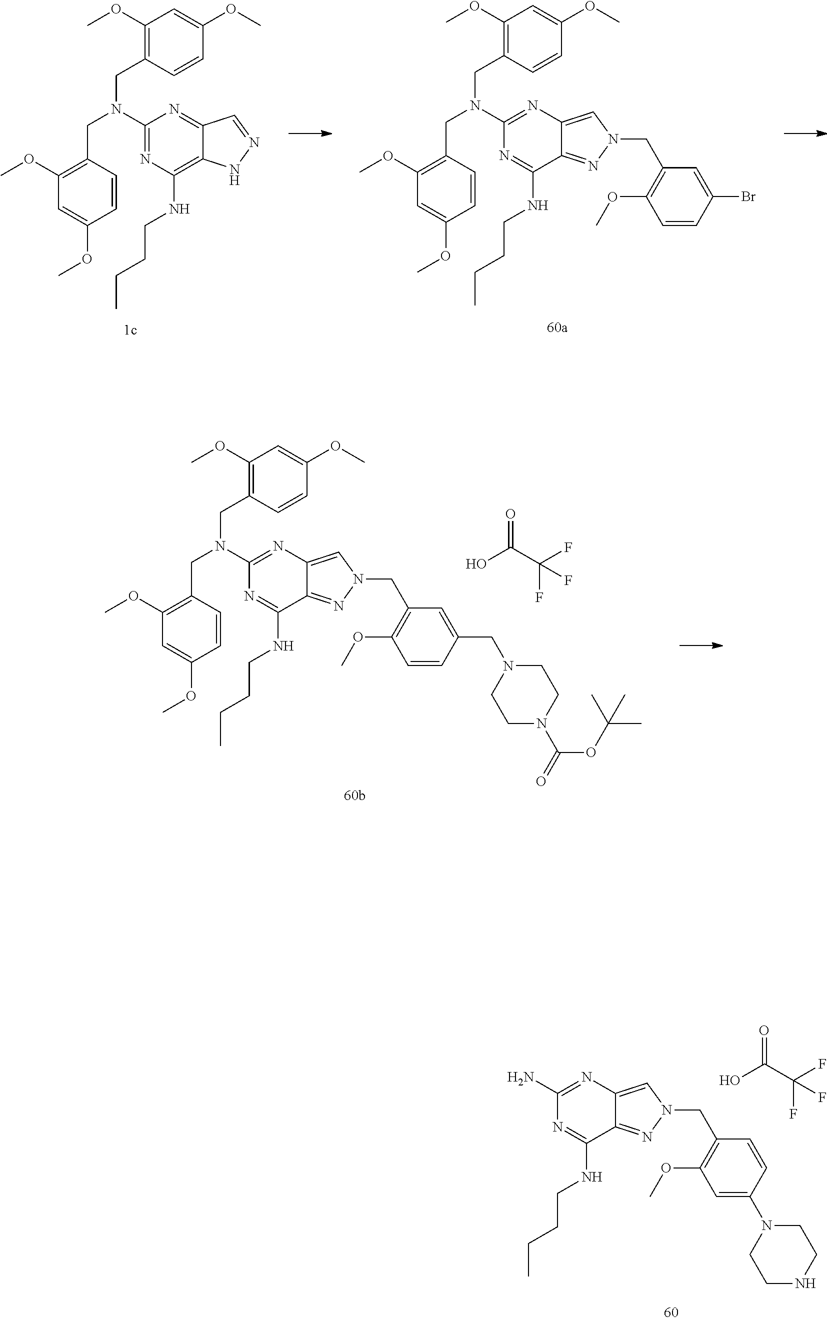

### Step A—Synthesis of Compound 60a

Compound 60a was made from compound 1c, using the method described in Example 7, step B, and substituting the appropriate reactants and/or reagents. MS: m/z=707.2 [M+H].

### Step B—Synthesis of Compound 60b

To a stirred solution of compound 60a (110 mg, 0.156 mmol) in THF (1.403 mL) was added potassium (4-tert-butoxycarbonylpiperazin-1-yl)methyltrifluoroborate (71.6 mg, 0.234 mmol), followed by chloro(2-dicyclohexylphosphino-2′,4′,6′-triisopropyl-1,1′-biphenyl)[2-(2′-amino-1,1′-biphenyl)]palladium(II) (12.27 mg, 0.016 mmol). To the resulting solution was added a solution of Cs₂CO₃ (102 mg, 0.312 mmol) in water (156 μl). The resulting reaction was then heated to 90° C., and allowed to stir at this temperature for 1 hour. The reaction mixture was cooled to room temperature and filtered. The collected solid was dried in vacuo, and purified using reverse phase HPLC (Waters XBridge C18 10 μm 19 mm*250 mm column, eluent 10-70% acetonitrile/water with 0.05% TFA) to provide compound 60b. MS: m/z=825.4 [M+H].

### Step C—Synthesis of Compound 60

To a stirred solution of compound 60b (111 mg, 0.118 mmol) in DCE (1180 μl) was added TFA (91 μl, 1.18 mmol), and the resulting reaction was allowed to stir at room temperature for 5 hours. The reaction mixture was concentrated in vacuo, and the resulting residue was purified using reverse phase HPLC (Waters XBridge C18 10 μm 19 mm*250 mm column, eluent 10-70% acetonitrile/water with 0.05% TFA) to provide compound 60. MS: m/z=425.2 [M+H]. ¹H NMR (500 MHz, Methanol-d₄) δ 7.84 (s, 1H), 7.52 (dd, J=8.5, 2.1 Hz, 1H), 7.41 (d, J=2.1 Hz, 1H), 7.15 (d, J=8.5 Hz, 1H), 5.53 (s, 2H), 4.25 (s, 2H), 3.91 (s, 3H), 3.64 (t, J=7.2 Hz, 2H), 3.55-3.48 (m, 4H), 3.40 (s, 4H), 1.69 (q, J=7.5 Hz, 2H), 1.48-1.39 (m, 2H), 0.99 (t, J=7.4 Hz, 3H).

The following compounds of the present invention were made using the methods described in Example 8 above, and substituting the appropriate reactants and/or reagents:

| | | MS | |
|---|---|---|---|
| Compound | Structure | [M + H] | ¹H NMR |
| 61 | 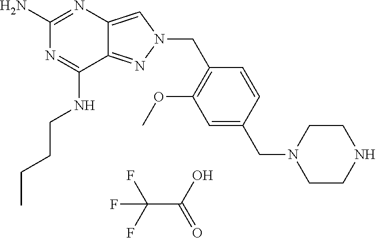 [SMILES: CCCCNc1nc(N)nc2cn(Cc3ccc(CN4CCNCC4)cc3OC)nc12 ; O=C(O)C(F)(F)F] | 425.2 | ¹H NMR (500 MHz, Methanol-d₄) δ 7.84 (s, 1H), 7.31 (d, J = 7.7 Hz, 1H), 7.20 (s, 1H), 7.08 (d, J = 7.6 Hz, 1H), 5.54 (s, 2H), 4.17 (s, 2H), 3.92 (s, 3H), 3.64 (t, J = 7.2 Hz, 2H), 3.51 − 3.44 (m, 4H), 3.28 (s, 4H), 1.76 − 1.58 (m, 2H), 1.50 − 1.36 (m, 2H), 0.99 (t, J = 7.4 Hz, 3H). |
| 118 | 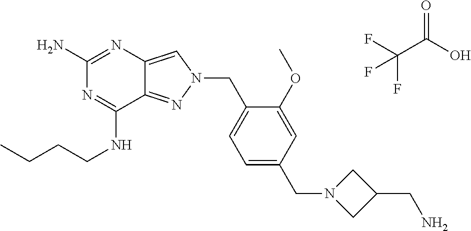 [SMILES: CCCCNc1nc(N)nc2cn(Cc3ccc(CN4CC(CN)C4)cc3OC)nc12 ; O=C(O)C(F)(F)F] | 425.3 | (300 MHz, CD₃OD) δ 7.87 (s, 1H), 7.30 − 7.27 (m, 2H), 7.11 − 7.01 (m, 1H), 5.52 (s, 2H), 4.44 (s, 2H), 4.36 − 4.05 (m, 4H), 3.98 (s, 3H), 3.64 − 3.59 (m, 2H), 3.35 − 3.10 (m, 3H), 1.71 − 1.61 (m, 2H), 1.44 − 1.34 (m, 2H), 0.98 (t, J = 9.0 Hz, 3H). |
| 139 | 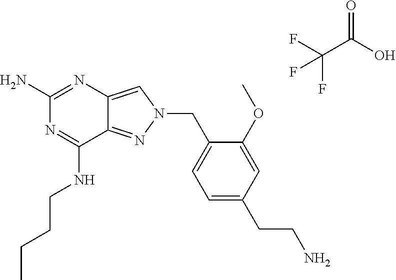 [SMILES: CCCCNc1nc(N)nc2cn(Cc3ccc(CCN)cc3OC)nc12 ; O=C(O)C(F)(F)F] | 370.2 | (400 MHz, CD₃OD) δ 7.81 − 7.79 (m, 1H), 7.25 − 7.23 (m, 1H), 6.96 − 6.95 (m, 1H), 6.89 − 6.87 (m, 1H), 5.50 (s, 2H), 3.88 (s, 3H), 3.63 − 3.59 (m, 2H), 3.20 − 3.18 (m, 2H), 2.97 − 2.93 (m, 2H), 1.70 − 1.68 (m, 2H), 1.45 − 1.42 (m, 2H), 1.01 − 0.98 (m, 3H). |
| 145 | 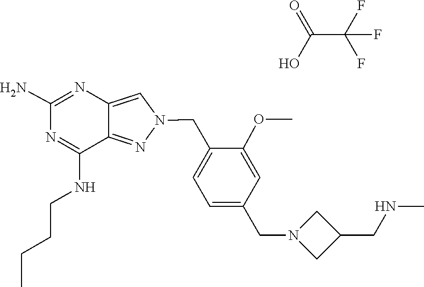 [SMILES: O=C(O)C(F)(F)F ; CCCCNc1nc(N)nc2cn(Cc3ccc(CN4CC(CNC)C4)cc3OC)nc12] | 439.3 | (400 MHz, CD₃OD) δ 7.87 (s, 1H), 7.31 (s, 1H), 7.17 (s, 1H), 7.07 (s, 1H), 5.56 (s, 2H), 4.42 (s, 2H), 4.30 − 4.20 (m, 2H), 4.15 − 4.10 (m, 2H), 3.93 (s, 3H), 3.67 − 3.62 (m, 2H), 3.40 − 3.30 (m, 2H), 3.30 − 3.20 (m, 1H), 2.72 (s, 3H), 1.80 − 1.60 (m, 2H), 1.50 − 1.30 (m, 2H), 1.00 − 0.90 (m, 3H). |
| 147 | 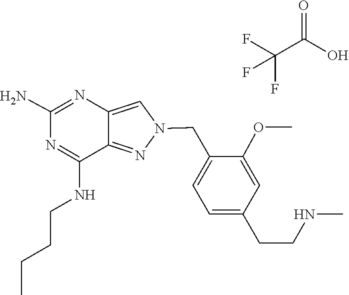 | 384.2 | (300 MHz, CD₃OD) δ 7.83 (s, 1H), 7.28 − 7.25 (m, 1H), 6.99 (s, 1H), 6.92 − 6.89 (m, 1H), 5.52 (s, 2H), 3.99 (s, 3H), 3.63 − 3.61 (m, 2H), 3.33 − 3.31 (m, 2H), 3.09 − 2.98 (m, 2H), 2.64 (s, 3H), 1.73 − 1.69 (m, 2H), 1.49 − 1.42 (m, 2H), 0.99 − 0.89 (m, 3H). |
| 148 | 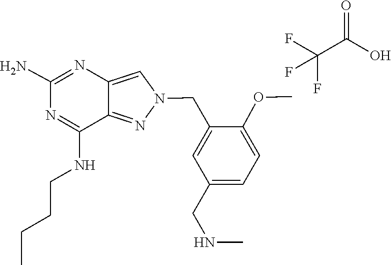 | 370.2 | (300 MHz, CD₃OD) δ 7.86 (s, 1H), 7.52 − 7.49 (m, 1H), 7.32 (s, 1H), 7.16 − 7.13 (m, 1H), 5.57 (s, 2H), 4.12 (s, 2H), 3.92 (s, 3H), 3.66 − 3.62 (m, 2H), 2.69 (s, 3H), 1.74 − 1.69 (m, 2H), 1.50 − 1.47 (m, 2H), 1.01 − 0.99 (m, 3H). |
| 149 | 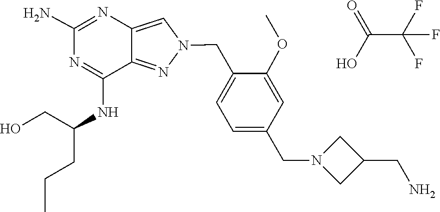 [SMILES: CCC[C@@H](CO)Nc1nc(N)nc2cn(Cc3ccc(CN4CC(CN)C4)cc3OC)nc12 ; O=C(O)C(F)(F)F] | 455.3 | (300 MHz, CD₃OD) δ 7.88 (s, 1H), 7.38 − 7.33 (m, 1H), 7.18 (s, 1H), 7.09 − 7.07 (m, 1H), 5.55 (s, 2H), 4.88 − 4.52 (m, 1H), 4.52 − 4.30 (m, 2H), 4.30 − 4.12 (m, 2H), 4.12 − 4.06 (m, 2H), 3.93 (s, 3H), 3.74 − 3.63 (m, 2H), 3.33 − 3.19 (m, 3H), 1.73 − 1.62 (m, 2H), 1.50 − 1.31 (m, 2H) 1.00 − |
| | | | 0.89 (m, 3H). |
| 151 | 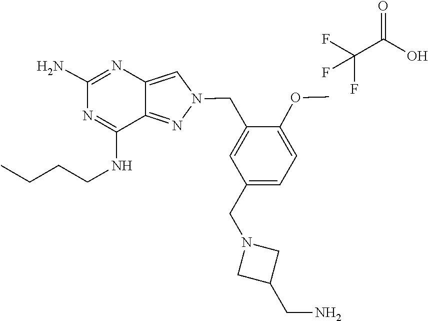 | 425.2 | (300 MHz, CD₃OD) 6 7.84 (s, 1H), 7.51 (s, 1H), 7.34 (s, 1H), 7.14 (s, 1H), 5.52 (s, 2H), 4.35 (s, 2H), 4.30 − 4.10 (m, 2H), 4.10 − 4.00 (m, 2H), 3.90 (s, 3H), 3.70 − 3.50 (m, 2H), 3.50 − 3.10 (m, 3H), 1.80 − 1.60 (m, 2H), 1.50 − 1.30 (m, 2H), 1.15 − 0.98 (m, 3H). |

### Example 9

### Preparation of Compound 62

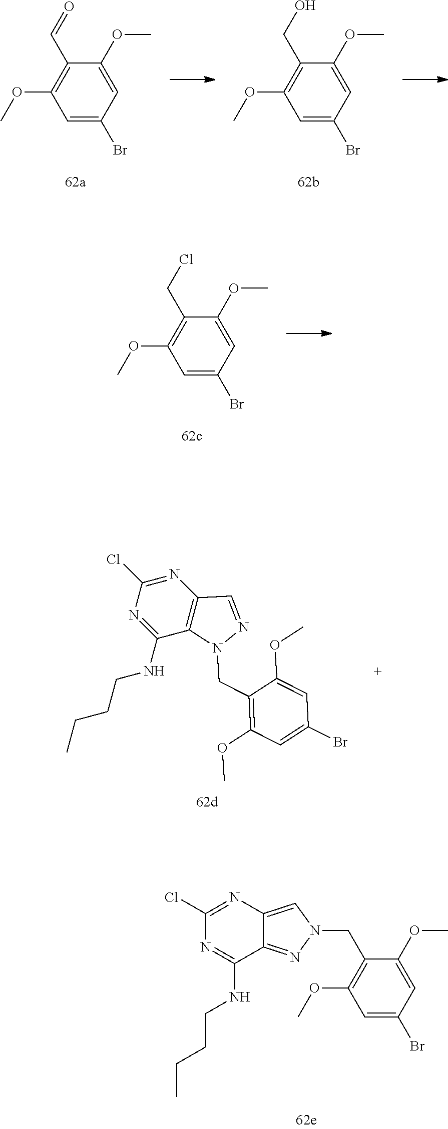

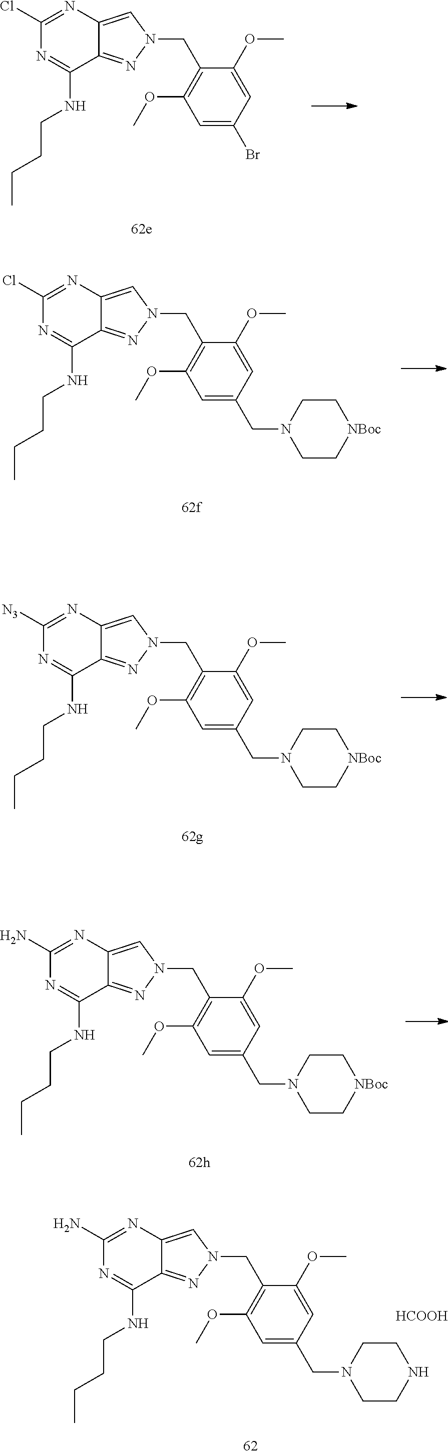 [SMILES: CCCCNc1nc(Cl)nc2cn(Cc3c(OC)cc(Br)cc3OC)nc12 ; CCCCNc1nc(Cl)nc2cn(Cc3c(OC)cc(CN4CCN(C(=O)OC(C)(C)C)CC4)cc3OC)nc12 ; CCCCNc1nc(N=[N+]=[N-])nc2cn(Cc3c(OC)cc(CN4CCN(C(=O)OC(C)(C)C)CC4)cc3OC)nc12 ; CCCCNc1nc(N)nc2cn(Cc3c(OC)cc(CN4CCN(C(=O)OC(C)(C)C)CC4)cc3OC)nc12 ; CCCCNc1nc(N)nc2cn(Cc3c(OC)cc(CN4CCNCC4)cc3OC)nc12 ; "HCOOH"]

### Step A—Synthesis of Compound 62b

To a solution of compound 62a (1 g, 4.08 mmol) in MeOH (20 mL) was added sodium borohydride (0.185 g, 4.90 mmol) at 0° C. under argon atmosphere. The resulting reaction was allowed to stir for 1 hour at 20° C., then was quenched with water (10 mL), and concentrated in vacuo. The residue obtained was extracted with ethyl acetate (3×100 mL), and the combined organic extracts were washed with brine (50 mL). The organic phase was collected, dried over sodium sulfate, and concentrated in vacuo to provide compound 62b which was used without further purification. MS: m/z=228.99 [M−OH].

### Step B—Synthesis of Compound 62c

To a solution of NCS (266 mg, 2.02 mmol) in DCM (10 mL), at 0° C. under argon atmosphere, was added dimethylsulfide (126 mg, 2.02 mmol) dropwise. The resulting reaction was allowed to stir for 5 minutes at 0° C., then cooled to −20° C., and a solution of compound 62b (500 mg, 2.02 mmol) in DCM (2 mL) was added. The reaction mixture was allowed to stir for 2 hours, during which time the reaction temperature warmed to 0° C. The reaction was quenched with cooled brine (30 mL), and extracted with diethyl ether (3×50 mL). The combined organic extracts were washed with cooled brine (20 mL), dried over anhydrous sodium sulfate, and filtered. The filtrate was concentrated in vacuo, and the resulting residue was washed with pentane (2×10 mL) to provide compound 62c, which was used without further purification. MS: m/z=229.1 [M−Cl].

### Step C—Synthesis of Compound 62d and 62e

Compounds 62d and 62e were made (in a ratio of 1:5) from compound 62c, using the method described in Example 3, step D, and substituting the appropriate reactants and/or reagents. MS: m/z=454.0 [M+H].

### Step D—Synthesis of Compound 62f

To a mixture of Compound 62e (150 mg, 0.330 mmol), potassium ((4-(tert-butoxycarbonyl)piperazin-1-yl)methyl)trifluoroborate (151 mg, 0.495 mmol), and K₃PO₄ (0.123 ml, 1.484 mmol) in a mixture of1,4-dioxane (3 mL), and water (0.3 mL) was added AmPhos Pd G₃ (11.68 mg, 0.016 mmol). The resulting reaction was put under argon atmosphere, heated to 80° C., and allowed to stir at this temperature for 3 hours. The reaction was quenched with water (20 mL), and extracted with ethyl acetate (60 mL). The collected organic phase was washed with brine (2×20 mL), dried over anhydrous sodium sulfate, and concentrated in vacuo. The residue obtained was purified using Prep-TLC (100% ethyl acetate) to provide compound 62f MS: m/z=574.3 [M+H].

### Step E—Synthesis of Compound 62g

Compound 62g was made from compound 62f, using the method described in Example 5, step E, and substituting the appropriate reactants and/or reagents. MS: m/z=581.3 [M+H].

### Step F—Synthesis of Compound 63h

Compound 62h was made from compound 62g, using the method described in Example 5, step F, and substituting the appropriate reactants and/or reagents. MS: m/z=555.3 [M+H].

### Step G—Synthesis of Compound 62

To a mixture of compound 62h (60 mg, 0.108 mmol) in DCM (1 mL) at 0° C. was added HCl (4 M in dioxane, 1 mL, 4.00 mmol), and the resulting mixture was allowed to stir for 1 hour at 20° C. The reaction mixture was concentrated in vacuo, and the resulting residue was purified using Prep-HPLC: Column: SunFire Prep C18 OBD Column, 19×150 mm 5 um 10 nm; Mobile Phase A: water (0.1% FA), Mobile Phase B: MeOH; Flow rate: 20 mL/minute; Gradient: 20% B to 35% B in 4.3 minutes; Detector: UV 210/254 nm; room temperature: 4.23 minutes to provide compound 62. MS: m/z=455.2 [M+H]. ¹H NMR (300 MHz, CD₃OD) δ 8.51 (s, 1H), 7.51 (s, 1H), 6.74 (s, 2H), 5.52 (s, 2H), 3.87 (s, 6H), 3.62-3.54 (m, 4H), 3.23-3.19 (m, 4H), 2.69-2.60 (m, 4H), 1.69-1.62 (m, 2H), 1.48-1.39 (m, 2H), 0.99-0.90 (m, 3H).

The following compounds of the present invention were made using methodology described in Example 9 above, and substituting the appropriate reactants and/or reagents:

| | | MS | |
|---|---|---|---|
| Compound | Structure | [M + H] | ¹H NMR |
| 63 | 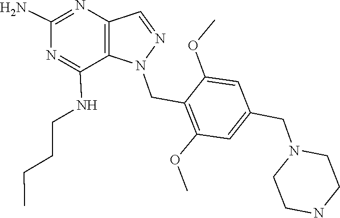 | 455.3 | (300 MHz, CD₃OD) δ 7.55 (s, 1H), 7.04 (s, 2H), 5.72 (s, 2H), 4.83 (s, 2H), 3.86 (s, 6H), 3.77 − 3.73 (m, 2H), 3.65 − 3.64 (m, 8H), 1.80 − 1.70 (m, 2H), 1.52 − 1.40 (m, 2H), 0.99 − 0.90 (m, 3H). |
| 64 | 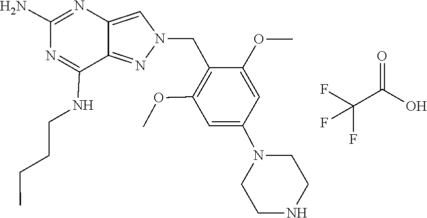 | 441.3 | (300 MHz, CD₃OD) δ 7.63 (s, 1H), 6.35 (s, 2H), 5.51 (s, 2H), 3.86 (s, 6H), 3.70 − 3.60 (m, 2H), 3.56 − 3.49 (m, 4H), 3.37 − 3.30 (m, 4H), 1.73 − 1.66 (m, 2H), 1.52 − 1.45 (m, 2H), 0.99 − 0.90 (m, 3H). |
| 65 | 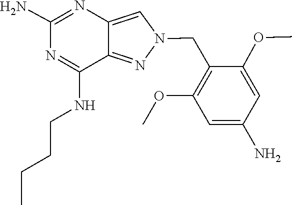 [SMILES: CCCCNc1nc(N)nc2cn(Cc3c(OC)cc(N)cc3OC)nc12] | 372.1 | (400 MHz, CDCl₃) δ 7.50 (s, 1H), 6.48 (s, 1H), 5.92 (s, 2H), 5.36 (s, 2H), 3.78 (s, 6H), 3.65 − 3.60 (m, 2H), 1.72 − 1.65 (m, 2H), 1.51 − 1.44 (m, 2H), 1.02 − 0.98 (m, 3H). |
| 66 | 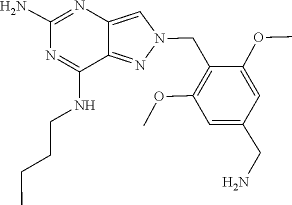 [SMILES: CCCCNc1nc(N)nc2cn(Cc3c(OC)cc(CN)cc3OC)nc12] | 386.1 | (300 MHz, CD₃OD) δ 7.66 (s, 1H), 6.83 (s, 2H), 5.53 (s, 2H), 4.11 (s, 2H), 3.91 (s, 6H), 3.62 − 3.58 (m, 2H), 1.72 − 1.63 (m, 2H), 1.49 − 1.37 (m, 2H), 1.00 − 0.96 (m, 3H). |
| 67 | 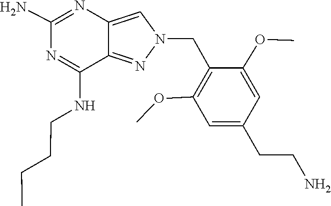 [SMILES: CCCCNc1nc(N)nc2cn(Cc3c(OC)cc(CCN)cc3OC)nc12] | 400.1 | (300 MHz, CDCl₃) δ 7.43 (s, 1H), 6.44 (s, 2H), 6.00 (s, 1H), 5.45 − 5.35 (m, 4H), 3.85 (s, 6H), 3.59 − 3.57 (m, 2H), 3.03 − 2.98 (m, 2H), 2.78 − 2.73 (m, 2H), 2.03 (s, 2H), 1.71 − 1.61 (m, 2H), 1.59 − 1.38 (m, 2H), 1.00 − 0.96 (m, 3H). |
| 112 | 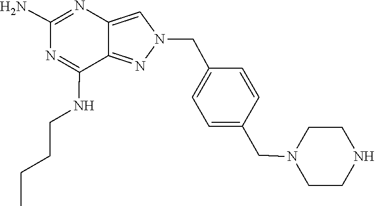 [SMILES: CCCCNc1nc(N)nc2cn(Cc3ccc(CN4CCNCC4)cc3)nc12] | 395.1 | (300 MHz, CD₃OD) δ 8.00 − 7.96 (m, 1H), 7.79 − 7.67 (m, 2H), 7.53 − 7.50 (m, 2H), 5.92 (s, 2H), 4.51 (s, 2H), 3.66 − 3.62 (m, 10H), 1.74 − 1.67 (m, 2H), 1.64 − 1.47 (m, 2H), 1.01 − 0.95 (m, 3H) |
| 123 | 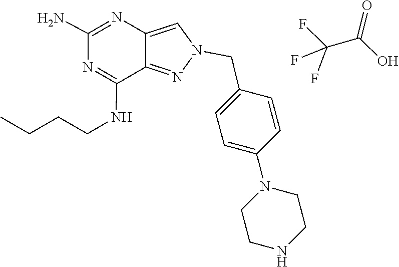 | 382.2 | (300 MHz, CD₃OD) δ 8.31 (s, 1H), 7.95 (s, 1H), 7.52 − 7.48 (m, 1H), 7.38 − 7.35 (m, 1H), 5.58 (s, 2H), 3.64 − 3.62 (m, 2H), 3.51 − 3.46 (m, 4H), 3.31 − 3.29 (m, 4H), 1.69 − 1.64 (m, 2H), 1.47 − 1.40 (m, 2H), 0.99 − 0.97 (m, 3H). |
| 152 | 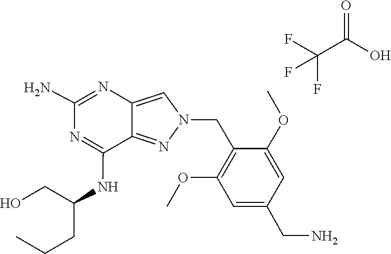 | 416.2 | (300 MHz, CD₃OD) δ 7.76 (s, 1H), 6.85 (s, 2H), 5.58 (s, 2H), 4.54 (s, 1H), 4.15 (s, 2H), 3.93 (s, 6H), 3.80 − 3.58 (m, 2H), 1.80 − 1.60 (m, 2H), 1.50 − 1.30 (m, 2H), 1.00 − 0.90 (m, 3H). |
| 166 | 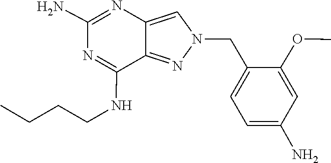 [SMILES: CCCCNc1nc(N)nc2cn(Cc3ccc(N)cc3OC)nc12] | 342.3 | (300 MHz, CD₃OD) δ 7.51 (s, 1H), 7.08 (s, 1H), 7.02 (s, 1H), 6.99 (s, 1H), 5.30 (m, 2H), 3.80 − 3.78 (m, 3H), 3.55 − 3.53 (m, 2H), 1.72 − 1.69 (m, 2H), 1.52 − 1.49 (m, 2H), 1.30 − 1.01 (m, 3H). |

### Example 10

### Preparation of Compound 68

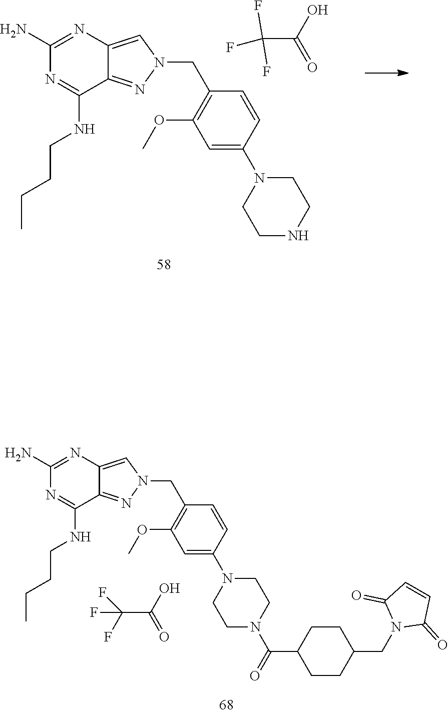

A mixture of N-[4-(-carboxycyclohexylmethyl)]maleimide (15.60 mg, 0.066 mmol), HATU (27.8 mg, 0.073 mmol), and DIEA (0.038 mL, 0.219 mmol) in DMF (1 mL) was allowed to stir for 10 minutes at 25° C. The reaction mixture was added to a solution of compound 58 (30 mg, 0.073 mmol), and DIEA (0.038 mL, 0.219 mmol) in DMF (0.200 mL). Then the resulting mixture was allowed to stir for 30 minutes at 25° C. The reaction was purified using RP-Flash chromatography (eluted with 0-100% acetonitrile in aq. 0.05% TFA) to provide compound 68. MS: m/z=630.4 [M+H]. ¹H NMR (500 MHz, Methanol-d₄) δ 7.72 (s, 1H), 7.20 (d, J=8.3 Hz, 1H), 6.83 (s, 2H), 6.63 (s, 1H), 6.58 (d, J=8.2 Hz, 1H), 5.42 (s, 2H), 3.86 (s, 3H), 3.77-3.72 (m, 4H), 3.64 (t, J=7.2 Hz, 2H), 3.38 (d, J=6.9 Hz, 3H), 3.26 (s, 2H), 3.22-3.17 (m, 2H), 2.67 (t, J=11.8 Hz, 1H), 1.85-1.64 (m, 8H), 1.55-1.38 (m, 2H), 1.12 (t, J=11.0 Hz, 2H), 0.99 (t, J=7.4 Hz, 3H).

The following compounds of the present invention were made using the methods described in Example 10 above, and substituting the appropriate reactants and/or reagents:

| | | MS | |
|---|---|---|---|
| Compound | Structure | [M + H] | ¹H NMR |
| 69 | 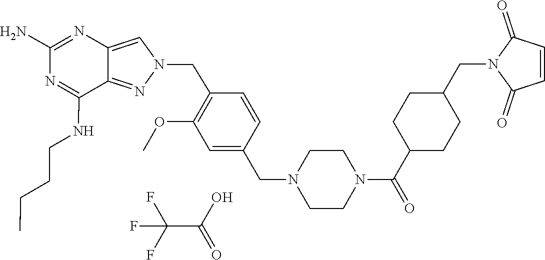 | 644.4 | (500 MHz, Methanol-d₄) δ 7.87 (s, 1H), 7.34 − 7.32 (m, 1H), 7.22 (s, 1H), 7.11 − 7.09 (m, 1H), 6.82 (s, 2H), 5.56 (s, 2H), 4.33 (s, 2H), 3.94 (s, 3H), 3.64 − 3.62 (m, 4H), 3.46 − 3.45 (m, 2H), 3.37 − 3.33 (m, 4H), 3.24 − 3.21 (m, 2H), 2.62 − 2.60 (m, 1H), 1.85 − 1.62 (m, 5H), 1.44 − 1.40 (m, 8H), 0.99 − 0.97 (m, 3H) |
| 70 | 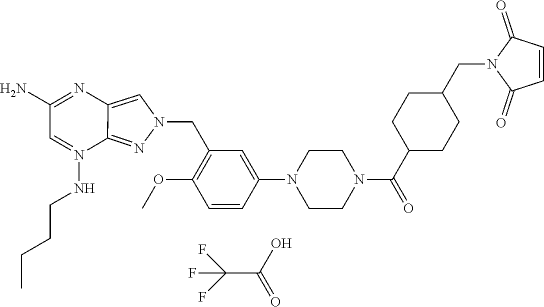 | 630.4 | (500 MHz, Methanol-d₄) δ 7.80 (s, 1H), 7.06 (d, J = 8.9 Hz, 1H), 7.02 − 6.96 (m, 2H), 6.83 (s, 2H), 5.49 (s, 2H), 3.83 (s, 3H), 3.74 (s, 5H), 3.64 (t, J = 7.2 Hz, 2H), 3.38 (d, J = 7.0 Hz, 2H), 3.09 (s, 2H), 3.04 (s, 2H), 2.66 (d, J = 17.1 Hz, 1H), 1.85 − 1.67 (m, 8H), 1.44 (dd, J = 14.7, 7.7 Hz, 2H), 1.11 (q, J = 12.1 Hz, 2H), 0.99 (t, J = 7.4 Hz, 3H). |
| 71 | 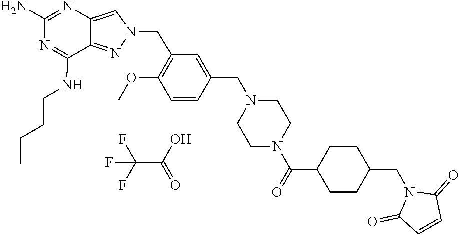 [SMILES: CCCCNc1nc(N)nc2cn(Cc3cc(CN4CCN(C(=O)C5CCC(CN6C(=O)C=CC6=O)CC5)CC4)ccc3OC)nc12 ; O=C(O)C(F)(F)F] | 644.2 | (500 MHz, Methanol-d₄) δ 7.86 (d, J = 4.3 Hz, 1H), 7.53 (d, J = 8.5 Hz, 1H), 7.35 (s, 1H), 7.23 − 7.10 (m, 1H), 6.83 (s, 2H), 5.55 (d, J = 3.1 Hz, 2H), 4.29 (d, J = 9.6 Hz, 2H), 3.93 (d, J = 3.3 Hz, 4H), 3.65 (t, J = 7.2 Hz, 2H), 3.42 − 3.35 (m, 2H), 3.19 (s, 4H), 2.62 (s, 1H), 1.85 − 1.65 (m, 9H), 1.48 − 1.40 (m, 5H), 1.10 (d, J = 12.2 Hz, 2H), 0.99 (t, J = 7.4 Hz, 3H). |
| 153 | 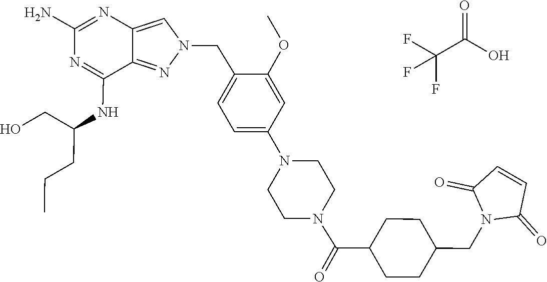 [SMILES: CCC[C@@H](CO)Nc1nc(N)nc2cn(Cc3ccc(N4CCN(C(=O)C5CCC(CN6C(=O)C=CC6=O)CC5)CC4)cc3OC)nc12 ; O=C(O)C(F)(F)F] | 660.7 | (300 MHz, CD₃OD) δ 7.75 (s, 1H), 7.22 − 7.19 (m, 2H), 6.82 (s, 1H), 6.63 (s, 2H), 5.42 (s, 2H), 4.55 − 4.52 (m, 1H), 3.85 (s, 3H), 3.74 − 3.64 (m, 6H), 3.38 − 3.20 (m, 6H), 2.67 − 2.63 (m, 1H), 1.83 − 1.62 (m, 7H), 1.49 − 1.38 (m, 4H), 1.13 − 0.95 (m, 5H). |

### Example 11

### Preparation of Compound 72

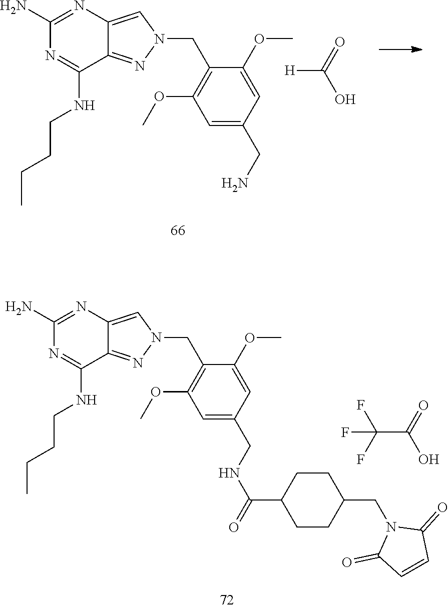

To a mixture of 4-((2,5-dioxo-2,5-dihydro-1H-pyrrol-1-yl)methyl) cyclohexane-1-carboxylic acid (43.8 mg, 0.184 mmol) in DMF (1.5 mL) was added HATU (94 mg, 0.246 mmol), and the resulting mixture was allowed to stir for 5 minutes at room temperature. DIEA (132 mg, 1.025 mmol) was added, followed by a solution of compound 66 (79 mg, 0.205 mmol) in DMF (0.2 mL), and the resulting reaction was allowed to stir for 1 hour at room temperature. The reaction mixture was then directly purified using RP-Flash chromatography using 20%-60% gradient of acetonitrile in 0.05% aq. TFA to provide compound 72. MS: m/z=605.2 [M+H]. ¹H NMR (300 MHz, CD₃OD) δ 7.63 (s, 1H), 6.80 (s, 2H), 6.62 (s, 2H), 5.52 (s, 2H), 4.36-4.34 (m, 2H), 3.85 (s, 6H), 3.63-3.54 (m, 4H), 2.24-2.17 (m, 1H), 1.88-1.61 (m, 9H), 1.54-1.38 (m, 4H), 1.00-0.91 (m, 3H).

The following compounds of the present invention were made using methodology described in Example 11 above, and substituting the appropriate reactants and/or reagents:

| | | MS | |
|---|---|---|---|
| Compound | Structure | [M + H] | ¹H NMR |
| 73 | 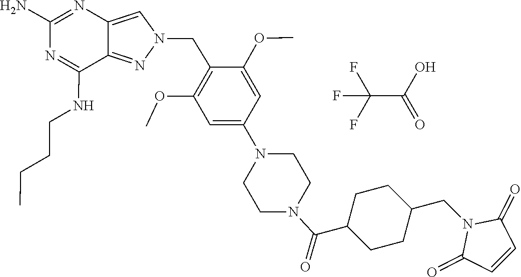 [SMILES: CCCCNc1nc(N)nc2cn(Cc3c(OC)cc(N4CCN(C(=O)C5CCC(CN6C(=O)C=CC6=O)CC5)CC4)cc3OC)nc12 ; O=C(O)C(F)(F)F] | 660.2 | (300 MHz, d₆-DMSO + D₂O) δ 7.57 (s, 1H), 6.97 (s, 2H), 6.25 (s, 2H), 5.33 (s, 2H), 3.86 (s, 6H), 3.66 − 3.61 (m, 2H), 3.27 − 3.20 (m, 8H), 2.57 − 2.51 (m, 2H), 1.69 − 1.54 (m, 7H), 1.35 − 1.25 (m, 4H), 1.08 − 0.89 (m, 6H). |
| 74 | 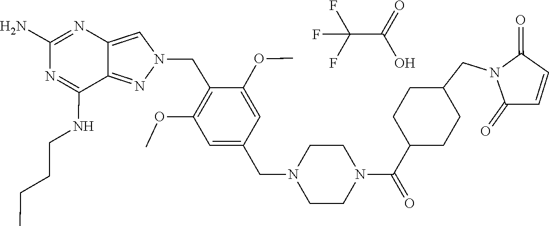 | 674.3 | (300 MHz, CD₃OD) δ 7.75 (s, 1H), 6.90 (s, 2H), 6.82 (s, 2H), 5.58 (s, 2H), 4.36 (s, 2H), 3.93 (s, 6H), 3.65 − 3.60 (m, 2H), 3.37 − 3.31 (m, 10H), 2.67 − 2.59 (m, 1H), 2.04 − 1.97 (m, 1H), 1.88 − 1.63 (m, 8H), 1.54 − 1.38 (m, 4H), 1.00 − 0.91 (m, 3H). |
| 75 | 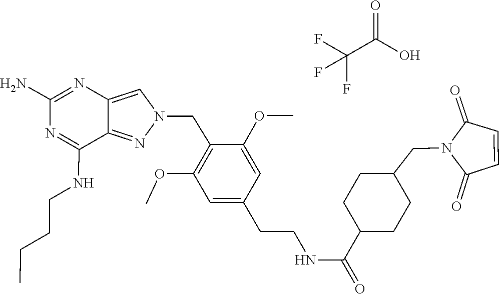 [SMILES: CCCCNc1nc(N)nc2cn(Cc3c(OC)cc(CCNC(=O)C4CCC(CN5C(=O)C=CC5=O)CC4)cc3OC)nc12 ; O=C(O)C(F)(F)F] | 619.2 | (300 MHz, CD₃OD) δ 7.63 (s, 1H), 6.81 (s, 2H), 6.66 (s, 2H), 5.52 (s, 2H), 3.85 (s, 6H), 3.65 − 3.60 (m, 2H), 3.45 − 3.40 (m, 4H), 2.82 − 2.64 (m, 2H), 2.04 − 1.58 (m, 7H), 1.53 − 1.24 (m, 5H), 1.00 − 0.91 (m, 5H). |
| 120 | 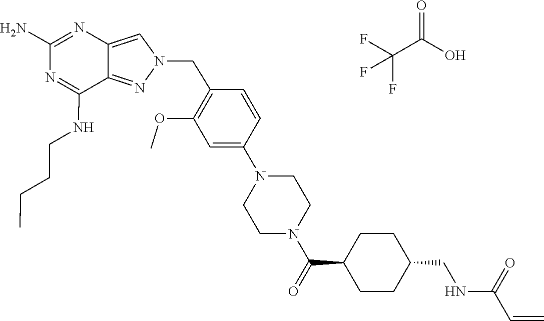 [SMILES: C=CC(=O)NC[C@H]1CC[C@H](C(=O)N2CCN(c3ccc(Cn4cc5nc(N)nc(NCCCC)c5n4)c(OC)c3)CC2)CC1 ; O=C(O)C(F)(F)F] | 604.3 | (300 MHz, CD₃OD) δ 7.79 (s, 1H), 7.22 − 7.18 (m, 1H), 6.64 − 6.59 (m, 2H), 6.26 − 6.23 (m, 2H), 5.68 − 5.54 (m, 1H), 5.42 (s, 2H), 3.87 (s, 3H), 3.74 − 3.66 (m, 4H), 3.63 − 3.61 (m, 2H), 3.33 − 3.13 (m, 6H), 2.68 (s, 1H), 1.96 − 2.27 (m, 10 H), 1.19 − 0.96 (m, 5H). |

### Example 12

### Preparation of Compound 76

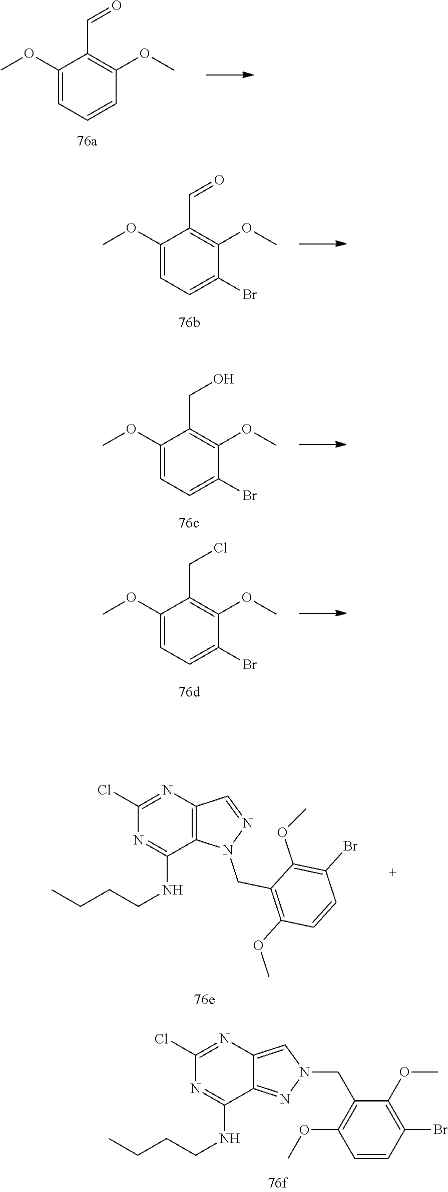

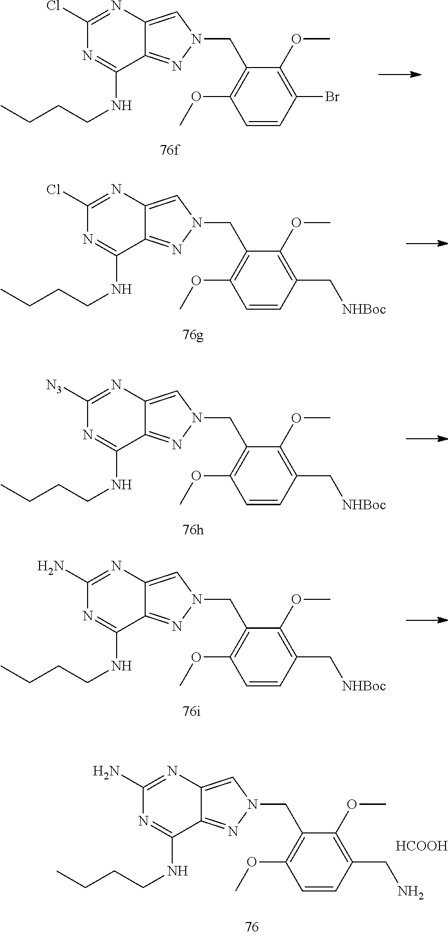

### Step A—Synthesis of Compound 76b

To a solution of compound 76a (1.5 g, 9.03 mmol) in acetonitrile (20 mL), at 20° C., was added NBS (1.77 g, 9.93 mmol). TMSCl (0.115 mL, 0.903 mmol) was then added dropwise, and the resulting reaction was allowed to stir for 1 hour at 20° C. Water (20 mL) was added, and the aqueous layer was collected and extracted with ethyl acetate (2×200 mL). The combined organic extracts were washed with brine (50 mL), dried over anhydrous sodium sulfate, filtered, and concentrated in vacuo. The residue obtained was purified using silica gel column chromatography (eluted with 0—30% ethyl acetate in petroleum ether) to provide compound 76b. MS: m/z=245.1 [M+H].

### Step B—Synthesis of Compound 76c

Compound 76c was made from compound 76b, using the method described in Example 9, step A, and substituting the appropriate reactants and/or reagents. MS: m/z=229.0 [M−OH].

### Step C—Synthesis of Compound 76d

Compound 76d was made from compound 76c, using the method described in Example 9, step B, and substituting the appropriate reactants and/or reagents. MS: m/z=229.1 [M−Cl].

### Step D—Synthesis of Compound 76e and 76f

A mixture of compounds 76e and 76f with ratio (1/9) were made from compound 76d, using the method described in Example 5, step D, and substituting the appropriate reactants and/or reagents. The mixture was not separated and used directly without further purification. MS: m/z=454.1 [M+H].

### Step E—Synthesis of Compound 76g

Compound 76g was made from the mixture of compounds 76e and 76f obtained in step D, using the method described in Example 9, step D, and substituting the appropriate reactants and/or reagents. MS: m/z=505.3 [M+H].

### Step F—Synthesis of Compound 76h

Compound 76h was made from compound 76g, using the method described in Example 5, step E, and substituting the appropriate reactants and/or reagents. MS: m/z=512.1 [M+H].

### Step G—Synthesis of Compound 76i

Compound 76i was made from compound 76h, using the method described in Example 9, step F, and substituting the appropriate reactants and/or reagents. MS: m/z=486.3 [M+H].

### Step H—Synthesis of Compound 76

Compound 76 was made from compound 76i, using the method described in Example 9, step G, and substituting the appropriate reactants and/or reagents. MS: m/z=386.1 [M+H]. ¹H NMR (300 MHz, CD₃OD) δ 8.56 (s, 1H), 7.76 (s, 1H), 7.50-7.47 (m, 1H), 6.98-6.89 (m, 1H), 5.58 (s, 2H), 4.12 (s, 2H), 3.91-3.85 (m, 6H), 3.62-3.53 (m, 2H), 1.70-1.61 (m, 2H), 1.47-1.28 (m, 2H), 0.99-0.83 (m, 3H).

The following compounds of the present invention were made using the methods described in Example 12 above, and substituting the appropriate reactants and/or reagents:

| | | MS | |
|---|---|---|---|
| Compound | Structure | [M + H] | ¹H NMR |
| 77 | 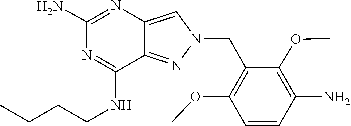 [SMILES: CCCCNc1nc(N)nc2cn(Cc3c(OC)ccc(N)c3OC)nc12] | 372.1 | (300 MHz, CDCl₃) δ 7.54 (s, 1H), 6.80 − 6.77 (m, 1H), 6.61 − 6.59 (m, 1H), 6.08 − 6.05 (m, 1H), 5.67 (s, 2H), 3.77 (s, 3H), 3.70 (s, 3H), 3.57 − 3.48 (m, 4H), 1.73 − 1.62 − (m, 2H), 1.51 − |
| | | | 1.41 (m, 2H), 1.00 − |
| | | | 0.96 (m, 3H). |
| 78 | 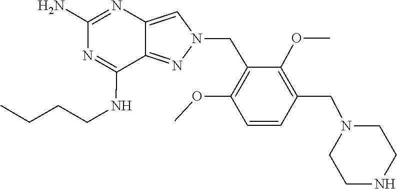 [SMILES: CCCCNc1nc(N)nc2cn(Cc3c(OC)ccc(CN4CCNCC4)c3OC)nc12] | 455.2 | (300 MHz, CD₃OD) δ 7.72 (s, 1H), 7.45 − 7.42 (m, 1H), 6.90 − 6.87 (m,lH), 5.55 (s, 2H), 3.85 (s, 3H), 3.80 (s, 3H), 3.61 − 3.57 (m, 4H), 3.20 − 3.16 (m, 4H), 2.71 − 2.68 (m, 4H), 1.71 − 1.64 (m, 2H), 1.47 − 1.37 (m, 2H), 0.99 − 0.91 (m, 3H). |
| 79 | 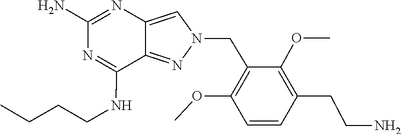 [SMILES: CCCCNc1nc(N)nc2cn(Cc3c(OC)ccc(CCN)c3OC)nc12] | 400.1 | (300 MHz, CDCl₃) δ 7.64 (s, 1H), 6.90 − 6.89 (m, 1H), 6.69 − 6.66 (m, 1H), 5.46 (s, 2H), 3.90 (s, 3H), 3.81 (s, 3H), 3.77 − 3.24 (m, 6H), 2.96 − 2.88 (m, 2H), 1.68 − 1.61 (m, 2H), 1.48 − |
| | | | 1.41 (m, 2H), 0.98 − |
| | | | 0.94 (m, 3H). |

### Example 13

### Preparation of Compound 80

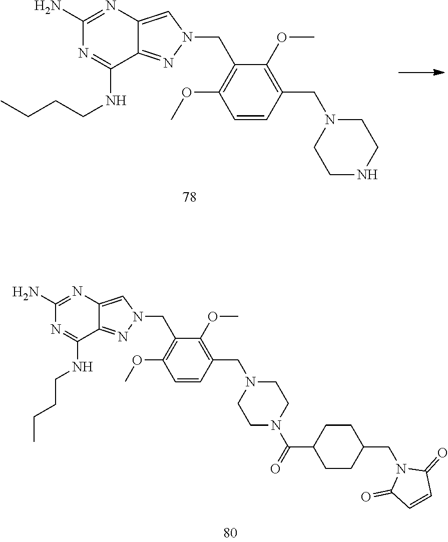 [SMILES: CCCCNc1nc(N)nc2cn(Cc3c(OC)ccc(CN4CCNCC4)c3OC)nc12 ; CCCCNc1nc(N)nc2cn(Cc3c(OC)ccc(CN4CCN(C(=O)C5CCC(CN6C(=O)C=CC6=O)CC5)CC4)c3OC)nc12]

Compound 80 was made from compound 78, using the method described in Example 11, and substituting the appropriate reactants and/or reagents. MS: m/z=674.2 [M+H]. ¹H NMR (300 MHz, CDCl₃) δ 7.48 (s, 1H), 7.41 (d, J=8.4 Hz, 1H), 6.76-6.72 (m, 3H), 5.51 (s, 2H), 3.88 (s, 3H), 3.72 (s, 3H), 3.62-3.36 (m, 11H), 2.45-2.37 (m, 5H), 1.84-1.42 (m, 11H), 1.00-0.89 (m, 5H).

The following compounds of the present invention were made using methodology described in Example 13 above, and substituting the appropriate reactants and/or reagents:

| | | MS | |
|---|---|---|---|
| Compound | Structure | [M + H] | ¹H NMR |
| 81 | 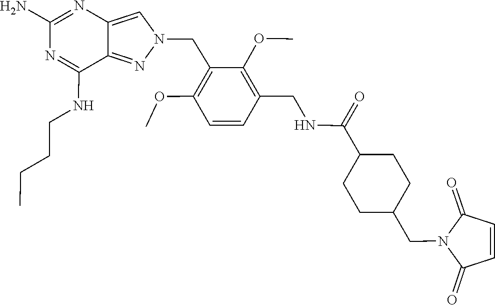 [SMILES: CCCCNc1nc(N)nc2cn(Cc3c(OC)ccc(CNC(=O)C4CCC(CN5C(=O)C=CC5=O)CC4)c3OC)nc12] | 605.1 | (300 MHz, CD₃OD) δ 7.78 (s, 1H), 7.32 − 7.29 (m, 1H), 6.87 − 6.81 (m, 3H), 5.55 (s, 2H), 4.37 (s, 2H), 3.89 (s, 3H), 3.78 (s, 3H), 3.64 − 3.54 (m, 3H), 3.22 − 3.15 (m, 2H), 2.24 − 2.14 (m, 1H), 1.86 − 1.70 (m, 6H), 1.45 − 1.38 (m, 4H), 1.11 − 0.95 (m, 5H). |
| 82 | 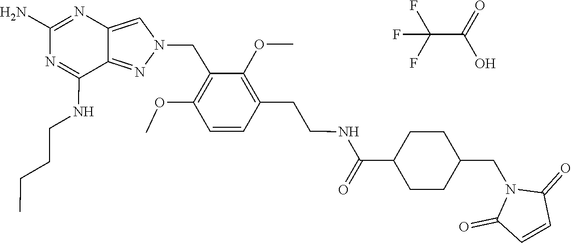 [SMILES: CCCCNc1nc(N)nc2cn(Cc3c(OC)ccc(CCNC(=O)C4CCC(CN5C(=O)C=CC5=O)CC4)c3OC)nc12 ; O=C(O)C(F)(F)F] | 619.4 | (300 MHz, CD₃OD) δ 7.74 (s, 1H), 7.31 − 7.28 (m, 1H), 6.84 − 6.81 (m, 3H), 5.57 (s, 2H), 3.83 (s, 3H), 3.79 (s, 3H), 3.68 − 3.60 (m, 2H), 3.55 − 3.37 (m, 3H), 2.82 − 2.78 (m, 2H), 2.06 − 1.97 (m, 1H), 1.73 − 1.57 (m, 7H), 1.49 − 1.28 (m, 5H), 1.01 − 0.95 (m, 5H). |

### Example 14

### Preparation of Compound 83

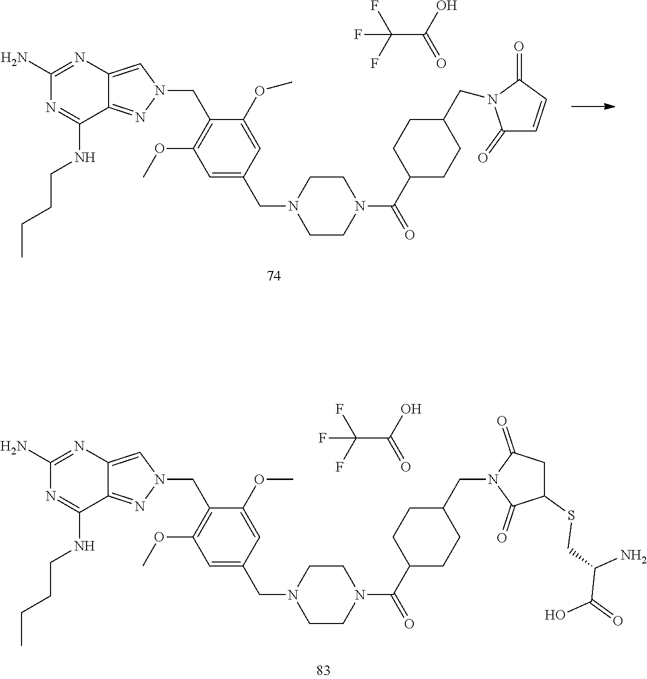

A mixture of compound 74 (30 mg, 0.038 mmol), and L-cysteine (6.92 mg, 0.057 mmol) in DMF (1 mL), at 20° C., was allowed to stir for 4 hours in the dark. The reaction mixture was filtered, and the filtrate was co-evaporated with toluene (3×5 mL) in vacuo, and the resulting residue was triturated with acetonitrile (2 mL) to provide compound 83. MS: m/z=795.5 [M+H]. ¹H NMR (300 MHz, CD₃OD) δ 7.68 (s, 1H), 6.83 (s, 2H), 5.54 (s, 2H), 4.05-3.96 (m, 3H), 3.88-3.85 (m, 7H), 3.64-3.59 (m, 5H), 3.38-3.21 (m, 3H), 3.09-3.02 (m, 4H), 2.87-2.69 (m, 3H), 2.59-2.52 (m, 2H), 1.77-1.62 (m, 6H), 1.46-1.29 (m, 5H), 1.05-0.95 (m, 5H).

The following compounds of the present invention were made using methodology described in Example 14 above, and substituting the appropriate reactants and/or reagents:

| Com- | | MS | |
|---|---|---|---|
| pound | Structure | [M + H] | ¹H NMR |
| 84 | 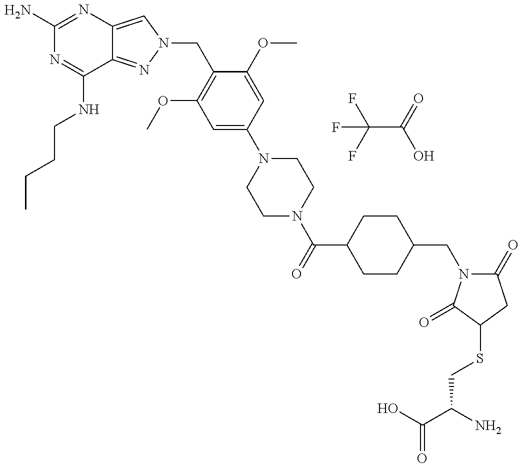 [SMILES: CCCCNc1nc(N)nc2cn(Cc3c(OC)cc(N4CCN(C(=O)C5CCC(CN6C(=O)CC(SC[C@H](N)C(=O)O)C6=O)CC5)CC4)cc3OC)nc12 ; O=C(O)C(F)(F)F] | 781.2 | (300 MHz, CD₃OD) δ 7.59 (s, 1H), 6.27 (s, 2H), 5.44 (s, 2H), 4.08- 3.97 (m, 2H), 3.88- 3.84 (m, 8H), 3.78- 3.74 (m, 5H), 3.68- 3.60 (m, 3H), 2.67- 2.55 (m, 1H), 1.81- 1.67 (m, 10H), 1.52- 1.48 (m, 5H), 1.23- 0.98 (m, 7H). |
| 85 | 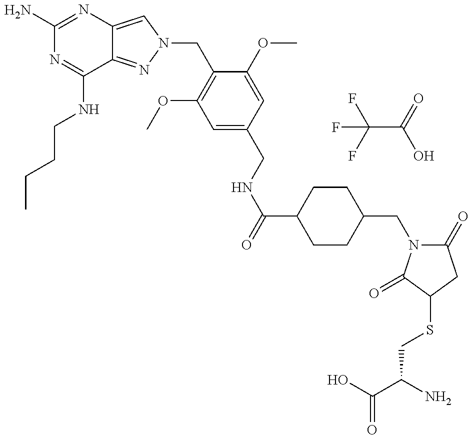 | 726.4 | (300 MHz, CD₃OD) δ 7.58 (s, 1H), 6.62 (s, 2H), 5.49 (s, 2H), 4.34 (s, 2H), 3.91 (s, 6H), 3.63-3.58 (m, 3H), 3.39-3.30 (m, 5H), 2.58-2.51 (m, 1H), 2.20-2.16 (m, 1H), 1.88-1.64 (m, 8H), 1.52-1.38 (m, 4H), 1.10-0.95 (m, 5H). |
| 86 | 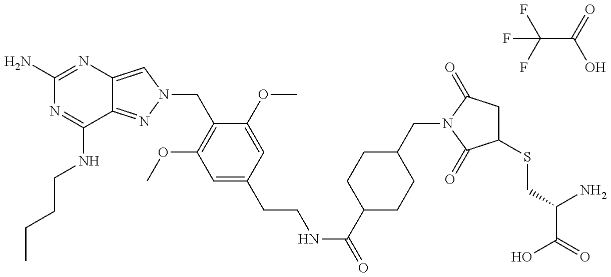 [SMILES: CCCCNc1nc(N)nc2cn(Cc3c(OC)cc(CCNC(=O)C4CCC(CN5C(=O)CC(SC[C@H](N)C(=O)O)C5=O)CC4)cc3OC)nc12 ; O=C(O)C(F)(F)F] | 740.2 | (300 MHz, CD₃OD) δ 7.65 (s, 1H), 6.59 (s, 2H), 5.51 (s, 2H), 4.05- 3.79 (m, 8H), 3.66- 3.56 (m, 2H), 3.47- 3.42 (m, 2H), 3.33- 3.28 (m, 3H), 3.08- 2.99 (m, 1H), 2.81- 2.75 (m, 2H), 2.61- 2.59 (m, 1H), 2.03- 1.98 (m, 1H), 1.72- 1.67 (m, 7H), 1.48- 1.30 (m, 5H), 1.04- 0.97 (m, 5H). |
| 87 | 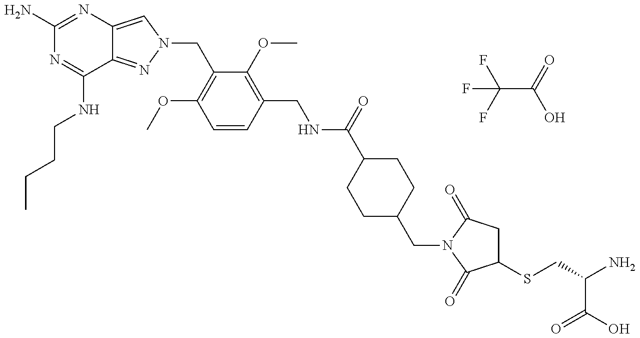 [SMILES: CCCCNc1nc(N)nc2cn(Cc3c(OC)ccc(CNC(=O)C4CCC(CN5C(=O)CC(SC[C@H](N)C(=O)O)C5=O)CC4)c3OC)nc12 ; O=C(O)C(F)(F)F] | 726.2 | (300 MHz, CD₃OD) δ 7.62 (s, 1H), 7.23 (d, J = 8.7 Hz, 1H), 6.77 (d, J = 8.4 Hz, 1H), 5.45 (s, 2H), 4.27 (s, 2H), 3.98- 3.78 (m, 1H), 3.73- 3.64 (m, 7H), 3.54- 3.49 (m, 2H), 3.25- 3.14 (m, 4H), 2.92- 2.90 (m, 1H), 2.50- 2.49 (m, 1H), 2.10- 2.00 (m, 1H), 1.75- 1.54 (m, 7H), 1.35- 1.28 (m, 4H), 0.91- 0.81 (m, 5H). |
| 88 | 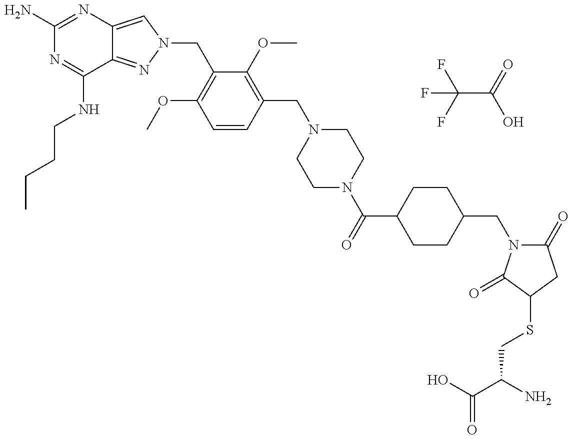 [SMILES: CCCCNc1nc(N)nc2cn(Cc3c(OC)ccc(CN4CCN(C(=O)C5CCC(CN6C(=O)CC(SC[C@H](N)C(=O)O)C6=O)CC5)CC4)c3OC)nc12 ; O=C(O)C(F)(F)F] | 795.2 | (300 MHz, CD₃OD) δ 7.82 (s, 1H), 7.59 (d, J = 8.7 Hz, 1H), 7.02 (d, J = 8.7 Hz, 1H), 5.59 (s, 2H), 4.34-4.02 (m, 4H), 3.89-3.85 (m, 6H), 3.66-3.58 (m, 3H), 3.44-3.16 (m, 8H), 2.61-2.50 (m, 2H), 2.20-1.90 (m, 1H), 1.77-1.61 (m, 10H), 1.44-1.36 (m, 4H), 1.11-0.94 (m, 5H). |
| 89 | 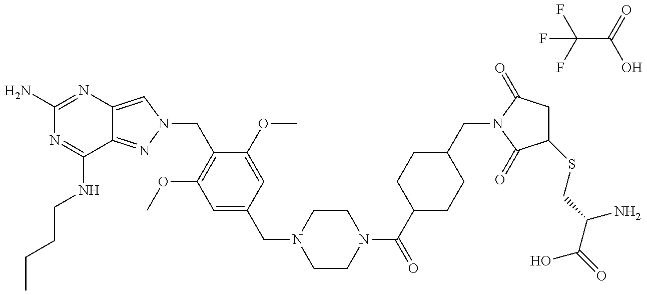 | 795.5 | (300 MHz, CD₃OD) δ 7.68 (s, 1H), 6.83 (s, 2H), 5.53 (s, 2H), 4.05- 3.96 (m, 3H), 3.88- 3.86 (m, 7H), 3.72- 3.64 (m, 3H), 3.63- 3.61 (m, 2H), 3.37- 3.35 (m, 2H), 3.01- 3.29 (m, 4H), 2.87- 2.85 (m, 3H), 2.59- 2.58 (m, 2H), 2.04- 2.01 (m, 1H), 1.77- 1.62 (m, 6H), 1.45- 1.32 (m, 5H), 1.28- 0.95 (m, 5H) |
| 90 | 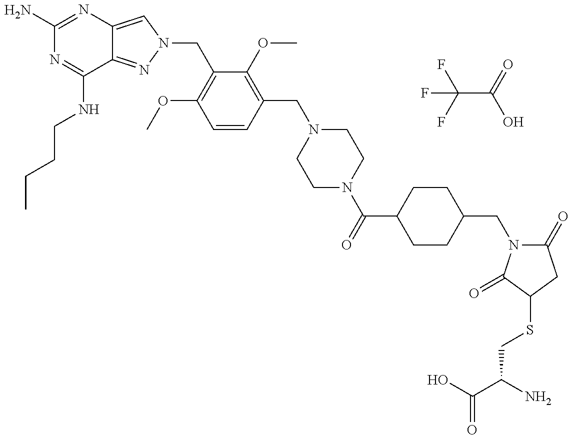 [SMILES: CCCCNc1nc(N)nc2cn(Cc3c(OC)ccc(CN4CCN(C(=O)C5CCC(CN6C(=O)CC(SC[C@H](N)C(=O)O)C6=O)CC5)CC4)c3OC)nc12 ; O=C(O)C(F)(F)F] | 795.2 | (300 MHz, CD₃OD) δ 7.82 (s, 1H), 7.60- 7.57 (m, 1H), 7.03- 7.00 (m, 1H), 4.86 (s, 2H), 4.35-4.02 (m, 4H), 3.89-3.85 (m, 8H), 3.70-3.61 (m, 3H), 3.58-3.16 (m, 8H), 2.62-2.59 (m, 2H), 2.30-1.95 (m, 1H), 1.78-1.61 (m, 7H), 1.44-1.37 (m, 5H), 1.12-0.94 (m, 5H) |
| 92 | 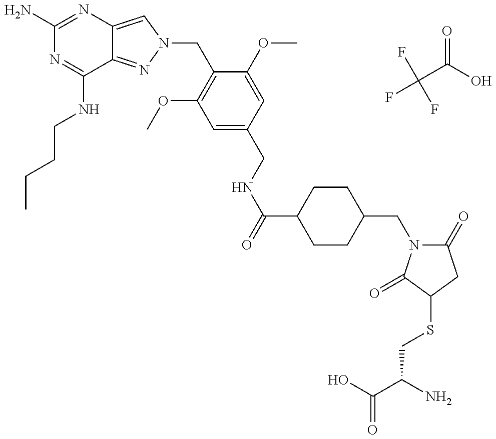 [SMILES: CCCCNc1nc(N)nc2cn(Cc3c(OC)cc(CNC(=O)C4CCC(CN5C(=O)CC(SC[C@H](N)C(=O)O)C5=O)CC4)cc3OC)nc12 ; O=C(O)C(F)(F)F] | 726.5 | (300 MHz, CD₃OD) δ 7.60-7.59 (m, 1H), 6.63 (s, 2H), 5.50 (m, 2H), 4.85-4.34 (m, 2H), 3.93-3.91 (m, 6H), 3.83-3.58 (m, 3H), 3.53-3.30 (m, 2H), 3.24-3.06 (m, 3H), 2.58-2.52 (m, 1H), 2.51-2.16 (m, 1H), 1.88-1.65 (m, 8H), 1.52-1.38 (m, 4H), 1.11-0.95 (m, 5H) |

### Example 15

### Preparation of Compound 93

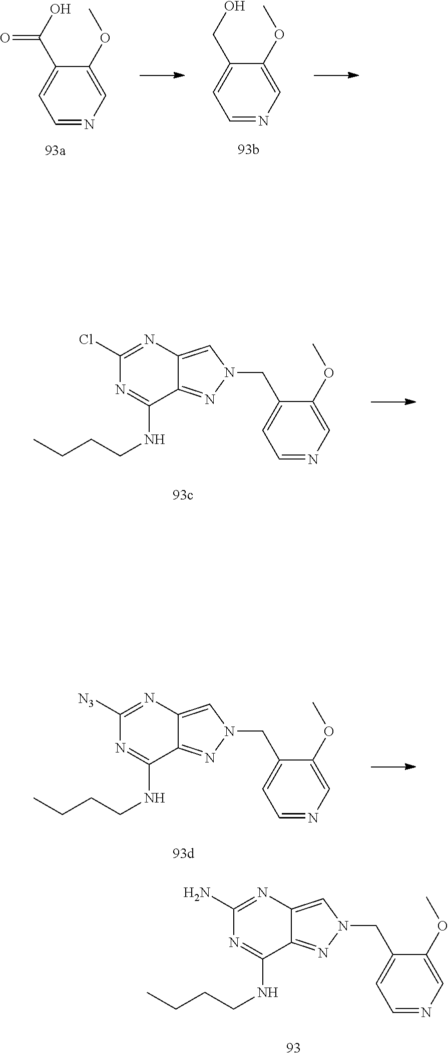

### Step A—Synthesis of Compound 93b

To a solution of 93a (500 mg, 3.27 mmol) in tetrahydrofuran (10 mL) was added borane (1M in tetrahydrofuran) (6.53 ml, 6.53 mmol) at 0° C., and the resulting mixture was allowed to stir for 3 hours at 65° C. The reaction was quenched with methanol, then concentrated in vacuo, and the resulting residue was purified using silica gel column chromatography (eluted with 0-60% ethyl acetate in petroleum ether) to provide compound 93b.

### Step B—Synthesis of Compound 93c

To a solution of N-butyl-5-chloro-1H-pyrazolo[4,3-d]pyrimidin-7-amine (200 mg, 0.886 mmol), compound 93b (185 mg, 1.329 mmol), and triphenylphosphine (302 mg, 1.152 mmol) in toluene (2 mL) was added (Z)-N-([(propan-2-yloxy)carbonyl]imino)(propan-2-yloxy)formamide (233 mg, 1.152 mmol) at 0° C., and the resulting mixture was allowed to stir for 2 hours at 25° C. The reaction mixture was concentrated in vacuo, and the resulting residue was purified using RP-flash chromatography (eluted with 0-70% acetonitrile in 10 mM aq. NH₄HCO₃) to provide compound 93c. MS: m/z=347.0 [M+H].

### Step C—Synthesis of Compound 93d

To a solution of compound 93c (120 mg, 0.346 mmol) in ethanol (2 mL), and acetic acid (0.5 mL) was added sodium azide (33.7 mg, 0.519 mmol) at 25° C., and the resulting mixture was allowed to stir for 2 hours at 100° C. The reaction mixture was concentrated in vacuo, and the resulting residue was purified using silica gel column chromatography (eluted with 0-45% ethyl acetate in petroleum ether) to provide compound 93d. MS: m/z=354.3 [M+H].

### Step D—Synthesis of Compound 93

To a mixture of compound 93d (100 mg, 0.283 mmol) in tetrahydrofuran (2 mL), and water (0.5 mL) was added trimethylphosphine (1M in tetrahydrofuran) (64.6 mg, 0.849 mmol), and allowed to stir for 15 hours at 50° C. under nitrogen atmosphere. The reaction mixture was concentrated in vacuo, and the resulting residue was purified using RP-flash chromatography (eluted with 0-51% acetonitrile in 10 mM aq. NH₄HCO₃) to provide compound 93. MS: m/z=328.05 [M+H]. ¹H NMR (300 MHz, d₆-DMSO) δ 8.31 (s, 1H), 8.22-8.18 (m, 1H), 7.56 (s, 1H), 6.75-6.69 (m, 1H), 5.75 (s, 1H), 5.45 (s, 2H), 4.72 (s, 2H), 3.99 (s, 3H), 3.64-3.53 (m, 2H), 1.75-1.58 (m, 2H), 1.53-1.37 (m, 2H), 1.03-0.93 (m, 3H).

The following compounds of the present invention were made using methodology described in Example 14 above, and substituting the appropriate reactants and/or reagents:

| | | MS | |
|---|---|---|---|
| Compound | Structure | [M + H] | ¹H NMR |
| 106 | 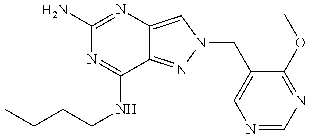 [SMILES: CCCCNc1nc(N)nc2cn(Cc3cncnc3OC)nc12] | 329.1 | (300 MHz, CDCl₃) δ 8.76 (s, 1H), 8.25 (s, 1H), 7.58 (s, 1H), 5.70 (s, 1H), 5.36 (s, 2H), 4.67 (s, 2H), 4.05 (s, 3H), 3.65-3.50 (m, 2H), 1.74-1.58 (m, 2H), 1.52-1.37 (m, 2H), 1.02-0.92 (m, |
| | | | 3H) |
| 111 | 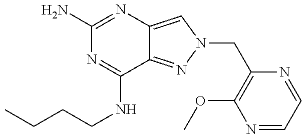 [SMILES: CCCCNc1nc(N)nc2cn(Cc3nccnc3OC)nc12] | 329.2 | (300 MHz, CD₃OD) δ 8.05-7.98 (m, 2H), 7.62 (s, 1H), 5.51 (s, 2H), 3.93-3.91 (m, 3H), 3.42-3.38 (m, 2H), 1.59-1.49 (m, 2H), 1.38-1.26 (m, 2H), 0.89-0.85 (m, 3H). |
| 121 | 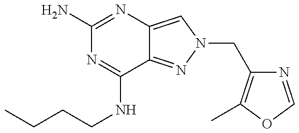 [SMILES: CCCCNc1nc(N)nc2cn(Cc3ncoc3C)nc12] | 302.1 | (300 MHz, CDCl₃) δ 7.74 (s, 1H), 7.56 (s, 1H), 5.84-5.82 (m, 1H), 5.29 (s, 2H), 4.88- 4.85 (m, 2H), 3.58- 3.56 (m, 2H), 2.32- 2.25 (m, 3H), 1.73- 1.71 (m, 2H), 1.51- |
| | | | 1.48 (m, 2H), 1.01- |
| | | | 0.99 (m, 3H). |
| 122 | 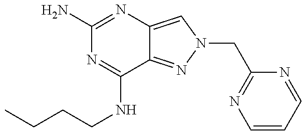 [SMILES: CCCCNc1nc(N)nc2cn(Cc3ncccn3)nc12] | 299.1 | (300 MHz, CDCl₃) δ 8.74-8.72 (m, 2H), 7.66 (s, 1H), 7.26-7.24 (m, 1H), 5.93-5.91 (m, 1H), 5.68 (s, 2H), 4.81- 4.79 (m, 2H), 3.57- 3.54 (m, 2H), 1.65- 1.63 (m, 2H), 1.51- 1.48 (m, 2H), 0.99- |
| | | | 0.97 (m, 3H) |
| 140 | 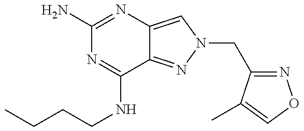 [SMILES: CCCCNc1nc(N)nc2cn(Cc3nocc3C)nc12] | 302.2 | (300 MHz, CDCl₃) δ 8.40 (s, 1H), 7.60 (s, 1H), 6.25 (s, 2H), 6.25- 5.99 (m, 1H), 5.59 (s, 2H), 3.66-3.59 (m, 2H), 2.20-1.70 (m, 3H), 1.67-1.53 (m, 2H), 1.51-1.41 (m, |
| | | | 2H), 1.38-1.14 (m, |
| | | | 3H). |
| 160 | 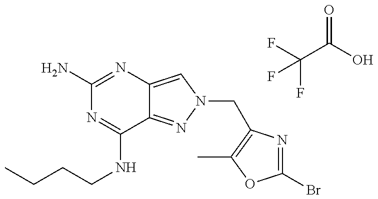 | 380.0 | (400 MHz, CD₃OD) δ 7.78 (s, 1H), 5.29 (s, 2H), 3.55-3.51 (m, 2H), 2.33 (s, 3H), 1.60- 1.54 (m, 2H), 1.35- 1.30 (m, 2H), 0.89- 0.86 (m, 3H). |
| 170 | 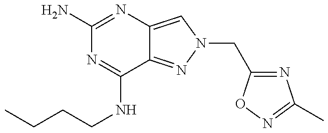 [SMILES: CCCCNc1nc(N)nc2cn(Cc3nc(C)no3)nc12] | 303.2 | (300 MHz, CDCl₃) δ 7.65 (s, 1H), 5.79 (s, 1H), 5.76-5.64 (m, 2H), 4.73 (s, 2H), 3.61- 3.57 (m, 2H), 2.44 (s, 3H), 1.67-1.65 (m, 2H), 1.49-1.47 (m, 2H), 1.01-0.96 (m, 3H). |
| 171 | 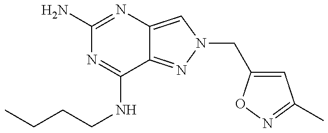 [SMILES: CCCCNc1nc(N)nc2cn(Cc3cc(C)no3)nc12] | 302.2 | (300 MHz, CDCl₃) δ 7.69 (s, 1H), 6.02- 6.01 (m, 1H), 5.79 (s, 1H), 5.51-5.50 (m, 2H), 4.82 (s, 2H), 3.61- 3.59 (m, 2H), 2.33- 2.29 (m, 3H), 1.73- 1.71 (m, 2H), 1.66- 1.63 (m, 2H), 1.01- |
| | | | 0.99 (m, 3H) |
| 172 | 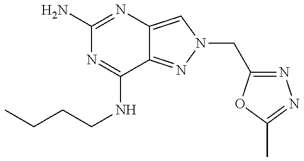 [SMILES: CCCCNc1nc(N)nc2cn(Cc3nnc(C)o3)nc12] | 303.3 | (300 MHz, CDCl₃) δ 7.62 (s, 1H), 5.86 (s, 1H), 5.62 (s, 2H), 4.78 (s, 2H), 3.58-3.56 (m, 2H), 2.52 (s, 3H), 1.71- 1.62 (m, 2H), 1.51- 1.39 (m, 2H), 1.00- 0.95 (m, 3H). |
| 173 | 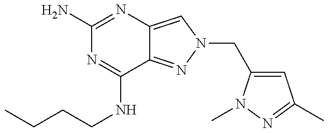 [SMILES: CCCCNc1nc(N)nc2cn(Cc3cc(C)nn3C)nc12] | 315.2 | (300 MHz, CDCl₃) δ 7.69 (s, 1H), 6.08- 6.06 (m, 1H), 5.74 (s, 1H), 5.62-5.50 (m, 2H), 4.77 (s, 2H), 3.74- 3.60 (m, 3H), 3.58- 3.46 (m, 2H), 2.25- 2.22 (m, 3H), 1.73- 1.71 (m, 2H), 1.66- |
| | | | 1.63 (m, 2H), 1.01- |
| | | | 0.99 (m, 3H). |
| 174 | 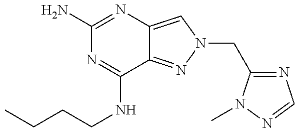 [SMILES: CCCCNc1nc(N)nc2cn(Cc3ncnn3C)nc12] | 302.2 | (300 MHz, DMSO) δ 7.89 (s, 1H), 7.80 (s, 1H), 7.55 (s, 1H), 5.72 (s, 2H), 5.55 (s, 2H), 3.89 (s, 3H), 3.41- 3.39 (m, 2H), 1.61- 1.51 (m, 2H), 1.37- 1.25 (m, 2H), 1.01- 0.97 (m, 3H). |
| 175 | 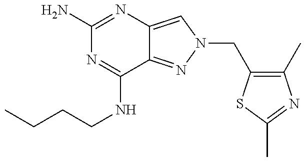 [SMILES: CCCCNc1nc(N)nc2cn(Cc3sc(C)nc3C)nc12] | 332.3 | (300 MHz, CDCl₃) δ 7.50 (s, 1H), 5.97 (s, 1H), 5.48 (s, 2H), 5.38 (s, 2H), 3.62 (s, 2H), 2.65 (s, 3H), 2.47 (s, 3H), 1.75-1.65 (m, 2H), 1.54-1.41 (m, 2H), 1.03-0.98 (m, 3H) |
| 177 | 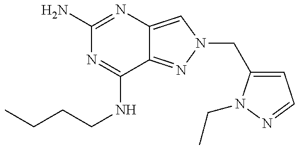 [SMILES: CCCCNc1nc(N)nc2cn(Cc3ccnn3CC)nc12] | 315.3 | (300 MHz, DMSO) δ 7.67-7.64 (m, 2H), 7.52 (s, 1H), 7.01 (s, 1H), 5.49 (s, 4H), 3.99- 3.91 (m, 2H), 3.40- 3.36 (m, 2H), 1.61- 1.56 (m, 2H), 1.54- 1.51 (m, 2H), 1.15- 1.01 (m, 3H), 0.95- 0.85 (m, 3H). |
| 179 | 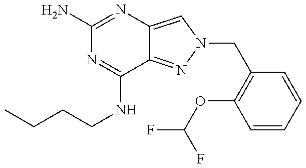 [SMILES: CCCCNc1nc(N)nc2cn(Cc3ccccc3OC(F)F)nc12] | 363.3 | (300 MHz, DMSO) δ 7.55 (s, 1H), 7.40- 7.39 (m, 1H), 7.37- 7.35 (m, 2H), 7.22- 7.17 (m, 1H), 6.81- 6.32 (m, 1H), 5.86 (s, 1H), 5.50 (s, 2H), 4.94 (s, 2H), 3.67-3.58 (m, 2H), 1.72-1.62 (m, 2H), 1.52-1.40 (m, 2H), 1.01-0.96 (m, 3H) |
| 180 | 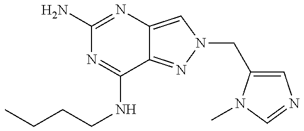 [SMILES: CCCCNc1nc(N)nc2cn(Cc3cncn3C)nc12] | 301.1 | (300 MHz, CDCl₃) δ 7.69 (s, 1H), 7.45 (s, 1H), 7.28 (s, 1H), 5.86- 5.85 (m, 1H), 5.43 (s, 2H), 5.02-5.00 (m, 2H), 3.62-3.59 (m, 2H), 3.51 (s, 3H), 1.69- 1.68 (m, 2H), 1.66- 1.64 (m, 2H), 1.02- |
| | | | 0.99 (m, 3H) |
| 181 | 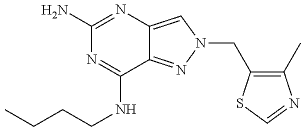 [SMILES: CCCCNc1nc(N)nc2cn(Cc3scnc3C)nc12] | 318.2 | (300 MHz, CDCl₃) δ 8.72 (s, 1H), 7.49 (s, 1H), 5.85 (s, 1H), 5.69 (s, 2H), 5.08 (s, 2H), 3.61-3.60 (m, 2H), 2.48 (s, 3H), 1.74-1.65 (m, 2H), 1.53-1.41 (m, 2H), 1.02-0.97 (m, 3H) |
| 182 | 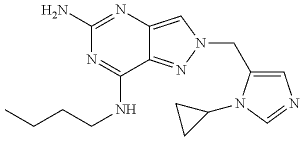 [SMILES: CCCCNc1nc(N)nc2cn(Cc3cncn3C3CC3)nc12] | 327.1 | (300 MHz, CDCl₃) δ 7.53 (s, 1H), 7.00 (m, 1H), 6.92-6.91 (m, 1H), 5.88 (s, 1H), 5.00 (s, 2H), 3.59-3.57 (m, 2H), 3.04-3.02 (m, 1H), 2.82-2.67 (m, 2H), 1.72-1.69 (m, 2H), 1.66-1.62 (m, |
| | | | 2H), 1.27-1.04 (m, |
| | | | 7H). |
| 186 | 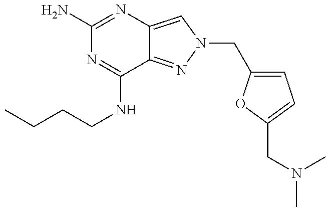 [SMILES: CCCCNc1nc(N)nc2cn(Cc3ccc(CN(C)C)o3)nc12] | 344.3 | (300 MHz, CDCl₃) δ 7.54 (s, 1H), 6.36- 6.35 (m, 1H), 6.20- 6.19 (m, 1H), 5.96 (s, 1H), 5.39 (s, 2H), 5.13 (s, 2H), 3.61-3.59 (m, 2H), 3.45 (s, 2H), 2.27 (s, 6H), 1.73-1.63 (m, 2H), 1.52-1.40 (m, 2H), 1.01-0.96 (m, 3H) |
| 188 | 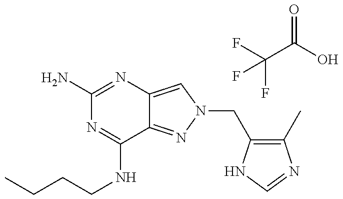 | 301.1 | (300 MHz, CD₃OD) δ 8.73 (s, 1H), 8.00 (s, 1H), 5.68 (s, 2H), 3.67- 3.55 (m, 2H), 2.45- 2.38 (m, 3H), 1.95- 1.89 (m, 2H), 1.67- 1.64 (m, 2H), 1.01- 0.99 (m, 3H) |
| 197 | 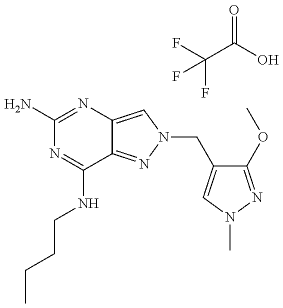 | 331.1 | (300 MHz, CD₃OD) δ 7.76 (s, 1H), 7.54 (s, 1H), 5.27 (s, 2H), 3.89 (s, 3H), 3.73 (s, 3H), 3.67-3.62 (m, 2H), 1.72-1.67 (m, 2H), 1.48-1.40 (m, 2H), 1.02-0.97 (m, 3H). |
| 206 | 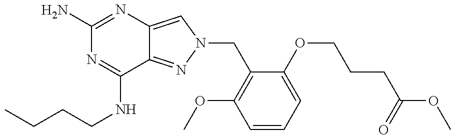 [SMILES: CCCCNc1nc(N)nc2cn(Cc3c(OC)cccc3OCCCC(=O)OC)nc12] | 443.3 | (300 MHz, CD₃OD) δ 7.47 (s, 1H), 7.37-7.31 (m, 1H), 6.74-6.67 (m, 2H), 5.52 (s, 2H), 4.07- 4.03 (m, 2H), 3.88 (s, 3H), 3.61 (s, 3H), 3.56- 3.52 (m, 2H), 2.42- 2.37 (m, 2H), 2.08- 1.91 (m, 2H), 1.71- |
| | | | 1.70 (m, 2H), 1.68- |
| | | | 1.65 (m, 2H), 0.99- |
| | | | 0.97 (m, 3H). |
| 207 | 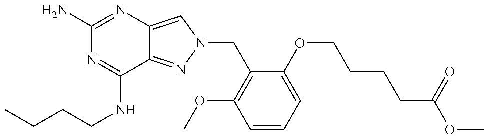 [SMILES: CCCCNc1nc(N)nc2cn(Cc3c(OC)cccc3OCCCCC(=O)OC)nc12] | 457.3 | (300 MHz, CD₃OD) δ 7.47 (s, 1H), 7.37-7.31 (m, 1H), 6.74-6.67 (m, 2H), 5.52 (s, 2H), 4.04- 4.00 (m, 2H), 3.88 (s, 3H), 3.62 (s, 3H), 3.55- 3.48 (m, 2H), 2.34- 2.29 (m, 2H), 1.82- 1.75 (m, 6H), 1.69- |
| | | | 1.62 (m, 2H), 0.99- |
| | | | 0.97 (m, 3H). |
| 212 | 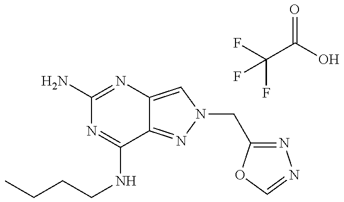 | 289.2 | (300 MHz, CD₃OD) δ 9.04 (s, 1H), 8.10 (s, 1H), 5.99 (s, 2H), 3.67- 3.65 (m, 2H), 1.74- 1.72 (m, 2H), 1.47- 1.44 (m, 2H), 1.01- 0.99 (m, 3H). |

### Example 16

### Preparation of Compound 94

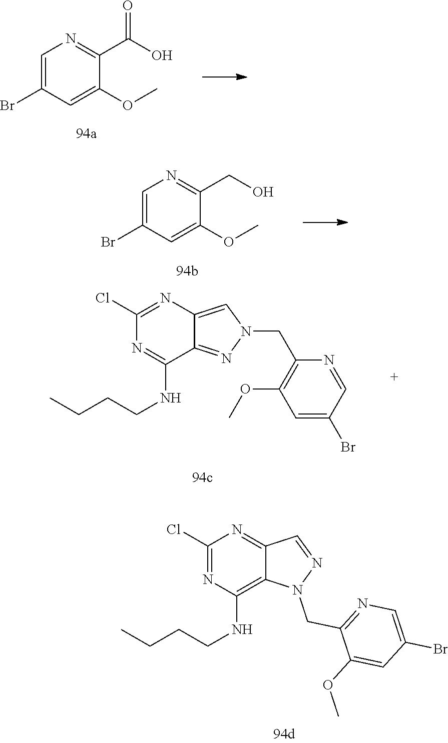 [SMILES: COc1cc(Br)cnc1C(=O)O ; COc1cc(Br)cnc1CO ; CCCCNc1nc(Cl)nc2cn(Cc3ncc(Br)cc3OC)nc12 ; CCCCNc1nc(Cl)nc2cnn(Cc3ncc(Br)cc3OC)c12]

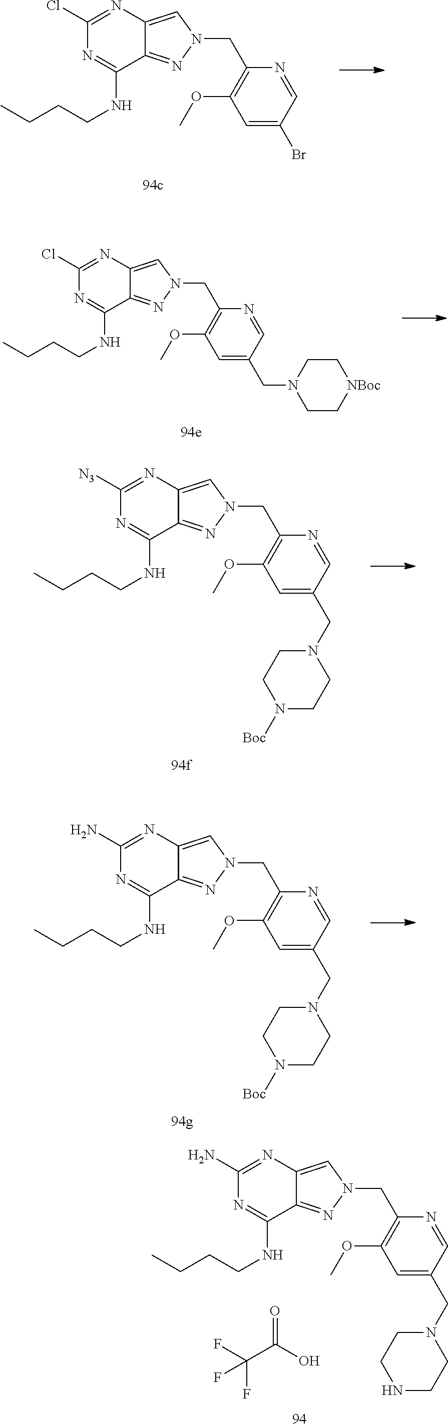 [SMILES: CCCCNc1nc(Cl)nc2cn(Cc3ncc(Br)cc3OC)nc12 ; CCCCNc1nc(Cl)nc2cn(Cc3ncc(CN4CCN(C(=O)OC(C)(C)C)CC4)cc3OC)nc12 ; CCCCNc1nc(N=[N+]=[N-])nc2cn(Cc3ncc(CN4CCN(C(=O)OC(C)(C)C)CC4)cc3OC)nc12 ; CCCCNc1nc(N)nc2cn(Cc3ncc(CN4CCN(C(=O)OC(C)(C)C)CC4)cc3OC)nc12 ; CCCCNc1nc(N)nc2cn(Cc3ncc(CN4CCNCC4)cc3OC)nc12 ; O=C(O)C(F)(F)F]

### Step A—Synthesis of Compound 94b

To a solution of compound 94a (500 mg, 2.155 mmol) in DME (2 mL) were added 4-methylmorpholine (218 mg, 2.155 mmol), and isobutyl chloroformate (294 mg, 2.155 mmol) at −10° C., and the resulting mixture was allowed to warm to room temperature over 30 minutes The solid was filtered and washed with DME (3 mL), the filtrate was treated with NaBH₄ (163 mg, 4.31 mmol), and the resulting mixture was allowed to stir at room temperature for 30 minutes MeOH (1 mL) was carefully added and the reaction mixture was allowed to stir for a further 30 minutes The volatiles were evaporated, and the resulting residue was dissolved in DCM (50 mL), washed with water (20 mL), and brine (20 mL), the organic phase was concentrated in vacuo. The resulting residue was purified using silica gel column chromatography (eluted with 0-40% ethyl acetate in petroleum ether) to provide compound 94b.

### Step B—Synthesis of Compound 94c and 94d

To a mixture of N-butyl-5-chloro-2H-pyrazolo[4,3-d]pyrimidin-7-amine (200 mg, 0.886 mmol), and compound 94b (232 mg, 1.063 mmol) in toluene (3 mL) under the argon atmosphere at 0° C. was added (tributylphosphoranylidene)acetonitrile (642 mg, 2.66 mmol), and the resulting mixture was allowed to stir for 3 hours at 100° C. The reaction mixture was poured into ethyl acetate (150 mL), and aq. NaHCO₃ (sat., 50 mL), the organic layer was washed brine (sat., 2×50 mL), dried over anhydrous Na₂SO₄, and concentrated in vacuo. The resulting residue was purified using RP-flash chromatography (eluted with 0-60% acetonitrile in 5 mM aq. NH₄HCO₃) to provide compound 94c and compound 94d. MS: m/z=425.0 [M+H].

### Step C—Synthesis of Compound 94e

A mixture of compound 94c (100 mg, 0.235 mmol), ((((4-(tert-butoxycarbonyl)piperazin-1-yl)methyl)boraneylidene)-13-fluoraneyl)potassium(III) fluoride (108 mg, 0.352 mmol), potassium phosphate tribasic (0.088 ml, 1.057 mmol), and bis(di-tert-butyl(4-dimethylaminophenyl)phosphine)dichloropalladium(ii) (8.32 mg, 0.012 mmol) in 1,4-dioxane (3 mL), and water (0.3 mL) was allowed to stir for 3 hours at 80° C. The reaction mixture was poured into ethyl acetate (100 mL), and water (50 mL), the organic layer was washed with brine (sat., 2×30 mL), dried over anhydrous Na₂SO₄, and concentrated in vacuo. The resulting residue was purified using silica gel column chromatography (eluted with 0-65% ethyl acetate in petroleum ether) to provide compound 94e. MS: m/z=545.4 [M+H].

### Step D—Synthesis of Compound 94f

A mixture of compound 94e (170 mg, 0.312 mmol), and sodium azide (30.4 mg, 0.468 mmol) in EtOH (3 mL), and acetic acid (0.6 mL) was allowed to stir for 3 hours at 100° C. The reaction mixture was concentrated in vacuo, and the resulting residue was diluted with ethyl acetate (80 mL), washed with aq. NaHCO₃ (sat., 25 mL), and brine (sat., 20 mL), dried over anhydrous Na₂SO₄, and concentrated to provide compound 94f. MS: m/z=552.4 [M+H].

### Step E—Synthesis of Compound 94g

To a mixture of compound 94f (110 mg, 0.199 mmol) in THF (2 mL), and water (0.5 mL) was added trimethylphosphine (1 M in THF, 0.598 mL, 0.598 mmol), and the resulting mixture was allowed to stir for 5 hours at 50° C. The reaction mixture was quenched with MeOH (2 mL), and concentrated in vacuo. The resulting residue was purified using silica gel column chromatography (eluted with 0-8% MeOH (contained 10% NH₄OH) in DCM) to provide compound 94g. MS: m/z=526.3 [M+H].

### Step F—Synthesis of Compound 94

To a mixture of compound 94g (90 mg, 0.171 mmol) in DCM (3 mL) at 0° C. was added 4 M HCl in dioxane (1 mL, 4.00 mmol), and the resulting mixture was allowed to stir for 1 hour at 28° C. The reaction mixture was concentrated in vacuo, and the resulting residue was purified using RP-Flash chromatography (eluted with 0-30% acetonitrile in 0.05% aq. TFA) to provide compound 94. MS: m/z=426.25 [M+H]. ¹H NMR (300 MHz, CD₃OD) δ 8.12 (s, 1H), 7.89 (s, 1H), 7.58 (s, 1H), 5.65 (s, 2H), 3.94-3.81 (m, 5H), 3.63-3.58 (m, 2H), 3.31-3.29 (m, 4H), 2.90-2.85 (m, 4H), 1.71-1.64 (m, 2H), 1.47-1.37 (m, 2H), 0.98 (t, J=7.2 Hz, 3H).

The following compounds of the present invention were made using methodology described in Example 16 above, and substituting the appropriate reactants and/or reagents:

| | | MS | |
|---|---|---|---|
| Compound | Structure | [M + H] | ¹H NMR |
| 99 | 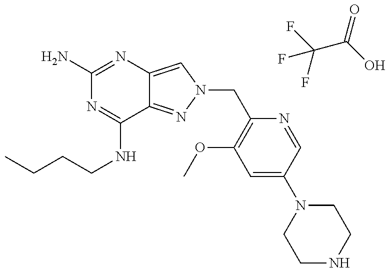 | 412.2 | (300 MHz, CD₃OD) δ 7.99-7.75 (m, 2H), 7.19 (s, 1H), 5.62 (s, 2H), 3.95 (s, 3H), 3.65- 3.62 (m, 6H), 3.42- 3.32 (m, 4H), 1.73- 1.68 (m, 2H), 1.63- 1.59 (m, 2H), 0.99- 0.97 (m, 3H) |
| 123 | 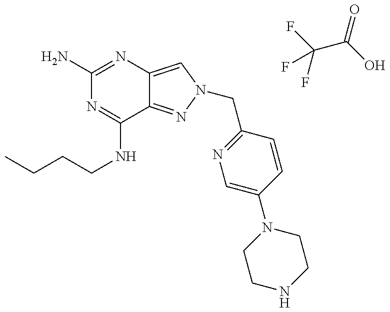 [SMILES: CCCCNc1nc(N)nc2cn(Cc3ccc(N4CCNCC4)cn3)nc12 ; O=C(O)C(F)(F)F] | 382.2 | (300 MHz, CD₃OD) δ 8.31 (s, 1H), 7.95 (s, 1H), 7.52-7.48 (m, 1H), 7.38-7.35 (m, 1H), 5.58 (s, 2H), 3.64- 3.62 (m, 2H), 3.51- 3.46 (m, 4H), 3.31- 3.29 (m, 4H), 1.69- 1.64 (m, 2H), 1.47- 1.40 (m, 2H), 0.99- 0.97 (m, 3H) |
| 129 | 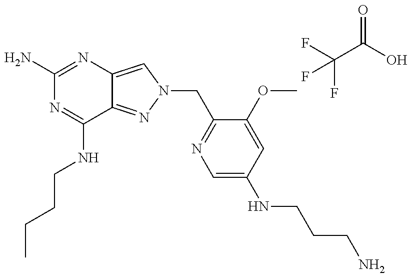 | 400.3 | (300 MHz, CD₃OD) δ 7.96 (s, 1H), 7.67 (s, 1H), 7.07 (s, 1H), 5.65 (s, 2H), 3.96 (s, 3H), 3.65-3.60 (m, 2H), 3.36-3.33 (m, 2H), 3.11-3.06 (m, 2H), 2.07-2.00 (m, 2H), 1.73-1.68 (m, 2H), 1.48-1.46 (m, 2H), 1.01-0.98 (m, 3H) |
| 138 | 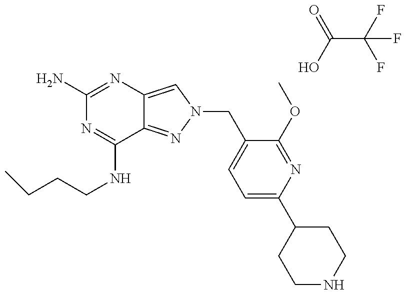 | 411.2 | (400 MHz, CD₃OD) δ 7.90-7.87 (m, 1H), 7.55-7.53 (m, 1H), 6.90-6.88 (m, 1H), 5.49 (s, 2H), 3.98 (s, 3H), 3.63-3.60 (m, 2H), 3.51-3.48 (m, 2H), 3.32-3.17 (m, 2H), 3.16-3.01 (m, 1H), 2.14-2.05 (m, 4H), 1.70-1.63 (m, 2H), 1.46-1.36 (m, 2H), 0.99-0.91 (m, 3H). |
| 142 | 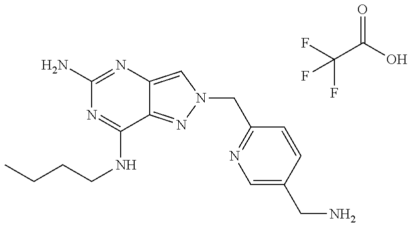 [SMILES: CCCCNc1nc(N)nc2cn(Cc3ccc(CN)cn3)nc12 ; O=C(O)C(F)(F)F] | 327.2 | (400 MHz, CD₃OD) δ 8.66 (s, 1H), 8.00-8.04 (m, 2H), 7.47-7.45 (m, 1H), 5.73 (s, 2H), 4.21 (s, 2H), 3.66-3.64 (m, 2H), 1.72-1.69 (m, 2H), 1.48-1.46 (m, 2H), 1.01-0.99 (m, 3H) |
| 144 | 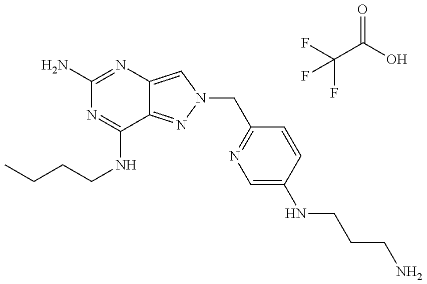 [SMILES: CCCCNc1nc(N)nc2cn(Cc3ccc(NCCCN)cn3)nc12 ; O=C(O)C(F)(F)F] | 370.2 | (400 MHz, CD₃OD) δ 8.04-8.02 (m, 1H), 7.99 (s, 1H), 7.51-7.50 (m, 1H), 7.40-7.39 (m, 1H), 5.65 (s, 2H), 3.64- 3.62 (m, 2H), 3.32- 3.30 (m, 2H), 3.09- 3.05 (m, 2H), 2.02- 1.98 (m, 2H), 1.73- 1.69 (m, 2H), 1.48- 1.46 (m, 2H), 1.01- 0.97 (m, 3H) |
| 159 | 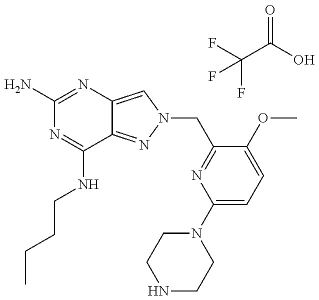 | 412.2 | (300 MHz, CD₃OD) δ 7.92 (s, 1H), 7.48- 7.45 (m, 2H), 5.59 (s, 2H), 3.86 (s, 3H), 3.63- 3.61 (m, 6H), 3.33- 3.32 (m, 4H), 1.73- 1.71 (m, 2H), 1.68- 1.67 (m, 2H), 1.03- 0.99 (m, 3H) |
| 162 | 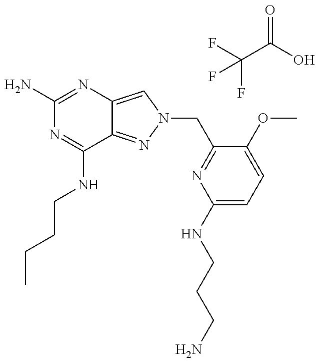 | 400.1 | (300 MHz, CD₃OD) δ 7.90-7.88 (m, 1H), 7.49 (s, 1H), 6.68- 6.65 (m, 1H), 5.54 (s, 2H), 3.85-3.82 (m, 3H), 3.66-3.64 (m, 2H), 3.55-3.41 (m, 2H), 3.09-3.02 (m, 2H), 1.97-1.92 (m, 2H), 1.88-1.74 (m, 2H), 1.49-1.47 (m, 2H), 1.01-0.91 (m, 3H) |

### Example 17

### Preparation of Compound 95

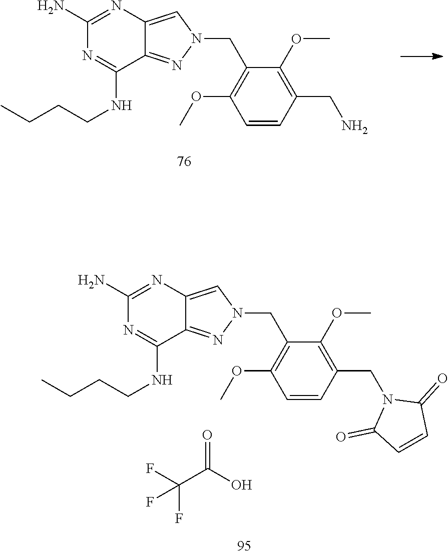

A mixture of compound 76 (30 mg, 0.078 mmol), and furan-2,5-dione (15.26 mg, 0.156 mmol) in acetic acid (0.5 mL) was stirred at 120° C. for 3 hours. The reaction mixture was concentrated in vacuo, and the resulting residue was purified using prep-HPLC with the following condition: Column: SunFire Prep C¹⁸ OBD Column, 19×150 mm 5 um 10 nm; Mobile Phase A: Water (0.05% TFA), Mobile Phase B: acetonitrile; Flow rate: 20 mL/min; Gradient: 15% B to 50% B in 4.3 min; Detector: UV 254 nm; RT: 4.02 minutes to provide compound 95. MS: m/z=466.3 [M+H]. ¹H NMR (300 MHz, CD₃OD) δ 7.75 (s, 1H), 7.25-7.22 (m, 1H), 6.93-6.84 (m, 3H), 5.57 (s, 2H), 4.73 (s, 2H), 3.95-3.84 (m, 6H), 3.65-3.61 (m, 2H), 1.71-1.62 (m, 2H), 1.59-1.45 (m, 2H), 1.02-0.93 (m, 3H).

The following compounds of the present invention were made using methodology described in Example 17 above, and substituting the appropriate reactants and/or reagents:

| | | MS | |
|---|---|---|---|
| Compound | Structure | [M + H] | ¹H NMR |
| 103 | 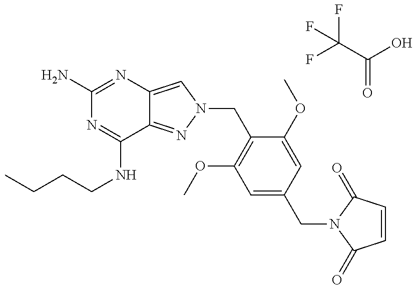 | 466.1 | (300 MHz, CDCl₃) δ 7.44 (s, 1H), 6.74-6.60 (m, 4H), 5.48 (s, 2H), 4.71 (s, 2H), 3.84 (s, 6H), 3.65-3.59 (m, 2H), 1.73-1.60 (m, 2H), 1.50-1.35 (m, 2H), 1.5-0.89 (m, 3H). |
| 104 | 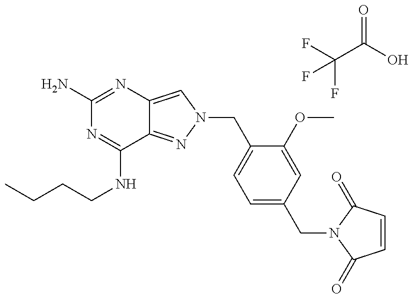 | 436.3 | (300 MHz, CDCl₃) δ 7.59 (s, 1H), 7.26- 7.13 (m, 1H), 7.00- 6.96 (m, 2H), 6.72 (s, 2H), 6.60-6.49 (m, 1H), 5.45 (s, 2H), 4.67 (s, 2H), 3.85 (s, 3H), 3.65-3.61 (m, 2H), 1.76-1.70 (m, 2H), 1.65-1.63 (m, 2H), 0.98 (t, J = 7.5 Hz, 3H) |
| 110 | 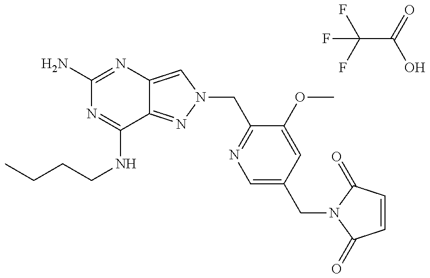 | 437.2 | (300 MHz, d₆-DMSO + D₂O) δ 7.97-7.89 (m, 2H), 7.34 (s, 1H), 6.96- 6.95 (m, 2H), 5.58 (s, 2H), 4.63 (s, 2H), 3.82 (s, 3H), 3.50-3.46 (m, 2H), 1.57-1.52 (m, 2H), 1.31-1.24 (m, 2H), 0.88-0.84 (m, 3H) |
| 127 | 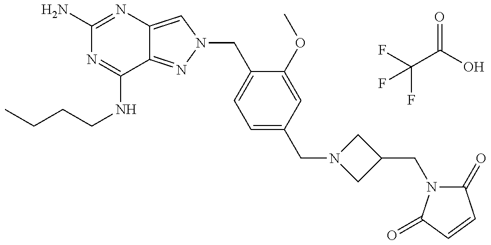 [SMILES: CCCCNc1nc(N)nc2cn(Cc3ccc(CN4CC(CN5C(=O)C=CC5=O)C4)cc3OC)nc12 ; O=C(O)C(F)(F)F] | 505.3 | (300 MHz, CD₃OD) δ 7.85 (s, 1H), 7.29-7.27 (m, 1H), 7.13 (s, 1H), 7.05-7.02 (m, 1H), 6.83 (s, 2H), 5.52 (s, 2H), 4.35-3.99 (m, 9H), 3.74-3.70 (m, 2H), 3.59-3.53 (m, 2H), 3.11-3.07 (m, 1H), 1.71-1.61 (m, 2H), 1.44-1.34 (m, 2H), 0.98 (t, J = 9.0 Hz, 3H) |
| 143 | 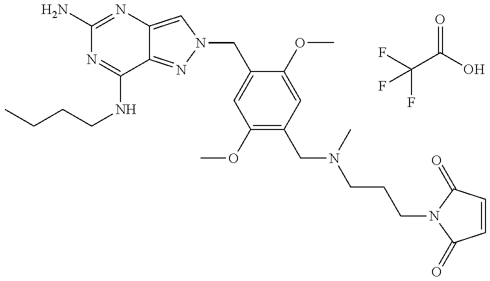 [SMILES: CCCCNc1nc(N)nc2cn(Cc3cc(OC)c(CN(C)CCCN4C(=O)C=CC4=O)cc3OC)nc12 ; O=C(O)C(F)(F)F] | 537.3 | (300 MHz, CD₃OD) δ 7.76 (s, 1H), 6.88 (s, 2H), 6.82 (s, 2H), 5.60 (s, 2H), 4.34 (s, 2H), 3.94 (s, 6H), 3.65- 3.58 (m, 4H), 3.32 (s, 2H), 2.85 (s, 3H), 2.06- 2.05 (m, 2H), 1.73- 1.64 (m, 2H), 1.49- 1.37 (m, 2H), 1.01- 0.98 (m, 3H) |

### Example 18

### Preparation of Compound 96

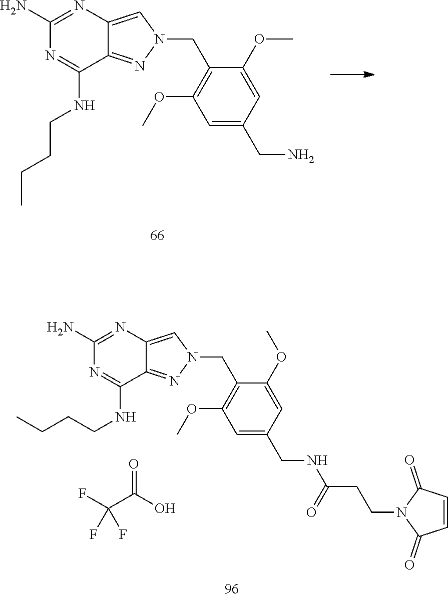

To a stirred mixture of 3-(2,5-dioxo-2,5-dihydro-1H-pyrrol-1-yl)propanoic acid (21.94 mg, 0.130 mmol), and HATU (49.3 mg, 0.130 mmol) in DMF (0.2 mL) at 0° C. under nitrogen atmosphere was added dropwise DIEA (0.068 ml, 0.389 mmol), and the resulting mixture was allowed to stir at 25° C. for 15 minutes to form mixture A. To a separated flask was added compound 66 (50 mg, 0.130 mmol) in DMF (0.3 mL) at 25° C. under nitrogen atmosphere, DIEA (0.045 ml, 0.259 mmol) was added dropwise and the resulting mixture was allowed to stir for 15 minutes at 25° C. to form mixture B. The mixture A was added dropwise into mixture B at 0° C., and the resulting mixture was allowed to stir at 25° C. for 30 minutes The reaction was concentrated in vacuo, and the resulting residue was purified using prep-HPLC with the following condition: Column: SunFire Prep C¹⁸ OBD Column, 19×150 mm 5 um 10 nm; Mobile Phase A: Water (0.05% TFA), Mobile Phase B: acetonitrile; Flow rate: 20 mL/min; Gradient: 20% B to 50% B in 4.3 min; Detector: UV 210/254 nm; RT: 4.02 minutes to provide compound 96. MS: m/z=537.1 [M+H]. ¹H NMR (300 MHz, CDCl₃) δ 7.61 (s, 1H), 6.68 (s, 2H), 6.60-6.56 (m, 2H), 5.98-5.97 (m, 1H), 5.49 (s, 2H), 4.43-4.40 (m, 2H), 3.91-3.84 (m, 8H), 3.66-3.59 (m, 2H), 2.63-2.58 (m, 2H), 1.64-1.41 (m, 2H), 1.25-1.23 (m, 2H), 1.01-0.92 (m, 3H).

The following compounds of the present invention were made using methodology described in Example 18 above, and substituting the appropriate reactants and/or reagents:

| | | MS | |
|---|---|---|---|
| Compound | Structure | [M + H] | ¹H NMR |
| 105 | 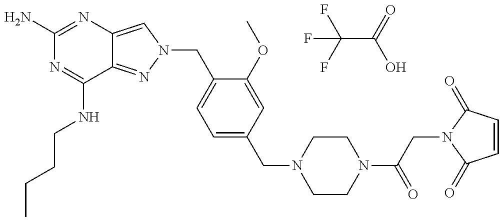 | 562.1 | (300 MHz, CD₃OD) δ 7.87 (s, 1H), 7.33-7.30 (m, 1H), 7.21 (s, 1H), 7.10-7.08 (m, 1H), 6.91 (s, 2H), 5.54 (s, 2H), 4.46 (s, 2H), 4.33- 4.32 (m, 2H), 3.98- 3.87 (m, 6H), 3.68- 3.52 (m, 2H), 3.31- 3.22 (m, 5H), 1.72- 1.69 (m, 2H), 1.66- 1.61 (m, 2H), 0.99- 0.97 (m, 3H) |
| 119 | 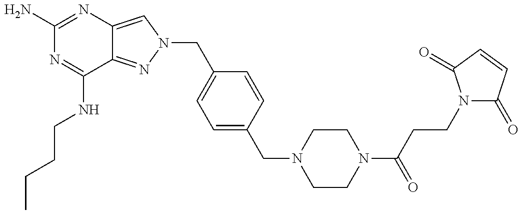 [SMILES: CCCCNc1nc(N)nc2cn(Cc3ccc(CN4CCN(C(=O)CCN5C(=O)C=CC5=O)CC4)cc3)nc12] | 546.2 | (300 MHz, CD₃CN) δ 7.83-7.73 (m, 1H), 7.73-7.50 (m, 1H), 7.41-7.35 (m, 4H), 6.79-6.76 (m, 2H), 5.60 (s, 2H), 4.20 (s, 2H), 3.84-3.57 (m, 8H), 3.20-3.07 (m, 5H), 2.78-2.61 (m, 3H), 1.69-1.62 (m, 2H), 1.44-1.34 (m, 2H), 1.00-0.90 (m, 3H). |

### Example 19

### Preparation of Compound 97

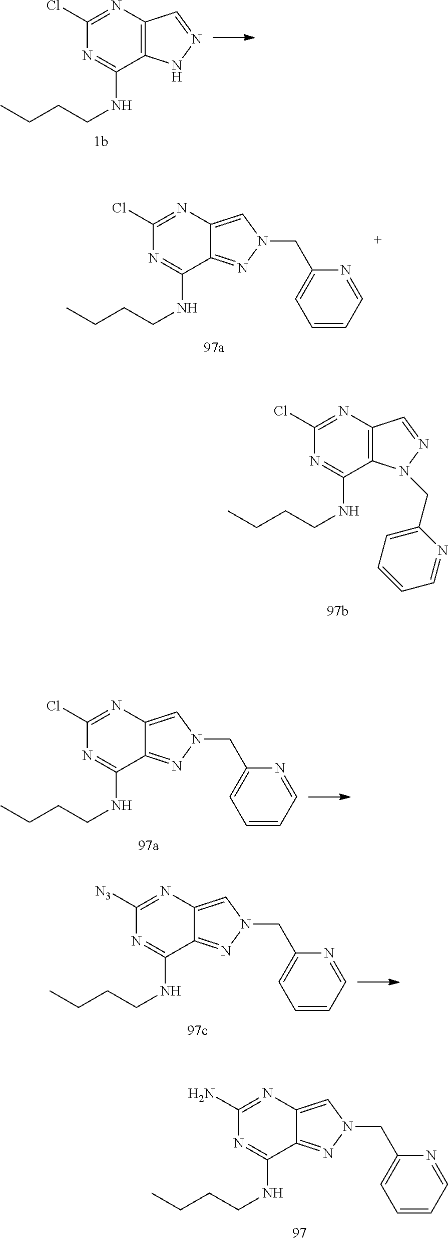 [SMILES: CCCCNc1nc(Cl)nc2cn[nH]c12 ; CCCCNc1nc(Cl)nc2cn(Cc3ccccn3)nc12 ; CCCCNc1nc(Cl)nc2cnn(Cc3ccccn3)c12 ; CCCCNc1nc(Cl)nc2cn(Cc3ccccn3)nc12 ; CCCCNc1nc(N=[N+]=[N-])nc2cn(Cc3ccccn3)nc12 ; CCCCNc1nc(N)nc2cn(Cc3ccccn3)nc12]

### Step A—Synthesis of Compound 97a and 97b

To a mixture of compound 1b (50 mg, 0.222 mmol), and potassium carbonate (92 mg, 0.665 mmol) in acetonitrile (1.5 mL) was added 2-(bromomethyl)pyridine hydrobromide (67.2 mg, 0.266 mmol) at 25° C., and the resulting mixture was allowed to stir at 80° C. for 2 hours . The reaction mixture was concentrated in vacuo, and the resulting residue was purified using prep-TLC, eluted with (1/1) petroleum ether/EA to provide compound 97a and compound 97b. MS: m/z=317.1 [M+H].

### Step B—Synthesis of Compound 97c

A mixture of compound 97a (30 mg, 0.095 mmol), and sodium azide (9.23 mg, 0.142 mmol) in EtOH (0.8 mL), and acetic acid (0.2 mL) was stirred at 100° C. for 15 hours. The reaction was concentrated in vacuo, and the resulting residue was purified using prep-TLC, eluted with ethyl acetate to provide compound 97c. MS: m/z=324.10 [M+H].

### Step C—Synthesis of Compound 97

To a mixture of compound 97c (37 mg, 0.114 mmol) in THF (1 mL), and water (0.2 mL) was added trimethylphosphine (0.030 ml, 0.343 mmol). The resulting mixture was allowed to stir at 50° C. for 2 hours. The reaction was quenched by MeOH (0.1 mL), and concentrated in vacuo, and the resulting residue was purified using RP-flash chromatography (eluted with 0-100% acetonitrile in 5 mM aq. NH₄HCO₃) to provide compound 97. MS: m/z=298.15 [M+H]. ¹H NMR (300 MHz, d₆-DMSO) δ 8.55-8.53 (m, 1H), 7.82-7.74 (m, 2H), 7.61-7.59 (m, 1H), 7.34-7.29 (m, 1H), 7.06-7.03 (m, 1H), 5.56-5.01 (m, 4H), 3.42-3.37 (m, 2H), 1.58-1.53 (m, 2H), 1.50-1.27 (m, 2H), 0.91-0.89 (m, 3H).

The following compounds of the present invention were made using methodology described in Example 19 above, and substituting the appropriate reactants and/or reagents:

| | | MS | |
|---|---|---|---|
| Compound | Structure | [M + H] | ¹H NMR |
| 100 | 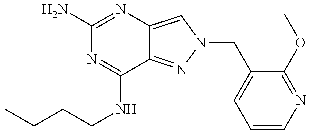 [SMILES: CCCCNc1nc(N)nc2cn(Cc3cccnc3OC)nc12] | 328.3 | (300 MHz, CDCl₃) δ 8.15-8.13 (m, 1H), 7.58 (s, 1H), 7.26- 7.22 (m, 1H), 6.87- 6.83 (m, 1H), 5.86 (s, 1H), 5.38 (s, 2H), 5.09 (s, 2H), 3.99 (s, 3H), 3.58-3.42 (m, 2H), 1.68-1.61 (m, 2H), |
| | | | 1.48-1.42 (m, 2H), |
| | | | 0.99-0.88 (m, 3H) |
| 101 | 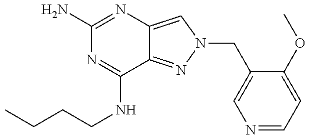 [SMILES: CCCCNc1nc(N)nc2cn(Cc3cnccc3OC)nc12] | 328.2 | (300 MHz, CD₃OD) δ 8.69-8.66 (m, 1H), 8.36 (s, 1H), 7.74 (s, 1H), 7.53-7.51 (m, 1H), 5.85 (s, 2H), 4.09 (s, 3H), 3.77-3.72 (m, 2H), 1.79-1.74 (m, 2H), 1.71-1.50 (m, 2H), 1.01-0.90 (m, |
| | | | 3H) |
| 102 | 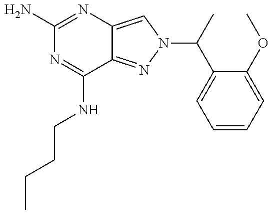 [SMILES: CCCCNc1nc(N)nc2cn(C(C)c3ccccc3OC)nc12] | 341.1 | (300 MHz, CDCl₃) δ 7.54 (s, 1H), 7.31-7.25 (m, 1H), 7.04-7.02 (m, 1H), 6.93-6.88 (m, 2H), 6.06-5.99 (m, 1H), 5.83 (s, 1H), 4.77 (s, 2H), 3.85 (s, 3H), 3.60-3.57 (m, 2H), 1.96-1.89 (m, 3H), 1.72-1.62 (m, 2H), 1.51-1.39 (m, 2H), 1.00-0.98 (m, 3H). |

### Example 20

### Preparation of Compound 98

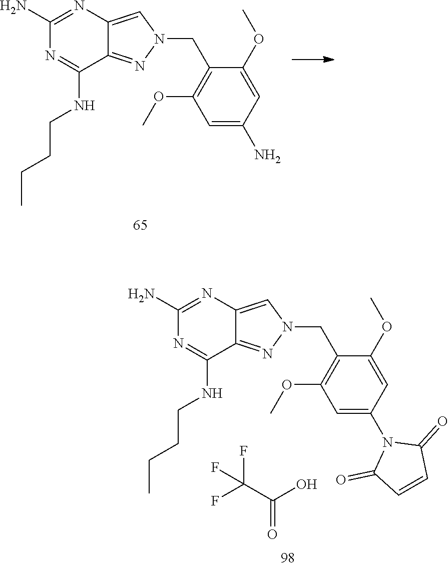

Compound 98 was made from compound 65, using the method described in Example 17, and substituting the appropriate reactants and/or reagents. MS: m/z=452.1 [M+H]. ¹H NMR (300 MHz, CDCl₃) δ 14.25 (s, 1H), 7.60 (s, 1H), 6.88 (s, 2H), 6.67 (s, 2H), 5.53 (s, 2H), 3.86 (s, 6H), 3.65-3.59 (m, 2H), 1.73-1.63 (m, 2H), 1.51-1.39 (m, 2H), 1.01-0.98 (m, 3H).

### Example 21

### Preparation of Compound 109

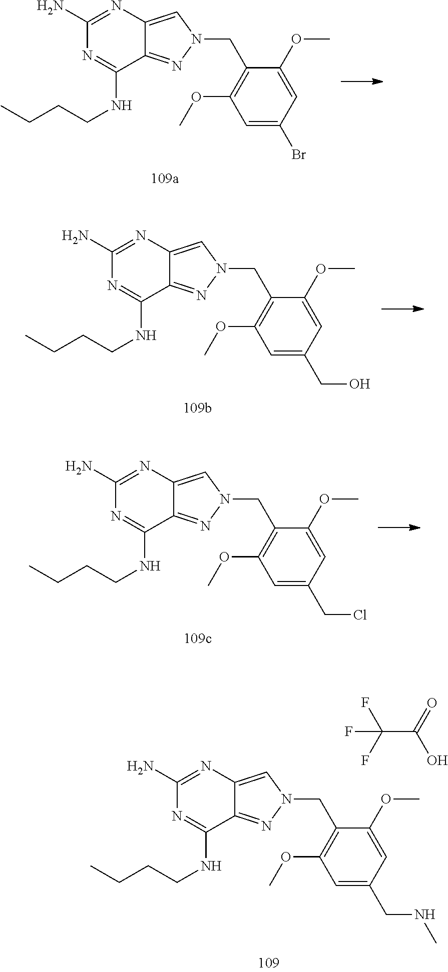

### Step A—Synthesis of Compound 109b

To a mixture of compound 109a (300 mg, 0.689 mmol), and (tripropylstannyl)methanol (442 mg, 1.585 mmol) in 1,4-dioxane (18 mL) was added Pd(Ph₃P)₄ (80 mg, 0.069 mmol) under argon atmosphere. The resulting mixture was allowed to stir for 3 hours at 100° C. The mixture was diluted with DCM (50 mL), washed with water (3×20 mL), brine (sat., 20 mL), dried over MgSO₄, and concentrated in vacuo, and the resulting residue was purified using silica gel column chromatography, eluted with 0-10% methanol in DCM to provide a yellow crude product which was further purified using RP-flash chromatography (eluted with 0-50% acetonitrile in 5 mM aq. ammonium bicarbonate) to provide compound 109b. MS: m/z=387.2 [M+H].

### Step B—Synthesis of Compound 109c

To a mixture of compound 109b (40 mg, 0.104 mmol) in DCM (1 mL) was added SOCl₂ (0.015 mL, 0.207 mmol) at 0° C. under argon atmosphere, and the resulting mixture was allowed to stir for 1 hour at 25° C., and concentrated in vacuo to provide compound 109c. MS: m/z=405.3 [M+H].

### Step C—Synthesis of Compound 109

To a mixture of compound 109c (40 mg, 0.099 mmol) in THF (0.2 mL) were added DIEA (0.345 mL, 1.976 mmol), and methanamine (0.988 mL, 1.976 mmol) (2 M in THF) at 0° C. under argon atmosphere. The resulting mixture was allowed to stir for 16 hours at 50° C. The mixture was concentrated in vacuo, and the resulting residue was purified using Prep-HPLC with the following condition: Column: SunFire Prep C18 OBD Column, 19×150 mm 5 um 10 nm; Mobile Phase A: Water (0.05% TFA), Mobile Phase B: acetonitrile; Flow rate: 20 mL/min; Gradient: 10% B to 50% B in 4.3 min; Detector: UV 210/254 nm; RT: 3.89 minutes to provide compound 109. MS: m/z=400.2 [M+H]. ¹H NMR (300 MHz, CD₃OD) δ 7.73 (s, 1H), 6.87 (s, 2H), 5.57 (s, 2H), 4.21 (s, 2H), 3.93 (s, 6H), 3.66-3.61 (m, 2H), 2.76 (s, 3H), 1.74-1.64 (m, 2H), 1.50-1.37 (m, 2H), 1.02-0.97 (m, 3H).

### Example 22

### Preparation of Compound 114

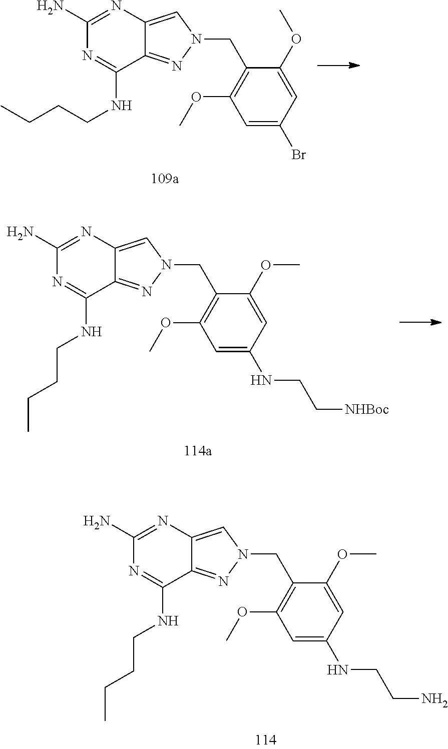 [SMILES: CCCCNc1nc(N)nc2cn(Cc3c(OC)cc(Br)cc3OC)nc12 ; CCCCNc1nc(N)nc2cn(Cc3c(OC)cc(NCCNC(=O)OC(C)(C)C)cc3OC)nc12 ; CCCCNc1nc(N)nc2cn(Cc3c(OC)cc(NCCN)cc3OC)nc12]

### Step A—Synthesis of Compound 114a

A mixture of compound 109a (150 mg, 0.345 mmol), NiBr₂·glyme (5.32 mg, 0.017 mmol), (Ir[dF(CF₃)ppy]₂(dtbpy))PF₆ (0.077 mg, 0.069 μmol), and DABCO (77 mg, 0.689 mmol) in dimethylacetamide (3 mL) was degassed with Ar for 5 minutes, then stirred for 15 hours under blue LED at 28° C. The reaction mixture was poured into ethyl acetate (100 mL), washed with water (3×20 mL), dried over anhydrous Na₂SO₄, and concentrated in vacuo. The resulting residue was purified using silica gel column chromatography, eluted with 0-10% DCM in MeOH (15% NH₄OH) to provide compound 114a. MS: m/z=515.4 [M+H].

### Step B—Synthesis of Compound 114

To a mixture of compound 114a (40 mg, 0.058 mmol) in DCM (4 mL) at 0° C. was added HCl (4 M in dioxane, 1 mL, 4.00 mmol), and the resulting mixture was allowed to stir for 2 hours at 28° C. The reaction mixture was quenched with 7 M NH₃ in MeOH (0.5 mL), and concentrated in vacuo. The resulting residue was purified using RP-Flash, eluted with 0-26% acetonitrile in 5 mM aq. NH₄HCO₃) to provide compound 114. MS: m/z=415.2 [M+H]. ¹H NMR (300 MHz, CD₃OD) δ 7.40 (s, 1H), 5.97 (s, 2H), 5.34 (s, 2H), 3.81 (s, 6H), 3.53-3.49 (m, 2H), 3.31-3.30 (m, 2H), 2.92-2.90 (m, 2H), 1.68-1.63 (m, 2H), 1.49-1.36 (m, 2H), 1.01-0.98 (m, 3H).

The following compounds of the present invention were made using methodology described in Example 22 above, and substituting the appropriate reactants and/or reagents:

| | | MS | |
|---|---|---|---|
| Compound | Structure | [M + H] | ¹H NMR |
| 210 | 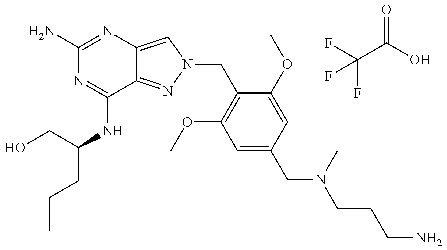 | 487.3 | (300 MHz, CD₃OD) δ 7.71 (s, 1H), 6.90 (s, 2H), 5.56 (s, 2H), 4.54- 4.53 (m, 1H), 4.51- 4.50 (m, 2H), 3.93 (s, 6H), 3.89-3.71 (m, 2H), 3.12-3.05 (m, 3H), 2.87-2.85 (m, 4H), 2.22-2.16 (m, 2H), 1.69-1.60 (m, 2H), 1.47-1.37 (m, 2H), 1.00-0.91 (m, 3H) |

### Example 23

### Preparation of Compound 115

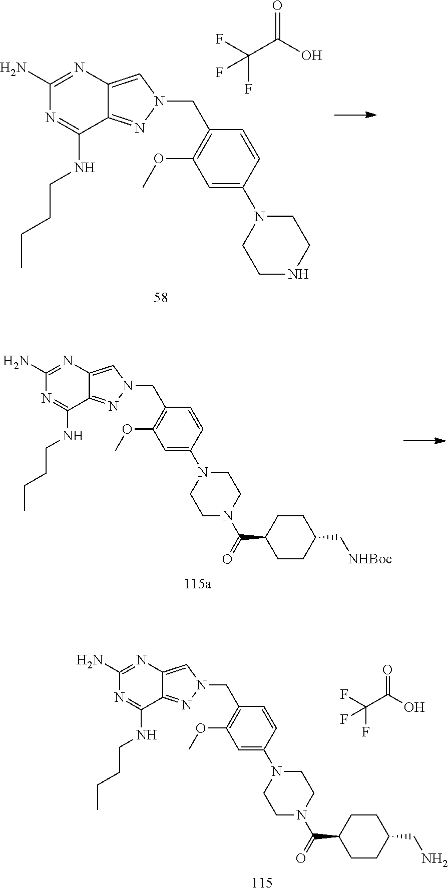 [SMILES: CCCCNc1nc(N)nc2cn(Cc3ccc(N4CCNCC4)cc3OC)nc12 ; O=C(O)C(F)(F)F ; CCCCNc1nc(N)nc2cn(Cc3ccc(N4CCN(C(=O)[C@H]5CC[C@H](CNC(=O)OC(C)(C)C)CC5)CC4)cc3OC)nc12 ; CCCCNc1nc(N)nc2cn(Cc3ccc(N4CCN(C(=O)[C@H]5CC[C@H](CN)CC5)CC4)cc3OC)nc12 ; O=C(O)C(F)(F)F]

### Step A—Synthesis of Compound 115a

A mixture of (1r,4r)-4-(((tert-butoxycarbonyl)amino)methyl)cyclohexane-1-carboxylic acid (22.08 mg, 0.086 mmol), HATU (32.6 mg, 0.086 mmol), and DIPEA (0.037 mL, 0.214 mmol) in DMF (0.4 mL) was allowed to stir for 10 minutes at 28° C. The reaction mixture was added to a solution of compound 58 (45 mg, 0.086 mmol), and DIEA (0.037 mL, 0.214 mmol) in DMF (0.400 mL), and the resulting mixture was allowed to stir for 20 minutes at 28° C. The reaction mixture was then directly purified using RP-flash chromatography (eluted with 0-35% acetonitrile in 0.1% aq. TFA) to provide compound 115a. MS: m/z=650.3 [M+H].

### Step B—Synthesis of Compound 115

To a mixture of compound 115a (30 mg, 0.046 mmol) in DCM (2 mL) at 0° C. was added TFA (0.5 mL), and the resulting mixture was allowed to stir for 2 hours at 28° C. The reaction mixture was concentrated in vacuo, and the resulting residue was purified using RP-flash chromatography (eluted 0-30% acetonitrile in water 0.05% aq. TFA) to provide compound 115. MS: m/z=550.3 [M+H]. ¹H NMR (300 M, CD₃OD) δ 7.72 (s, 1H), 7.20-7.17 (m, 1H), 6.62-6.54 (m, 2H), 5.39 (s, 2H). 3.84 (s, 3H), 3.74-3.63 (m, 4H), 3.61-3.59 (m, 2H), 3.31-3.20 (m, 4H), 2.81-2.68 (m, 3H), 1.91-1.84 (m, 4H), 1.71-1.36 (m, 7H), 1.21-1.09 (m, 2H), 0.97-0.95 (m, 3H).

### Example 24

### Preparation of Compound 125

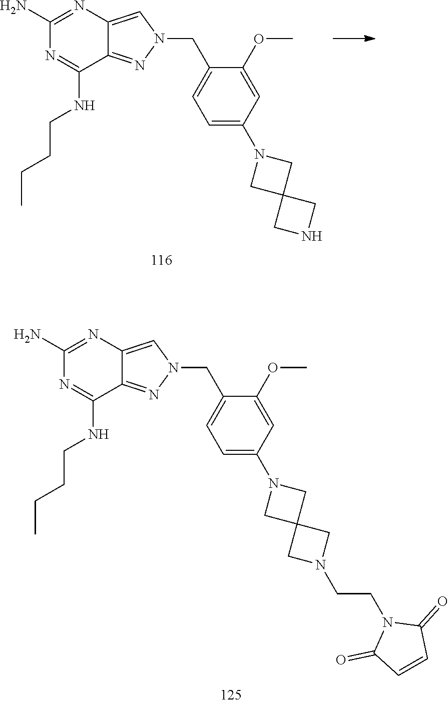 [SMILES: CCCCNc1nc(N)nc2cn(Cc3ccc(N4CC5(CNC5)C4)cc3OC)nc12 ; CCCCNc1nc(N)nc2cn(Cc3ccc(N4CC5(CN(CCN6C(=O)C=CC6=O)C5)C4)cc3OC)nc12]

To a mixture of compound 116 (10 mg, 0.024 mmol) in MeOH (2 mL) was added 2-(2,5-dioxo-2,5-dihydro-1H-pyrrol-1-yl)acetaldehyde (13.17 mg, 0.095 mmol) under nitrogen atmosphere, and the resulting mixture was allowed to stir for 30 minutes at 0° C. Then sodium cyanoborohydride (7.44 mg, 0.118 mmol) was added, and the resulting mixture was allowed to stir at 0° C. for 2 hours. The reaction was purified using Prep-HPLC using the following conditions: Column: SunFire Prep C18 OBD Column, 19×150 mm 5 um 10 nm; Mobile Phase A: Water (0.1% FA), Mobile Phase B: acetonitrile; Flow rate: 20 mL/min; Gradient: 15% B to 30% B in 8 min; Detector: UV 210/254 nm; RT: 7.85 minutes to provide compound 125. MS: m/z=546.3 [M+H]. ¹H NMR (300 MHz, CD₃CN) δ 7.57-7.48 (m, 2H), 7.11-7.08 (m, 1H), 6.56 (s, 2H), 6.05-6.02 (m, 2H), 5.33 (s, 2H), 4.27 (s, 4H), 4.04 (s, 4H), 3.82-3.80 (m, 3H), 3.70-3.56 (m, 4H), 3.35-3.32 (m, 2H), 1.73-1.61 (m, 2H), 1.60-1.44 (m, 2H), 1.00-0.90 (m, 3H).

The following compounds of the present invention were made using methodology described in Example 24 above, and substituting the appropriate reactants and/or reagents:

| | | MS | |
|---|---|---|---|
| Compound | Structure | [M + H] | ¹H NMR |
| 134 | 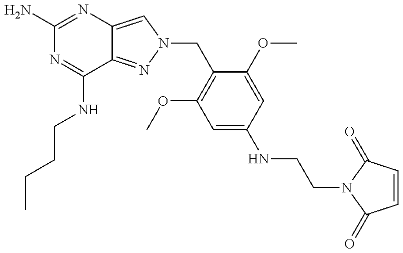 [SMILES: CCCCNc1nc(N)nc2cn(Cc3c(OC)cc(NCCN4C(=O)C=CC4=O)cc3OC)nc12] | 495.4 | (300 MHz, CD₃OD) δ 7.43 (s, 1H), 6.74 (s, 2H), 6.50 (s, 1H), 5.84 (s, 1H), 5.33 (s, 2H), 4.20 (s, 2H), 3.82-3.75 (m, 8H), 3.68-3.67 (m, 2H), 1.72-1.63 (m, 2H), 1.53-1.45 (m, 2H), 1.02-0.98 (m, 3H) |

### Example 25

### Preparation of Compound 128

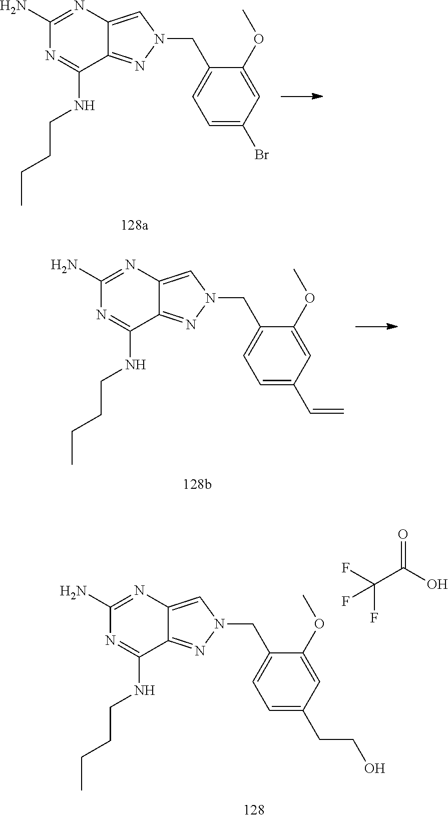

### Step A—Synthesis of Compound 128b

A mixture of compound 128a (100 mg, 0.247 mmol), trifluoro(vinyl)-14-borane, potassium salt (49.6 mg, 0.370 mmol), bis(di-tert-butyl(4-dimethylaminophenyl)phosphine) dichloropalladium(ii) (175 mg, 0.247 mmol), and potassium phosphate tribasic (52.4 mg, 0.247 mmol) in 1,4-dioxane (2 mL), and water (0.2 mL) was allowed to stir for 3 hours at 80° C. The reaction mixture was poured into a mixture of ethyl acetate (100 mL), and water (50 mL). The isolated organic layer was washed with brine (sat., 2×30 mL), dried over anhydrous Na₂SO₄, and concentrated in vacuo. The resulting residue was purified using prep-TLC, eluted with 0-10% MeOH in DCM to provide compound 128b. MS: m/z=353.3 [M+H].

### Step B—Synthesis of Compound 128

To a stirred mixture of compound 128b (30 mg, 0.085 mmol) in THF (1 mL) was added 9-borabicyclo[3.3.1]nonane (12.46 mg, 0.102 mmol) at 0° C. under nitrogen atmosphere, and the resulting reaction was allowed to stir at 25° C. for 16 hours. The reaction was quenched by MeOH (1.5 mL), and the resulting mixture was allowed to stir for 1 hour. Then 2 N aq. NaOH (1.5 mL), and 30% H₂O₂ (6 mL) were added, and the resulting mixture was allowed to stir for 4 hours. The reaction mixture was concentrated in vacuo, and the resulting residue was purified using prep-HPLC: Column: SunFire Prep C18 OBD Column, 19×150 mm 5 um 10 nm; Mobile Phase A: Water (0.05% TFA), Mobile Phase B: acetonitrile; Flow rate: 20 mL/min; Detector: UV 254 nm, Gradient: 25% B to 50% B in 6 min; RT: 5.58 minutes to provide compound 128. MS: m/z=371.2 [M+H]. ¹H NMR (300 MHz, CD₃OD) δ 7.78 (s, 1H), 7.21-7.18 (m, 1H), 6.97 (s, 1H), 6.87-6.84 (m, 1H), 5.49 (s, 2H), 3.88 (s, 3H), 3.77-3.75 (m, 2H), 3.66-3.63 (m, 2H), 2.86-2.82 (m, 2H), 1.73-1.64 (m, 2H), 1.49-1.44 (m, 2H), 1.01-0.93 (m, 3H).

### Example 26

### Preparation of Compound 134

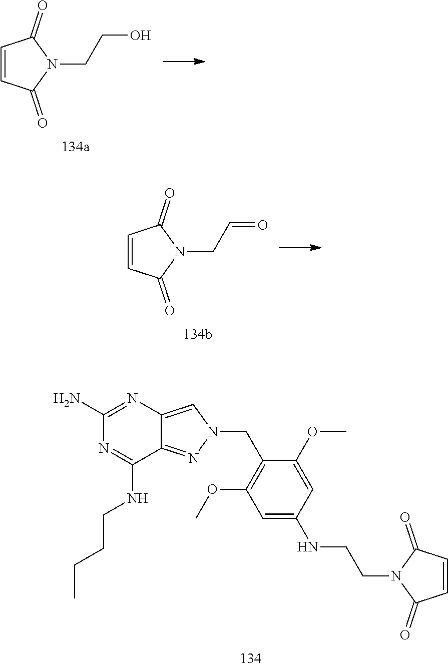 [SMILES: O=C1C=CC(=O)N1CCO ; O=CCN1C(=O)C=CC1=O ; CCCCNc1nc(N)nc2cn(Cc3c(OC)cc(NCCN4C(=O)C=CC4=O)cc3OC)nc12]

### Step A—Synthesis of Compound 134b

To a mixture of compound 134a (300 mg, 2.12 mmol) in DCM (10 mL) at 0° C. was added Dess-Martin Periodinane (1.35 g, 3.2 mmol), and the reaction was allowed to warm to room temperature over 3 hours. The solid was filtered out and washed with DCM (20 mL), and the filtrate was concentrated in vacuo to provide compound 134b as which was used without purification.

### Step B—Synthesis of Compound 134

To a mixture of 2-(4-amino-2,6-dimethoxybenzyl)-N⁷-butyl-2H-pyrazolo[4,3-d]pyrimidine-5,7-diamine (20 mg, 0.054 mmol), and compound 134b (30.0 mg, 0.162 mmol) in MeOH (1.5 mL) was added NaBH₃CN (16.92 mg, 0.269 mmol) at 0° C., and the resulting mixture was allowed to stir for 2 hours at 28° C. The reaction mixture was directly purified using Prep-HPLC Column: SunFire Prep C18 OBD Column, 19×150 mm 5 um 10 nm; Mobile Phase A: Water (10 mM ammonium acetate), Mobile Phase B: acetonitrile; Flow rate: 20 mL/min; Gradient: 25% B to 50% B in 6 minutes, Detector: UV 210/254 nm to provide compound 134. MS: m/z=495.4 [M+H]. ¹H NMR (300 MHz, CDCl₃) δ 7.43 (s, 1H), 6.74 (s, 2H), 6.50 (s, 1H), 5.84 (s, 2H), 5.33 (s, 2H), 4.20 (s, 1H), 3.82-3.75 (m, 8H), 3.68-3.67 (m, 2H), 3.62-3.58 (m, 2H), 1.72-1.63 (m, 2H), 1.53-1.45 (m, 2H),1.02-0.98 (m, 3H).

### Example 27

### Preparation of Compound 141

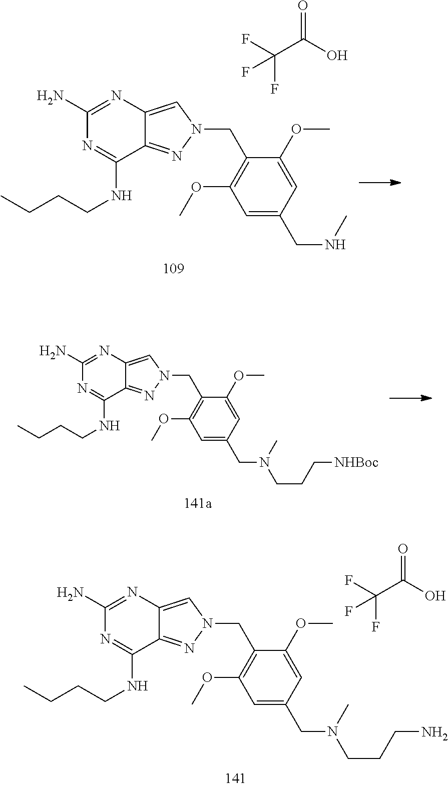

### Step A—Synthesis of Compound 141a

A mixture of compound 109 (60 mg, 0.117 mmol), tert-butyl (3-bromopropyl)carbamate (36.2 mg, 0.152 mmol), and K₂CO₃ (40.4 mg, 0.292 mmol) in acetonitrile (1.5 mL) was allowed to stir for 3 hours at 80° C. The reaction mixture was filtered, and the filtrate was concentrated in vacuo. The resulting residue was purified using prep-TLC (DCM/MeOH=10:1) to provide compound 141a. MS: m/z=557.4 [M+H].

### Step B—Synthesis of Compound 141

To a mixture of compound 141a (65 mg, 0.117 mmol) in DCM (2 mL) at 0° C. was added 2,2,2-trifluoroacetic acid (0.5 mL, 0.117 mmol), and the resulting mixture was allowed to stir for 2 hours at 25° C. The reaction mixture was concentrated in vacuo, and the resulting residue was purified using RP-Flash, eluted with 0-30% acetonitrile in water (0.05% TFA) to provide compound 141. MS: m/z=457.3 [M+H]. ¹H NMR (300 MHz, CD₃OD) δ 7.75 (s, 1H), 6.92 (s, 2H), 5.59 (s, 2H), 4.37 (s, 2H), 3.93 (s, 6H), 3.65-3.60 (m, 2H), 3.34-3.31 (m, 2 H), 3.08-3.03 (m, 2H), 2.84 (s, 3H), 2.26-2.16 (m, 2H), 1.73-1.69 (m, 2H), 1.49-1.42 (m, 2H), 1.02-0.98 (m, 3H).

### Example 28

### Preparation of Compound 154

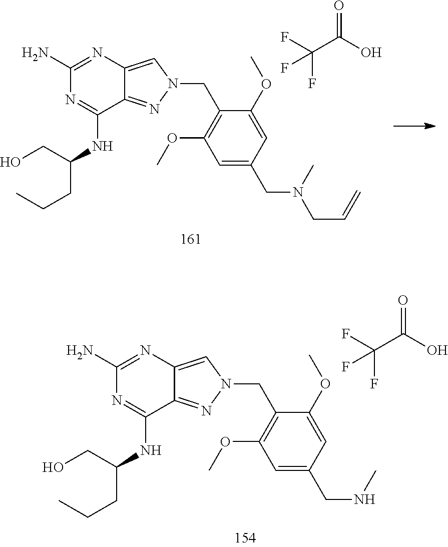

To a mixture of compound 161 (60 mg, 0.128 mmol), and tetrakis(triphenylphosphine)palladium(0) (7.38 mg, 6.39 μmol) in THF (1 mL) was added phenylsilane (34.6 mg, 0.319 mmol) at 25° C. The reaction mixture was allowed to stir at 35° C. for 2 hours. The resulting residue was purified using RP-Flash chromatography (eluted with 0-43% acetonitrile in aq. 0.05% TFA) to provide compound 154. MS: m/z=430.2 [M+H]. ¹H NMR (300 MHz, CD₃OD) δ 7.77 (s, 1H), 6.87 (s, 2H), 5.58 (s, 2H), 4.21 (s, 2H), 3.93 (s, 7H), 3.73-3.61 (m, 2H), 2.76 (s, 3H), 1.73-1.62 (m, 2H), 1.62-1.46 (m, 2H), 1.03-0.90 (m, 3H).

The following compounds of the present invention were made using methodology described in Example 28 substituting the appropriate reactants and/or reagents:

| | | MS | |
|---|---|---|---|
| Compound | Structure | [M + H] | ¹H NMR |
| 155 | 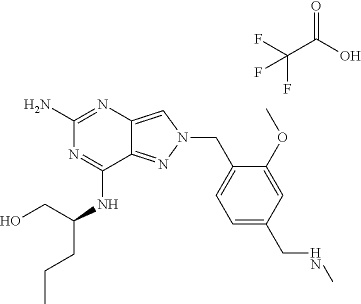 | 400.1 | (300 MHz, CD₃OD) δ 7.89 − 7.85 (m, 1H), 7.33 − 7.31 (m, 1H), 7.19 (s, 1H), 7.18 − 7.09 (m, 1H), 5.56 (s, 2H), 4.53 − 4.51 (m, 1H), 4.20 − 4.15 (m, 2H), 3.93 (s, 3H), 3.72 − 3.70 (m, 2H), 2.73 (s, 3H), 1.72 − 1.69 (m, 2H), 1.64 − 1.60 (m, 2H), 0.97 − 0.89 (m, 3H). |
| 163 | 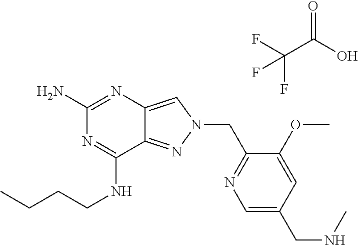 | 371.1 | (400 MHz, CD₃OD) δ 8.21 (s, 1H), 7.95 (s, 1H), 7.65 (s, 1H), 5.73 (s, 2H), 4.28 (s, 2H), 3.99 (s, 3H), 3.65 − 3.63 (m, 2H), 2.80 (s, 3H), 1.73 − 1.63 (m, 2H), 1.49 − 1.36 (m, 2H), 0.98 − 0.90 (m, 3H) |
| 164 | 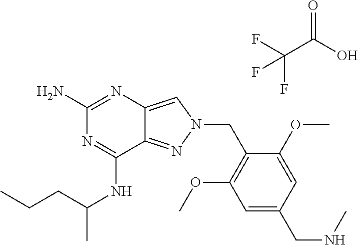 | 414.3 | (400 MHz, CD₃OD) δ 7.75 (s, 1H), 6.87 (s, 2H), 5.58 (s, 2H), 4.56 − 4.51 (m, 1H), 4.21 (s, 2H), 3.93 (s, 6H), 2.76 − 2.75 (m, 3H), 1.70 − 1.61 (m, 2H), 1.44 − 1.38 (m, 2H), 1.31 − 1.28 (m, 3H), 0.99 − 0.94 (m, 3H) |
| 165 | 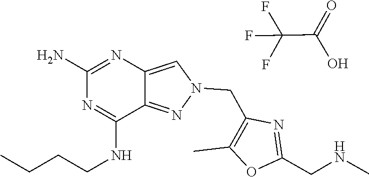 | 345.2 | (300 MHz, CD₃OD) δ 7.89 (s, 1H), 5.36 (s, 2H), 3.68 − 3.63 (m, 2H), 3.10 (s, 5H), 2.36 (s, 3H), 1.72 − 1.68 (m, 2H), 1.48 − 1.40 (m, 2H), 1.02 − 0.97 (m, 3H). |
| 189 | 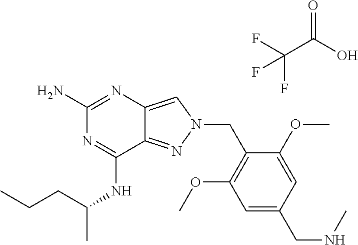 | 414.3 | (400 MHz, CD₃OD) δ 7.76 (s, 1H), 6.89 − 6.87 (m, 2H), 5.57 (s, 2H), 4.56 − 4.52 (m, 1H), 4.21 (s, 2H), 3.93 (s, 6H), 2.76 − 2.74 (m, 3H), 1.69 − 1.58 (m, 2H), 1.42 − 1.36 (m, 2H), 1.29 − 1.25 (m, 3H), 0.98 − 0.92 (m, 3H) |
| 190 | 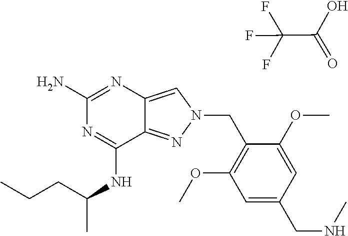 [SMILES: O=C(O)C(F)(F)F ; CCC[C@H](C)Nc1nc(N)nc2cn(Cc3c(OC)cc(CNC)cc3OC)nc12] | 414.1 | (400 MHz, CD₃OD) δ 7.77 (s, 1H), 6.90 − 6.87 (m, 2H), 5.57 (s, 2H), 4.56 − 4.52 (m, 1H), 4.21 (s, 2H), 3.93 (s, 6H), 2.84 − 2.76 (m, 3H), 1.69 − 1.60 (m, 2H), 1.42 − 1.38 (m, 2H), 1.29 − 1.25 (m, 3H), 0.98 − 0.92 (m, 3H) |
| 195 | 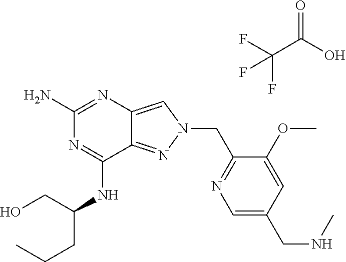 | 401.1 | (300 MHz, CD₃OD) δ 8.21 (s, 1H), 7.95 (s, 1H), 7.66 (s, 1H), 5.72 (s, 2H), 4.56 (s, 1H), 4.28 (s, 2H), 3.99 (s, 3H), 3.73 − 3.62 (m, 2H), 2.77 (s, 3H), 1.72 − 1.64 (m, 2H), 1.63 − 1.40 (m, 2H), 1.00 − 0.88 (m, 3H) |

### Example 29

### Preparation of Compound 156

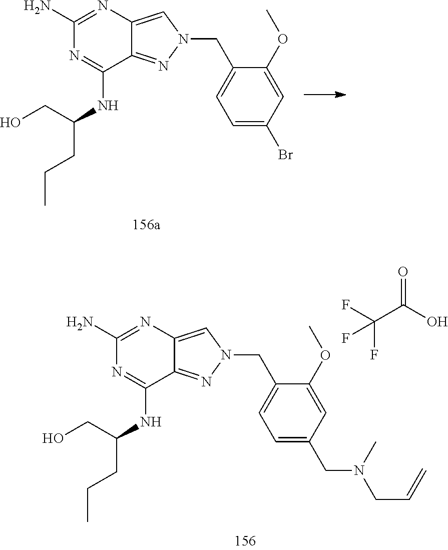

To a mixture of compound 156a (8.7 mg, 0.020 mmol, 8.62% yield), potassium (((tert-butoxycarbonyl)amino)methyl)trifluoroborate (218 mg, 0.919 mmol), dicyclohexyl(2′,4′,6′-triisopropyl-[1,1′-biphenyl]-2-yl)phosphane (6.57 mg, 0.014 mmol) in THF (1.2 mL), and water (0.3 mL) were added cesium carbonate (225 mg, 0.689 mmol), and diacetoxypalladium (1.547 mg, 6.89 μmol) at 25° C. The resulting reaction was allowed to stir for 15 hours at 80° C., then cooled to room temperature, and concentrated in vacuo. The resulting residue was purified using RP-flash chromatography (eluted with 0-36% acetonitrile in aq. 0.05% TFA) to provide compound 156. MS: m/z=440.2 [M+H]. ¹H NMR (300 MHz, CD₃OD) δ 7.89-7.85 (m, 1H), 7.35 (s, 1H), 7.22-7.12 (m, 1H), 7.10-7.09 (m, 1H), 6.02-5.98 (m, 1H), 5.66-5.57 (m, 4H), 4.58-4.56 (m, 1H), 4.55-4.53 (m, 2H), 3.94-3.81 (m, 3H), 3.80-3.73 (m, 2H), 3.68-3.66 (m, 2H), 2.77 (s, 3H), 1.69-1.64 (m, 2H), 1.47-1.43 (m, 2H), 1.00-0.95 (m, 3H).

The following compounds of the present invention were made using methodology described in Example 29 substituting the appropriate reactants and/or reagents:

| | | MS | |
|---|---|---|---|
| Compound | Structure | [M + H] | 1H NMR |
| 157 | 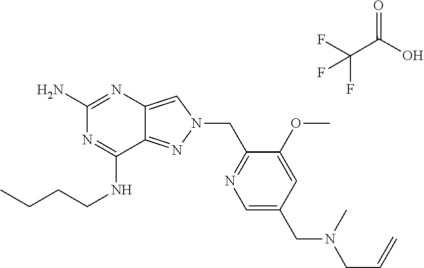 | 411.2 | (400 MHz, CD₃OD) δ 8.23 (s, 1H), 7.94 (s, 1H), 7.70 (s, 1H), 6.05 − 6.00 (m, 1H), 5.72 − 5.63 (m, 4H), 4.50 − 4.40 (m, 2H), 3.99 (s, 3H), 3.90 − 3.86 (m, 2H), 3.65 − 3.61 (m, 2H), 2.80 (s, 3H), 1.70 − 1.64 (m, 2H), 1.46 − 1.40 (m, 2H), 1.00 − 0.96 (m, 3H) |
| 158 | 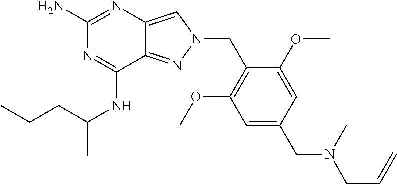 [SMILES: C=CCN(C)Cc1cc(OC)c(Cn2cc3nc(N)nc(NC(C)CCC)c3n2)c(OC)c1] | 454.3 | (400 MHz, CD₃OD) δ 7.66 (s, 1H), 6.75 (s, 2H), 5.99 − 5.98 (m, 1H), 5.53 (s, 2H), 5.30 − 5.24 (m, 2H), 4.52 − 4.50 (m, 1H), 3.89 (s, 6H), 3.58 (s, 2H), 3.13 − 3.10 (m, 2H), 2.26 (s, 3H), 1.69 − 1.64 (m, 2H), 1.56 − 1.45 (m, 2H), 1.32 − 1.27 (m, 3H), 0.99 − 0.90 (m, 3H) |
| 161 | 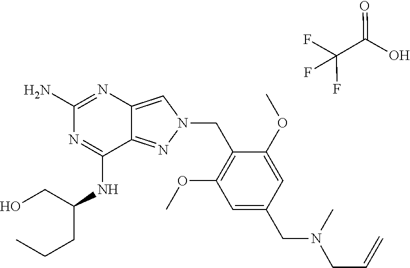 [SMILES: O=C(O)C(F)(F)F ; C=CCN(C)Cc1cc(OC)c(Cn2cc3nc(N)nc(N[C@H](CO)CCC)c3n2)c(OC)c1] | 470.2 | (300 MHz, CD₃OD) δ 7.79 (s, 1H), 6.91 (s, 2H), 6.10 − 6.01 (m, 1H), 5.68 − 5.59 (m, 4H), 4.57 − 4.52 (m, 1H), 4.50 − 4.23 (m, 2H), 3.93 (s, 6H), 3.89 − 3.80 (m, 2H), 3.74 − 3.62 (m, 2H), 2.80 (s, 3H), 1.71 − 1.60 (m, 2H), 1.60 − 1.36 (m, 2H), 1.03 − 0.93 (m, 3H) |
| 169 | 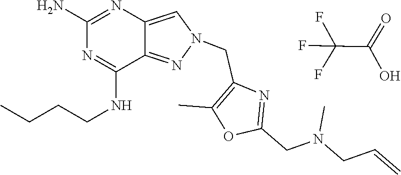 | 385.3 | (300 MHz, CD₃OD) δ 7.95 (s, 1H), 5.98 − 5.91 (m, 1H), 5.63 − 5.57 (m, 2H), 5.47 (s, 2H), 4.47 (s, 2H), 3.87 − 3.84 (m, 2H), 3.67 − 3.62 (m, 2H), 2.89 (s, 3H), 2.49 (s, 3H), 1.75 − 1.62 (m, 2H), 1.49 − 1.30 (m, 2H), 1.02 − 0.97 (m, 3H) |
| 183 | 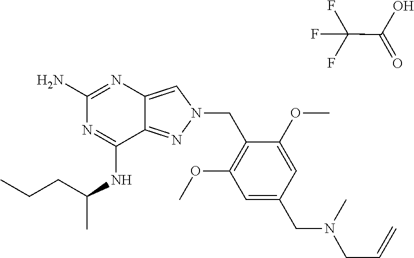 [SMILES: O=C(O)C(F)(F)F ; C=CCN(C)Cc1cc(OC)c(Cn2cc3nc(N)nc(N[C@@H](C)CCC)c3n2)c(OC)c1] | 454.2 | (400 MHz, CD₃OD) δ 7.52 (s, 1H), 6.73 (s, 2H), 6.02 − 5.93 (m, 1H), 5.67 − 5.56 (m, 2H), 5.31 − 5.20 (m, 2H), 4.38 − 4.31 (m, 1H), 3.94 − 3.87 (m, 6H), 3.56 − 3.51 (m, 2H), 3.09 − 3.05 (m, 2H), 2.25 − 2.21 (m, 3H), 1.64 − 1.55 (m, 2H), 1.49 − 1.41 (m, 2H), 1.38 − 1.22 (m, 3H), 0.97 − 0.89 (m, 3H) |
| 185 | 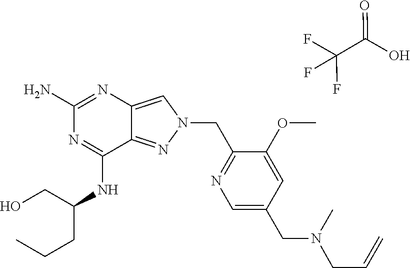 | 441.3 | (400 MHz, CD₃OD) δ 8.23 (s, 1H), 7.96 (s, 1H), 7.69 (s, 1H), 6.03 − 6.00 (m, 1H), 5.75 (s, 2H), 5.70 − 5.65 (m, 2H), 4.60 − 4.50 (m, 1H), 4.40 (s, 2H), 4.00 (s, 3H), 3.90 − 3.80 (m, 2H), 3.80 − 3.60 (m, 2H), 2.80 (s, 3H), 1.80 − 1.55 (m, 2H), 1.55 − 1.30 (m, 2H), 1.03 − 0.95 (m, 3H). |
| 187 | 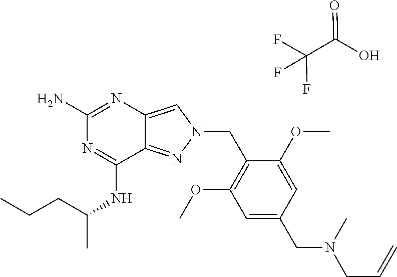 | | (400 MHz, CD₃OD) δ 7.77 (s, 1H), 6.91 (s, 2H), 6.08 − 6.01 (m, 1H), 5.67 − 5.56 (m, 4H), 4.54 − 4.52 (m, 2H), 3.94 − 3.90 (m, 9H), 3.81 − 3.78 (m, 3H), 1.66 − 1.58 (m, 2H), 1.44 − 1.38 (m, 2H), 1.29 − 1.27 (m, 3H), 0.98 − 0.93 (m, 3H). |

### Example 30

### Preparation of Compound 167

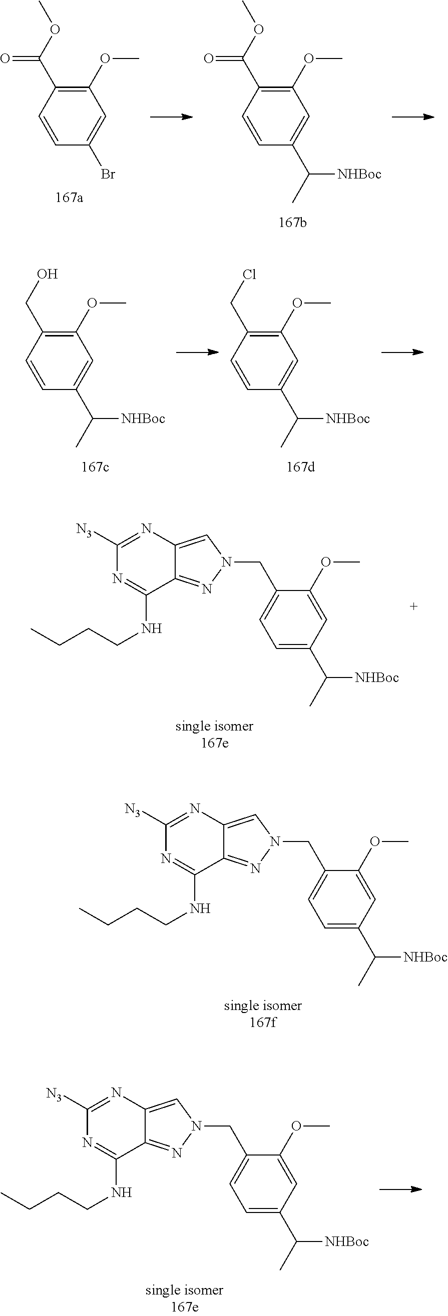

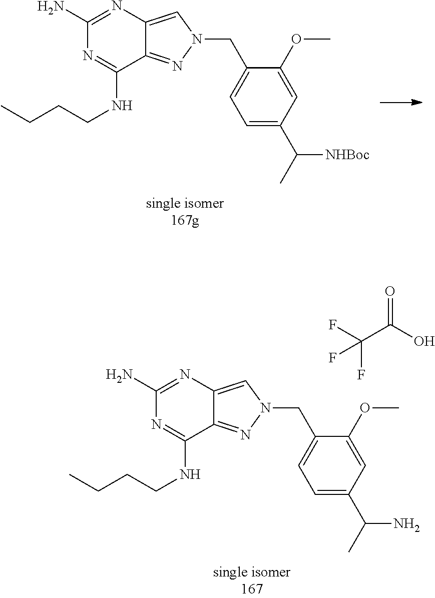

### Step A—Synthesis of Compound 167b

A mixture of methyl compound 167a (300 mg, 1.224 mmol), BOC-DL-ALA-OH (695 mg, 3.67 mmol), (Ir[dF(CF₃)ppy]₂(dtbpy))PF₆ (13.73 mg, 0.012 mmol), nickel(II) chloride ethylene glycol dimethyl ether complex (26.9 mg, 0.122 mmol), 4,4′-di-tert-butyl-2,2′-bipyridine (49.3 mg, 0.184 mmol), and cesium carbonate (1197 mg, 3.67 mmol) in DMF (4.5 mL) was degassed by bubbling argon stream for 20 minutes, then irradiated with 34 W blue LED lamp for 48 hours. The reaction was diluted with water (30 mL), extracted with ethyl acetate (3×50 mL), and the organic layer was washed with brine (sat., 30 mL), dried over anhydrous Na₂SO₄, and concentrated in vacuo. The resulting residue was purified using silica gel column chromatography (eluted with 0-30% ethyl acetate in petroleum ether) to provide compound 167b. MS: m/z=310.3 [M+H].

### Step B—Synthesis of Compound 167c

To a mixture of compound 167b (270 mg, 0.873 mmol) in THF (3 mL) at 0° C. was added dropwise LiAlH₄ (33.1 mg, 0.873 mmol), and the resulting mixture was allowed to stir for 2 hours at 0° C. The reaction was quenched with 0.05 mL water, 0.05 mL 15% aq. NaOH, then 0.15 mL water, filtered, and the filtrate was concentrated in vacuo. The resulting residue was purified using silica gel column chromatography (eluted with 0-45% ethyl acetate in petroleum ether) to provide compound 167c. MS: m/z=299.3 [M+H₂O].

### Step C—Synthesis of Compound 167d

To a mixture of NCS (111 mg, 0.832 mmol) in DCM (1 mL) was added methyl sulfide (25 mg, 0.402 mmol) at 0° C. under argon atmosphere. The reaction mixture was cooled to −20° C., and a solution of compound 167c (180 mg, 0.640 mmol) in DCM (2 mL) was added dropwise. The resulting mixture was allowed to stir for 2 hours, allowing the temperature reach to 0° C., during which time all the solid precipitate had dissolved, to provide a clear solution. The clear solution was poured over cold brine and extracted twice with diethyl ether. The combined organic layers were washed twice with cold brine (neutral pH), dried over Na₂SO₄, filtered, and concentrated in vacuo to provide compound 167d. MS: m/z=317.3 [M+H₂O].

### Step D—Synthesis of Compound 167e and 167f

A solution of compound 167d (190 mg, 0.634 mmol), 5-azido-N-butyl-1H-pyrazolo[4,3-d]pyrimidin-7-amine (110 mg, 0.475 mmol), and potassium carbonate (175 mg, 1.268 mmol) in DMF (2 mL) was allowed to stir for 15 hours at 25° C. under argon atmosphere. The reaction was quenched with water (30 mL), extracted with ethyl acetate (3×50 mL), the organic layer was washed with brine (sat., 30 mL), dried over anhydrous Na₂SO₄, concentrated in vacuo, and the resulting residue was purified using silica gel column chromatography (eluted with 0-11% MeOH in DCM) to provide the racemate. The racemate was separated by Chiral-HPLC with the following condition: Column: (R, R)-WHELK-O, 2.11*25 cm, 5 μm; Mobile Phase A: MTBE (0.5% 2 M NH₃-MeOH)-HPLC, Mobile Phase B: EtOH-HPLC; Flow rate: 20 mL/min; Gradient: 50% B to 50% B in 10 min; UV detector: 220/254 nm; RT1 (min): 5.561 to provide compound 167e and compound 167f. MS: m/z=496.3 [M+H].

### Step E—Synthesis of Compound 167g

Compound 167g was made from compound 167e, using the method described in Example 5, step F, and substituting the appropriate reactants and/or reagents. MS: m/z=470.3 [M+H].

### Step F—Synthesis of Compound 167

Compound 167g was made from compound 167e, using the method described in Example 23, step B, and substituting the appropriate reactants and/or reagents. MS: m/z=370.2 [M+H]. ¹H NMR (400 MHz, CD₃OD) δ 7.84 (s, 1H), 7.33-7.31 (m, 1H), 7.16 (s, 1H), 7.07-7.05 (m, 1H), 5.53 (s, 2H), 4.49-4.47 (m, 1H), 3.94 (s, 3H), 3.65-3.62 (m, 2H), 1.70-1.67 (m, 5H), 1.46-1.42 (m, 2H), 1.01-0.99 (m, 3H).

The following compounds of the present invention were made using methodology described in Example 30 substituting the appropriate reactants and/or reagents:

| | | MS | |
|---|---|---|---|
| Compound | Structure | [M + H] | ¹H NMR |
| 168 | 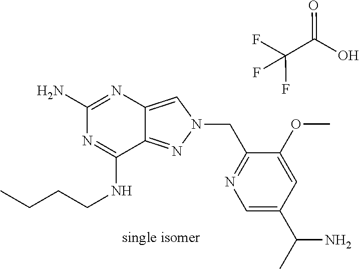 | 370.2 | (400 MHz, CD₃OD) δ 7.84 (s, 1H), 7.33 − 7.31 (m, 1H), 7.16 (s, 1H), 7.07 − 7.05 (m, 1H), 5.53 (s, 2H), 4.49 − 4.47 (m, 1H), 3.93 (s, 3H), 3.65 − 3.62 (m, 2H), 1.71 − 1.65 (m, 5H), 1.47 − 1.41 (m, 2H), 1.01 − 0.98 (m, 3H) |
| 176 | 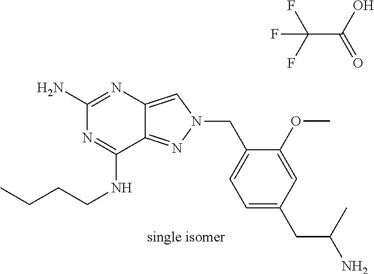 [SMILES: O=C(O)C(F)(F)F ; CCCCNc1nc(N)nc2cn(Cc3ccc(CC(C)N)cc3OC)nc12] | 384.2 | (400 MHz, CD₃OD) δ 7.69 (s, 1H), 7.15 − 7.13 (m, 1H), 6.84 − 6.83 (m, 1H), 6.77 − 6.75 (m, 1H), 5.38 (s, 2H), 3.78 (s, 3H), 3.53 − 3.48 (m, 2H), 3.46 − 3.44 (m, 1H), 2.90 − 2.88 (m, 1H), 2.75 − 2.73 (m, 1H), 1.59 − 1.56 (m, 2H), 1.37 − 1.32 (m, 2H), 1.18 − 1.12 (m, 3H), 0.92 − 0.86 (m, 3H) |
| 178 | 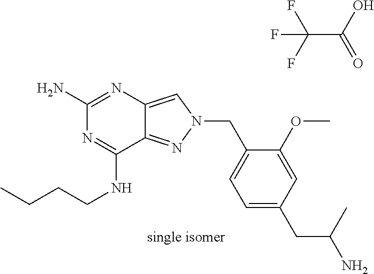 [SMILES: O=C(O)C(F)(F)F ; CCCCNc1nc(N)nc2cn(Cc3ccc(CC(C)N)cc3OC)nc12] | 384.2 | (400 MHz, CD₃OD) δ 7.70 (s, 1H), 7.15 − 7.13 (m, 1H), 6.84 − 6.83 (m, 1H), 6.76 − 6.74 (m, 1H), 5.39 (s, 2H), 3.78 (s, 3H), 3.53 − 3.50 (m, 2H), 3.21 − 3.20 (m, 1H), 2.88 − 2.85 (m, 1H), 2.75 − 2.72 (m, 1H), 1.59 − 1.56 (m, 2H), 1.32 − 1.30 (m, 2H), 1.20 − 1.14 (m, 3H), 0.90 − 0.81 (m, 3H). |
| 193 | 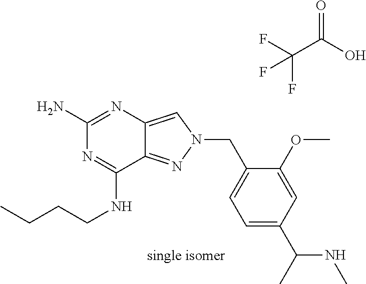 [SMILES: O=C(O)C(F)(F)F ; CCCCNc1nc(N)nc2cn(Cc3ccc(C(C)NC)cc3OC)nc12] | 384.2 | (400 MHz, CD₃OD) δ 7.89 (s, 1H), 7.35 − 7.33 (m, 1H), 7.15 (s, 1H), 7.07 − 7.05 (m, 1H), 5.57 (s, 2H), 4.36 − 4.33 (m, 1H), 3.95 (s, 3H), 3.76 − 3.69 (m, 2H), 2.60 (s, 3H), 1.70 − 1.63 (m, 5H), 1.48 − 1.39 (m, 2H), 1.01 − 0.97 (m, 3H). |
| 194 | 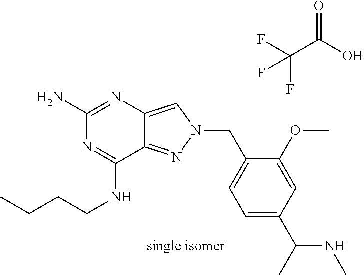 | 384.2 | (400 MHz, CD₃OD) δ 7.89 (s, 1H), 7.35 − 7.33 (m, 1H), 7.15 (s, 1H), 7.07 − 7.05 (m, 1H), 5.57 (s, 2H), 4.35 − 4.31 (m, 1H), 3.98 (s, 3H), 3.65 − 3.62 (m, 2H), 2.60 (s, 3H), 1.72 − 1.64 (m, 5H), 1.49 − 1.38 (m, 2H), 1.01 − 0.96 (m, 3H). |
| 196 | 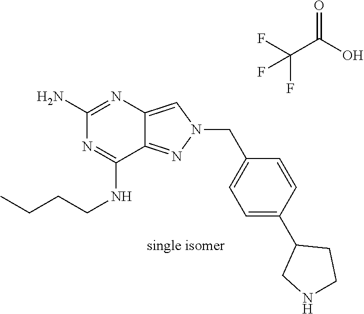 [SMILES: O=C(O)C(F)(F)F ; CCCCNc1nc(N)nc2cn(Cc3ccc(C4CCNC4)cc3)nc12] | 366.2 | (300 MHz, CD₃OD) δ 7.89 (s, 1H), 7.41 (s, 4H), 5.55 (s, 2H), 3.74 − 3.52 (m, 5H), 3.43 − 3.41 (m, 1H), 3.23 − 3.16 (m, 1H), 2.49 − 2.44 (m, 1H), 2.13 − 2.06 (m, 1H), 1.74 − 1.64 (m, 2H), 1.47 − 1.40 (m, 2H), 1.02 − 0.97 (m, 3H). |
| 198 | 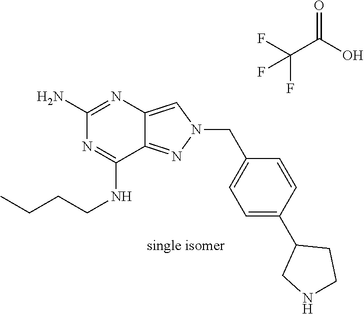 [SMILES: O=C(O)C(F)(F)F ; CCCCNc1nc(N)nc2cn(Cc3ccc(C4CCNC4)cc3)nc12] | 366.2 | (300 MHz, CD₃OD) δ 7.89 (s, 1H), 7.41 (s, 4H), 5.55 (s, 2H), 3.74 − 3.52 (m, 5H), 3.43 − 3.41 (m, 1H), 3.23 − 3.16 (m, 1H), 2.49 − 2.44 (m, 1H), 2.13 − 2.06 (m, 1H), 1.74 − 1.64 (m, 2H), 1.47 − 1.40 (m, 2H), 1.02 − 0.97 (m, 3H). |
| 200 | 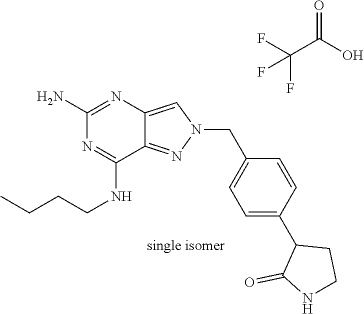 [SMILES: O=C(O)C(F)(F)F ; CCCCNc1nc(N)nc2cn(Cc3ccc(C4CCNC4=O)cc3)nc12] | 380.3 | (300 MHz, CD₃OD) δ 7.90 (s, 1H), 7.34 − 7.31 (m, 4H), 5.54 (s, 2H), 3.73 − 3.70 (m, 3H), 3.67 − 3.62 (m, 2H), 2.67 − 2.56 (m, 1H), 2.25 − 2.15 (m, 1H), 1.74 − 1.72 (m, 2H), 1.69 − 1.67 (m, 2H), 1.02 − 0.99 (m, 3H). |
| 201 | 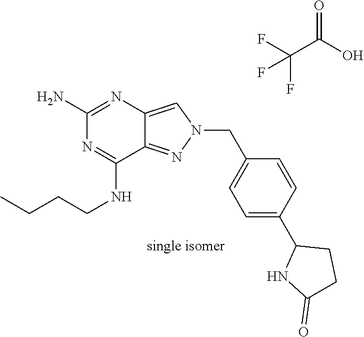 [SMILES: O=C(O)C(F)(F)F ; CCCCNc1nc(N)nc2cn(Cc3ccc(C4CCC(=O)N4)cc3)nc12] | 380.1 | (300 MHz, CD₃OD) δ 7.98 (s, 1H), 7.40 − 7.37 (m, 4H), 5.55 (s, 2H), 4.91 − 4.86 (m, 1H), 3.67 − 3.56 (m, 2H), 2.67 − 2.65 (m, 1H), 2.57 − 2.54 (m, 2H), 1.95 − 1.93 (m, 1H), 1.89 − 1.87 (m, 2H), 1.67 − 1.65 (m, 2H), 1.02 − 0.99 (m, 3H) |
| 202 | 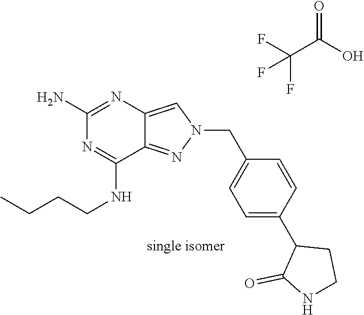 [SMILES: O=C(O)C(F)(F)F ; CCCCNc1nc(N)nc2cn(Cc3ccc(C4CCNC4=O)cc3)nc12] | 380.3 | (300 MHz, CD₃OD) δ 7.90 (s, 1H), 7.34 − 7.31 (m, 4H), 5.54 (s, 2H), 3.73 − 3.70 (m, 3H), 3.67 − 3.62 (m, 2H), 2.67 − 2.56 (m, 1H), 2.25 − 2.15 (m, 1H), 1.74 − 1.72 (m, 2H), 1.69 − 1.67 (m, 2H), 1.02 − 0.99 (m, 3H) |
| 203 | 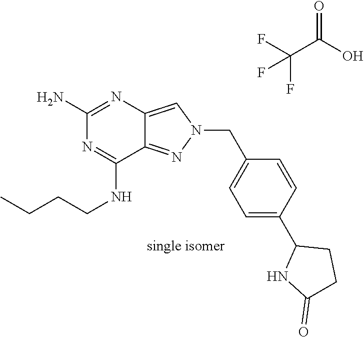 [SMILES: O=C(O)C(F)(F)F ; CCCCNc1nc(N)nc2cn(Cc3ccc(C4CCC(=O)N4)cc3)nc12] | 380.1 | (300 MHz, CD₃OD) δ 7.98 (s, 1H), 7.40 − 7.37 (m, 4H), 7.33 (s, 1H), 5.55 − 5.53 (m, 2H), 3.67 − 3.56 (m, 2H), 2.67 − 2.65 (m, 1H), 2.57 − 2.54 (m, 2H), 1.95 − 1.93 (m, 1H), 1.89 − 1.87 (m, 2H), 1.67 − 1.65 (m, 2H), 1.02 − 0.99 (m, 3H) |
| 204 | 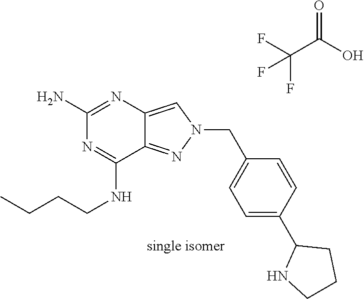 [SMILES: O=C(O)C(F)(F)F ; CCCCNc1nc(N)nc2cn(Cc3ccc(C4CCCN4)cc3)nc12] | 366.2 | (300 MHz, CD₃OD) δ 7.98 (s, 1H), 7.53 − 7.50 (m, 4H), 5.61 (s, 2H), 4.67 − 4.64 (m, 1H), 3.67 − 3.65 (m, 2H), 3.46 − 3.45 (m, 2H), 2.50 − 2.49 (m, 2H), 2.48 − 2.46 (m, 2H), 1.69 − 1.67 (m, 2H), 1.50 − 1.47 (m, 2H), 1.01 − 0.99 (m, 3H) |
| 205 | 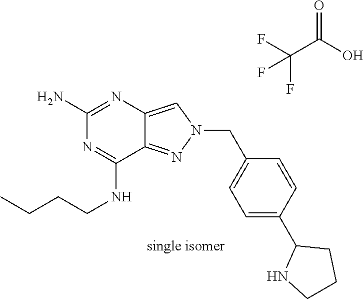 [SMILES: O=C(O)C(F)(F)F ; CCCCNc1nc(N)nc2cn(Cc3ccc(C4CCCN4)cc3)nc12] | 366.2 | (300 MHz, CD₃OD) δ 7.98 (s, 1H), 7.53 − 7.50 (m, 4H), 5.61 (s, 2H), 4.67 − 4.64 (m, 1H), 3.67 − 3.65 (m, 2H), 3.46 − 3.45 (m, 2H), 2.50 − 2.49 (m, 2H), 2.47 − 2.46 (m, 2H), 1.69 − 1.67 (m, 2H), 1.50 − 1.47 (m, 2H), 1.01 − 0.99 (m, 3H) |
| 209 | 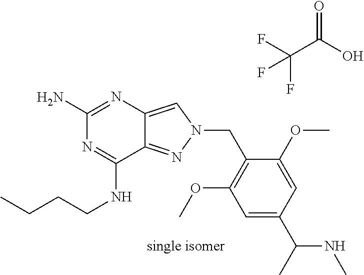 | 414.2 | (300 MHz, CD₃OD) δ 7.74 (s, 1H), 6.84 (s, 2H), 5.57 (s, 2H), 4.84 − 4.30 (m, 1H), 3.93 (s, 6H), 3.67 − 3.61 (m, 2H), 2.63 (s, 3H), 1.73 − 1.64 (m, 5H), 1.50 − 1.40 (m, 2H), 1.02 − 0.98 (m, 3H) |
| 211 | 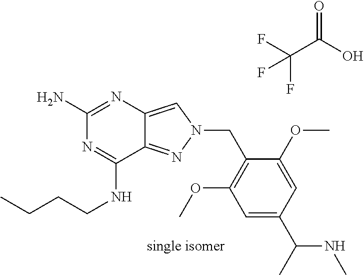 | 414.2 | (300 MHz, CD₃OD) δ 7.74 (s, 1H), 6.84 (s, 2H), 5.57 (s, 2H), 4.84 − 4.30 (m, 1H), 3.93 (s, 6H), 3.67 − 3.61 (m, 2H), 2.63 (s, 3H), 1.73 − 1.64 (m, 5H), 1.50 − 1.40 (m, 2H), 1.02 − 0.98 (m, 3H). |
| 214 | 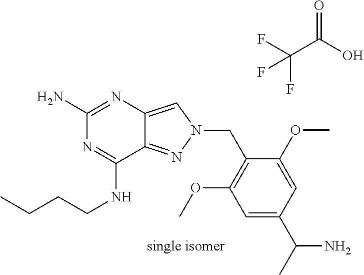 | 400.2 | (300 MHz, CD₃OD) δ 7.72 (s, 1H), 6.84 (s, 2H), 5.56 (s, 2H), 4.50 − 4.48 (s, 1H), 3.98 − 3.93 (m, 6H), 3.65 − 3.63 (m, 2H), 1.67 − 1.65 (m, 5H), 1.50 − 1.47 (m, 2H), 1.02 − 1.00 (m, 3H) |
| 215 | 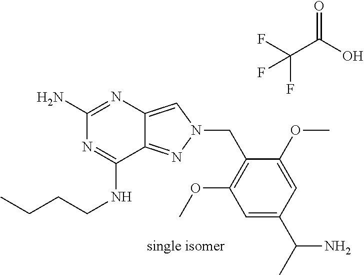 | 400.2 | (300 MHz, CD₃OD) δ 7.73 (s, 1H), 6.84 (s, 2H), 5.56 (s, 2H), 4.50 − 4.48 (s, 1H), 3.93 − 3.86 (m, 6H), 3.63 − 3.61 (m, 2H), 1.73 − 1.71 (m, 5H), 1.59 − 1.55 (m, 2H), 1.02 − 0.99 (m, 3H) |
| 216 | 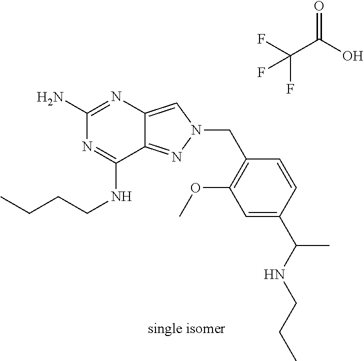 [SMILES: O=C(O)C(F)(F)F ; CCCCNc1nc(N)nc2cn(Cc3ccc(C(C)NCCC)cc3OC)nc12] | 412.3 | (300 MHz, CD₃OD) δ 7.87 (s, 1H), 7.35 − 7.33 (m, 1H), 7.19 (s, 1H), 7.09 − 7.07 (m, 1H), 5.55 (s, 2H), 4.43 − 4.36 (m, 1H), 3.95 (s, 3H), 3.67 − 3.56 (m, 2H), 2.97 − 2.88 (m, 1H), 2.78 − 2.69 (m, 1H), 1.77 − 1.69 (m, 7H), 1.60 − 1.50 (m, 2H), 1.00 − 0.90 (m, 6H). |
| 217 | 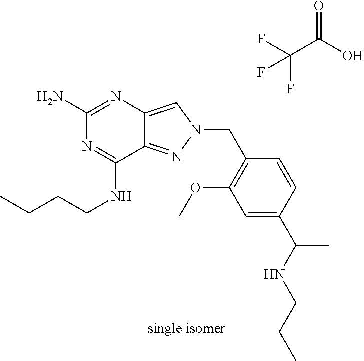 [SMILES: O=C(O)C(F)(F)F ; CCCCNc1nc(N)nc2cn(Cc3ccc(C(C)NCCC)cc3OC)nc12] | 412.3 | (300 MHz, CD₃OD) δ 7.88 (s, 1H), 7.35 − 7.33 (m, 1H), 7.19 (s, 1H), 7.10 − 7.07 (m, 1H), 5.78 (s, 2H), 4.39 (s, 1H), 3.95 (s, 3H), 3.67 − 3.62 (m, 2H), 2.97 − 2.88 (m, 1H), 2.78 − 2.69 (m, 1H), 1.72 − 1.67 (m, 7H), 1.55 − 1.40 (m, 2H), 1.05 − 0.95 (m, 6H) |
| 219 | 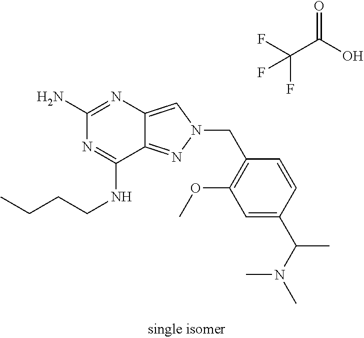 [SMILES: O=C(O)C(F)(F)F ; CCCCNc1nc(N)nc2cn(Cc3ccc(C(C)N(C)C)cc3OC)nc12] | 398.3 | (300 MHz, CD₃OD) δ 7.90 (s, 1H), 7.35 − 7.33 (m, 1H), 7.20 − 7.19 (m, 1H), 7.12 − 7.09 (m, 1H), 5.58 (s, 2H), 4.50 − 4.43 (m, 1H), 3.94 (s, 3H), 3.66 − 3.61 (m, 2H), 3.01 − 2.73 (m, 6 H), 1.75 − 1.54 (m, 5H), 1.50 − 1.35 (m, 2H), 1.05 − 0.99 (m, 3H) |
| 220 | 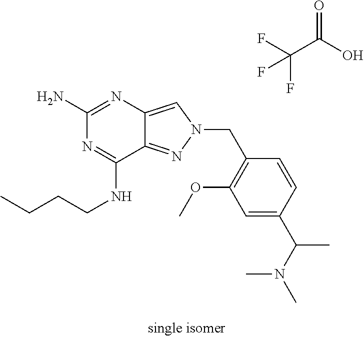 [SMILES: O=C(O)C(F)(F)F ; CCCCNc1nc(N)nc2cn(Cc3ccc(C(C)N(C)C)cc3OC)nc12] | 398.3 | (300 MHz, CD₃OD) δ 7.99 (s, 1H), 7.35 − 7.33 (m, 1H), 7.20 − 7.19 (m, 1H), 7.12 − 7.09 (m, 1H), 5.58 (s, 2H), 4.50 − 4.43 (m, 1H), 3.98 (s, 3H), 3.66 − 3.61 (m, 2H), 2.91 − 2.73 (m, 6H), 1.75 − 1.55 (m, 5H), 1.50 − 1.35 (m, 2H), 1.05 − 0.98 (m, 3H) |
| 221 | 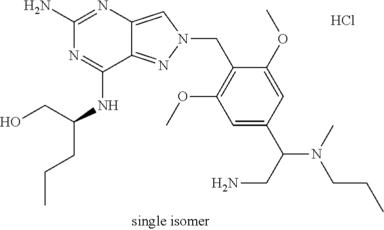 [SMILES: CCC[C@@H](CO)Nc1nc(N)nc2cn(Cc3c(OC)cc(C(CN)N(C)CCC)cc3OC)nc12 ; Cl] | 501.4 | (300 MHz, CD₃OD) δ 7.89 (s, 1H), 7.16 (s, 2H), 5.63 (s, 2H), 4.85 − 4.50 (m, 8H), 4.10 − 3.85 (m, 2H), 3.85 − 3.60 (m, 2H), 3.30 − 2.70 (m, 5H), 2.05 − 1.75 (m, 2H), 1.75 − 1.60 (m, 2H), 1.50 − 1.30 (m, 2H), 1.20 − 0.85 (m, 6H). |
| 222 | 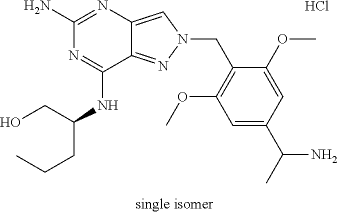 [SMILES: CCC[C@@H](CO)Nc1nc(N)nc2cn(Cc3c(OC)cc(C(C)N)cc3OC)nc12 ; Cl] | 430.3 | (300 MHz, CD₃OD) δ 7.79 (s, 1H), 6.85 (s, 2H), 5.59 (s, 2H), 4.60 − 4.45 (m, 2H), 3.95 (s, 6H), 3.75 − 3.60 (m, 2H), 1.90 − 1.60 (m, 5H), 1.50 − 1.30 (m, 2H), 1.00 − 0.90 (m, 3H). |
| 223 | 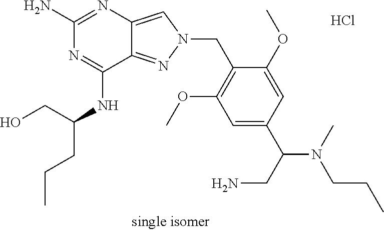 [SMILES: CCC[C@@H](CO)Nc1nc(N)nc2cn(Cc3c(OC)cc(C(CN)N(C)CCC)cc3OC)nc12 ; Cl] | 501.4 | (300 MHz, CD₃OD) δ 7.89 (s, 1H), 7.20 (s, 2H), 5.60 (s, 2H), 4.85 − 4.50 (m, 8H), 4.10 − 3.80 (m, 2H), 3.80 − 3.60 (m, 2H), 3.30 − 2.60 (m, 5H), 2.00 − 1.75 (m, 2H), 1.75 − 1.60 (m, 2H), 1.50 − 1.30 (m, 2H), 1.15 − 0.90 (m, 6H) |
| 224 | 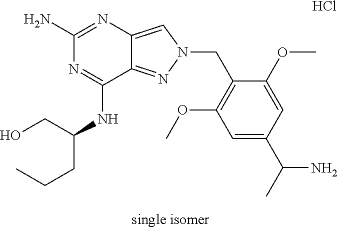 [SMILES: Cl ; CCC[C@@H](CO)Nc1nc(N)nc2cn(Cc3c(OC)cc(C(C)N)cc3OC)nc12] | 430.3 | (300 MHz, CD₃OD) δ 7.80 (s, 1H), 6.85 (s, 2H), 5.60 (s, 2H), 4.60 − 4.40 (m, 2H), 3.90 (s, 6H), 3.75 − 3.55 (m, 2H), 1.80 − 1.60 (m, 5H), 1.55 − 1.35 (m, 2H), 1.00 − 0.90 (m, 3H) |
| 225 | 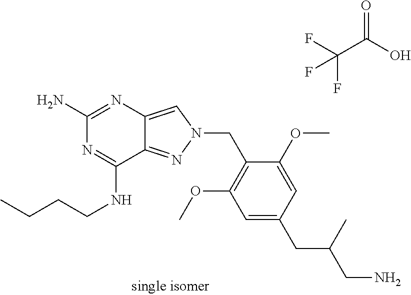 | 414.2 | (300 MHz, CD₃OD) δ 7.71 (s, 1H), 6.65 (s, 2H), 5.48 (s, 2H), 3.90 (s, 6H), 3.65 − 3.56 (m, 3H), 3.01 − 2.84 (m, 2H), 1.71 − 1.59 (m, 2H), 1.54 − 1.40 (m, 2H), 1.33 − 1.21 (m, 3H), 1.02 − 0.99 (m, 3H) |
| 226 | 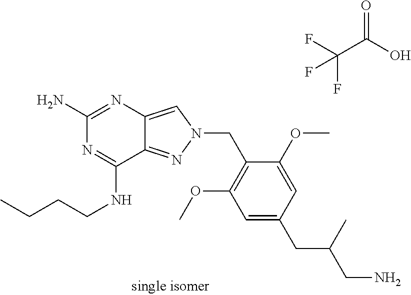 | 414.2 | (300 MHz, CD₃OD) δ 7.71 (s, 1H), 6.64 (s, 2H), 5.54 (s, 2H), 4.90 (s, 6H), 3.65 − 3.55 (m, 3H), 2.91 − 2.84 (m, 2H), 1.73 − 1.66 (m, 2H), 1.59 − 1.51 (m, 2H), 1.47 − 1.31 (m, 3H), 1.02 − 0.98 (m, 3H). |
| 227 | 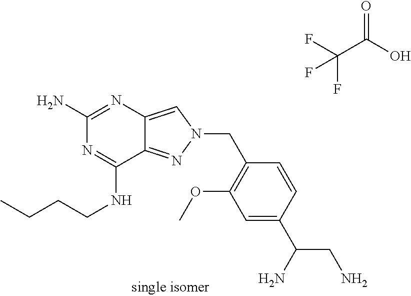 [SMILES: O=C(O)C(F)(F)F ; CCCCNc1nc(N)nc2cn(Cc3ccc(C(N)CN)cc3OC)nc12] | 385.2 | (300 MHz, CD₃OD) δ 7.86 (s, 1H), 7.41 − 7.38 (m, 1H), 7.27 (s, 1H), 7.16 − 7.13 (m, 1H), 5.56 (s, 2H), 4.69 − 4.64 (m, 1H), 3.97 (s, 3H), 3.64 − 3.58 (m, 4H), 1.69 − 1.66 (m, 2H), 1.45 − 1.41 (m, 2H), 1.01 − 0.09 (m, 3H). |
| 228 | 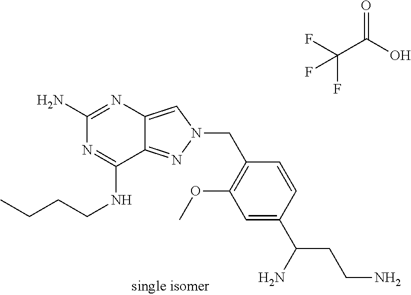 [SMILES: O=C(O)C(F)(F)F ; CCCCNc1nc(N)nc2cn(Cc3ccc(C(N)CCN)cc3OC)nc12] | 399.2 | (300 MHz, CD₃OD) δ 7.87 (s, 1H), 7.34 (s, 1H), 7.34 − 7.33 (m, 1H), 7.21 − 7.20 (m, 1H), 5.55 (s, 2H), 4.44 − 4.42 (m, 1H), 3.96 (s, 3H), 3.66 − 3.64 (m, 2H), 2.97 − 2.95 (m, 1H), 2.71 − 2.70 (m, 1H), 2.43 − 2.40 (m, 2H), 1.74 − 1.73 (m, 2H), 1.65 − 1.63 (m, 2H), 1.01 − 0.99 (m, 3H) |
| 229 | 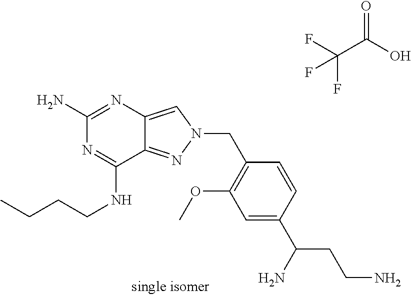 [SMILES: O=C(O)C(F)(F)F ; CCCCNc1nc(N)nc2cn(Cc3ccc(C(N)CCN)cc3OC)nc12] | 399.2 | (300 MHz, CD₃OD) δ 7.84 (s, 1H), 7.34 (s, 1H), 7.34 − 7.33 (m, 1H), 7.21 − 7.20 (m, 1H), 5.52 (s, 2H), 4.44 − 4.42 (m, 1H), 3.96 (s, 3H), 3.66 − 3.64 (m, 2H), 2.97 − 2.95 (m, 1H), 2.71 − 2.70 (m, 1H), 2.43 − 2.40 (m, 2H), 1.74 − 1.73 (m, 2H), 1.65 − 1.63 (m, 2H), 1.01 − 0.99 (m, 3H) |
| 231 | 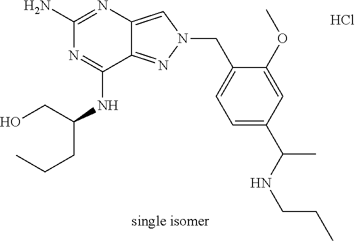 [SMILES: CCCNC(C)c1ccc(Cn2cc3nc(N)nc(N[C@H](CO)CCC)c3n2)c(OC)c1 ; Cl] | 442.4 | (300 MHz, CD₃OD) δ 7.92 (s, 1H), 7.37 − 7.34 (m, 1H), 7.33 − 7.26 (m, 1H), 7.25 − 7.10 (m, 1H), 5.57 (s, 2H), 4.56 − 4.54 (m, 1H), 4.50 − 4.37 (m, 1H), 3.96 (s, 3H), 3.74 − 3.67 (m, 2H), 2.97 − 2.88 (m, 1H), 2.78 − 2.68 (m, 1H), 1.76 − 1.56 (m, 7H), 1.55 − 1.30 (m, 2H), 1.00 − 0.88 (m, 6H). |
| 232 | 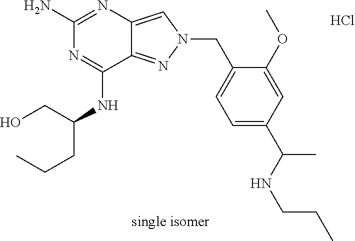 [SMILES: CCCNC(C)c1ccc(Cn2cc3nc(N)nc(N[C@H](CO)CCC)c3n2)c(OC)c1 ; Cl] | 442.4 | (300 MHz, CD3OD) δ 7.92 (s, 1H), 7.37 − 7.34 (m, 1H), 7.33 − 7.26 (m, 1H), 7.25 − 7.10 (m, 1H), 5.57 (s, 2H), 4.56 − 4.54 (m, 1H), 4.50 − 4.37 (m, 1H), 3.96 (s, 3H), 3.74 − 3.67 (m, 2H), 2.97 − 2.88 (m, 1H), 2.78 − 2.68 (m, 1H), 1.76 − 1.56 (m, 7H), 1.55 − 1.30 (m, 2H), 1.00 − 0.88 (m, 6H) |
| 233 | 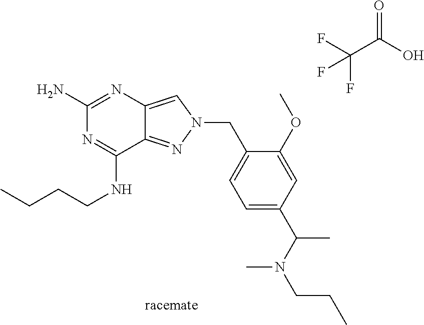 [SMILES: O=C(O)C(F)(F)F ; CCCCNc1nc(N)nc2cn(Cc3ccc(C(C)N(C)CCC)cc3OC)nc12] | 426.4 | (400 MHz, CD₃OD) δ 7.88 (s, 1H), 7.36 − 7.33 (m, 1H), 7.19 (s, 1H), 7.14 − 7.05 (m, 1H), 5.58 (s, 2H), 4.71 − 4.52 (m, 1H), 3.96 (s, 3H), 3.69 − 3.59 (m, 2H), 3.19 − 3.10 (m, 1H), 3.04 − 2.96 (m, 1H), 2.89 − 2.83 (m, 2H), 2.78 − 2.70 (m, 1H), 1.96 − 1.68 (m, 7H), 1.56 − 1.34 (m, 2H), 1.09 − 0.76 (m, 6H) |
| 234 | 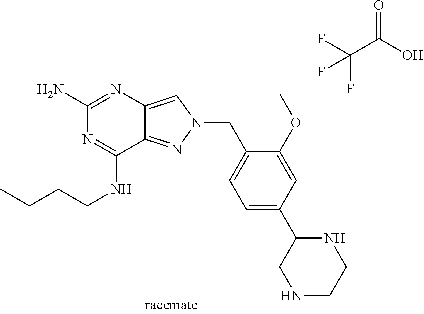 [SMILES: O=C(O)C(F)(F)F ; CCCCNc1nc(N)nc2cn(Cc3ccc(C4CNCCN4)cc3OC)nc12] | 411.2 | (300 MHz, CD₃OD) δ 7.89 (s, 1H), 7.34 − 7.32 (m, 1H), 7.24 (s, 1H), 7.12 − 7.09 (m, 1H), 5.57 (s, 2H), 4.54 − 4.50 (m, 1H), 3.97 (s, 3H), 3.68 − 3.32 (m, 8H), 1.73 − 1.68 (m, 2H), 1.49 − 1.37 (m, 2H), 1.01 − 0.96 (m, 3H). |
| 235 | 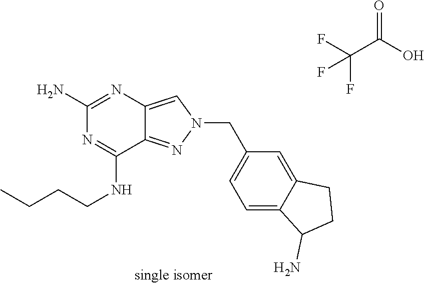 [SMILES: O=C(O)C(F)(F)F ; CCCCNc1nc(N)nc2cn(Cc3ccc4c(c3)CCC4N)nc12] | 352.2 | (300 MHz, CD₃OD) δ 7.91 (s, 1H), 7.53 − 7.50 (m, 1H), 7.36 − 7.33 (m, 2H), 5.58 (s, 2H), 4.80 − 4.79 (m, 1H), 3.67 − 3.62 (m, 2H), 3.32 − 3.11 (m, 1H), 3.04 − 2.94 (m, 1H), 2.68 − 2.56 (m, 1H), 2.16 − 2.09 (m, 1H), 1.74 − 1.65 (m, 2H), 1.50 − 1.37 (m, 2H), 1.02 − 0.97 (m, 3H). |
| 236 | 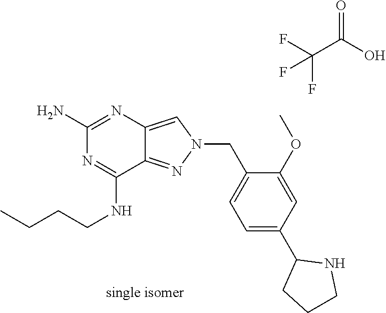 | 396.3 | (300 MHz, CD₃OD) δ 7.85 (s, 1H), 7.34 − 7.31 (m, 1H), 7.17 (s, 1H), 7.13 − 7.05 (m, 1H), 5.57 (s, 2H), 4.68 − 4.61 (m, 1H), 3.95 (s, 3H), 3.69 − 3.62 (m, 2H), 3.52 − 3.49 (m, 2H), 2.53 − 2.51 (m, 1H), 2.39 − 2.15 (m, 3H), 1.74 − 1.68 (m, 2H), 1.46 − 1.37 (m, 2H), 1.08 − 0.95 (m, 3H) |
| 237 | 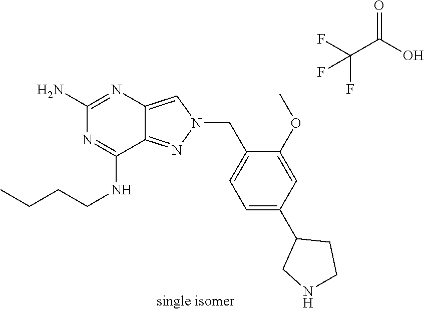 [SMILES: O=C(O)C(F)(F)F ; CCCCNc1nc(N)nc2cn(Cc3ccc(C4CCNC4)cc3OC)nc12] | 396.2 | (300 MHz, CD₃OD) δ 7.83 (s, 1H), 7.31 − 7.29 (m, 1H), 7.25 − 7.02 (m, 2H), 5.52 (s, 2H), 3.90 (s, 3H), 3.75 − 3.64 (m, 5H), 3.41 − 3.39 (m, 1H), 3.27 − 3.23 (m, 1H), 2.50 (s, 1H), 2.28 − 2.15 (m, 1H), 1.73 − 1.68 (m, 2H), 1.56 − 1.47 (m, 2H), 1.01 − 0.95 (m, 3H). |
| 238 | 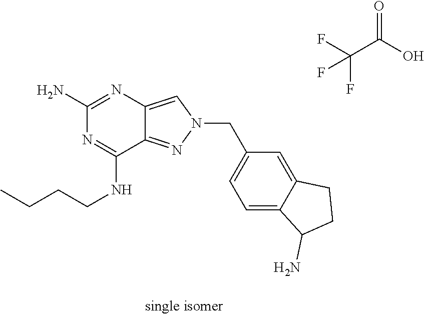 [SMILES: O=C(O)C(F)(F)F ; CCCCNc1nc(N)nc2cn(Cc3ccc4c(c3)CCC4N)nc12] | 352.3 | (300 MHz, CD₃OD) δ 7.91 (s, 1H), 7.53 − 7.50 (m, 2H), 7.36 − 7.33 (m, 2H), 5.58 (s, 2H), 4.80 − 4.79 (m, 1H), 3.67 − 3.62 (m, 2H), 3.04 − 2.94 (m, 1H), 2.68 − 2.56 (m, 1H), 2.16 − 2.09 (m, 1H), 1.74 − 1.65 (m, 2H), 1.50 − 1.37 (m, 2H), 1.02 − 0.97 (m, 3H). |
| 239 | 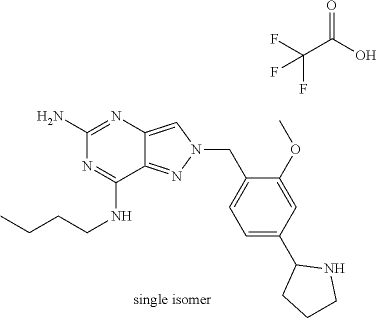 | 396.3 | (300 MHz, CD₃OD) δ 7.85 (s, 1H), 7.34 − 7.31 (m, 1H), 7.15 (s, 1H), 7.10 − 7.08 (m, 1H), 5.57 (s, 2H), 4.88 − 4.62 (m, 1H), 3.94 (s, 3H), 3.66 − 3.61 (m, 2H), 3.55 − 3.51 (m, 2H), 2.53 − 2.49 (m, 1H), 2.46 − 2.28 (m, 3H), 1.73 − 1.71 (m, 2H), 1.54 − 1.39 (m, 2H), 1.01 − 0.95 (m, 3H) |
| 240 | 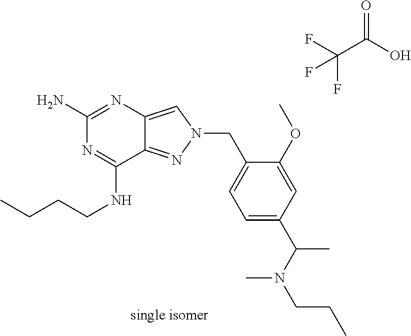 | 426.4 | (300 MHz, CD₃OD) δ 7.87 (s, 1H), 7.40 − 7.30 (m, 1H), 7.20 − 7.15 (m, 1H), 7.15 − 7.05 (m, 1H), 5.57 (s, 2H), 4.60 − 4.50 (m, 1H), 3.93 (s, 3H), 3.70 − 3.60 (m, 2H), 3.25 − 3.00 (m, 1H), 2.85 (s, 3H), 2.75 − 2.60 (m, 1H), 1.80 − 1.65 (m, 7H), 1.55 − 1.30 (m, 2H), 1.05 − 0.85 (m, 6H) |
| 241 | 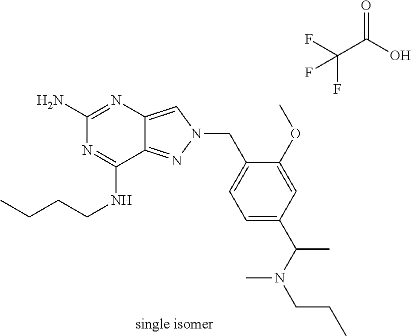 | 426.4 | (300 MHz, CD₃OD) δ 7.87 (s, 1H), 7.40 − 7.30 (m, 1H), 7.20 − 7.15 (m, 1H), 7.15 − 7.05 (m, 1H), 5.57 (s, 2H ), 4.60 − 4.50 (m, 1H), 3.93 (s, 3H), 3.70 − 3.60 (m, 2H), 3.25 − 3.00 (m, 1H), 2.86 (s, 3H), 2.75 − 2.60 (m, 1H), 1.80 − 1.65 (m, 7H), 1.55 − 1.30 (m, 2H), 1.05 − 0.85 (m, 6H). |

### Example 31

### Preparation of Compound 208

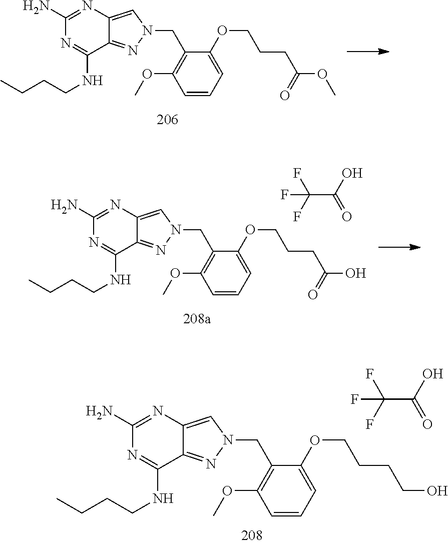

### Step A—Synthesis of Compound 208a

To a mixture of compound 206 (290 mg, 0.655 mmol) in MeOH (1.0 mL), THF (1.0 mL), and water (1.0 mL) was added sodium hydroxide (52.4 mg, 1.311 mmol) at 0° C., and the resulting mixture was allowed to stir for 2 hours at 25° C. The reaction mixture was concentrated in vacuo, and the resulting residue was purified using RP-Flash (eluted with 0-30% acetonitrile in aq. 0.05% TFA) to provide compound 208a. MS: m/z=429.2 [M+H].

### Step B—Synthesis of Compound 208

To a mixture of compound 208a (30 mg, 0.055 mmol) in THF (1 mL) was added LiAlH₄ (1 M in THF, 0.276 mL, 0.276 mmol) at 0° C. The resulting mixture was allowed to stir for 3 hours at 20° C. The reaction mixture was quenched by water (0.005 mL), 15% aq. NaOH (0.005 mL), and water (0.015 mL), filtered, and the filtrate was concentrated in vacuo. The resulting residue was purified using prep-HPLC: Column: SunFire Prep C18 OBD Column, 19×150 mm, 5 μm 10 nm; Mobile Phase A: water (0.05% TFA), Mobile Phase B: acetonitrile; Flow rate: 25 mL/min; Gradient: 20% B to 45% B in 6.8 minutes, Wave Length: 254 nm; RT1 (min): 5.37 to provide compound 208. MS: m/z=415.2 [M+H]. ¹H NMR (300 MHz, CD₃OD) δ 7.65 (s, 1H), 7.39-7.33 (m, 1H), 6.74-6.71 (m, 2H), 5.57 (s, 2H), 4.10-4.00 (m, 2H), 3.88 (s, 3H), 3.65-3.59 (m, 4H), 1.88-1.81 (m, 2H), 1.74-1.66 (m, 4H), 1.50-1.39 (m, 2H), 1.01-0.97 (m, 3H).

### Example 32

### Preparation of Compound 218

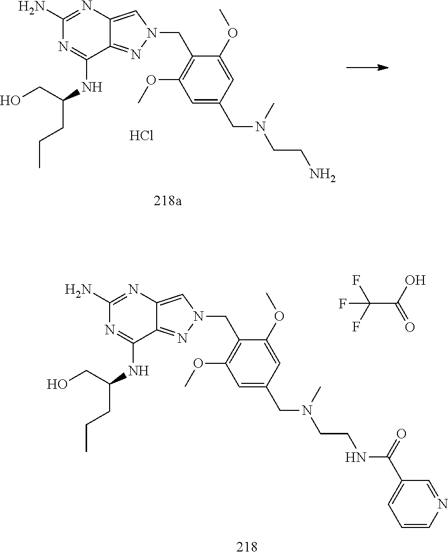 [SMILES: CCC[C@@H](CO)Nc1nc(N)nc2cn(Cc3c(OC)cc(CN(C)CCN)cc3OC)nc12 ; Cl ; CCC[C@@H](CO)Nc1nc(N)nc2cn(Cc3c(OC)cc(CN(C)CCNC(=O)c4cccnc4)cc3OC)nc12 ; O=C(O)C(F)(F)F]

Compound 218 was made from compound 218a, using the method described in Example 10, and substituting the appropriate reactants and/or reagents. MS: m/z=578.3 [M+H]. ¹H NMR (300 MHz, CD₃OD) δ 9.02 (s, 1H), 8.78 (s, 1H), 8.30-8.27 (m, 1H), 7.77 (s, 1H), 7.61 (s, 1H), 6.95 (s, 2H), 5.59 (s, 2H), 4.54-4.52 (m, 3H), 3.94-3.87 (m, 8H), 3.73-3.69 (m, 2H), 3.65-3.61 (m, 2H), 3.33-3.30 (m, 3H), 1.70-1.68 (m, 2H), 1.42-1.40 (m, 2H), 0.96-0.94 (m, 3H).

### Example 33

### Preparation of Compound 230

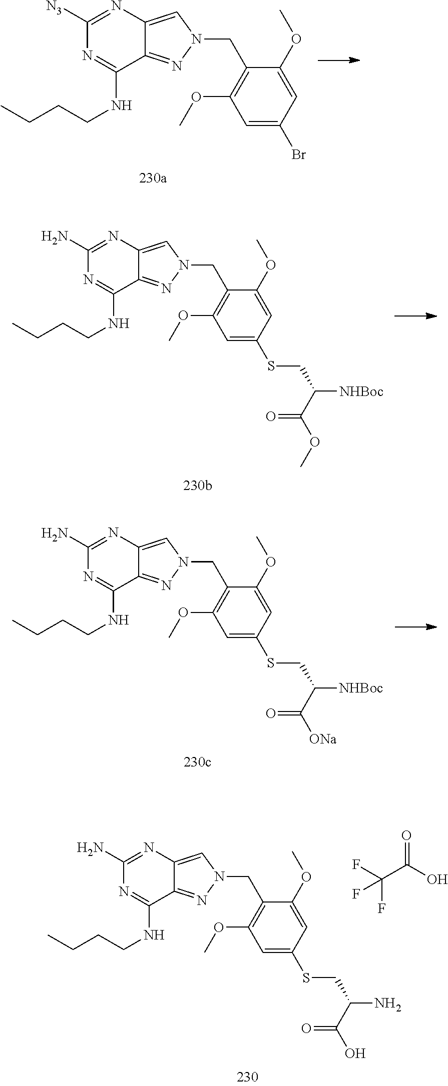

### Step A—Synthesis of Compound 230b

A mixture of compound 230a (30 mg, 0.065 mmol), [Ni(dtbbpy)(H₂O)(H₂O)₄]Cl₂ (0.260 mg, 0.650 μmol), methyl (tert-butoxycarbonyl)-L-cysteinate (30.6 mg, 0.130 mmol), tris(2,2′-bipyridine)ruthenium(II) hexafluorophosphate (55.9 mg, 0.065 mmol), and ammonium bis(catechol) silicate (28.7 mg, 0.085 mmol) in DMF (1 mL) was degassed by bubbling argon stream for 20 minutes, then irradiated with a 34 W blue LED lamp for 48 hours. The reaction mixture was directly purified using RP-Flash chromatography (eluted with 0-35% acetonitrile in aq. 0.05% TFA) to provide compound 230b. MS: m/z=590.15 [M+H].

### Step B—Synthesis of Compound 230c

To a mixture of compound 230b (80 mg, 0.136 mmol) in THF (0.3 mL), and MeOH (0.3 mL), and water (0.3 mL) was added NaOH (8.14 mg, 0.203 mmol). The resulting mixture was allowed to stir for 3 hours at 20° C., then the reaction mixture was directly concentrated in vacuo to provide compound 230c, which was used without further purification. MS (ESI, m/z): 576.4 [M+H].

### Step C—Synthesis of Compound 230

Compound 230 was made from compound 230c, using the method described in Example 23, step B, and substituting the appropriate reactants and/or reagents. MS: m/z=476.1 [M+H]. ¹H NMR (300 MHz, CD₃OD) δ 7.72 (s, 1H), 6.88 (s, 2H), 5.53 (s, 2H), 4.10-4.07 (m, 1H), 3.98 (s, 6H), 3.79-3.61 (m, 1H), 3. 58-3.48 (m, 3H), 1.74-1.66 (m, 2H), 1.50-1.40 (m, 2H), 1.02-0.98 (m, 3H).

### Example 34

### Human TLR7 Reporter Assay

HEK-Blue™ hTLR7 cells (Invivogen, San Diego, Calif.) were maintained at 37° C./5% CO₂/90% relative humidity in Dulbecco's Modified Eagle's Medium with 10% fetal bovine serum, 100 units/mL penicillin, 100 μg/mL streptomycin, 100 μg/mL normocin, 10 μg/mL blasticidin, and 100 μg/mL zeocin. HEK-Blue™ hTLR7 cells were maintained at 10-90% confluence and used before passage 20. For this assay, test compounds were dissolved in DMSO, and 10-point serial 3-fold dilution series in DMSO were prepared in Echo Qualified 384-well Polypropylene Microplates (Labcyte, San Jose, Calif.). Assay plates (384-well flat, clear bottom, black polystyrene TC-treated microtiter plates; Corning, Corning, N.Y.) were prepared by dispensing 150 nL of test compounds, high control (CL097; Invivogen, San Diego, Calif.), and low control (DMSO) by ECHO acoustic dispenser (Labcyte, San Jose Calif.) followed by addition of 5 μL of HEK-Blue™ Detection assay media (Invivogen, San Diego, Calif.). HEK-Blue™ hTLR7 cells in HEK-Blue™ Detection assay media (20,000 cells per well in 45 μL of media) were added to the assay plate and incubated for 16 hours at 37° C./5% CO₂/90% relative humidity. For detection, assay plates were removed from the incubator, allowed to cool to ambient temperature, centrifuged at 200×g for 1 minutes, and read with an EnSpire Multimode Plate Reader (Perkin Elmer, Waltham, Mass.) for absorbance at 620 nm. Test compound effects were normalized to the window defined by the controls, CL097 (6 μM), and DMSO. Calculated % effects were fit using a 4-parameter algorithm, and EC₅₀ was reported.

### Example 35

### Human TLR8 Reporter Assay

HEK-Blue™ hTLR8 cells (Invivogen, San Diego, Calif.) were maintained at 37° C./5% CO₂/90% relative humidity in Dulbecco's Modified Eagle's Medium with 10% fetal bovine serum, 100 units/mL penicillin, 100 μg/mL streptomycin, 100 μg/mL normocin, 10 μg/mL blasticidin, and 100 μg/mL zeocin. HEK-Blue™ hTLR8 cells were maintained at 10-90% confluence and used before passage 20. For this assay, test compounds were dissolved in DMSO, and 10-point serial 3-fold dilution series in DMSO were prepared in Echo Qualified 384-well Polypropylene Microplates (Labcyte, San Jose, Calif.). Assay plates (384-well flat, clear bottom, black polystyrene TC-treated microtiter plates; Corning, Corning, N.Y.) were prepared by dispensing 150 nL of test compounds, high control (tl8-506; Invivogen, San Diego, Calif.), and low control (DMSO) by ECHO acoustic dispenser (Labcyte, San Jose Calif.) followed by addition of 5 μL of HEK-Blue™ Detection assay media (Invivogen, San Diego, Calif.). HEK-Blue™ hTLR8 cells in HEK-Blue™ Detection assay media (20,000 cells per well in 45 μL of media) were added to the assay plate and incubated for 16 hours at 37° C./5% CO₂/90% relative humidity. For detection, assay plates were removed from the incubator, allowed to cool to ambient temperature, centrifuged at 200×g for 1 minutes, and read with an EnSpire Multimode Plate Reader (Perkin Elmer, Waltham, Mass.) for absorbance at 620 nm. Test compound effects were normalized to the window defined by the controls, tl8-506 (1 μM), and DMSO. Calculated % effects were fit using a 4-parameter algorithm, and EC₅₀ was reported.

Illustrative compounds of the present invention were tested in one or more of the above assays and results are provided in the table below:

| | hTLR7^{a} | hTLR8^{b} |
|---|---|---|
| Compound | EC₅₀ (nM) | EC₅₀ (nM) |
| 1 | 665 | 4900 |
| 2 | 1090 | 4210 |
| 3 | 1220 | >29900 |
| 4 | 578 | 6540 |
| 5 | 72.7 | 328 |
| 7 | 2520 | >29900 |
| 8 | 50.9 | 546 |
| 13 | 3040 | 26100 |
| 14 | 54.2 | 3830 |
| 15 | 291 | >29900 |
| 16 | 86.2 | >9980 |
| 17 | 398 | >29900 |
| 18 | 315 | >29900 |
| 19 | 435 | >29900 |
| 24 | 2450 | >29900 |
| 26 | 464 | 1890 |
| 27 | 1120 | 4380 |
| 28 | 214 | 1010 |
| 29 | 2770 | 28200 |
| 36 | 3100 | >29900 |
| 37 | 1030 | >9980 |
| 38 | 263 | 1690 |
| 39 | 239 | 748 |
| 40 | 125 | 813 |
| 41 | 68.6 | 710 |
| 42 | >29900 | 2980 |
| 43 | 1370 | >9980 |
| 44 | 858 | 3930 |
| 46 | 353 | 1084 |
| 47 | 8588 | >29940 |
| 48 | 7717 | >29940 |
| 49 | 7294 | >29940 |
| 50 | 843 | >29940 |
| 51 | 4732 | >29940 |
| 52 | 2802 | >29940 |
| 53 | 858 | >29940 |
| 54 | 496 | >29940 |
| 56 | 369 | >9980 |
| 58 | 228 | 345 |
| 59 | 964 | 2640 |
| 60 | 1940 | 3080 |
| 61 | 344 | 438 |
| 62 | 840 | 1490 |
| 63 | 50.8 | 2410 |
| 64 | 518 | 348 |
| 65 | 119 | 1020 |
| 66 | 493 | 199 |
| 67 | 281 | 392 |
| 68 | 190 | 3640 |
| 69 | 243 | 1490 |
| 70 | >1110 | 908 |
| 71 | 267 | 3040 |
| 72 | 597 | >29940 |
| 74 | 3327 | >29940 |
| 76 | 3040 | 719 |
| 77 | 490 | 1530 |
| 78 | 2650 | 2560 |
| 79 | 4450 | 1810 |
| 81 | >29900 | 3330 |
| 89 | 2620 | 24600 |
| 90 | 2210 | 15100 |
| 91 | 35.8 | 291 |
| 92 | 2260 | 11574 |
| 93 | 120 | 611 |
| 94 | 2040 | 4960 |
| 95 | 3350 | 4330 |
| 96 | 1970 | 7840 |
| 97 | 265 | 2370 |
| 98 | 414 | >9980 |
| 99 | 633 | 1380 |
| 100 | 74.4 | 499 |
| 101 | 153 | 1260 |
| 102 | 1410 | >9980 |
| 103 | 97.9 | 422 |
| 104 | 292 | 2040 |
| 105 | 1790 | 6400 |
| 106 | 311 | 1700 |
| 107 | 213 | 3020 |
| 108 | 29.3 | 116 |
| 109 | 91.9 | 99.8 |
| 110 | 553 | 3680 |
| 111 | 81.8 | 597 |
| 112 | 1570 | 19900 |
| 113 | 19 | 206 |
| 114 | 529 | 4490 |
| 115 | 329 | 5590 |
| 116 | 988 | 4200 |
| 117 | 751 | 2200 |
| 118 | 249 | 144 |
| 119 | 6650 | 22500 |
| 120 | 112 | 2220 |
| 121 | 490 | 2320 |
| 122 | 288 | 1400 |
| 123 | 743 | 880 |
| 124 | 266 | >9980 |
| 125 | 1740 | 18700 |
| 126 | 707 | >9980 |
| 127 | 1240 | 3300 |
| 128 | 95.2 | 633 |
| 129 | 293 | 1360 |
| 130 | 370 | 734 |
| 131 | 680 | 1250 |
| 132 | 708 | 1490 |
| 133 | 114 | 161 |
| 134 | 1520 | 14400 |
| 135 | 343 | >9980 |
| 136 | 174 | 1340 |
| 137 | 439 | 692 |
| 138 | 191 | 1780 |
| 139 | 717 | 942 |
| 140 | 252 | 1720 |
| 141 | 409 | 413 |
| 142 | 354 | 3220 |
| 143 | 360 | >3330 |
| 144 | 170 | 3810 |
| 145 | 2270 | 6320 |
| 146 | 628 | 637 |
| 147 | 180 | 265 |
| 148 | 370 | 590 |
| 149 | 5180 | 1180 |
| 150 | 1620 | 1640 |
| 151 | 2260 | 869 |
| 152 | 185 | 122 |
| 153 | 1060 | 3140 |
| 154 | 1080 | 4150 |
| 155 | 2460 | 6530 |
| 156 | 76.8 | 153 |
| 157 | 55.9 | 370 |
| 158 | 21.8 | 178 |
| 159 | 1150 | 9550 |
| 160 | 373 | 8300 |
| 161 | 42.2 | 91 |
| 162 | 160 | 1010 |
| 163 | 476 | 809 |
| 164 | 110 | 168 |
| 165 | 1360 | 4680 |
| 166 | 93 | 632 |
| 167 | 357 | 179 |
| 168 | 223 | 231 |
| 169 | 1850 | 4060 |
| 170 | 1560 | 4430 |
| 171 | 516 | 2520 |
| 172 | 2790 | 3990 |
| 173 | 246 | 2730 |
| 174 | 1450 | 8320 |
| 175 | 112 | 1110 |
| 176 | 329 | 467 |
| 177 | 681 | 2810 |
| 178 | 294 | 455 |
| 179 | 226 | 1890 |
| 180 | 1140 | 6370 |
| 181 | 171 | 1500 |
| 182 | 929 | 3360 |
| 183 | 30.8 | 380 |
| 184 | 103 | 728 |
| 185 | 382 | 103 |
| 186 | 190 | 278 |
| 187 | 20 | 63.3 |
| 188 | 5440 | 12400 |
| 189 | 90 | 290 |
| 190 | 187 | 2210 |
| 191 | 541 | 860 |
| 192 | 653 | 1160 |
| 193 | 57.7 | 106 |
| 194 | 139 | 137 |
| 195 | 1590 | 785 |
| 196 | 456 | 756 |
| 197 | 277 | 1590 |
| 198 | 396 | 835 |
| 199 | 108 | 39.9 |
| 200 | 185 | 4900 |
| 201 | 539 | 3260 |
| 202 | 354 | 3470 |
| 203 | 526 | 11400 |
| 204 | 508 | 533 |
| 205 | 409 | 519 |
| 206 | 380 | 2640 |
| 207 | 806 | 2340 |
| 208 | 299 | 906 |
| 209 | 200 | 161 |
| 210 | 20100 | 15500 |
| 211 | 365 | 198 |
| 212 | 7750 | 8140 |
| 213 | 4750 | 2230 |
| 214 | 525 | 235 |
| 215 | 474 | 224 |
| 216 | 106 | 119 |
| 217 | 51.7 | 91.7 |
| 218 | 6360 | 1490 |
| 219 | 122 | 275 |
| 220 | 118 | 170 |
| 221 | 5090 | 3490 |
| 222 | 6280 | 1070 |
| 223 | 4840 | 1800 |
| 224 | 3650 | 2250 |
| 225 | 531 | 311 |
| 226 | 533 | 233 |
| 227 | 10700 | 10800 |
| 228 | 2370 | 7020 |
| 229 | 2270 | 11400 |
| 230 | 898 | 8840 |
| 231 | 619 | 48.9 |
| 232 | 363 | 78.1 |
| 233 | 32.3 | 456 |
| 234 | 1490 | 9820 |
| 235 | 361 | 2470 |
| 236 | 241 | 2870 |
| 237 | 616 | 7250 |
| 238 | 254 | 1880 |
| 239 | 154 | 2600 |
| 240 | 50.7 | 1090 |
| 241 | 36 | 725 |

^{a}= data generated using the assay described in Example 34
^{b}= data generated using the assay described in Example 35

### Uses of the Pyrazolo[4,3-d]Pyrimidine Derivative

### Treatment or Prevention of Cellular Proliferation Disorders

The present disclosure also relates to methods of treating a cellular proliferative disorder, said methods comprising administering to a subject in need thereof a Pyrazolo[4,3-d]Pyrimidine Derivative.

The Pyrazolo[4,3-d]Pyrimidine Derivatives disclosed herein are potentially useful in treating diseases or disorders including, but not limited to, cellular proliferative disorders. Cellular proliferation disorders include, but are not limited to, cancers, benign papillomatosis, and gestational trophoblastic diseases. The terms “cancer”, “cancerous”, or “malignant” refer to or describe the physiological condition in mammals that is typically characterized by unregulated cell growth.

In specific embodiments, the cellular proliferative disorder is selected from cancer, benign papillomatosis, benign neoplastic diseases and gestational trophoblastic diseases. In particular embodiments, the gestational trophoblastic disease is selected from the group consisting of hydatidiform moles, and gestational trophoblastic neoplasia (e.g., invasive moles, choriocarcinomas, placental-site trophoblastic tumors, and epithelioid trophoblastic tumors). In a particular embodiment, the cellular proliferative disorder being treated is cancer.

Accordingly, in one embodiment, the invention provides methods for treating cancer in a patient, the methods comprising administering to the patient an effective amount of a Pyrazolo[4,3-d]Pyrimidine Derivative. In a specific embodiment, the amount administered is effective to treat cancer in the patient. In another specific embodiment, the amount administered is effective to inhibit cancer cell replication or cancer cell metastasis in the patient.

In one embodiments, the present invention includes the use of the Pyrazolo[4,3-d]Pyrimidine Derivatives, or a pharmaceutically acceptable salt thereof, for the preparation of a medicament for the treatment of cancer.

In another embodiment, the present invention includes Pyrazolo[4,3-d]Pyrimidine Derivatives, for use in the treatment of cancer.

In one embodiment, the cancer is metastatic. In another embodiment, the cancer is relapsed. In another embodiment, the cancer is refractory. In yet another embodiment, the cancer is relapsed and refractory.

In one embodiment, the patient has previously received treatment for cancer. In another embodiment, the patient has not previously received treatment for cancer.

In one embodiment, the patient has previously received systemic treatment for cancer. In another embodiment, the patient has not previously received systemic treatment for cancer.

In other embodiments, the cancer is present in an adult patient; in additional embodiments, the cancer is present in a pediatric patient.

The compounds, compositions and methods provided herein are useful for the treatment of cancer. Cancers that may be treated using the compounds, compositions and methods disclosed herein include, but are not limited to: (1) Cardiac: sarcoma (angiosarcoma, fibrosarcoma, rhabdomyosarcoma, liposarcoma), myxoma, rhabdomyoma, fibroma, lipoma and teratoma; (2) Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma, non-small cell; (3) Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma), colon, colorectal, rectal; (4) Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); (5) Liver: hepatoma (hepatocellular carcinoma), cholangiocarcinoma, hepatoblastoma, angiosarcoma, hepatocellular adenoma, hemangioma; (6) Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; (7) Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiforme, oligodendroglioma, schwannoma, retinoblastoma, congenital tumors), spinal cord neurofibroma, meningioma, glioma, sarcoma); (8) Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; (9) Hematologic: blood (myeloid leukemia [acute and chronic], acute lymphoblastic leukemia, chronic lymphocytic leukemia, chronic myelomonocytic (CMML), myeloproliferative diseases, multiple myeloma, myelodysplastic syndrome), Hodgkin's disease, non-Hodgkin's lymphoma [malignant lymphoma]; (10) Skin: malignant melanoma, basal cell carcinoma, squamous cell carcinoma, Karposi's sarcoma, moles dysplastic nevi, lipoma, angioma, dermatofibroma, keloids, psoriasis; and (11) Adrenal glands: neuroblastoma. Examples of cancer that may be treated using the compounds, compositions and methods of the invention include thyroid cancer, anaplastic thyroid carcinoma, epidermal cancer, head and neck cancer (e.g., squamous cell cancer of the head and neck), sarcoma, tetracarcinoma, hepatoma and multiple myeloma.

The term “cancerous cell” as used herein, includes a cell afflicted by any one of the above-identified conditions.

In particular embodiments, the cancer is selected from brain and spinal cancers, cancers of the head and neck, leukemia and cancers of the blood, skin cancers, cancers of the reproductive system, cancers of the gastrointestinal system, liver and bile duct cancers, kidney and bladder cancers, bone cancers, lung cancers, metastatic microsatellite instability-high (MSI-H) cancer, mismatch repair deficient cancer, malignant mesothelioma, sarcomas, lymphomas, glandular cancers, thyroid cancers, heart tumors, germ cell tumors, malignant neuroendocrine (carcinoid) tumors, midline tract cancers, and cancers of unknown primary origin (i.e., cancers in which a metastasized cancer is found but the original cancer site is not known). In particular embodiments, the cancer is AIDS-related.

In one embodiment, the cancer is bladder cancer. In another embodiment, the cancer is breast cancer. In yet another embodiment, the cancer is NSCLC. In still another embodiment, the cancer is CRC. In another embodiment, the cancer is RCC. In another embodiment, the cancer is HCC. In one embodiment, the cancer is skin cancer. In another embodiment, the skin cancer is melanoma. In another embodiment, the cancer is ovarian cancer. In yet another embodiment, the cancer is pancreatic cancer. In another embodiment, the cancer is a primary or metastatic brain cancer. In still another embodiment, the cancer is CRC.

In one embodiment, the invention comprises a method of treating unresectable or metastatic melanoma in a human patient. In some embodiments, the method comprises treating resected high-risk stage III melanoma.

In one embodiment, the invention comprises a method of treating metastatic non-small cell lung cancer (NSCLC) in a human patient. In some embodiments, the NSCLC is non-squamous. In other embodiments, the NSCLC is squamous.

In some embodiments, the cancer exhibits high PD-L1 expression [(Tumor Proportion Score (TPS) ≥50%)] and was not previously treated with platinum-containing chemotherapy. In alternative embodiments, the patient has a tumor with PD-L1 expression (TPS ≥1%), and was previously treated with platinum-containing chemotherapy. In specific embodiments, the patient had disease progression on or after receiving platinum-containing chemotherapy.

In certain embodiments the PD-L1 TPS is determined by an FDA-approved test.

In certain embodiments of the method for treating NSCLC, the patient's tumor has no EGFR or ALK genomic aberrations.

In certain embodiments of the method for treating NSCLC, the patient's tumor has an EGFR or ALK genomic aberration and had disease progression on or after receiving treatment for the EGFR or ALK aberration(s) prior to receiving combination therapy of the invention.

In one embodiment, the invention comprises a method of treating recurrent or metastatic head and neck squamous cell cancer (HNSCC) in a human patient. In some embodiments, the patient was previously treated with platinum-containing chemotherapy. In certain embodiments, the patient had disease progression during or after platinum-containing chemotherapy.

In one embodiment, the invention comprises a method of treating refractory classical Hodgkin lymphoma (cHL) in a human patient. In certain embodiments, the patient has relapsed after 1, 2, 3 or more lines of therapy for cHL. In specific embodiments, the patient is an adult patient. In alternative embodiments the patient is a pediatric patient.

In one embodiment, the invention comprises a method of treating locally advanced or metastatic urothelial carcinoma in a human patient. In certain embodiments, the patient is not eligible for cisplatin-containing chemotherapy. In further embodiments, the patient has disease progression during or following platinum-containing chemotherapy or within 12 months of neoadjuvant or adjuvant treatment with platinum-containing chemotherapy. In specific embodiments, the patient's tumor expresses PD-L1 (CPS ≥10).

In one embodiment, the invention comprises a method of treating unresectable or metastatic, microsatellite instability-high (MSI-H) or mismatch repair deficient solid tumors in a human patient. In specific embodiments, the patient had disease progression following prior anti-cancer treatment.

In one embodiment, the invention comprises a method of treating unresectable or metastatic, microsatellite instability-high (MSI-H) or mismatch repair deficient colorectal cancer in a human patient. In specific embodiments, the patient had disease progression following prior treatment with a fluoropyrimidine, oxaliplatin, and irinotecan.

In one embodiment, the invention comprises a method of treating recurrent locally advanced or metastatic gastric cancer or recurrent locally advanced or metastatic gastroesophageal junction adenocarcinoma in a human patient. In specific embodiments, the patient's tumor expresses PD-L1 [Combined Positive Score (CPS) ≥1]. In some embodiments, the patient has disease progression on or after two or more prior lines of therapy including fluoropyrimidine- and platinum-containing chemotherapy. In some embodiments, the patient has disease progression on or after two or more prior lines of therapy including HER2/neu-targeted therapy.

In one embodiment, the invention comprises a method of treating non-Hodgkin lymphoma in a human patient. In certain embodiments, the non-Hodgkin lymphoma is primary mediastinal large B-cell lymphoma.

In one embodiment, the invention comprises a method of treating breast cancer in a human patient. in specific embodiments, the breast cancer is triple negative breast cancer. In other specific embodiments, the breast cancer is ER+/HER2− breast cancer.

In one embodiment, the invention comprises a method of treating cancer in a human patient comprising, wherein the patient has a tumor with a high mutational burden.

In specific embodiments, the cancer is selected from brain and spinal cancers. In particular embodiments, the brain and spinal cancer is selected from the group consisting of anaplastic astrocytomas, glioblastomas, astrocytomas, and estheosioneuroblastomas (also known as olfactory blastomas). In particular embodiments, the brain cancer is selected from the group consisting of astrocytic tumor (e.g., pilocytic astrocytoma, subependymal giant-cell astrocytoma, diffuse astrocytoma, pleomorphic xanthoastrocytoma, anaplastic astrocytoma, astrocytoma, giant cell glioblastoma, glioblastoma, secondary glioblastoma, primary adult glioblastoma, and primary pediatric glioblastoma), oligodendroglial tumor (e.g., oligodendroglioma, and anaplastic oligodendroglioma), oligoastrocytic tumor (e.g., oligoastrocytoma, and anaplastic oligoastrocytoma), ependymoma (e.g., myxopapillary ependymoma, and anaplastic ependymoma); medulloblastoma, primitive neuroectodermal tumor, schwannoma, meningioma, atypical meningioma, anaplastic meningioma, pituitary adenoma, brain stem glioma, cerebellar astrocytoma, cerebral astorcytoma/malignant glioma, visual pathway and hypothalmic glioma, and primary central nervous system lymphoma. In specific instances of these embodiments, the brain cancer is selected from the group consisting of glioma, glioblastoma multiforme, paraganglioma, and suprantentorial primordial neuroectodermal tumors (sPNET). In one embodiment, the brain or spinal cancer is a metastatic brain tumor or tumors.

In specific embodiments, the cancer is selected from cancers of the head and neck, including recurrent or metastatic head and neck squamous cell carcinoma (HNSCC), nasopharyngeal cancers, nasal cavity and paranasal sinus cancers, hypopharyngeal cancers, oral cavity cancers (e.g., squamous cell carcinomas, lymphomas, and sarcomas), lip cancers, oropharyngeal cancers, salivary gland tumors, cancers of the larynx (e.g., laryngeal squamous cell carcinomas, rhabdomyosarcomas), and cancers of the eye or ocular cancers. In particular embodiments, the ocular cancer is selected from the group consisting of intraocular melanoma and retinoblastoma.

In specific embodiments, the cancer is selected from leukemia and cancers of the blood. In particular embodiments, the cancer is selected from the group consisting of myeloproliferative neoplasms, myelodysplastic syndromes, myelodysplastic/myeloproliferative neoplasms, acute myeloid leukemia (AML), myelodysplastic syndrome (MDS), chronic myelogenous leukemia (CML), myeloproliferative neoplasm (MPN), post-MPN AML, post-MDS AML, del(5q)-associated high risk MDS or AML, blast-phase chronic myelogenous leukemia, angioimmunoblastic lymphoma, acute lymphoblastic leukemia, Langerans cell histiocytosis, hairy cell leukemia, and plasma cell neoplasms including plasmacytomas and multiple myelomas. Leukemias referenced herein may be acute or chronic.

In specific embodiments, the cancer is selected from skin cancers. In particular embodiments, the skin cancer is selected from the group consisting of melanoma, squamous cell cancers, and basal cell cancers. In specific embodiments, the skin cancer is unresectable or metastatic melanoma.

In specific embodiments, the cancer is selected from cancers of the reproductive system. In particular embodiments, the cancer is selected from the group consisting of breast cancers, cervical cancers, vaginal cancers, ovarian cancers, endometrial cancers, prostate cancers, penile cancers, and testicular cancers. In specific instances of these embodiments, the cancer is a breast cancer selected from the group consisting of ductal carcinomas and phyllodes tumors. In specific instances of these embodiments, the breast cancer may be male breast cancer or female breast cancer. In some instances of these embodiments, the breast cancer is triple-negative breast cancer. In other instances, the breast cancer is ER+/HER2− breast cancer. In specific instances of these embodiments, the cancer is a cervical cancer selected from the group consisting of squamous cell carcinomas and adenocarcinomas. In specific instances of these embodiments, the cancer is an ovarian cancer selected from the group consisting of epithelial cancers.

In specific embodiments, the cancer is selected from cancers of the gastrointestinal system. In particular embodiments, the cancer is selected from the group consisting of esophageal cancers, gastric cancers (also known as stomach cancers), gastrointestinal carcinoid tumors, pancreatic cancers, gall bladder cancers, colorectal cancers, and anal cancer. In instances of these embodiments, the cancer is selected from the group consisting of esophageal squamous cell carcinomas, esophageal adenocarcinomas, gastric adenocarcinomas, gastrointestinal carcinoid tumors, gastrointestinal stromal tumors, gastric lymphomas, gastrointestinal lymphomas, solid pseudopapillary tumors of the pancreas, pancreatoblastoma, islet cell tumors, pancreatic carcinomas including acinar cell carcinomas and ductal adenocarcinomas, gall bladder adenocarcinomas, colorectal adenocarcinomas, microsatellite stable colorectal cancer, advanced microsatellite stable colorectal cancer, metastatic microsatellite stable colorectal cancer and anal squamous cell carcinomas.

In specific embodiments, the cancer is selected from liver and bile duct cancers. In particular embodiments, the cancer is liver cancer (also known as hepatocellular carcinoma). In particular embodiments, the cancer is bile duct cancer (also known as cholangiocarcinoma); in instances of these embodiments, the bile duct cancer is selected from the group consisting of intrahepatic cholangiocarcinoma and extrahepatic cholangiocarcinoma.

In specific embodiments, the cancer is selected from kidney and bladder cancers.

In particular embodiments, the cancer is a kidney cancer selected from the group consisting of renal cell cancer, Wilms tumors, and transitional cell cancers. In particular embodiments, the cancer is a bladder cancer selected from the group consisting of urothelial carcinoma (a transitional cell carcinoma), squamous cell carcinomas, and adenocarcinomas.

In specific embodiments, the cancer is selected from bone cancers. In particular embodiments, the bone cancer is selected from the group consisting of osteosarcoma, malignant fibrous histiocytoma of bone, Ewing sarcoma, chordoma (cancer of the bone along the spine).

In specific embodiments, the cancer is selected from lung cancers. In particular embodiments, the lung cancer is selected from the group consisting of non-small cell lung cancer, small cell lung cancers, bronchial tumors, and pleuropulmonary blastomas.

In specific embodiments, the cancer is selected from malignant mesothelioma. In particular embodiments, the cancer is selected from the group consisting of epithelial mesothelioma and sarcomatoids.

In specific embodiments, the cancer is selected from sarcomas. In particular embodiments, the sarcoma is selected from the group consisting of central chondrosarcoma, central and periosteal chondroma, fibrosarcoma, clear cell sarcoma of tendon sheaths, and Kaposi's sarcoma.

In specific embodiments, the cancer is selected from lymphomas. In particular embodiments, the cancer is selected from the group consisting of Hodgkin lymphoma (e.g., classical Hodgkin refractory lymphoma), non-Hodgkin lymphoma (e.g., diffuse large B-cell lymphoma, follicular lymphoma, mycosis fungoides, Sezary syndrome, primary central nervous system lymphoma), cutaneous T-cell lymphomas, primary central nervous system lymphomas.

In specific embodiments, the cancer is selected from glandular cancers. In particular embodiments, the cancer is selected from the group consisting of adrenocortical cancer (also known as adrenocortical carcinoma or adrenal cortical carcinoma), pheochromocytomas, paragangliomas, pituitary tumors, thymoma, and thymic carcinomas.

In specific embodiments, the cancer is selected from thyroid cancers. In particular embodiments, the thyroid cancer is selected from the group consisting of medullary thyroid carcinomas, papillary thyroid carcinomas, and follicular thyroid carcinomas.

In specific embodiments, the cancer is selected from germ cell tumors. In particular embodiments, the cancer is selected from the group consisting of malignant extracranial germ cell tumors and malignant extragonadal germ cell tumors. In specific instances of these embodiments, the malignant extragonadal germ cell tumors are selected from the group consisting of nonseminomas and seminomas.

In specific embodiments, the cancer is selected from heart tumors. In particular embodiments, the heart tumor is selected from the group consisting of malignant teratoma, lymphoma, rhabdomyosacroma, angiosarcoma, chondrosarcoma, infantile fibrosarcoma, and synovial sarcoma.

In embodiments, the cancer is a metastatic tumor, for example, liver metastases from colorectal cancer or pancreatic cancer; and brain metastases from lung or breast cancer.

In embodiments, the cancer is selected from the group consisting of solid tumors and lymphomas. In particular embodiments, the cancer is selected from the group consisting of advanced or metastatic solid tumors and lymphomas. In more particular embodiments, the cancer is selected from the group consisting of malignant melanoma, head and neck squamous cell carcinoma, breast adenocarcinoma, and lymphomas. In aspects of such embodiments, the lymphomas are selected from the group consisting of diffuse large B-cell lymphoma, follicular lymphoma, mantle cell lymphoma, small lymphocytic lymphoma, mediastinal large B-cell lymphoma, splenic marginal zone B-cell lymphoma, extranodal marginal zone B-cell lymphoma of mucosa-associated lymphoid tissue (malt), nodal marginal zone B-cell lymphoma, lymphoplasmacytic lymphoma, primary effusion lymphoma, Burkitt lymphoma, anaplastic large cell lymphoma (primary cutaneous type), anaplastic large cell lymphoma (systemic type), peripheral T-cell lymphoma, angioimmunoblastic T-cell lymphoma, adult T-cell lymphoma/leukemia, nasal type extranodal NK/T-cell lymphoma, enteropathy-associated T-cell lymphoma, gamma/delta hepatosplenic T-cell lymphoma, subcutaneous panniculitis-like T-cell lymphoma, mycosis fungoides, and Hodgkin lymphoma.

In particular embodiments, the cancer is classified as stage III cancer or stage IV cancer. In some instances of these embodiments, the cancer is not surgically resectable.

### Compositions and Administration

When administered to a patient, a Pyrazolo[4,3-d]Pyrimidine Derivative can be administered as a component of a pharmaceutical composition that comprises a pharmaceutically acceptable excipient. Accordingly, in one embodiment, the present invention provides pharmaceutical compositions comprising an effective amount of a Pyrazolo[4,3-d]Pyrimidine Derivative, and one or more pharmaceutically acceptable carriers or excipients.

The Pyrazolo[4,3-d]Pyrimidine Derivatives are useful in preparing a medicament that is useful in treating a cellular proliferative disorder. In one embodiment, the Pyrazolo[4,3-d]Pyrimidine Derivatives are also useful for preparing a medicament that is useful in treating cancer.

In the pharmaceutical compositions and methods of the present invention, the active ingredients will typically be administered in admixture with suitable carrier materials suitably selected with respect to the intended form of administration, i.e., oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms), and the like. Solid form preparations include powders, tablets, dispersible granules, capsules, cachets and suppositories. Powders and tablets may be comprised of from about 0.5 to about 95 percent inventive composition. Tablets, powders, cachets and capsules can be used as solid dosage forms suitable for oral administration.

Moreover, when desired or needed, suitable binders, lubricants, disintegrating agents and coloring agents may also be incorporated in the mixture. Suitable binders include starch, gelatin, natural sugars, corn sweeteners, natural and synthetic gums such as acacia, sodium alginate, carboxymethylcellulose, polyethylene glycol and waxes. Suitable lubricants include boric acid, sodium benzoate, sodium acetate, sodium chloride, and the like. Disintegrants include starch, methylcellulose, guar gum, and the like. Sweetening and flavoring agents, and preservatives may also be included where appropriate.

Also included are solid form preparations which are intended to be converted, shortly before use, to liquid form preparations for either oral or parenteral administration. Such liquid forms include solutions, suspensions and emulsions.

For preparing suppositories, a low melting wax such as a mixture of fatty acid glycerides or cocoa butter is first melted, and the active ingredient is dispersed homogeneously therein. The molten homogeneous mixture is then poured into convenient sized molds, allowed to cool, and thereby solidify.

Additionally, the pharmaceutical compositions of the present invention may be formulated in sustained release form to provide the rate-controlled release of any one or more of the components or active ingredients to optimize therapeutic effects, i.e., anticancer activity and the like. Suitable dosage forms for sustained release include layered tablets containing layers of varying disintegration rates or controlled release polymeric matrices impregnated with the active components, and shaped in tablet form or capsules containing such impregnated or encapsulated porous polymeric matrices.

In one embodiment, the Pyrazolo[4,3-d]Pyrimidine Derivative is administered orally. In another embodiment, the Pyrazolo[4,3-d]Pyrimidine Derivative is administered orally in a capsule. In another embodiment, the Pyrazolo[4,3-d]Pyrimidine Derivative is administered orally in a tablet.

In another embodiment, the Pyrazolo[4,3-d]Pyrimidine Derivative is administered intravenously.

In another embodiment, the Pyrazolo[4,3-d]Pyrimidine Derivative is administered via subcutaneous injection.

In another embodiment, the Pyrazolo[4,3-d]Pyrimidine Derivative is administered via intertumoral injection.

In another embodiment, the Pyrazolo[4,3-d]Pyrimidine Derivative is administered topically. In a specific embodiment, the Pyrazolo[4,3-d]Pyrimidine Derivative is formulated as a cream that can be applied topically.

In still another embodiment, the Pyrazolo[4,3-d]Pyrimidine Derivative is administered sublingually.

In one embodiment, a pharmaceutical preparation comprising a Pyrazolo[4,3-d]Pyrimidine Derivative is in unit dosage form. In such form, the preparation is subdivided into unit doses containing effective amounts of the active components.

Compositions can be prepared using techniques such as conventional mixing, granulating or coating methods; and by using solid dispersion based upon the guidance provided herein. In one embodiment, the present compositions can contain from about 0.1% to about 99% of a Pyrazolo[4,3-d]Pyrimidine Derivative by weight or volume. In various embodiments, the present compositions can contain, in one embodiment, from about 1% to about 70%, or from about 5% to about 60%, or from about 10% to about 50% of a Pyrazolo[4,3-d]Pyrimidine Derivative by weight or volume.

In one embodiment, the present invention provides compositions comprising a Pyrazolo[4,3-d]Pyrimidine Derivative, a pharmaceutically acceptable carrier, and one or more additional therapeutic agents. In another embodiment, the present invention provides compositions comprising a Pyrazolo[4,3-d]Pyrimidine Derivative, a pharmaceutically acceptable carrier, and one additional therapeutic agents. In another embodiment, the present invention provides compositions comprising a Pyrazolo[4,3-d]Pyrimidine Derivative, a pharmaceutically acceptable carrier, and two additional therapeutic agents.

The quantity of a Pyrazolo[4,3-d]Pyrimidine Derivative in a unit dose of preparation may be varied or adjusted from about 1 mg to about 2500 mg. In various embodiments, the quantity is from about 10 mg to about 1000 mg, 1 mg to about 500 mg, 1 mg to about 100 mg, 1 mg to about 50 mg, 1 mg to about 20 mg, and 1 mg to about 10 mg.

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR), e.g., the Physicians' Desk Reference, 64^{th} Edition, 2010 (published by PDR Network, LLC at Montvale, N.J. 07645-1725), presently accessible through www.pdr.net; the disclosures of which are incorporated herein by reference thereto.

If the patient is responding, or is stable, after completion of the therapy cycle, the therapy cycle can be repeated according to the judgment of the skilled clinician. Upon completion of multiple therapy cycles, the patient can be continued on the Pyrazolo[4,3-d]Pyrimidine Derivatives at the same dose that was administered in the treatment protocol. This maintenance dose can be continued until the patient progresses, or can no longer tolerate the dose (in which case the dose can be reduced and the patient can be continued on the reduced dose).

The doses and dosage regimen of the additional therapeutic agent(s) used in the combination therapies of the present invention for the treatment of cellular proliferative disorders can be determined by the attending clinician, taking into consideration the approved doses and dosage regimen in the package insert; the age, sex and general health of the patient; and the type and severity of the cellular proliferative disorder. When administered in combination with one or more additional therapeutic agents, the Pyrazolo[4,3-d]Pyrimidine Derivative, and the additional therapeutic agent(s) can be administered simultaneously (i.e., in the same composition or in separate compositions one right after the other) or sequentially. This is particularly useful when the components of the combination are given on different dosing schedules, e.g., one component is administered once daily and another component is administered every six hours, or when the preferred pharmaceutical compositions are different, e.g., one is a tablet and one is a capsule. A kit comprising the separate dosage forms can therefore be advantageous.

The attending clinician, in judging whether treatment is effective at the dosage administered, will consider the general well-being of the patient as well as more definite signs such as relief of cancer-related symptoms (e.g., pain), inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

Generally, a total daily dosage of a Pyrazolo[4,3-d]Pyrimidine Derivative alone, or when administered as combination therapy, can range from about 1 to about 2500 mg per day, although variations will necessarily occur depending on the target of therapy, the patient and the route of administration. In one embodiment, the dosage is from about 10 to about 1000 mg/day, administered in a single dose or in 2-4 divided doses. In another embodiment, the dosage is from about 1 to about 500 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 mg/day, administered in a single dose or in 2-4 divided doses. In another embodiment, the dosage is from about 500 to about 1500 mg/day, administered in a single dose or in 2-4 divided doses. In still another embodiment, the dosage is from about 500 to about 1000 mg/day, administered in a single dose or in 2-4 divided doses. In yet another embodiment, the dosage is from about 100 to about 500 mg/day, administered in a single dose or in 2-4 divided doses.

For convenience, the total daily dosage may be divided and administered in portions during the day if desired. In one embodiment, the daily dosage is administered in one portion. In another embodiment, the total daily dosage is administered in two divided doses over a 24-hour period. In another embodiment, the total daily dosage is administered in three divided doses over a 24-hour period. In still another embodiment, the total daily dosage is administered in four divided doses over a 24-hour period.

The amount and frequency of administration of a Pyrazolo[4,3-d]Pyrimidine Derivative will be regulated according to the judgment of the attending clinician considering such factors as age, condition and size of the patient as well as severity of the symptoms being treated.

### Combination Therapy

In one aspect, the present methods for treating a cellular proliferative disorder can further comprise the administration of one or more additional therapeutic agents that are other than a Pyrazolo[4,3-d]Pyrimidine Derivative.

Accordingly, in one embodiment, the present invention provides methods for treating a cellular proliferative disorder in a patient, the method comprising administering to the patient: (i) a Pyrazolo[4,3-d]Pyrimidine Derivative, or a pharmaceutically acceptable salt thereof, and (ii) at least one additional therapeutic agent that is other than a Pyrazolo[4,3-d]Pyrimidine Derivative, wherein the amounts administered are together effective to treat a cellular proliferative disorder. In one embodiment, the cellular proliferative disorder treated is cancer.

When administering a combination therapy of the invention to a patient, therapeutic agents in the combination, or a pharmaceutical composition or compositions comprising therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for non-limiting illustration purposes, the Pyrazolo[4,3-d]Pyrimidine Derivative, and an additional therapeutic agent may be present in fixed amounts (dosage amounts) in a single dosage unit (e.g., a capsule, a tablet and the like).

In one embodiment, the Pyrazolo[4,3-d]Pyrimidine Derivative is administered during a time when the additional therapeutic agent(s) exert their prophylactic or therapeutic effect, or vice versa.

In another embodiment, the Pyrazolo[4,3-d]Pyrimidine Derivative, and the additional therapeutic agent(s) are administered in doses commonly employed when such agents are used as monotherapy for treating cancer.

In another embodiment, the Pyrazolo[4,3-d]Pyrimidine Derivative, and the additional therapeutic agent(s) are administered in doses lower than the doses commonly employed when such agents are used as monotherapy for treating cancer.

In one embodiment, the Pyrazolo[4,3-d]Pyrimidine Derivative, and the additional therapeutic agent(s) are present in the same composition. In one embodiment, this composition is suitable for oral administration. In another embodiment, this composition is suitable for intravenous administration. In another embodiment, this composition is suitable for intertumoral administration. In another embodiment, this composition is suitable for subcutaneous administration. In still another embodiment, this composition is suitable for parenteral administration. (none of these types of administration would be preferred for these compounds.)

Cancers and proliferative disorders that can be treated or prevented using the combination therapy methods of the present invention include, but are not limited to, those listed above.

The Pyrazolo[4,3-d]Pyrimidine Derivative, and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of therapy without reducing the efficacy of therapy. Accordingly, in one embodiment, the Pyrazolo[4,3-d]Pyrimidine Derivative, and the additional therapeutic agent(s) act synergistically and are administered in doses lower than the doses commonly employed when such agents are used as monotherapy for treating cancer.

In one embodiment, the administration of the Pyrazolo[4,3-d]Pyrimidine Derivative, and the additional therapeutic agent(s) may inhibit the resistance of cancer to these agents.

The Pyrazolo[4,3-d]Pyrimidine Derivatives may be used in combination with one or more other active agents (collectively referred to herein as “additional therapeutic agents”), including but not limited to, other therapeutic agents that are used in the prevention, treatment, control, amelioration, or reduction of risk of a particular disease or condition (e.g., cancer). In one embodiment, a Pyrazolo[4,3-d]Pyrimidine Derivative is combined with one or more other therapeutic agents for use in the prevention, treatment, control amelioration, or reduction of risk of a particular disease or condition for which the Pyrazolo[4,3-d]Pyrimidine Derivatives are useful. Such other active agents may be administered, by a route and in an amount commonly used therefor, contemporaneously or sequentially with a compound of the present disclosure.

Combinations of the Pyrazolo[4,3-d]Pyrimidine Derivatives with one or more anticancer agents are within the scope of the invention. Examples of such additional anticancer agents can be found in Cancer Principles and Practice of Oncology by V. T. Devita and S. Hellman (editors), 9^{th} edition (May 16, 2011), Lippincott Williams & Wilkins Publishers. A person of ordinary skill in the art would be able to discern which combinations of additional therapeutic agents would be useful based on the particular characteristics of the drugs and the cancer involved. Such additional therapeutic agents include the following: estrogen receptor modulators, programmed cell death protein 1 (PD-1) inhibitors, programmed death-ligand 1 (PD-L1) inhibitors, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, HIV protease inhibitors, reverse transcriptase inhibitors, inhibitors of cell proliferation and survival signaling, bisphosphonates, aromatase inhibitors, siRNA therapeutics, γ-secretase inhibitors, agents that interfere with receptor tyrosine kinases (RTKs) and agents that interfere with cell cycle checkpoints.

The additional therapeutic agents, and classes of additional therapeutic agents, disclosed below herein, are all useful in the combination therapies of the present invention.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

In the treatment of breast cancer (e.g., postmenopausal and premenopausal breast cancer, e.g., hormone-dependent breast cancer) the compound of formula (1) may be used with an effective amount of at least one antihormonal agent selected from the group consisting of: (a) aromatase inhibitors, (b) antiestrogens, and (c) LHRH analogues; and optionally an effective amount of at least one chemotherapeutic agent. Examples of aromatase inhibitors include but are not limited to: Anastrozole (e.g., Arimidex), Letrozole (e.g., Femara), Exemestane (Aromasin), Fadrozole and Formestane (e.g., Lentaron). Examples of antiestrogens include but are not limited to: Tamoxifen (e.g., Nolvadex), Fulvestrant (e.g., Faslodex), Raloxifene (e.g., Evista), and Acolbifene.

Examples of LHRH analogues include but are not limited to: Goserelin (e.g., Zoladex) and Leuprolide (e.g., Leuprolide Acetate, such as Lupron or Lupron Depot). Examples of additional thereapeutic agents useful in the present compositions and methods include, but are not limited to, the following cancer chemotherapeutic agents: Trastuzumab (e.g., Herceptin), Gefitinib (e.g., Iressa), Erlotinib (e.g., Erlotinib HCl, such as Tarceva), Bevacizumab (e.g., Avastin), Cetuximab (e.g., Erbitux), and Bortezomib (e.g., Velcade).

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl) retinamide, and N-4-carboxyphenyl retinamide.

“Cytotoxic/cytostatic agents” refers to compounds which cause cell death or inhibit cell proliferation primarily by interfering directly with the cell's functioning or inhibit or interfere with cell myosis, including alkylating agents, tumor necrosis factors, intercalators, hypoxia activatable compounds, microtubule inhibitors/microtubule-stabilizing agents, inhibitors of mitotic kinesins, histone deacetylase inhibitors, inhibitors of kinases involved in mitotic progression, inhibitors of kinases involved in growth factor and cytokine signal transduction pathways, antimetabolites, biological response modifiers, hormonal/anti-hormonal therapeutic agents, haematopoietic growth factors, monoclonal antibody targeted therapeutic agents, topoisomerase inhibitors, proteosome inhibitors, ubiquitin ligase inhibitors, and aurora kinase inhibitors.

Examples of cytotoxic/cytostatic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine)platinum, benzylguanine, glufosfamide, GPX100, (trans, trans, trans)-bis-mu-(hexane- 1,6-diamine)-mu-[diamine-platinum(II)]bis[diamine(chloro)platinum (II)]tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycarminomycin, annamycin, galarubicin, elinafide, MEN10755, 4-demethoxy-3-deamino-3-aziridinyl-4-methylsulphonyl-daunorubicin (see WO 00/50032), Raf kinase inhibitors (such as Bay43-9006) and mTOR inhibitors (such as Wyeth's CCI-779).

An example of a hypoxia activatable compound is tirapazamine.

Examples of proteosome inhibitors include but are not limited to lactacystin and MLN-341 (Velcade).

Examples of microtubule inhibitors/microtubule-stabilizing agents include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl) benzene sulfonamide, anhydrovinblastine, TDX258, the epothilones (see for example U.S. Pat. Nos. 6,284,781 and 6,288,237) and BMS188797. In an example the epothilones are not included in the microtubule inhibitors/microtubule-stabilising agents.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H,15H)dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(dimethylamino)ethyl]-N-methylamino]ethyl]-5-[4-hydro0xy-3,5-dimethoxyphenyl]-5,5a,6,8,8a,9-hexohydrofuro(3′,4′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoguinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c] quinolin-7-one, and dimesna.

Examples of inhibitors of mitotic kinesins, and in particular the human mitotic kinesin KSP, are described in Publications WO03/039460, WO03/050064, WO03/050122, WO03/049527, WO03/049679, WO03/049678, WO04/039774, WO03/079973, WO03/099211, WO03/105855, WO03/106417, WO04/037171, WO04/058148, WO04/058700, WO04/126699, WO05/018638, WO05/019206, WO05/019205, WO05/018547, WO05/017190, US2005/0176776. In an example inhibitors of mitotic kinesins include, but are not limited to inhibitors of KSP, inhibitors of MKLP1, inhibitors of CENP-E, inhibitors of MCAK and inhibitors of Rab6-KIFL.

Examples of “histone deacetylase inhibitors” include, but are not limited to, SAHA, TSA, oxamflatin, PXD101, MG98 and scriptaid. Further reference to other histone deacetylase inhibitors may be found in the following manuscript; Miller, T. A. et al. J. Med. Chem. 46(24):5097-5116 (2003).

“Inhibitors of kinases involved in mitotic progression” include, but are not limited to, inhibitors of aurora kinase, inhibitors of Polo-like kinases (PLK; in particular inhibitors of PLK-1), inhibitors of bub-1 and inhibitors of bub-R1. An example of an “aurora kinase inhibitor” is VX-680 (tozasertib).

“Antiproliferative agents” include antisense RNA and DNA oligonucleotides such as G3139, ODN698, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-flurouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine, 3-aminopyridine-2-carboxaldehyde thiosemicarbazone and trastuzumab.

Examples of monoclonal antibody targeted therapeutic agents include those therapeutic agents which have cytotoxic agents or radioisotopes attached to a cancer cell specific or target cell specific monoclonal antibody. In one embodiment, a monoclonal antibody targeted therapeutic agent is Bexxar.

“HMG-CoA reductase inhibitor” refers to inhibitors of 3-hydroxy-3-methylglutaryl-CoA reductase. Examples of HMG-CoA reductase inhibitors that may be used include but are not limited to lovastatin, simvastatin, pravastatin, Fluvastatin, atorvastatin, rosuvastatin and cerivastatin. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefore the use of such salts, esters, open-acid and lactone forms is included within the scope of the invention.

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase). For an example of the role of a prenyl-protein transferase inhibitor on angiogenesis see European J. of Cancer, Vol. 35, No. 9, pp. 1394-1401 (1999).

“Angiogenesis inhibitor” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α, interleukin-12, pentosan polysulfate, cyclooxygenase inhibitors, including nonsteroidal anti-inflammatories (NSAIDs) like aspirin and ibuprofen as well as selective cyclooxy-genase-2 inhibitors like celecoxib and rofecoxib, steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin II antagonists.

Other examples of angiogenesis inhibitors useful in the present combinations include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide, CM101, squalamine, combretastatin, RPI4610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416), or a pharmaceutically acceptable salt thereof.

Additional therapeutic agents that modulate or inhibit angiogenesis and may also be used in combination with the Pyrazolo[4,3-d]Pyrimidine Derivatives, include agents that modulate or inhibit the coagulation and fibrinolysis systems (see review in Clin. Chem. La. Med. 38:679-692 (2000)). Examples of such agents include, but are not limited to, heparin, low molecular weight heparins and carboxypeptidase U inhibitors (also known as inhibitors of active thrombin activatable fibrinolysis inhibitor [TAFIa]).

Further examples of angiogenesis inhibitors include a tyrosine kinase inhibitor, an inhibitor of epidermal-derived growth factor, an inhibitor of fibroblast-derived growth factor, an inhibitor of platelet derived growth factor, an MMP (matrix metalloprotease) inhibitor, an integrin blocker, interferon-α, interleukin-12, pentosan polysulfate, a cyclooxygenase inhibitor, carboxyamidotriazole, combretastatin A-4, squalamine, 6-O-chloroacetyl-carbonyl)-fumagillol, thalidomide, angiostatin, troponin-1, or an antibody to VEGF.

“Agents that interfere with cell cycle checkpoints” refers to compounds that inhibit protein kinases that transduce cell cycle checkpoint signals, thereby sensitizing the cancer cell to DNA damaging agents. Such agents include inhibitors of ATR, ATM, the CHK1 and CHK2 kinases and cdk and cdc kinase inhibitors and are specifically exemplified by 7-hydroxystaurosporin, flavopiridol, CYC202 (Cyclacel) and BMS-387032.

“Agents that interfere with receptor tyrosine kinases (RTKs)” refers to compounds that inhibit RTKs and therefore mechanisms involved in oncogenesis and tumor progression. Such agents include inhibitors of c-Kit, Eph, PDGF, Flt3 and c-Met. Further agents include inhibitors of RTKs as described by Bume-Jensen and Hunter, Nature, 411:355-365, 2001. Specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9,12-epoxy-1H-diindolo[1,2,3-fg:3′,2′,1′-kl]pyrrolo[3,4-i][1,6]benzodiazocin-1-one, SH268, genistein, STI571, CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, STI571A, N-4-chlorophenyl-4-(4-pyridylmethyl)-1-phthalazinamine, and EMD121974, or a pharmaceutically acceptable salt thereof.

“Inhibitors of cell proliferation and survival signaling pathway” refers to compounds that inhibit signal transduction cascades downstream of cell surface receptors. Such agents include inhibitors of serine/threonine kinases (including but not limited to inhibitors of Akt such as described in WO 02/083064, WO 02/083139, WO 02/083140, US 2004-0116432, WO 02/083138, US 2004/0102360, WO 03/086404, WO 03/086279, WO 03/086394, WO 03/084473, WO 03/086403, WO 2004/041162, WO 2004/096131, WO 2004/096129, WO 2004/096135, WO 2004/096130, WO 2005/100356, WO 2005/100344, U.S. Pat. Nos. 7,454,431, 7,589,068), inhibitors of Raf kinase (for example BAY-43-9006), inhibitors of MEK (for example CI-1040 and PD-098059), inhibitors of mTOR (for example Wyeth CCI-779), and inhibitors of PI3K (for example LY294002).

The invention also encompasses combination therapies comprising NSAIDs which are selective COX-2 inhibitors. For purposes of the specification NSAIDs which are selective inhibitors of COX-2 are defined as those which possess a specificity for inhibiting COX-2 over COX-1 of at least 100-fold as measured by the ratio of IC₅₀ for COX-2 over IC₅₀ for COX-1 evaluated by cell or microsomal assays. Inhibitors of COX-2 that are useful in the present methods are: 3-phenyl-4-(4-(methylsulfonyl)phenyl)-2-(5H)-furanone; and 5-chloro-3-(4-methylsulfonyl)-phenyl-2-(2-methyl-5-pyridinyl)pyridine; or a pharmaceutically acceptable salt thereof. Compounds that have been described as specific inhibitors of COX-2 and are therefore also useful in the present invention include, but are not limited to, the following: rofecoxib, etoricoxib, parecoxib, BEXTRA® and CELEBREX® or a pharmaceutically acceptable salt thereof.

As used herein, “integrin blockers” refers to compounds which selectively antagonize, inhibit or counteract binding of a physiological ligand to the αvβ3 integrin, to compounds which selectively antagonize, inhibit or counteract binding of a physiological ligand to the αvβ5 integrin, to compounds which antagonize, inhibit or counteract binding of a physiological ligand to both the αvβ3 integrin and the αvβ5 integrin, and to compounds which antagonize, inhibit or counteract the activity of the particular integrin(s) expressed on capillary endothelial cells. The term also refers to antagonists of the αvβ6, αvβ8, α1β1, α2β1, α5β1, α6β1 and α6β4 integrins. The term also refers to antagonists of any combination of αvβ3, αvβ5, αvβ6, αvβ8, α1β1, α2β1, α5β1, α6β1 and α6β4 integrins.

Combinations with additional therapeutic agents, other than anti-cancer agents, are also contemplated in the instant methods. For example, combinations of the Pyrazolo[4,3-d]Pyrimidine Derivatives with PPAR-γ (i.e., PPAR-gamma) agonists and PPAR-δ (i.e., PPAR-delta) agonists are useful in the treatment of certain malignancies. PPAR-γ and PPAR-δ are the nuclear peroxisome proliferator-activated receptors γ and δ. PPAR-γ agonists have been shown to inhibit the angiogenic response to VEGF in vitro; both troglitazone and rosiglitazone maleate inhibit the development of retinal neovascularization in mice (Arch. Ophthamol. 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, G1262570, PNU182716, DRF552926, 2-[(5,7-dipropyl-3-trifluoromethyl-1,2-benzisoxazol-6-yl)oxy]-2-methylpropionic acid (disclosed in U.S. Ser. No. 09/782,856), and 2(R)-7-(3-(2-chloro-4-(4-fluorophenoxy) phenoxy)propoxy)-2-ethylchromane-2-carboxylic acid (disclosed in U.S. Ser. Nos. 60/235,708 and 60/244,697), or a pharmaceutically acceptable salt thereof.

Another embodiment of the instant invention is the use of the Pyrazolo[4,3-d]Pyrimidine Derivatives in combination with gene therapy for the treatment of cancer. For an overview of genetic strategies to treating cancer see Hall et al., (Am. J. Hum. Genet. 61:785-789, 1997) and Kufe et al., (Cancer Medicine, 5th Ed, pp 876-889, B C Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998; 5(8):1105-13), and interferon gamma (J. Immunol. 2000; 164:217-222).

The Pyrazolo[4,3-d]Pyrimidine Derivatives may also be administered in combination with an inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar), or a pharmaceutically acceptable salt thereof.

A Pyrazolo[4,3-d]Pyrimidine Derivative may also be administered with an immunologic-enhancing drug, such as levamisole, isoprinosine and Zadaxin, or a pharmaceutically acceptable salt thereof.

A Pyrazolo[4,3-d]Pyrimidine Derivative may also be useful for treating or preventing cancer in combination with P450 inhibitors including: xenobiotics, quinidine, tyramine, ketoconazole, testosterone, quinine, methyrapone, caffeine, phenelzine, doxorubicin, troleandomycin, cyclobenzaprine, erythromycin, cocaine, furafyline, cimetidine, dextromethorphan, ritonavir, indinavir, amprenavir, diltiazem, terfenadine, verapamil, cortisol, itraconazole, mibefradil, nefazodone and nelfinavir, or a pharmaceutically acceptable salt thereof.

A Pyrazolo[4,3-d]Pyrimidine Derivative may also be useful for treating or preventing cancer in combination with Pgp and/or BCRP inhibitors including: cyclosporin A, PSC833, GF120918, cremophorEL, fumitremorgin C, Ko132, Ko134, Iressa, Imatnib mesylate, EKI-785, Cl1033, novobiocin, diethylstilbestrol, tamoxifen, resperpine, VX-710, tryprostatin A, flavonoids, ritonavir, saquinavir, nelfinavir, omeprazole, quinidine, verapamil, terfenadine, ketoconazole, nifidepine, FK506, amiodarone, XR9576, indinavir, amprenavir, cortisol, testosterone, LY335979, OC144-093, erythromycin, vincristine, digoxin and talinolol, or a pharmaceutically acceptable salt thereof.

A Pyrazolo[4,3-d]Pyrimidine Derivative may also be useful for treating or preventing cancer, including bone cancer, in combination with bisphosphonates, including but not limited to: etidronate (Didronel), pamidronate (Aredia), alendronate (Fosamax), risedronate (Actonel), zoledronate (Zometa), ibandronate (Boniva), incadronate or cimadronate, clodronate, EB-1053, minodronate, neridronate, piridronate and tiludronate including any and all pharmaceutically acceptable salts, derivatives, hydrates and mixtures thereof.

A Pyrazolo[4,3-d]Pyrimidine Derivative may also be useful for treating or preventing breast cancer in combination with aromatase inhibitors. Examples of aromatase inhibitors include but are not limited to: anastrozole, letrozole and exemestane, or a pharmaceutically acceptable salt thereof.

A Pyrazolo[4,3-d]Pyrimidine Derivative may also be useful for treating or preventing cancer in combination with siRNA therapeutics.

The Pyrazolo[4,3-d]Pyrimidine Derivatives may also be administered in combination with γ-secretase inhibitors and/or inhibitors of NOTCH signaling. Such inhibitors include compounds described in WO 01/90084, WO 02/30912, WO 01/70677, WO 03/013506, WO 02/36555, WO 03/093252, WO 03/093264, WO 03/093251, WO 03/093253, WO 2004/039800, WO 2004/039370, WO 2005/030731, WO 2005/014553, U.S. Ser. No. 10/957,251, WO 2004/089911, WO 02/081435, WO 02/081433, WO 03/018543, WO 2004/031137, WO 2004/031139, WO 2004/031138, WO 2004/101538, WO 2004/101539 and WO 02/47671 (including LY-450139), or a pharmaceutically acceptable salt thereof.

In one embodiment, specific anticancer agents useful in the present combination therapies include, but are not limited to: pembrolizumab (Keytruda®) abarelix (Plenaxis depot®); aldesleukin (Prokine®); Aldesleukin (Proleukin®); Alemtuzumabb (Campath®); alitretinoin (Panretin®); allopurinol (Zyloprim®); altretamine (Hexalen®); amifostine (Ethyol®); anastrozole (Arimidex®); arsenic trioxide (Trisenox®); asparaginase (Elspar®); azacitidine (Vidaza®); bevacuzimab (Avastin®); bexarotene capsules (Targretin®); bexarotene gel (Targretin®); bleomycin (Blenoxane®); bortezomib (Velcade®); busulfan intravenous (Busulfex®); busulfan oral (Myleran®); calusterone (Methosarb®); capecitabine (Xeloda®); carboplatin (Paraplatin®); carmustine (BCNU®, BiCNU®); carmustine (Gliadel®); carmustine with Polifeprosan 20 Implant (Gliadel Wafer®); celecoxib (Celebrex®); cetuximab (Erbitux®); chlorambucil (Leukeran®); cisplatin (Platinol®); cladribine (Leustatin®, 2-CdA®); clofarabine (Clolar®); cyclophosphamide (Cytoxan®, Neosar®); cyclophosphamide (Cytoxan Injection®); cyclophosphamide (Cytoxan Tablet®); cytarabine (Cytosar-U®); cytarabine liposomal (DepoCyt®); dacarbazine (DTIC-Dome®); dactinomycin, actinomycin D (Cosmegen®); Darbepoetin alfa (Aranesp®); daunorubicin liposomal (DanuoXome®); daunorubicin, daunomycin (Daunorubicin®); daunorubicin, daunomycin (Cerubidine®); Denileukin diftitox (Ontak®); dexrazoxane (Zinecard®); docetaxel (Taxotere®); doxorubicin (Adriamycin PFS®); doxorubicin (Adriamycin®, Rubex®); doxorubicin (Adriamycin PFS Injection®); doxorubicin liposomal (Doxil®); dromostanolone propionate (Dromostanolone®); dromostanolone propionate (Masterone injection®); Elliott's B Solution (Elliott's B Solution®); epirubicin (Ellence®); Epoetin alfa (epogen®); erlotinib (Tarceva®); estramustine (Emcyt®); etoposide phosphate (Etopophos®); etoposide, VP-16 (Vepesid®); exemestane (Aromasin®); Filgrastim (Neupogen®); floxuridine (intraarterial) (FUDR®); fludarabine (Fludara®); fluorouracil, 5-FU (Adrucil®); fulvestrant (Faslodex®); gefitinib (Iressa®); gemcitabine (Gemzar®); gemtuzumab ozogamicin (Mylotarg®); goserelin acetate (Zoladex Implant®); goserelin acetate (Zoladex®); histrelin acetate (Histrelin implant®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); irinotecan (Camptosar®); lenalidomide (Revlimid®); letrozole (Femara®); leucovorin (Wellcovorin Leucovorin®); Leuprolide Acetate (Eligard®); levamisole (Ergamisol®); lomustine, CCNU (CeeBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex tabs®); methotrexate (Methotrexate®); methoxsalen (Uvadex®); mitomycin C (Mutamycin®); mitotane (Lysodren®); mitoxantrone (Novantrone®); nandrolone phenpropionate (Durabolin-50®); nelarabine (Arranon®); Nofetumomab (Verluma®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); pegademase (Adagen (Pegademase Bovine)®); pegaspargase (Oncaspar®); Pegfilgrastim (Neulasta®); pemetrexed disodium (Alimta®); pentostatin (Nipent®); pipobroman (Vercyte®); plicamycin, mithramycin (Mithracin®); porfimer sodium (Photofrin®); procarbazine (Matulane®); quinacrine (Atabrine®); Rasburicase (Elitek®); Rituximab (Rituxan®); Ridaforolimus; sargramostim (Leukine®); Sargramostim (Prokine®); sorafenib (Nexavar®); streptozocin (Zanosar®); sunitinib maleate (Sutent®); talc (Sclerosol); tamoxifen (Nolvadex®); temozolomide (Temodar®); teniposide, VM-26 (Vumon®); testolactone (Teslac®); thioguanine, 6-TG (Thioguanine®); thiotepa (Thioplex®); topotecan (Hycamtin®); toremifene (Fareston®); Tositumomab (Bexxar®); Tositumomab/I-131 tositumomab (Bexxar®); Trastuzumab (Herceptin®); tretinoin, ATRA (Vesanoid®); Uracil Mustard (Uracil Mustard Capsules®), valrubicin (Valstar®); vinblastine (Velban®); vincristine (Oncovin®); vinorelbine (Navelbine®); vorinostat (Zolinza®) and zoledronate (Zometa®), or a pharmaceutically acceptable salt thereof.

Thus, the scope of the instant invention encompasses the use of the Pyrazolo[4,3-d]Pyrimidine Derivatives in combination with a second compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, PPAR-γ agonists, PPAR-δ agonists, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, a bisphosphonate, an aromatase inhibitor, an siRNA therapeutic, γ-secretase and/or NOTCH inhibitors, agents that interfere with receptor tyrosine kinases (RTKs), an agent that interferes with a cell cycle checkpoint, and any of the therapeutic agents listed above.

Yet another example of the invention is a method of treating cancer that comprises administering a therapeutically effective amount of a Pyrazolo[4,3-d]Pyrimidine Derivative in combination with paclitaxel or trastuzumab.

The therapeutic combination disclosed herein may be used in combination with one or more other active agents, including but not limited to, other anti-cancer agents that are used in the prevention, treatment, control, amelioration, or reduction of risk of a particular disease or condition (e.g., cell-proliferation disorders). In one embodiment, a Pyrazolo[4,3-d]Pyrimidine Derivative is combined with one or more other anti-cancer agents for use in the prevention, treatment, control amelioration, or reduction of risk of a particular disease or condition for which the Pyrazolo[4,3-d]Pyrimidine Derivatives are useful. Such other active agents may be administered, by a route and in an amount commonly used therefor, prior to, contemporaneously, or sequentially with a compound of the present disclosure.

The instant invention also includes a pharmaceutical composition useful for treating or preventing cancer that comprises a therapeutically effective amount of a Pyrazolo[4,3-d]Pyrimidine Derivative and a second compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, a PPAR-γ agonist, a PPAR-δ agonist, an inhibitor of cell proliferation and survival signaling, a bisphosphonate, an aromatase inhibitor, an siRNA therapeutic, γ-secretase and/or NOTCH inhibitors, agents that interfere with receptor tyrosine kinases (RTKs), an agent that interferes with a cell cycle checkpoint, and any of the therapeutic agents listed above.

The invention further relates to a method of treating cancer in a human patient comprising administration of a Pyrazolo[4,3-d]Pyrimidine Derivative and a PD-1 antagonist to the patient. The compound of the invention and the PD-1 antagonist may be administered concurrently or sequentially.

In particular embodiments, the PD-1 antagonist is an anti-PD-1 antibody, or antigen binding fragment thereof. In alternative embodiments, the PD-1 antagonist is an anti-PD-L1 antibody, or antigen binding fragment thereof. In some embodiments, the PD-1 antagonist is an anti-PD-1 antibody, independently selected from pembrolizumab, nivolumab, cemiplimab, sintilimab, tislelizumab, atezolizumab (MPDL3280A), camrelizumab and toripalimab. In other embodiments, the PD-L1 antagonist is an anti-PD-L1 antibody independently selected from atezolizumab, durvalumab and avelumab.

In one embodiments, the PD-1 antagonist is pembrolizumab. In particular sub-embodiments, the method comprises administering 200 mg of pembrolizumab to the patient about every three weeks. In other sub-embodiments, the method comprises administering 400 mg of pembrolizumab to the patient about every six weeks.

In further sub-embodiments, the method comprises administering 2 mg/kg of pembrolizumab to the patient about every three weeks. In particular sub-embodiments, the patient is a pediatric patient.

In some embodiments, the PD-1 antagonist is nivolumab. In particular sub-embodiments, the method comprises administering 240 mg of nivolumab to the patient about every two weeks. In other sub-embodiments, the method comprises administering 480 mg of nivolumab to the patient about every four weeks.

In some embodiments, the PD-1 antagonist is cemiplimab. In particular embodiments, the method comprises administering 350 mg of cemiplimab to the patient about every 3 weeks.

In some embodiments, the PD-1 antagonist is atezolizumab. In particular sub-embodiments, the method comprises administering 1200 mg of atezolizumab to the patient about every three weeks.

In some embodiments, the PD-1 antagonist is durvalumab. In particular sub-embodiments, the method comprises administering 10 mg/kg of durvalumab to the patient about every two weeks.

In some embodiments, the PD-1 antagonist is avelumab. In particular sub-embodiments, the method comprises administering 800 mg of avelumab to the patient about every two weeks.

When the Pyrazolo[4,3-d]Pyrimidine Derivatives are administered in combination with an anti-human PD-1 antibody (or antigen-binding fragment thereof), the anti-human PD-1 antibody (or antigen-binding fragment thereof) may be administered either simultaneously with, or before or after, the Pyrazolo[4,3-d]Pyrimidine Derivative. Either of the anti-human PD-1 antibody (or antigen-binding fragment thereof), and/or Pyrazolo[4,3-d]Pyrimidine Derivative of the present invention, or a pharmaceutically acceptable salt thereof, may be administered separately, by the same or different route of administration, or together in the same pharmaceutical composition as the other agent(s). The weight ratio of the anti-human PD-1 antibody (or antigen-binding fragment thereof) to Pyrazolo[4,3-d]Pyrimidine Derivative of the present invention, may be varied and will depend upon the therapeutically effective dose of each agent. Generally, a therapeutically effective dose of each will be used. Combinations including at least one anti-human PD-1 antibody (or antigen-binding fragment thereof), a Pyrazolo[4,3-d]Pyrimidine Derivative of the present invention, and optionally other active agents will generally include a therapeutically effective dose of each active agent. In such combinations, the anti-human PD-1 antibody (or antigen-binding fragment thereof), the Pyrazolo[4,3-d]Pyrimidine Derivative, and other active agents may be administered separately or in conjunction. In addition, the administration of one element may be prior to, concurrent with, or subsequent to the administration of other agent(s).

In one embodiment, this disclosure provides an anti-human PD-1 antibody (or antigen-binding fragment thereof), and/or Pyrazolo[4,3-d]Pyrimidine Derivative, and at least one other active agent as a combined preparation for simultaneous, separate or sequential use in treating cancer.

The disclosure also provides the use of a Pyrazolo[4,3-d]Pyrimidine Derivative of the present invention, for treating cancer, where the patient has previously (e.g., within 24-hours) been treated with an anti-human PD-1 antibody (or antigen-binding fragment thereof). The disclosure also provides the use of an anti-human PD-1 antibody (or antigen-binding fragment thereof) for treating a cellular proliferative disorder, where the patient has previously (e.g., within 24-hours) been treated with a Pyrazolo[4,3-d]Pyrimidine Derivative of the present invention.

The present disclosure further relates to methods of treating cancer, said method comprising administering to a subject in need thereof a combination therapy that comprises (a) a Pyrazolo[4,3-d]Pyrimidine Derivative of the present invention, and (b) an anti-human PD-1 antibody (or antigen-binding fragment thereof); wherein the anti-human PD-1 antibody (or antigen-binding fragment thereof) is administered once every 21 days.

Additionally, the present disclosure relates to methods of treating cancer, said method comprising administering to a subject in need thereof a combination therapy that comprises: (a) a Pyrazolo[4,3-d]Pyrimidine Derivative of the present invention, and (b) an anti-human PD-1 antibody (or antigen-binding fragment thereof. In specific embodiments, the cancer occurs as one or more solid tumors or lymphomas. In further specific embodiments, the cancer is selected from the group consisting of advanced or metastatic solid tumors and lymphomas. In still further specific embodiments, the cancer is selected from the group consisting of malignant melanoma, head and neck squamous cell carcinoma, MSI-H cancer, MMR deficient cancer, non-small cell lung cancer, urothelial carcinoma, gastric or gastroesophageal junction adenocarcinoma, breast adenocarcinoma, and lymphomas. In additional embodiments, the lymphoma is selected from the group consisting of diffuse large B-cell lymphoma, follicular lymphoma, mantle cell lymphoma, small lymphocytic lymphoma, mediastinal large B-cell lymphoma, splenic marginal zone B-cell lymphoma, extranodal marginal zone B-cell lymphoma of mucosa-associated lymphoid tissue (malt), nodal marginal zone B-cell lymphoma, lymphoplasmacytic lymphoma, primary effusion lymphoma, Burkitt lymphoma, anaplastic large cell lymphoma (primary cutaneous type), anaplastic large cell lymphoma (systemic type), peripheral T-cell lymphoma, angioimmunoblastic T-cell lymphoma, adult T-cell lymphoma/leukemia, nasal type extranodal NK/T-cell lymphoma, enteropathy-associated T-cell lymphoma, gamma/delta hepatosplenic T-cell lymphoma, subcutaneous panniculitis-like T-cell lymphoma, mycosis fungoides, and Hodgkin lymphoma. In particular embodiments, the cellular proliferative disorder is a cancer that has metastasized, for example, a liver metastases from colorectal cancer. In additional embodiments, the cellular proliferative disorder is a cancer is classified as stage III cancer or stage IV cancer. In instances of these embodiments, the cancer is not surgically resectable.

In embodiments of the methods disclosed herein, the anti-human PD-1 antibody (or antigen binding fragment thereof) is administered by intravenous infusion or subcutaneous injection.

In one embodiment, the present invention provides compositions comprising a Pyrazolo[4,3-d]Pyrimidine Derivative, a pharmaceutically acceptable carrier, and an anti-human PD-1 antibody (or antigen-binding fragment thereof).

In another embodiment, the present invention provides compositions comprising a Pyrazolo[4,3-d]Pyrimidine Derivative, a pharmaceutically acceptable carrier, and pembrolizumab.

In one embodiment, the present invention provides compositions comprising a Pyrazolo[4,3-d]Pyrimidine Derivative, a pharmaceutically acceptable carrier, and two additional therapeutic agents, one of which is an anti-human PD-1 antibody (or antigen-binding fragment thereof), and the other of which is independently selected from the group consisting of anticancer agents.

A compound of the present invention may be employed in conjunction with anti-emetic agents to treat nausea or emesis, including acute, delayed, late-phase, and anticipatory emesis, which may result from the use of a compound of the present invention, alone or with radiation therapy. For the prevention or treatment of emesis, a compound of the present invention may be used in conjunction with other anti-emetic agents, especially neurokinin-1 receptor antagonists, 5HT3 receptor antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or others such as disclosed in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. In another example, conjunctive therapy with an anti-emesis agent selected from a neurokinin-1 receptor antagonist, a 5HT3 receptor antagonist and a corticosteroid is disclosed for the treatment or prevention of emesis that may result upon administration of the Pyrazolo[4,3-d]Pyrimidine Derivatives.

A Pyrazolo[4,3-d]Pyrimidine Derivative may also be administered with an agent useful in the treatment of anemia. Such an anemia treatment agent is, for example, a continuous erythropoiesis receptor activator (such as epoetin alfa).

A Pyrazolo[4,3-d]Pyrimidine Derivative may also be administered with an agent useful in the treatment of neutropenia. Such a neutropenia treatment agent is, for example, a hematopoietic growth factor which regulates the production and function of neutrophils such as a human granulocyte colony stimulating factor, (G-CSF). Examples of a G-CSF include filgrastim.

The Pyrazolo[4,3-d]Pyrimidine Derivatives may be useful when co-administered with other treatment modalities, including but not limited to, radiation therapy, surgery, and gene therapy. Accordingly, in one embodiment, the methods of treating cancer described herein, unless stated otherwise, can optionally include the administration of an effective amount of radiation therapy. For radiation therapy, γ-radiation is preferred.

The methods of treating cancers described herein can optionally include the administration of an effective amount of radiation (i.e., the methods of treating cancers described herein optionally include the administration of radiation therapy).

The methods of treating cancer described herein include methods of treating cancer that comprise administering a therapeutically effective amount of a Pyrazolo[4,3-d]Pyrimidine Derivative in combination with radiation therapy and/or in combination with a second compound selected from: an estrogen receptor modulator, an androgen receptor modulator, a retinoid receptor modulator, a cytotoxicytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, PPAR-γ agonists, PPAR-δ agonists, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, a bisphosphonate, an aromatase inhibitor, an siRNA therapeutic, γ-secretase and/or NOTCH inhibitors, agents that interfere with receptor tyrosine kinases (RTKs), an agent that interferes with a cell cycle checkpoint, and any of the additional therapeutic agents listed herein.

Additional embodiments of the disclosure include the pharmaceutical compositions, combinations, uses and methods set forth in above, wherein it is to be understood that each embodiment may be combined with one or more other embodiments, to the extent that such a combination is consistent with the description of the embodiments. It is further to be understood that the embodiments provided above are understood to include all embodiments, including such embodiments as result from combinations of embodiments.

### Kits

In one aspect, the present invention provides a kit comprising a therapeutically effective amount of a Pyrazolo[4,3-d]Pyrimidine Derivative, or a pharmaceutically acceptable salt, solvate or ester of said compound and a pharmaceutically acceptable carrier, vehicle or diluent.

In another aspect the present invention provides a kit comprising an amount of a Pyrazolo[4,3-d]Pyrimidine Derivative, and an amount of at least one additional therapeutic agent listed above, wherein the amounts of the two or more active ingredients result in a desired therapeutic effect. In one embodiment, the Pyrazolo[4,3-d]Pyrimidine Derivative, and the one or more additional therapeutic agents are provided in the same container. In one embodiment, the Pyrazolo[4,3-d]Pyrimidine Derivative, and the one or more additional therapeutic agents are provided in separate containers.

1. A compound having the formula (I): 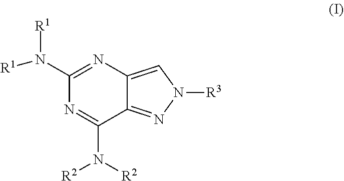
or a pharmaceutically acceptable salt thereof,
wherein:
each occurrence of R¹ is independently selected from H and C₁-C₆ alkyl;
each occurrence of R² is independently selected from H, C₁-C₈ alkyl, —(C₁-C₆ alkylene)-O—(C₁-C₆ alkyl), C₁-C₈ hydroxyalkyl, C₃-C₇ cycloalkyl, wherein said C₃-C₇ cycloalkyl group can be optionally substituted with one or more R⁴ groups, which can be the same or different; or two R² groups, together with the nitrogen atom to which they are attached, can join to form a 5- to 7-membered monocyclic heterocycloalkyl group, wherein said 5- to 7-membered monocyclic heterocycloalkyl group can be optionally substituted with one or more R⁴ groups, which can be the same or different;
R³ is selected from C₁-C₈ alkyl, —C₁-C₈ aminoalkyl, —(CH₂)ₙ-phenyl, —(CH₂)ₙ—(C₃-C₇ cycloalkyl), —(CH₂)ₙ-(4 to 7-membered monocyclic heterocycloalkyl), —(CH₂)ₙ-(5- or 6-membered monocyclic heteroaryl), and —CH₂-(7- to 10-membered bicyclic heteroaryl), wherein the phenyl moiety of said benzyl group and the phenyl moiety of said —(CH₂)ₙ-phenyl group, the 4 to 7-membered monocyclic heterocycloalkyl moiety of said —(CH₂)ₙ-(4 to 7-membered monocyclic heterocycloalkyl); and the 5- or 6-membered monocyclic heteroaryl moiety of said —(CH₂)ₙ-(5- or 6-membered monocyclic heteroaryl) group, can be optionally substituted with one or more R⁵ groups, which can be the same or different; the 7- to 10-membered bicyclic heteroaryl moiety of said —(CH₂)ₙ-(7- to 10-membered bicyclic heteroaryl) group can be optionally substituted with one or more R⁶ groups, which can be the same or different; and the C₃-C₇ cycloalkyl moiety of said —(CH₂)ₙ—(C₃-C₇ cycloalkyl) group can be optionally substituted with one or more R⁷ groups, which can be the same or different;
each occurrence of R⁴ is independently selected from C₁-C₈ alkyl, C₁-C₈ hydroxyalkyl, halo, and —OH;
each occurrence of R⁵ is independently selected from C₁-C₆ alkyl, C₃-C₇ cycloalkyl, C₁-C₆ aminoalkyl, —O—(C₁-C₆ alkyl), C₁-C₆ hydroxyalkyl, —O—(C₁-C₆ hydroxyalkyl), —O—(C₁-C₆ alkylene)-C(O)OR⁸, —O—(C₁-C₆ haloalkyl), —S—CH₂CH(NH)—C(O)OR⁸, NHC(O)—(C₁-C₆ alkyl), C₁-C₆ haloalkyl, halo, —(CH₂)ₙ-(4 to 7-membered monocyclic heterocycloalkyl), -6- to 11-membered spirocyclic bicyclic heterocycloalkyl, —N(R⁸)₂, —(C₁-C₃ alkylene)ₙ-N(R⁸)₂, —C(O)—(C₁-C₃ alkylene)-R^{C}, —(C₁-C₃ alkylene)ₙ-N(R⁸)—(C₁-C₃ alkylene)ₙ-R^{C}, —(C₁-C₃ alkylene)ₙ-NHC(O)—(C₁-C₃ alkylene)-R^{C}, —(C₁-C₃ alkylene)-(C₁-C₆ aminoalkyl), —CH(N(R⁸)₂)(C₁-C₆ aminoalkyl), —(C₁-C₃ alkylene)ₙ-N(R⁸)—(C₁-C₆ aminoalkyl), —(C₁-C₃ alkylene)ₙ-N(R⁸)—(C₁-C₃ alkylene)-NHC(O)-(5- or 6-membered monocyclic heteroaryl), R^{A}, R^{B}, R^{C}, and R^{D}, wherein said 6- to 11-membered spirocyclic bicyclic heterocycloalkyl group can be optionally substituted with —(C₁-C₃ alkylene)-(5- to 7-membered monocyclic heterocycloalkyl) or —(C₁-C₃ alkylene)-R^{C}, and the 4 to 7-membered monocyclic heterocycloalkyl moiety of said —(CH₂)ₙ-(4 to 7-membered monocyclic heterocycloalkyl) group can be optionally substituted with —(C₁-C₃ alkylene)ₙ-N(R⁸)₂ or —(C₁-C₃ alkylene)-R^{C};
each occurrence of R⁶ is independently selected from C₁-C₆ alkyl, C₃-C₇ cycloalkyl, C₁-C₆ aminoalkyl, —O—(C₁-C₆ alkyl), —O—(C₁-C₆ hydroxyalkyl), —O—(C₁-C₆ alkylene)-C(O)OR⁸, —O—(C₁-C₆ haloalkyl), C₁-C₆ hydroxyalkyl, halo, 6- to 11-membered spirocyclic bicyclic heterocycloalkyl, —N(R⁸)₂, —(C₁-C₃ alkylene)-N(R⁸)₂, —(C₁-C₃ alkylene)-(C₁-C₆ aminoalkyl), —(C₁-C₃ alkylene)-N(CH₃)—(C₁-C₆ aminoalkyl), —NH—(C₁-C₆ aminoalkyl), R^{A}, R^{B}, and R^{C}, wherein said 6- to 11-membered spirocyclic bicyclic heterocycloalkyl group can be optionally substituted with —(C₁-C₃ alkylene)-(5- to 7-membered monocyclic heterocycloalkyl);
each occurrence of R⁷ is independently selected from C₁-C₆ alkyl, C₃-C₇ cycloalkyl, C₁-C₆ aminoalkyl, —O—(C₁-C₆ alkyl), —O—(C₁-C₆ hydroxyalkyl), —O—(C₁-C₆ alkylene)-C(O)OR⁸, —O—(C₁-C₆ haloalkyl), C₁-C₆ hydroxyalkyl, halo, 6- to 11-membered spirocyclic bicyclic heterocycloalkyl, —N(R⁸)₂, —(C₁-C₃ alkylene)-N(R⁸)₂, —(C₁-C₃ alkylene)-(C₁-C₆ aminoalkyl), —(C₁-C₃ alkylene)-N(CH₃)—(C₁-C₆ aminoalkyl), —NH—(C₁-C₆ aminoalkyl), R^{A}, R^{B}, and R^{C}, wherein said 6- to 11-membered spirocyclic bicyclic heterocycloalkyl group can be optionally substituted with —(C₁-C₃ alkylene)-(5- to 7-membered monocyclic heterocycloalkyl);
each occurrence of R⁸ is independently selected from H and C₁-C₆ alkyl;
R^{A} is: 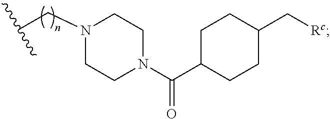
R^{B} is: 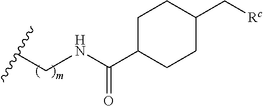
R^{C} is selected from C₁-C₆ aminoalkyl, —NHC(O)—(C₁-C₆) alkenyl, 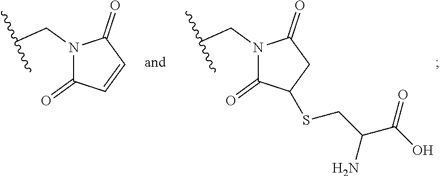 [SMILES: *CN1C(=O)C=CC1=O ; *CN1C(=O)CC(SCC(N)C(=O)O)C1=O]
R^{D} is: 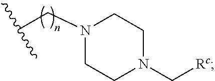
each occurrence of m is independently 1 or 2; and
each occurrence of n is independently 0 or 1.

2. The compound of claim 1, having the formula (Ia): 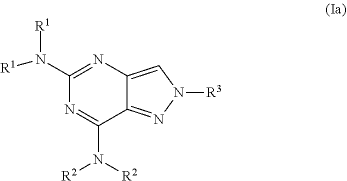
or a pharmaceutically acceptable salt thereof,
wherein:
each occurrence of R¹ is independently selected from H and C₁-C₆ alkyl;
each occurrence of R² is independently selected from H, C₁-C₈ alkyl, —(C₁-C₆ alkylene)-O—(C₁-C₆ alkyl), C₁-C₈ hydroxyalkyl, C₃-C₇ cycloalkyl, wherein said C₃-C₇ cycloalkyl group can be optionally substituted with one or more R⁴ groups, which can be the same or different; or two R² groups, together with the nitrogen atom to which they are attached, can join to form a 5- to 7-membered monocyclic heterocycloalkyl group, wherein said 5- to 7-membered monocyclic heterocycloalkyl group can be optionally substituted with one or more R⁴ groups, which can be the same or different;
R³ is selected from C₁-C₈ alkyl, C₁-C₈ aminoalkyl, benzyl, —(CH₂)₂-phenyl, —CH₂—(C₃-C₇ cycloalkyl), and —CH₂-(5- or 6-membered monocyclic heteroaryl), wherein the phenyl moiety of said benzyl group can be optionally substituted with one or more R⁵ groups, which can be the same or different; the phenyl moiety of said —(CH₂)₂-phenyl group can be optionally substituted with one or more R⁶ groups, which can be the same or different; the C₃-C₇ cycloalkyl moiety of said —CH₂—(C₃-C₇ cycloalkyl) group can be optionally substituted with one or more R⁷ groups, which can be the same or different; and the 5- or 6-membered monocyclic heteroaryl moiety of said —CH₂-(5- or 6-membered monocyclic heteroaryl) group can be optionally substituted with one or more R⁸ groups, which can be the same or different;
each occurrence of R⁴ is independently selected from C₁-C₈ alkyl, C₁-C₈ hydroxyalkyl, halo, and —OH;
each occurrence of R⁵ is independently selected from C₁-C₆ alkyl, —O—(C₁-C₆ alkyl), halo, —NH₂, C₁-C₆ aminoalkyl, —(CH₂)ₙ-(5- to 7-membered monocyclic heterocycloalkyl), R^{A}, and R^{B};
each occurrence of R⁶ is independently selected from C₁-C₆ alkyl, —O—(C₁-C₆ alkyl), halo, —NH₂, and C₁-C₆ aminoalkyl;
each occurrence of R⁷ is independently selected from C₁-C₆ alkyl, —O—(C₁-C₆ alkyl), halo, —NH₂, and C₁-C₆ aminoalkyl;
each occurrence of R⁸ is independently selected from C₁-C₆ alkyl, —O—(C₁-C₆ alkyl), halo, —NH₂, and C₁-C₆ aminoalkyl;
R^{A} is: 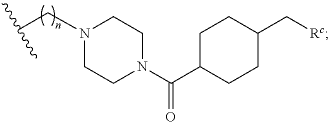
R^{B} is: 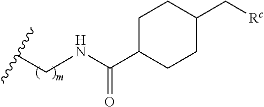
each R^{C} is independently selected from: 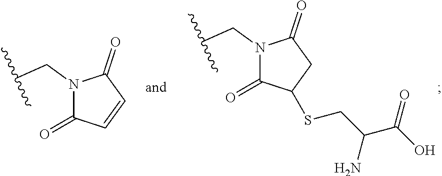 [SMILES: *CN1C(=O)C=CC1=O ; *CN1C(=O)CC(SCC(N)C(=O)O)C1=O]
each occurrence of m is independently 1 or 2; and
each occurrence of n is independently 0 or 1.

3. The compound of claim 1, or a pharmaceutically acceptable salt thereof, wherein one occurrence of R² is H, and the other occurrence of R² is selected from methyl, ethyl, isopropyl, isobutyl, n-butyl, n-pentyl, cyclopentyl, cyclohexyl, —(CH₂)₃—CH(CH₃)₂, —(CH₂)₂—CH(CH₃)₂, —(CH₂)₂OH, —(CH₂)₃OH, —(CH₂)₂OCH₃, —CH₂CH(OH)CH₃, —(CH₂)₂CH(OH)CH₃, —CH(CH₂CH₂CH₃)CH₂OH, —CH(CH₂CH₂CH₂CH₃)(CH₂CH₂OH), —CH(CH₃)CH₂CH₂CH₃, —CH₂CH(CH₃)CH₂CH₃, and —CH(CH₂CH₂CH₃)CH₂CH₂CH₂CH₃, wherein said cyclopentyl group, and said cyclohexyl group, can be optionally substituted with one group selected from —OH and —CH₂OH; or two R² groups, together with the nitrogen atom to which they are attached, can join to form a pyrrolidinyl group.

4. The compound of claim 1, or a pharmaceutically acceptable salt thereof, wherein R³ is benzyl or pyridyl, which can be optionally substituted with up to three groups, which can be the same or different, and are selected from methyl, methoxy, ethoxy, isopropoxy, Cl, F, —NH₂, —CH₂NH₂, —CH₂CH₂NH₂, pyrrolidinyl, —CH₂-pyrroldinyl, —CH₂NHCH₃, —CH(CH₃)NHCH₃, —CH(—NHCH₃)CH₂CH₂CH₃, —CH(CH₃)NHCH₂CH₂CH₃, —CH(—NHCH₃)CH₂CH₂NHC(O)-pyridyl, piperazinyl, —SCH₂CH(NH₂)C(O)OH,

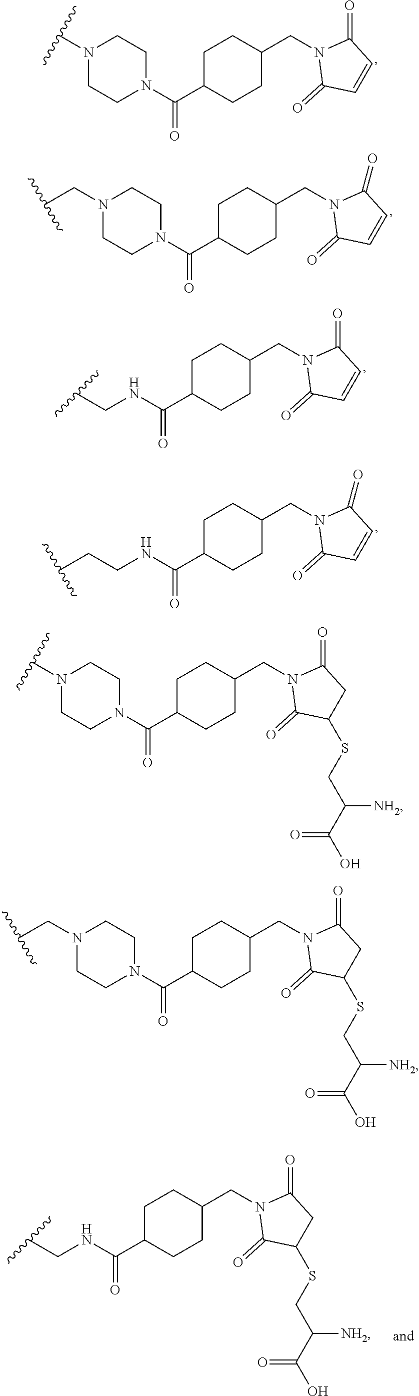

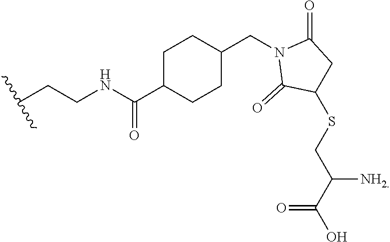 [SMILES: *CCNC(=O)C1CCC(CN2C(=O)CC(SCC(N)C(=O)O)C2=O)CC1]

5. The compound of claim 1, having the formula (Ib): 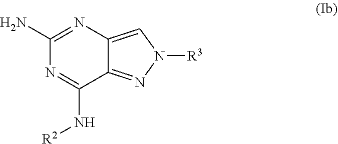
or a pharmaceutically acceptable salt thereof,
wherein:
R² is C₁-C₆ alkyl;
R³ is selected from benzyl, and —CH₂-(5- or 6-membered monocyclic heteroaryl), wherein the phenyl moiety of said benzyl group and the 5- or 6-membered monocyclic heteroaryl moiety of said —CH₂-(5- or 6-membered monocyclic heteroaryl) group can be optionally substituted with one or more groups, which can be the same or different and are each independently selected from —O—(C₁-C₆ alkyl), halo, —NH₂, C₁-C₆ aminoalkyl, and 5- to 7-membered monocyclic heterocycloalkyl).

6. The compound of claim 1, or a pharmaceutically acceptable salt thereof, wherein R³ is selected from: 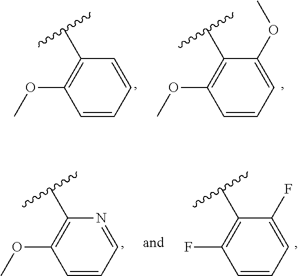
each of which groups can be optionally substituted with one group selected from —NH₂, —CH₂NH₂,

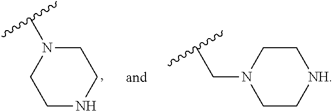 [SMILES: *N1CCNCC1 ; "," ; "and" ; *CN1CCNCC1]

7. The compound of claim 1, having the formula (Ic): 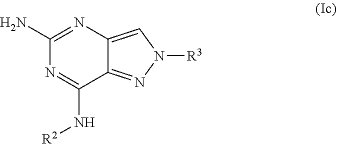
or a pharmaceutically acceptable salt thereof,
wherein:
R² is n-butyl or —CH(CH₂OH)CH₂CH₂CH₃; and
R³ is selected from: 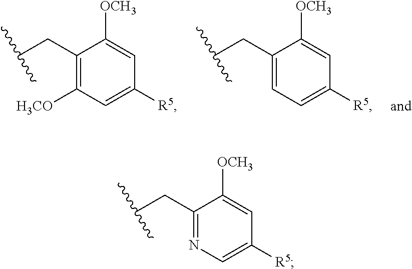
and
R⁵ is selected from —CH₂NH₂, —CH₂NHCH₃, —CH(CH₃)NHCH₃, —CH(—NHCH₃)CH₂CH₂CH₃, —CH(CH₃)NHCH₂CH₂CH₃, —CH(—NHCH₃)CH₂CH₂NHC(O)-pyridyl, piperazinyl, and —SCH₂CH(NH₂)C(O)OH.

8. The compound of claim 1, or a pharmaceutically acceptable salt thereof, wherein R² is n-butyl, or a pharmaceutically acceptable salt thereof.

9. The compound of claim 6, or a pharmaceutically acceptable salt thereof, wherein R³ is: 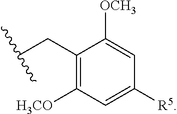
wherein R⁵ is selected from: —CH₂NH₂, —CH₂NHCH₃, —CH(CH₃)NHCH₃, —CH(—NHCH₃)CH₂CH₂CH₃, —CH(—NHCH₃)CH₂CH₂NHC(O)-pyridyl, and —SCH₂CH(NH₂)C(O)OH.

10. The compound of claim 6, or a pharmaceutically acceptable salt thereof, wherein R³ is: 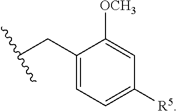
wherein R⁵ is selected from: —CH(CH₃)NHCH₂CH₂CH₃, and piperizinyl.

11. The compound of claim 6, or a pharmaceutically acceptable salt thereof, wherein R³ is:

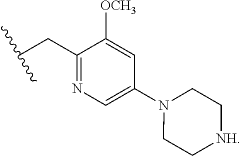 [SMILES: *Cc1ncc(N2CCNCC2)cc1OC]

12. A compound having the structure: 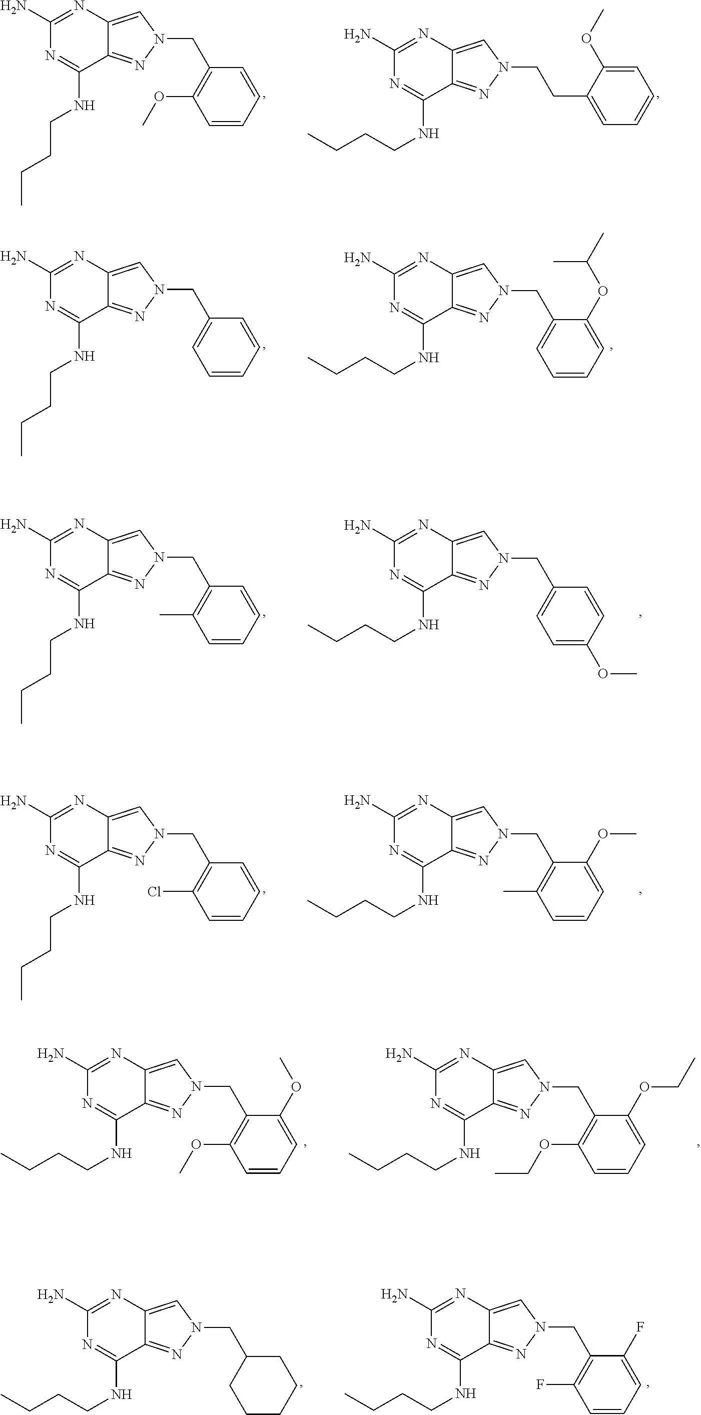 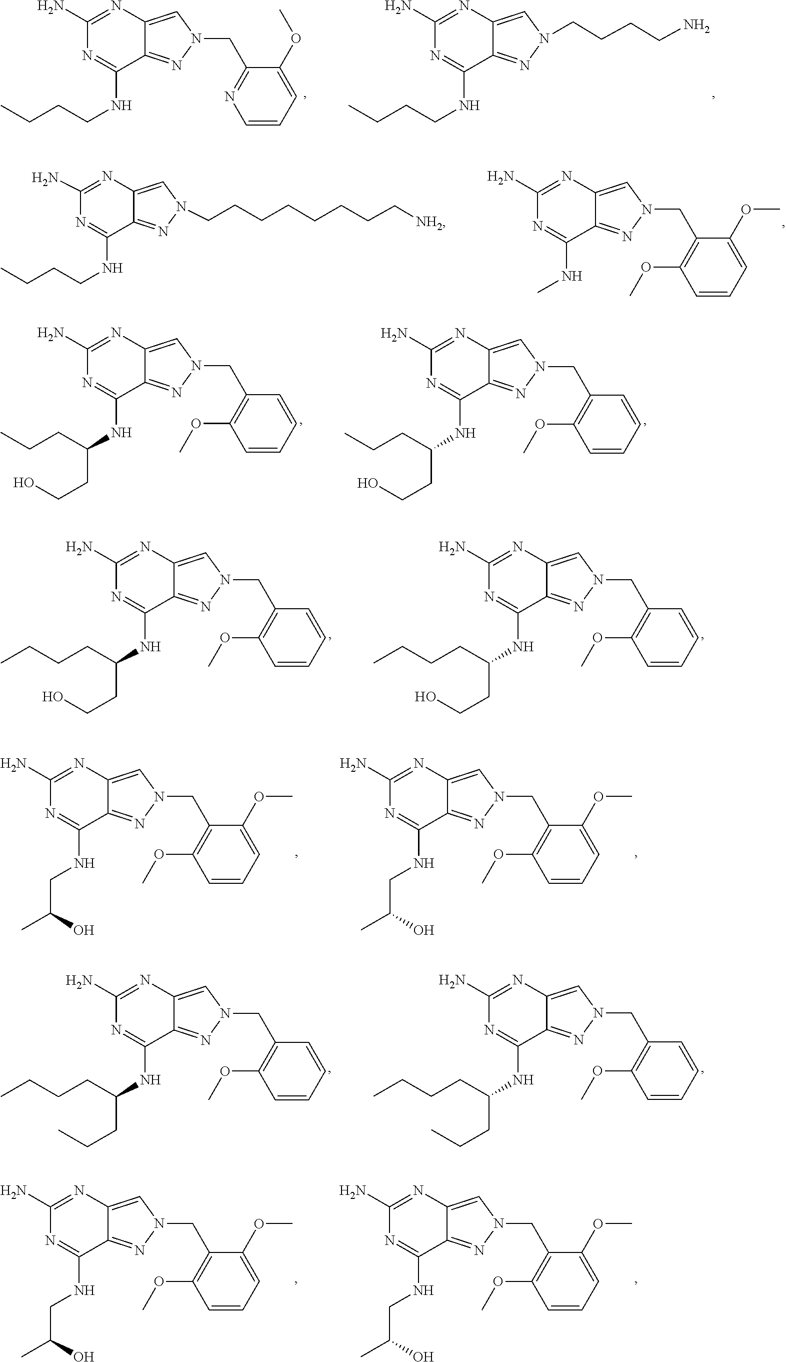 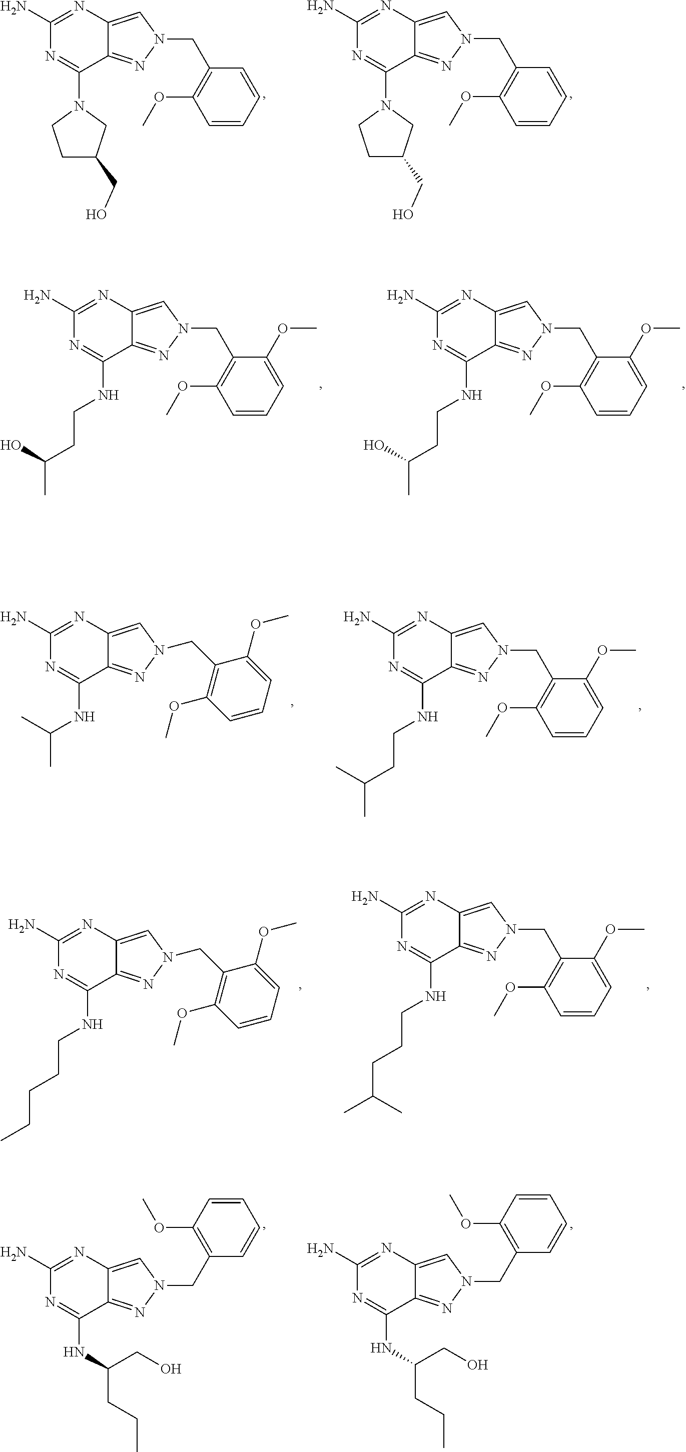 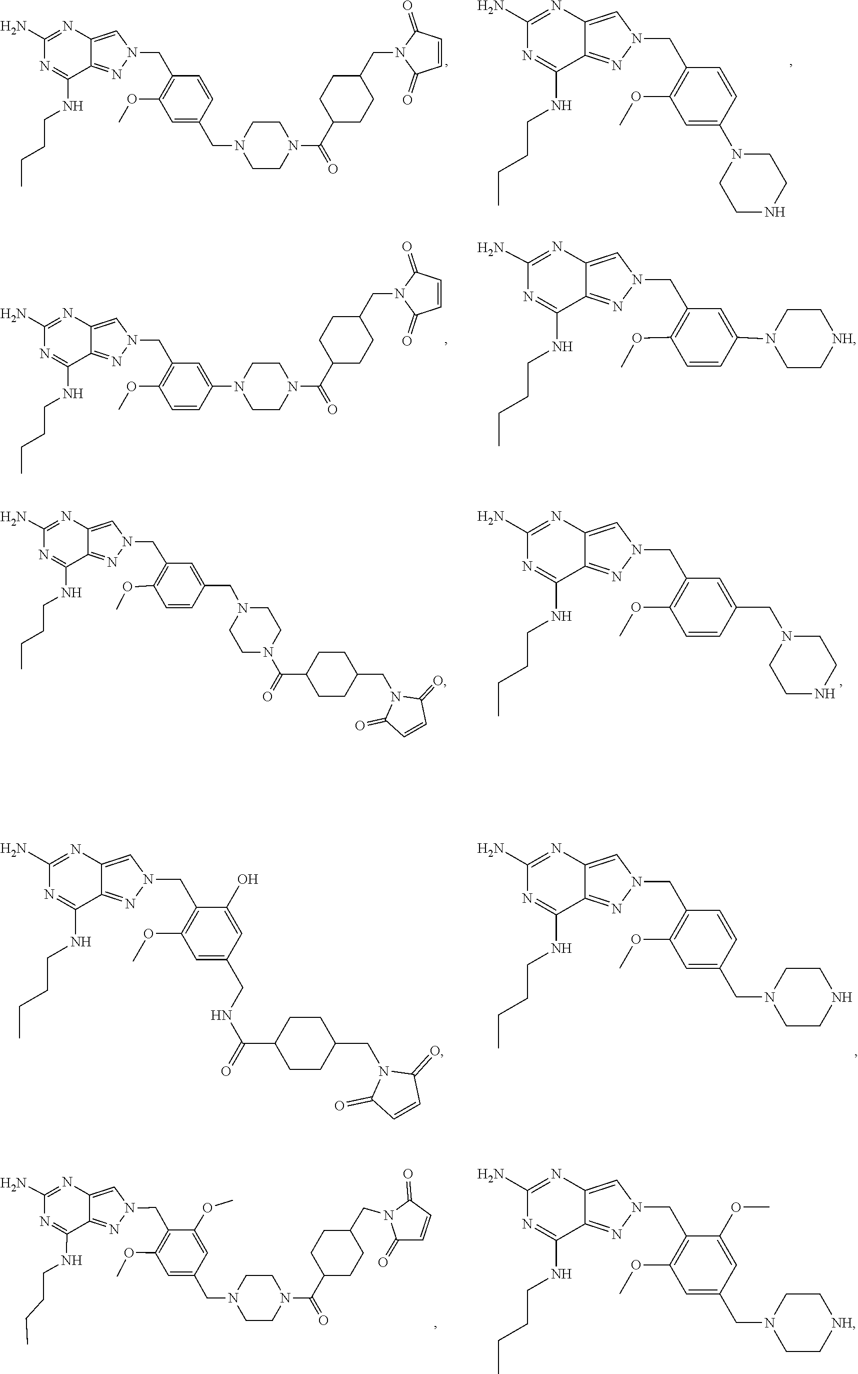 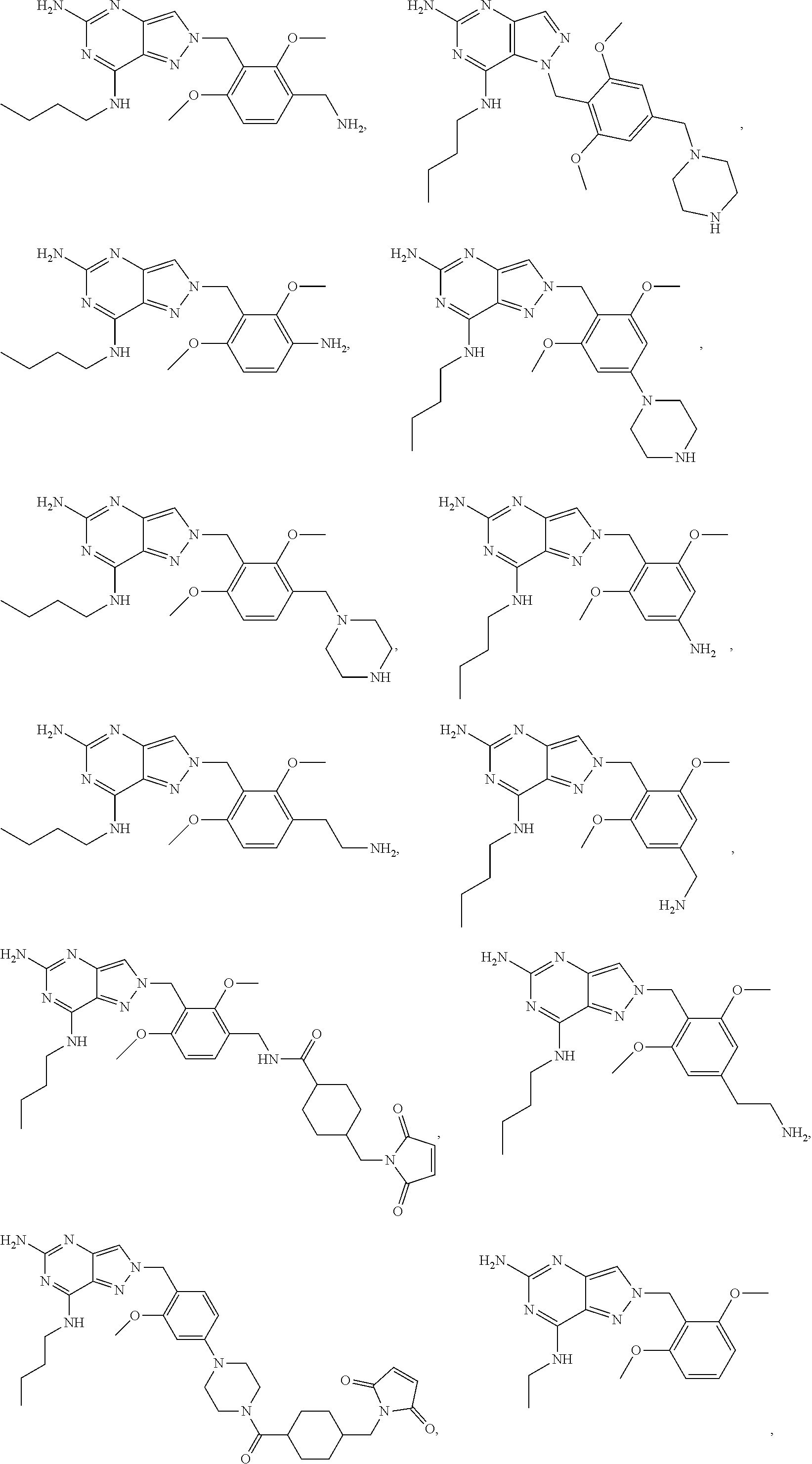 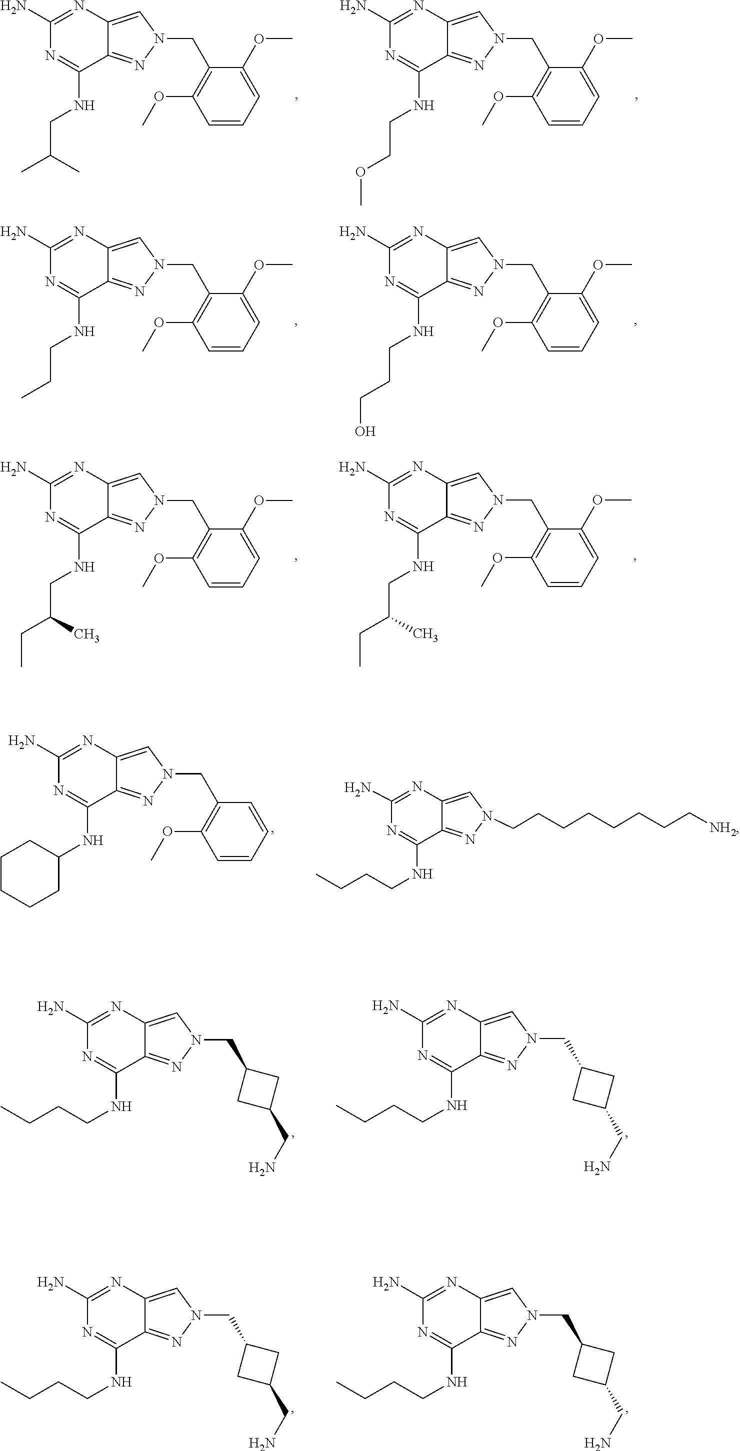 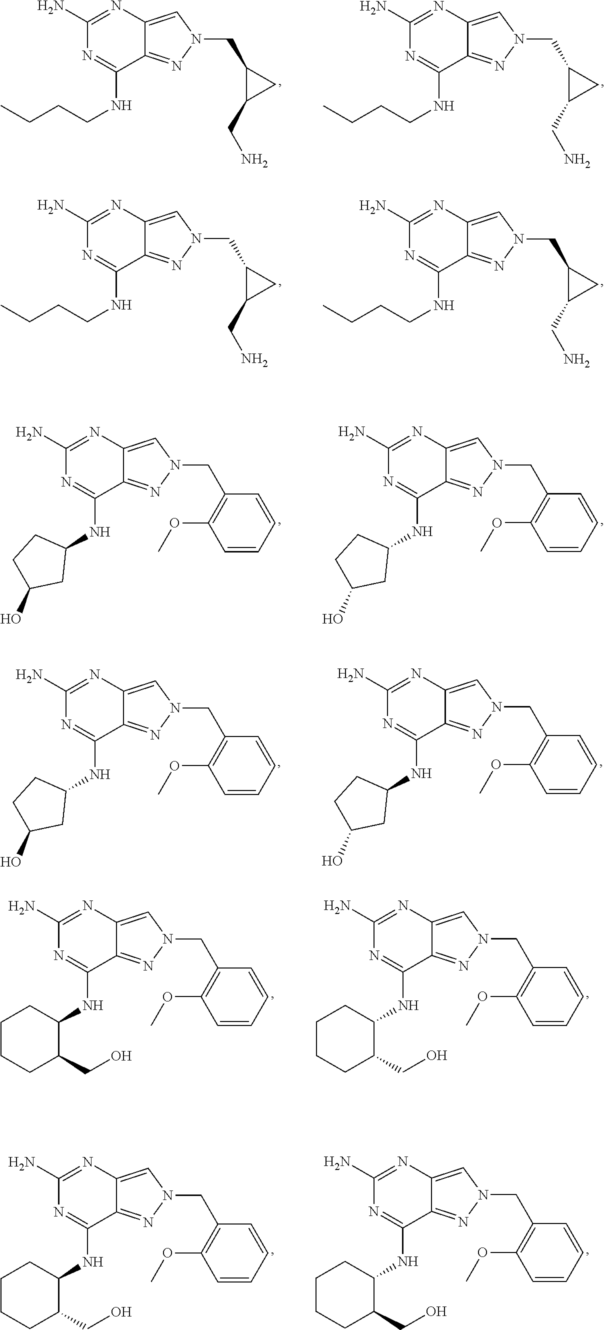 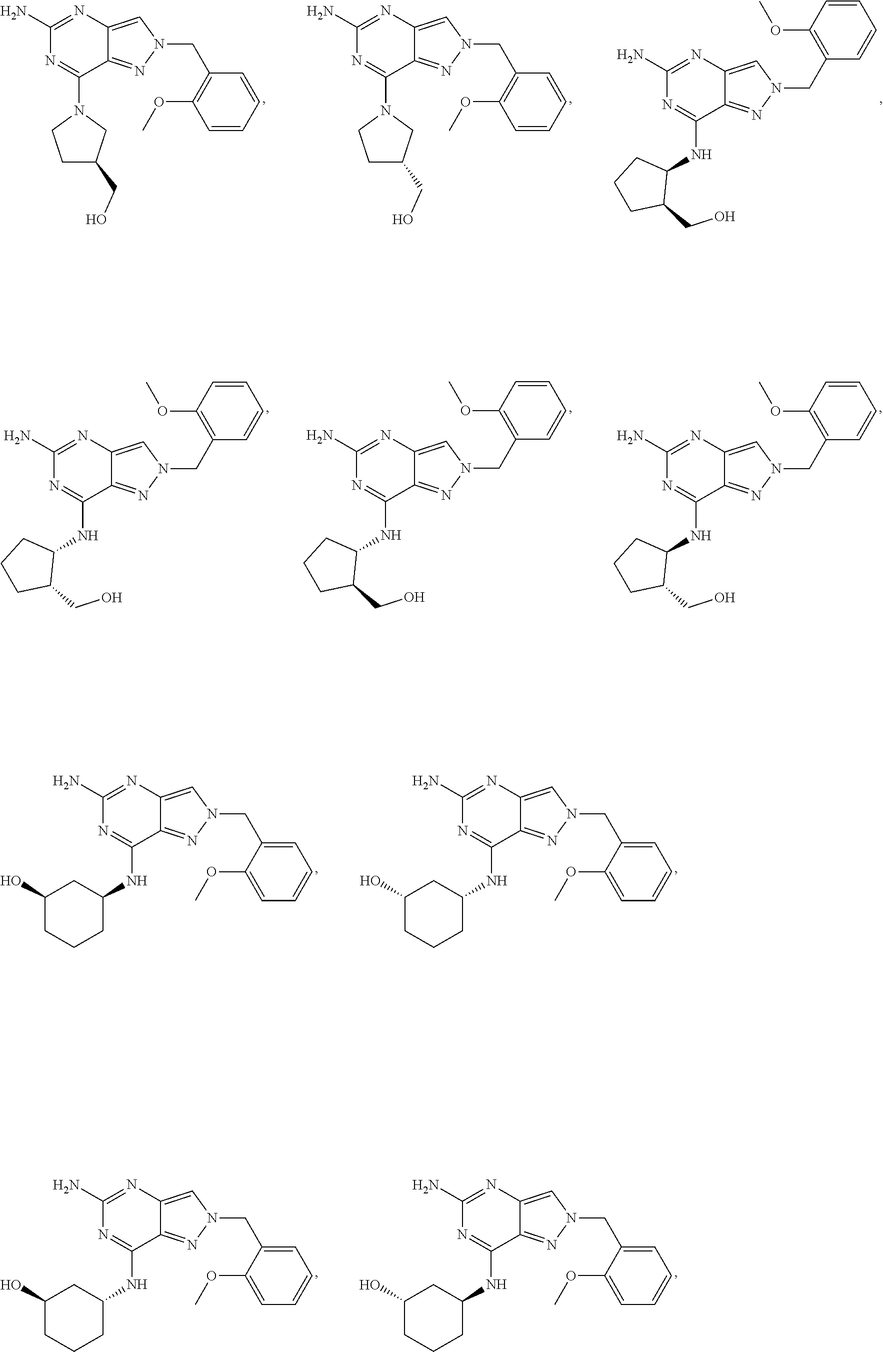 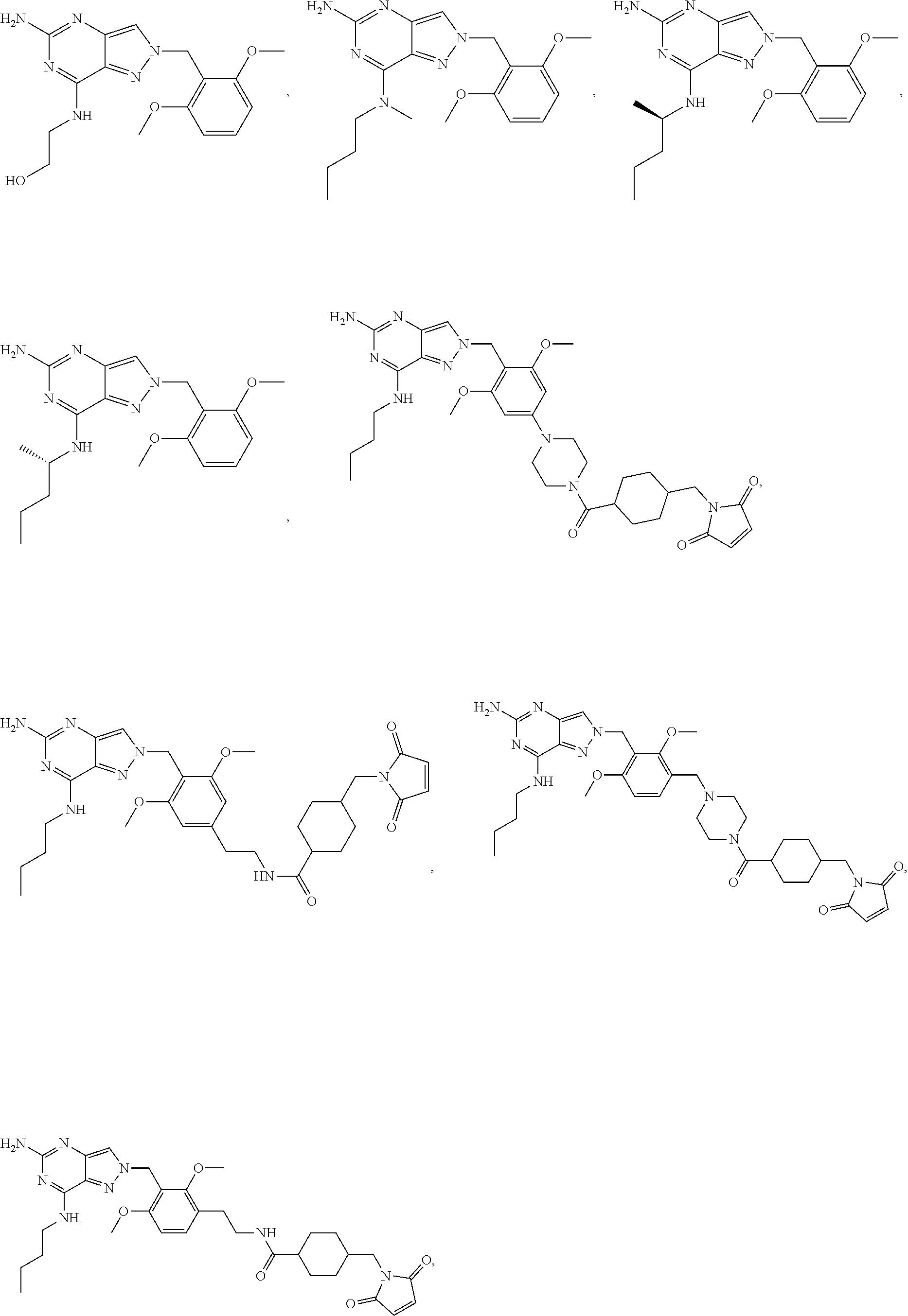 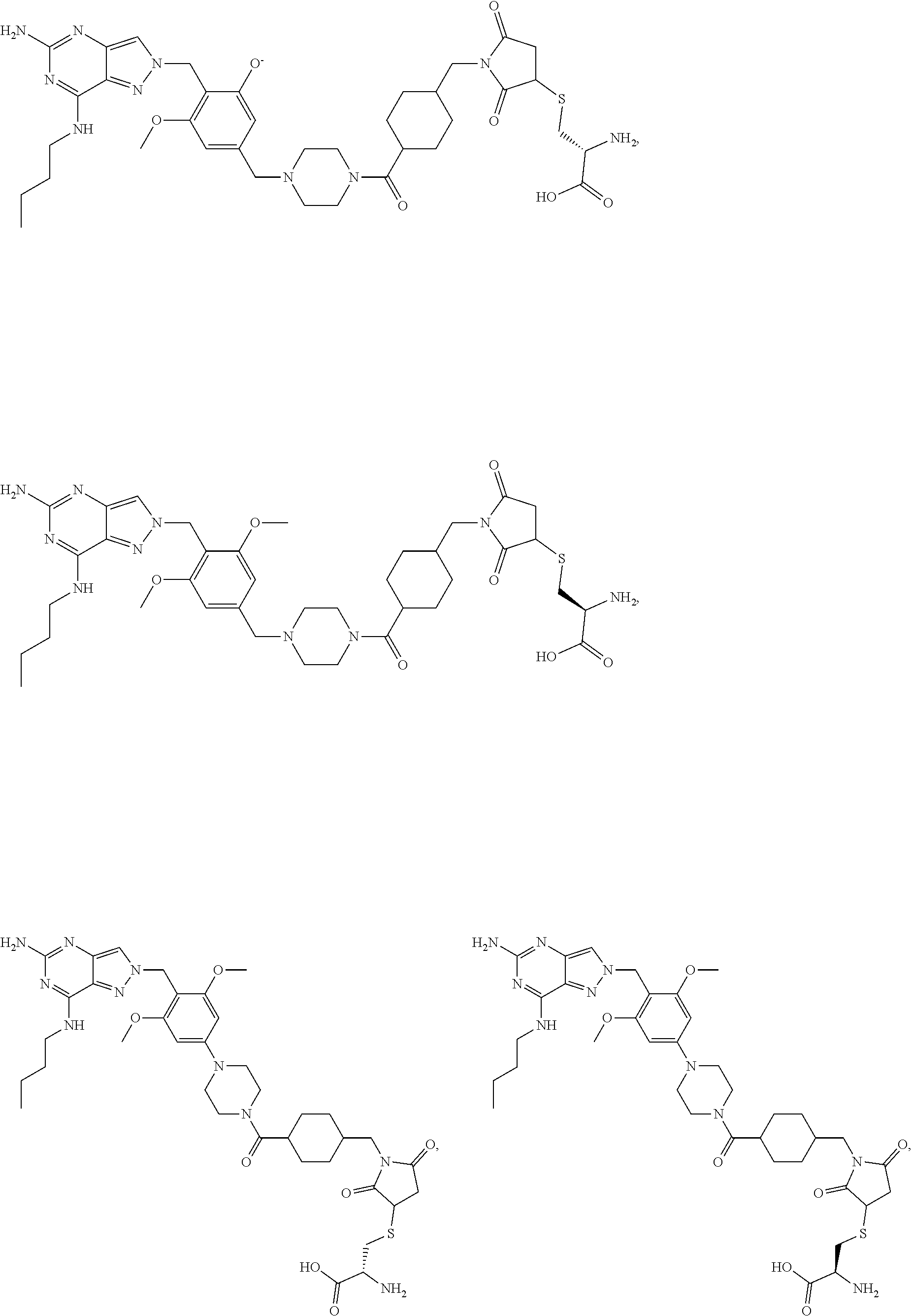 [SMILES: CCCCNc1nc(N)nc2cn(Cc3c([O-])cc(CN4CCN(C(=O)C5CCC(CN6C(=O)CC(SC[C@H](N)C(=O)O)C6=O)CC5)CC4)cc3OC)nc12 ; CCCCNc1nc(N)nc2cn(Cc3c(OC)cc(CN4CCN(C(=O)C5CCC(CN6C(=O)CC(SC[C@@H](N)C(=O)O)C6=O)CC5)CC4)cc3OC)nc12 ; CCCCNc1nc(N)nc2cn(Cc3c(OC)cc(N4CCN(C(=O)C5CCC(CN6C(=O)CC(SC[C@H](N)C(=O)O)C6=O)CC5)CC4)cc3OC)nc12 ; CCCCNc1nc(N)nc2cn(Cc3c(OC)cc(N4CCN(C(=O)C5CCC(CN6C(=O)CC(SC[C@@H](N)C(=O)O)C6=O)CC5)CC4)cc3OC)nc12] 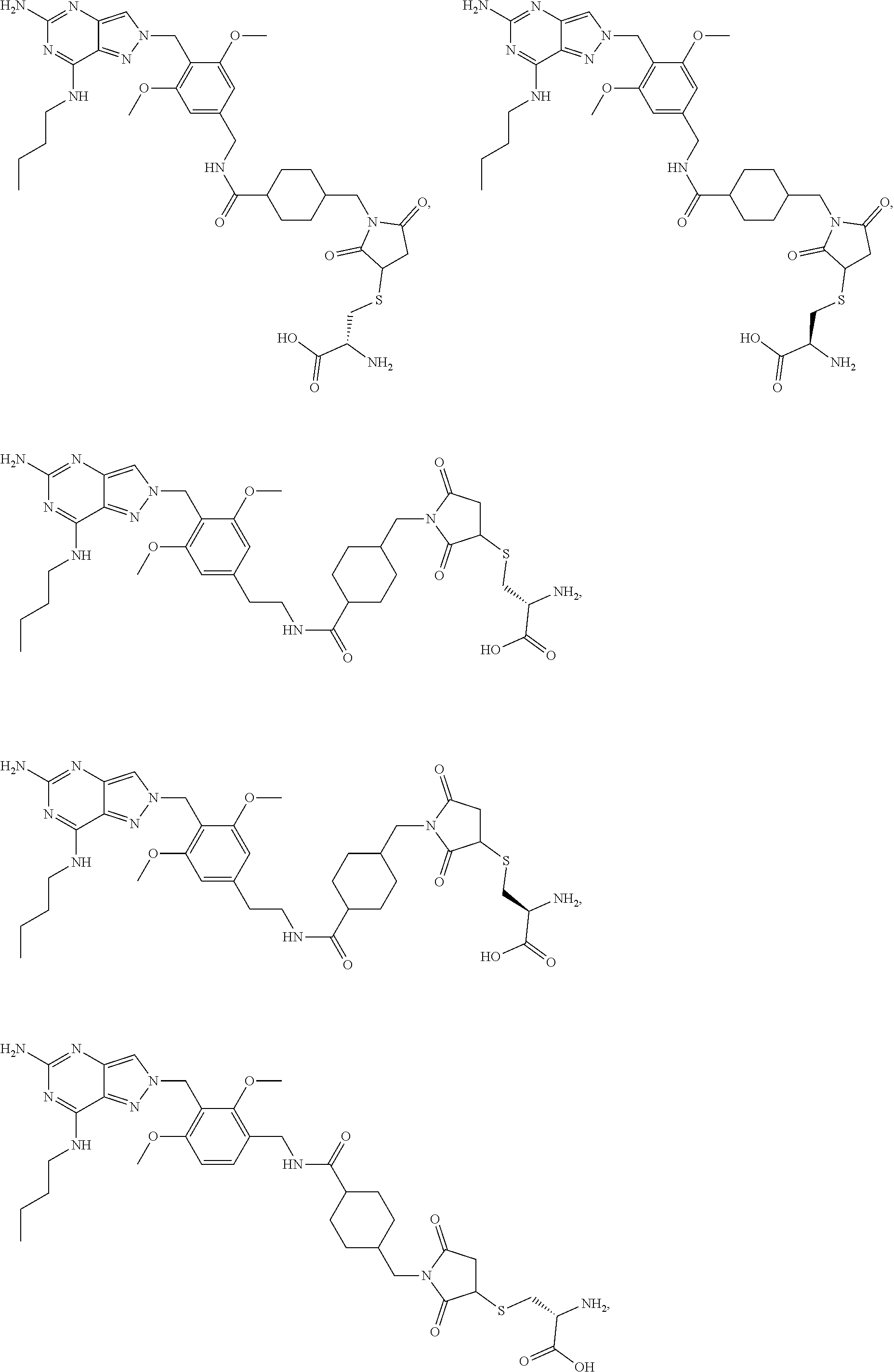 [SMILES: CCCCNc1nc(N)nc2cn(Cc3c(OC)cc(CNC(=O)C4CCC(CN5C(=O)CC(SC[C@H](N)C(=O)O)C5=O)CC4)cc3OC)nc12 ; CCCCNc1nc(N)nc2cn(Cc3c(OC)cc(CNC(=O)C4CCC(CN5C(=O)CC(SC[C@@H](N)C(=O)O)C5=O)CC4)cc3OC)nc12 ; CCCCNc1nc(N)nc2cn(Cc3c(OC)cc(CCNC(=O)C4CCC(CN5C(=O)CC(SC[C@H](N)C(=O)O)C5=O)CC4)cc3OC)nc12 ; CCCCNc1nc(N)nc2cn(Cc3c(OC)cc(CCNC(=O)C4CCC(CN5C(=O)CC(SC[C@@H](N)C(=O)O)C5=O)CC4)cc3OC)nc12 ; CCCCNc1nc(N)nc2cn(Cc3c(OC)ccc(CNC(=O)C4CCC(CN5C(=O)CC(SC[C@H](N)C(=O)O)C5=O)CC4)c3OC)nc12] 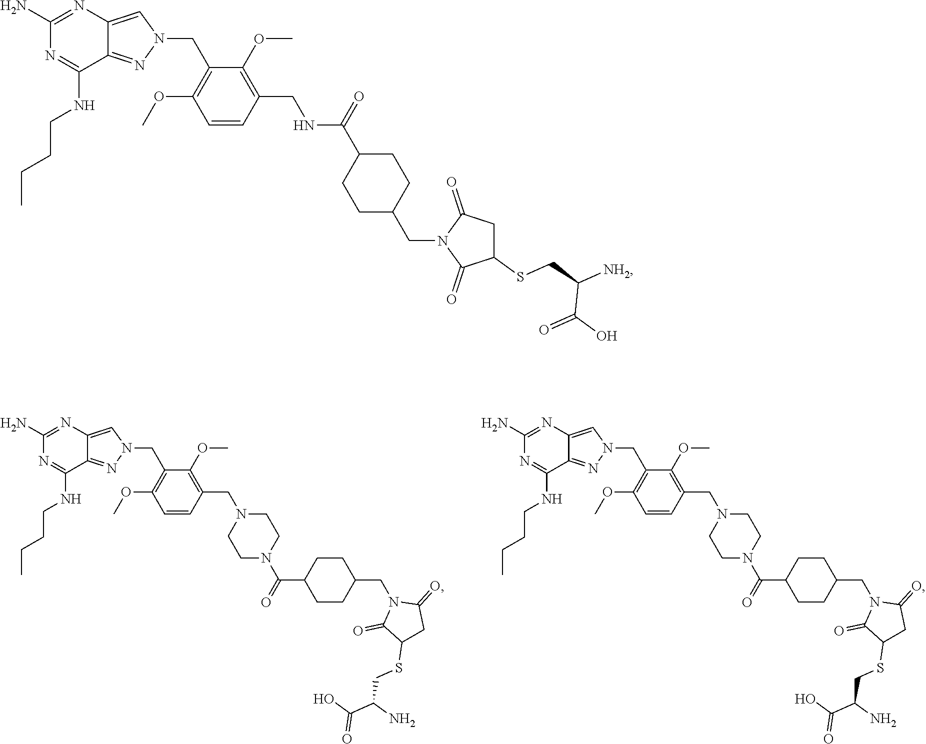 [SMILES: CCCCNc1nc(N)nc2cn(Cc3c(OC)ccc(CNC(=O)C4CCC(CN5C(=O)CC(SC[C@@H](N)C(=O)O)C5=O)CC4)c3OC)nc12 ; CCCCNc1nc(N)nc2cn(Cc3c(OC)ccc(CN4CCN(C(=O)C5CCC(CN6C(=O)CC(SC[C@H](N)C(=O)O)C6=O)CC5)CC4)c3OC)nc12 ; CCCCNc1nc(N)nc2cn(Cc3c(OC)ccc(CN4CCN(C(=O)C5CCC(CN6C(=O)CC(SC[C@@H](N)C(=O)O)C6=O)CC5)CC4)c3OC)nc12] 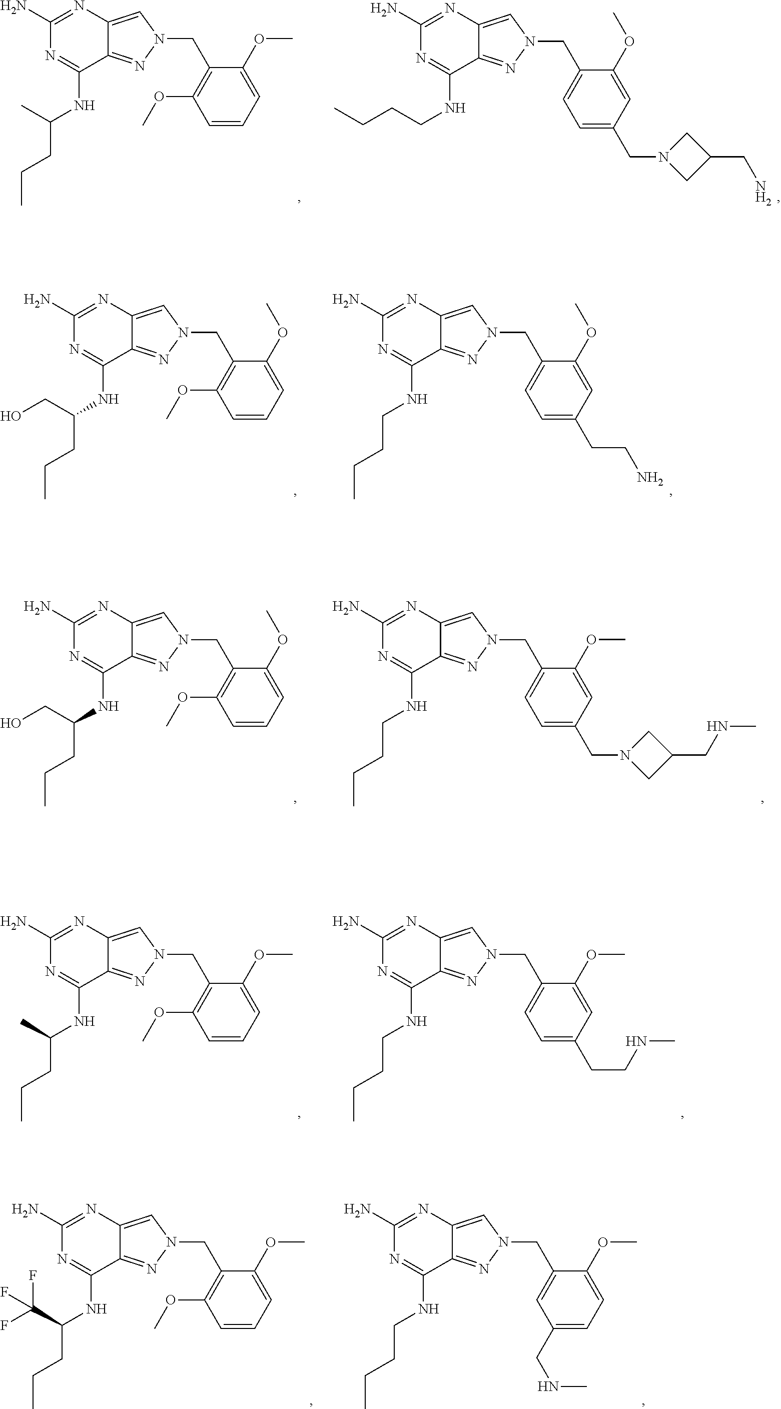 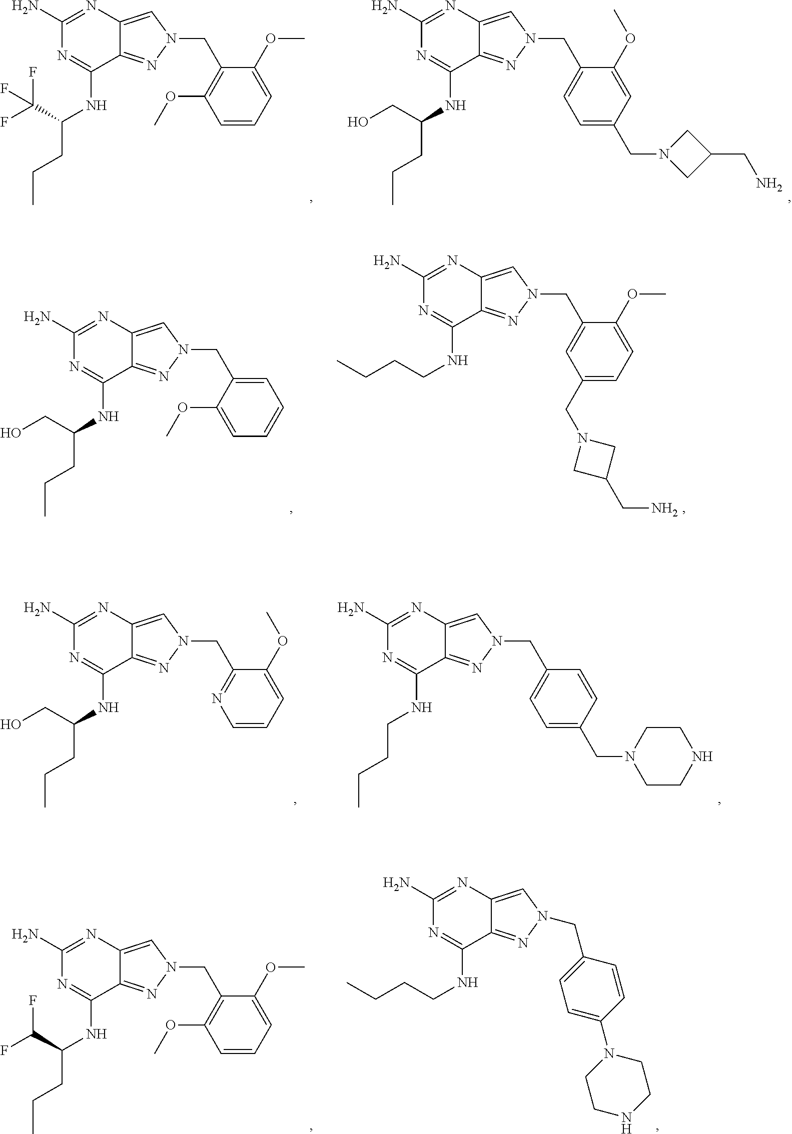 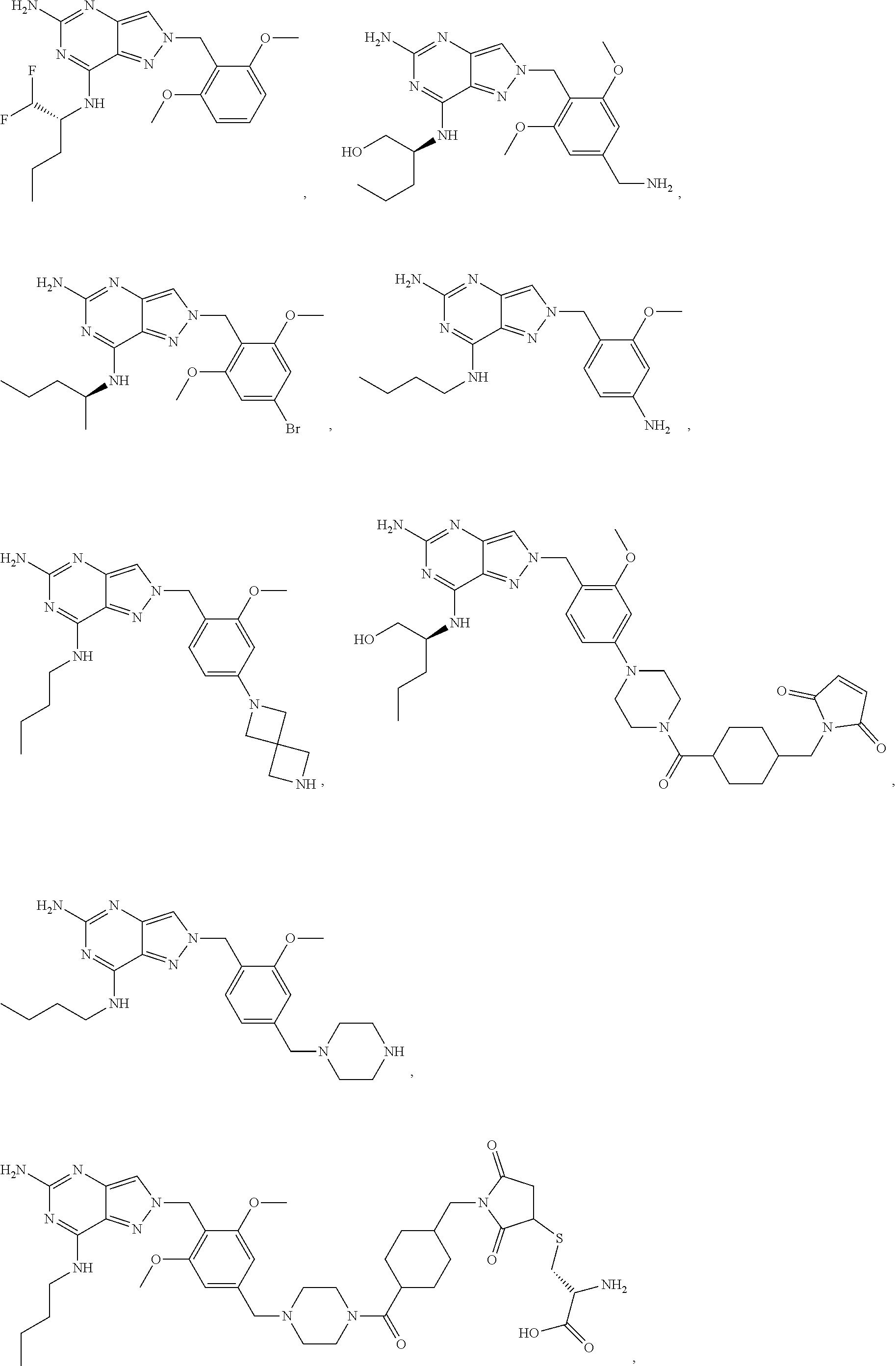 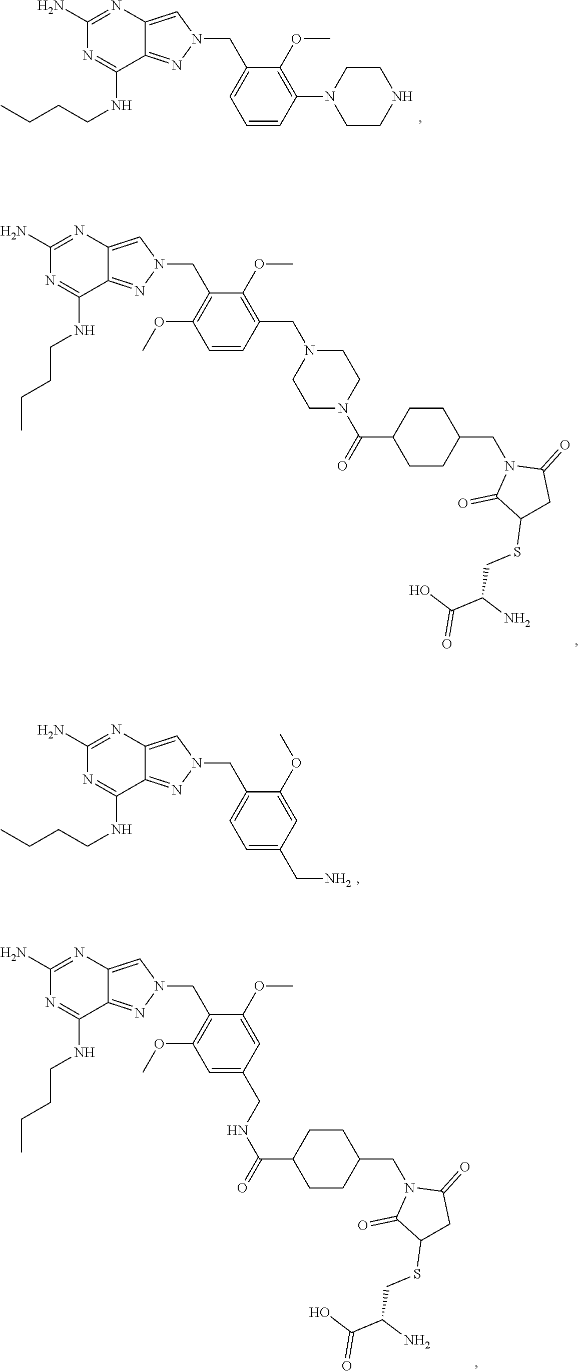 [SMILES: CCCCNc1nc(N)nc2cn(Cc3cccc(N4CCNCC4)c3OC)nc12 ; CCCCNc1nc(N)nc2cn(Cc3c(OC)ccc(CN4CCN(C(=O)C5CCC(CN6C(=O)CC(SC[C@H](N)C(=O)O)C6=O)CC5)CC4)c3OC)nc12 ; CCCCNc1nc(N)nc2cn(Cc3ccc(CN)cc3OC)nc12 ; CCCCNc1nc(N)nc2cn(Cc3c(OC)cc(CNC(=O)C4CCC(CN5C(=O)CC(SC[C@H](N)C(=O)O)C5=O)CC4)cc3OC)nc12] 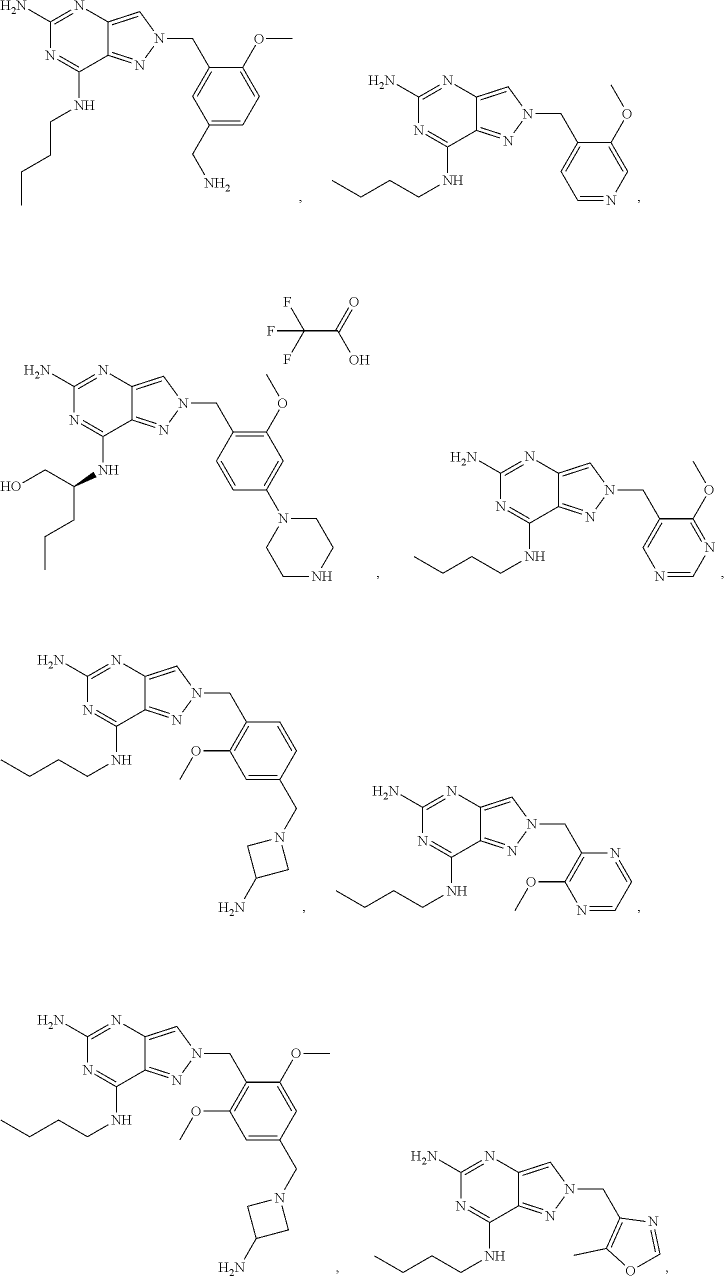 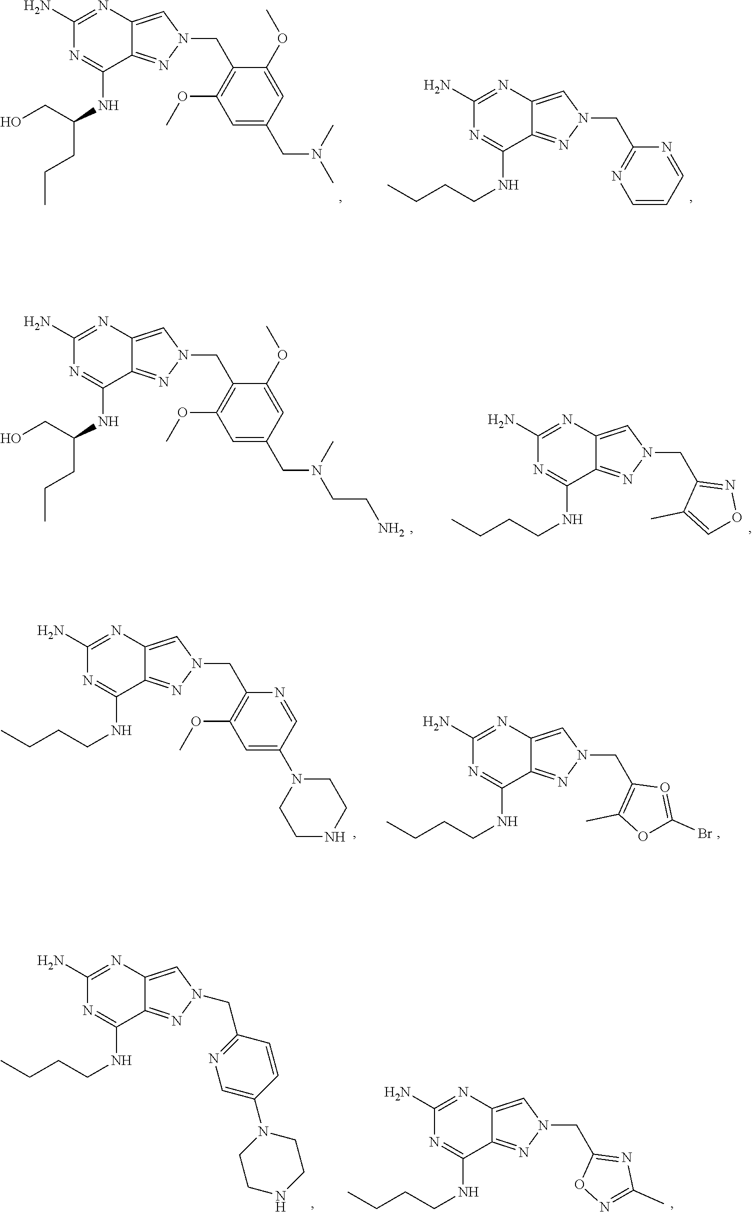 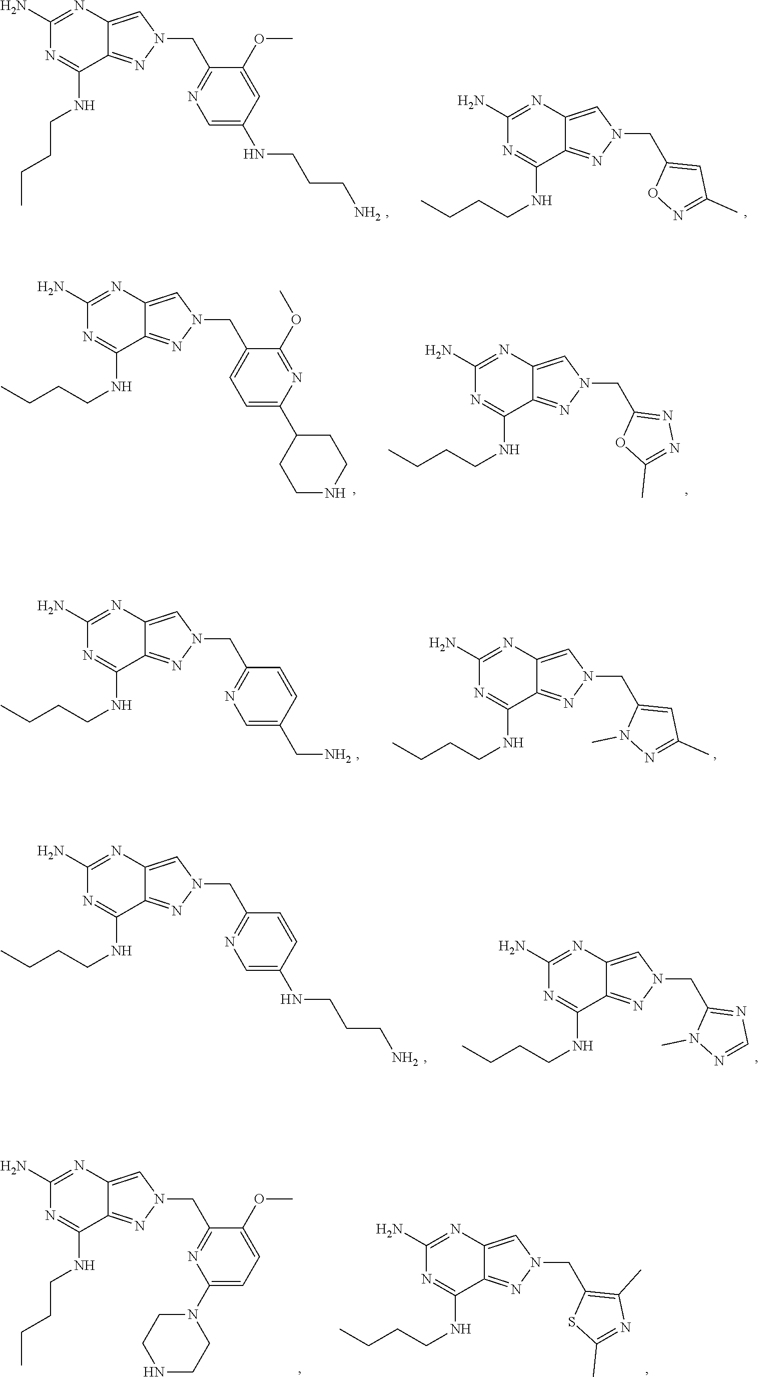 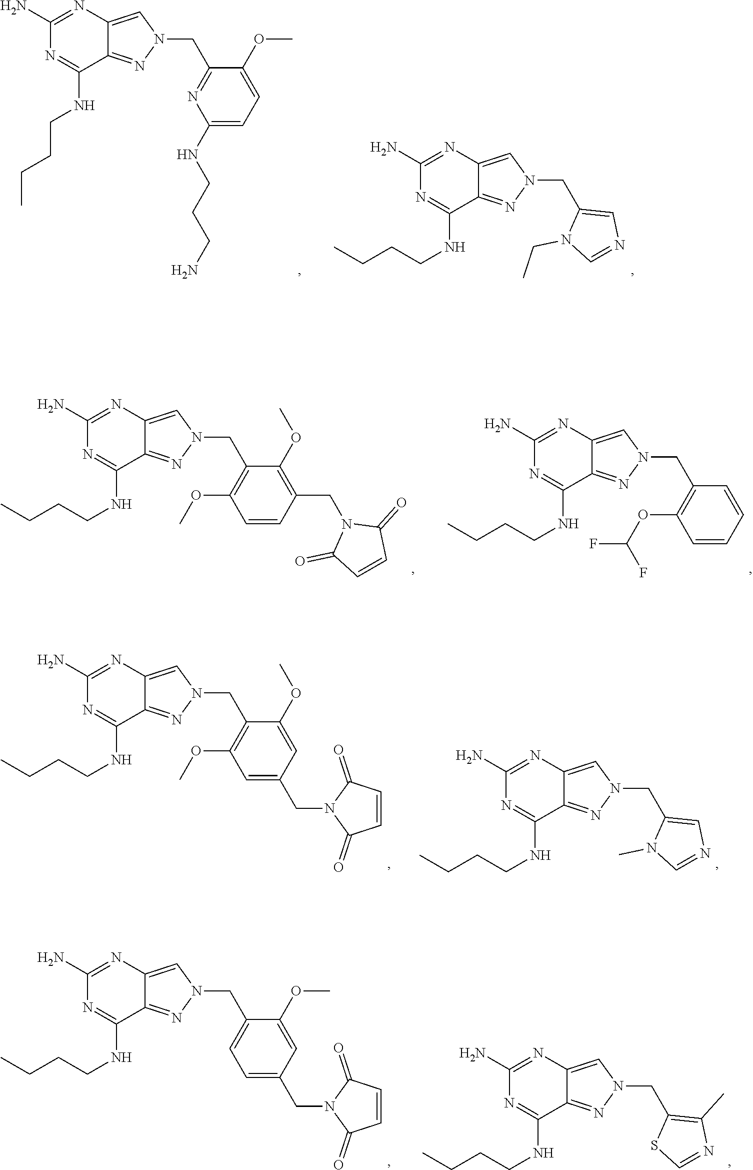 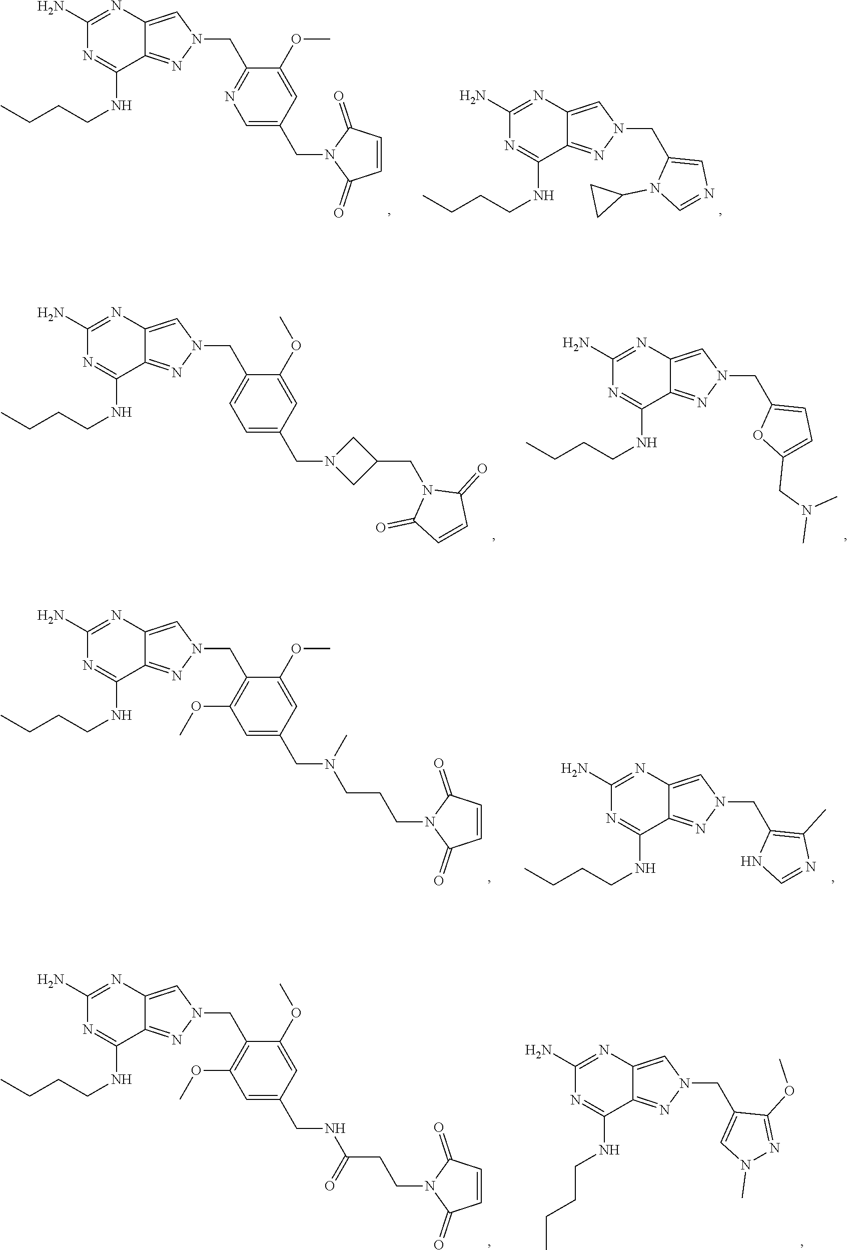 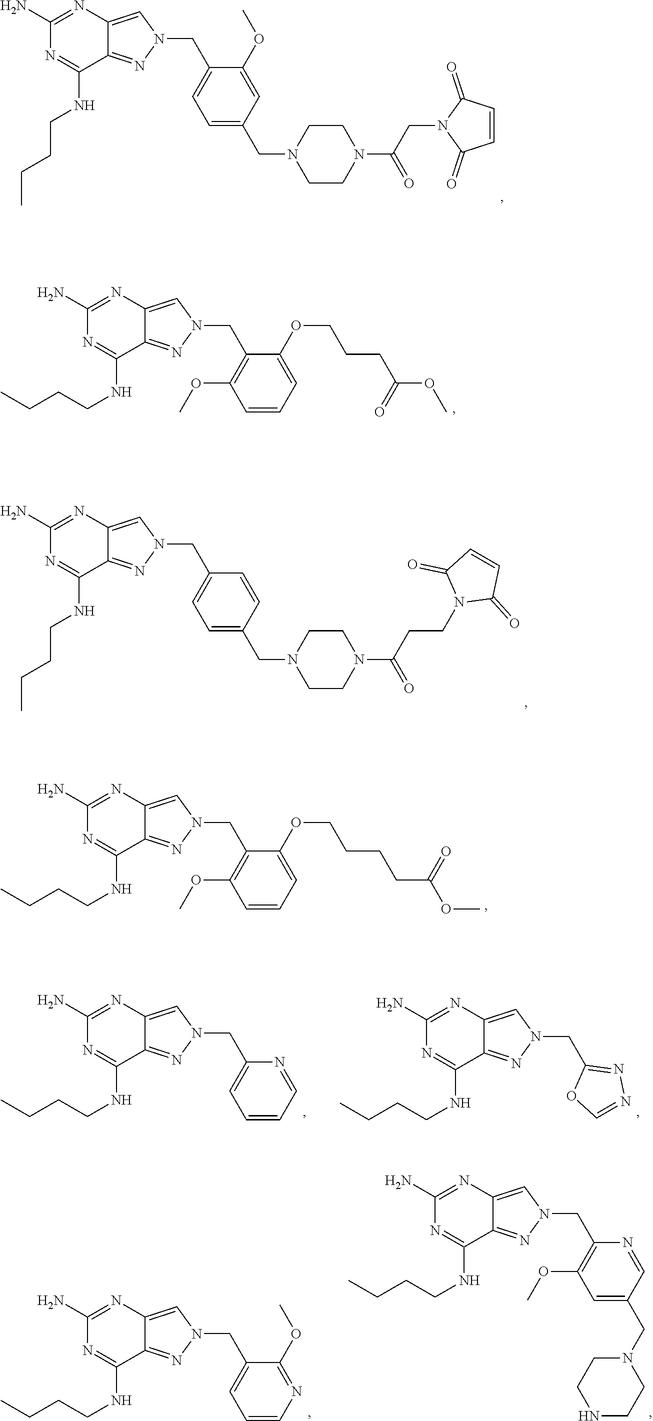 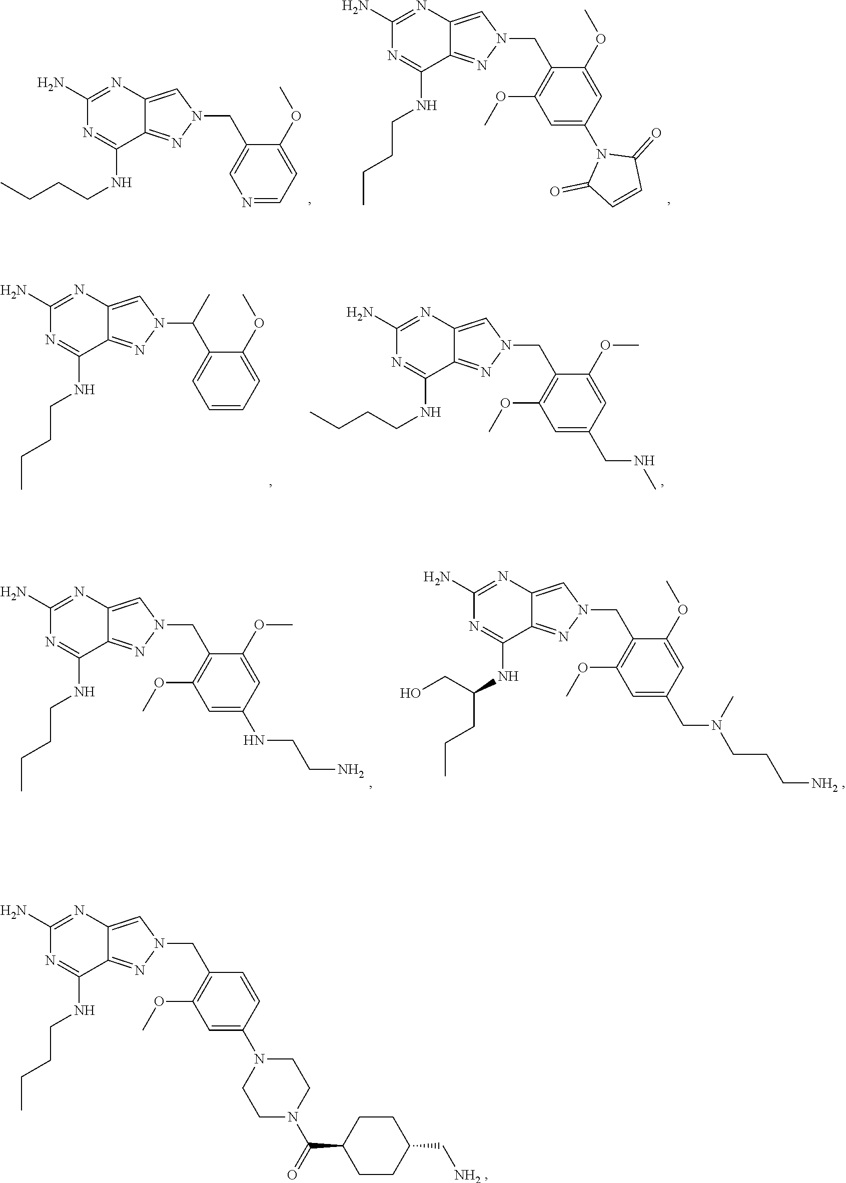 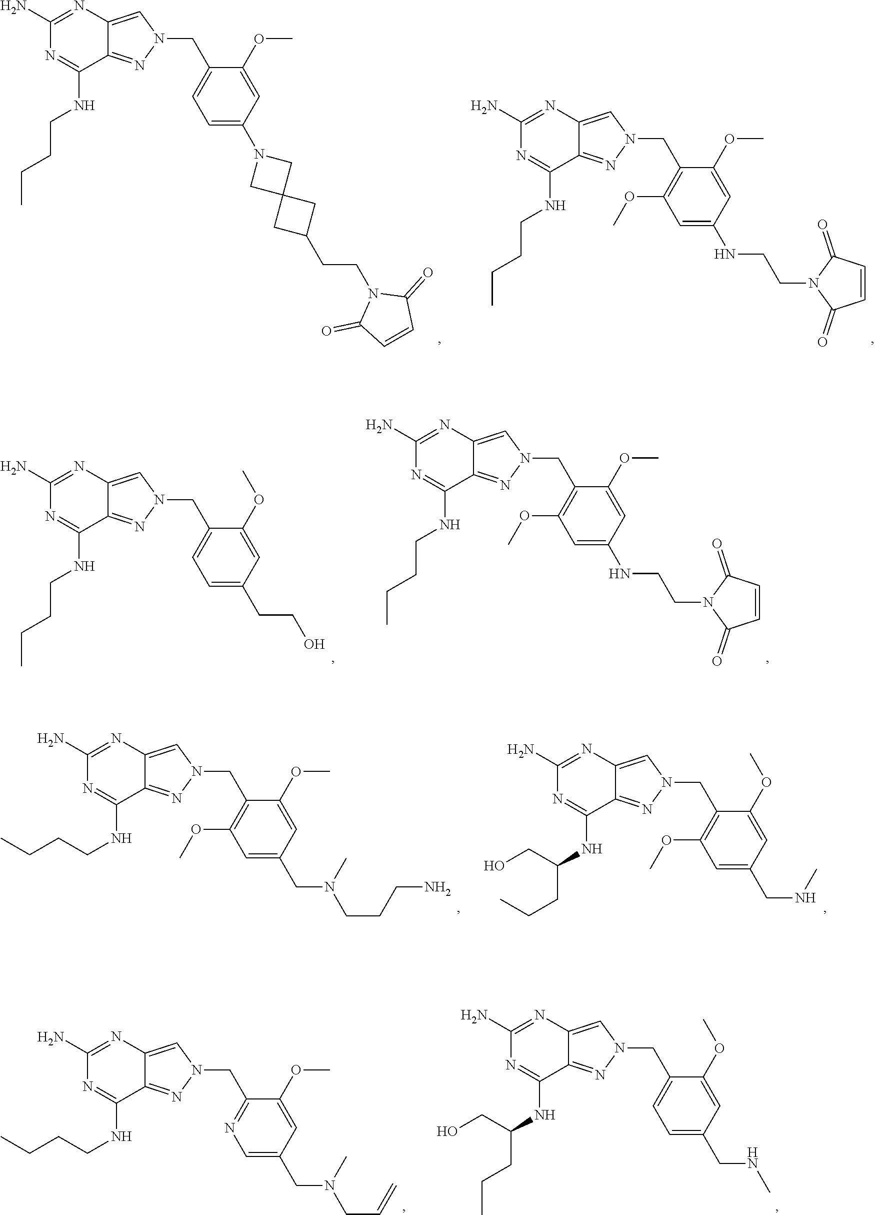 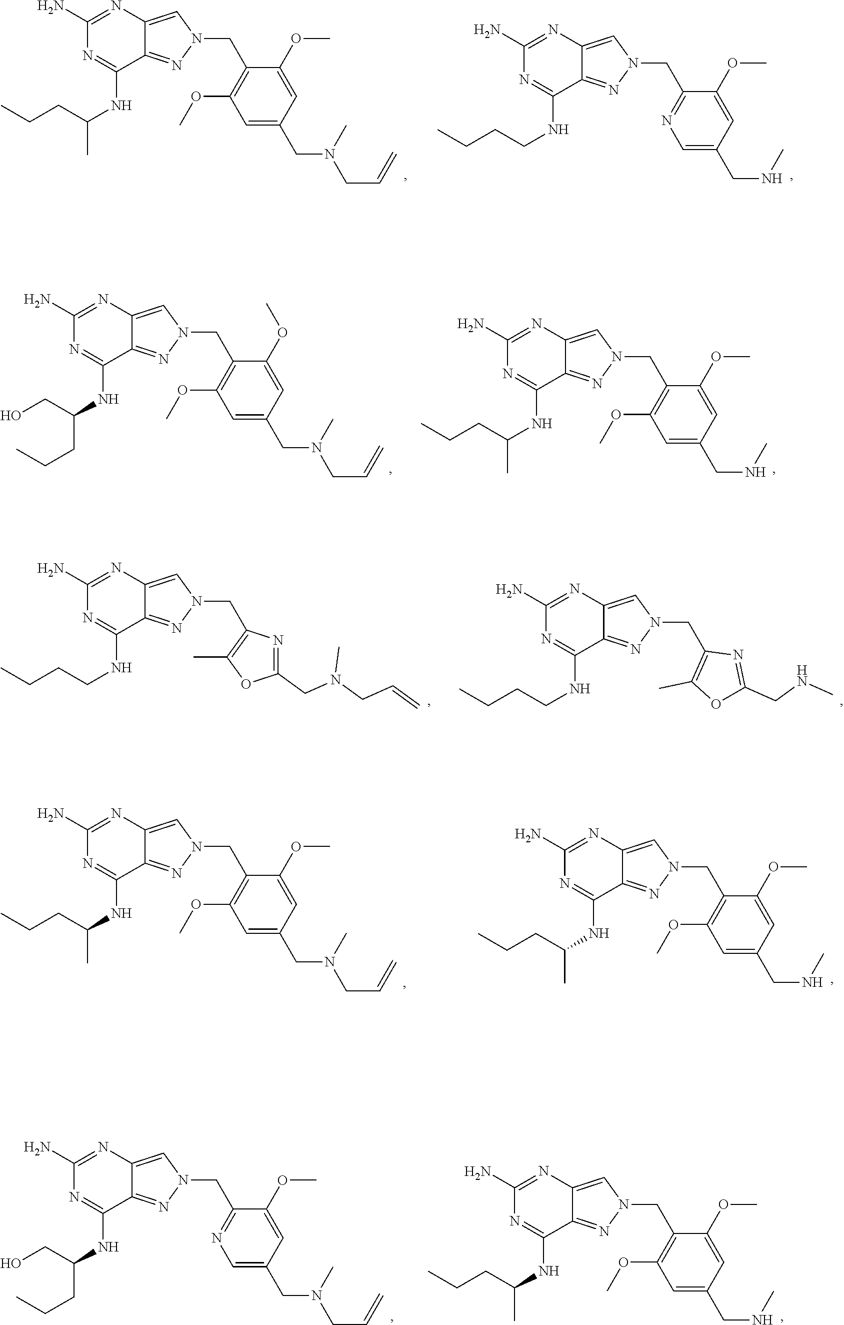 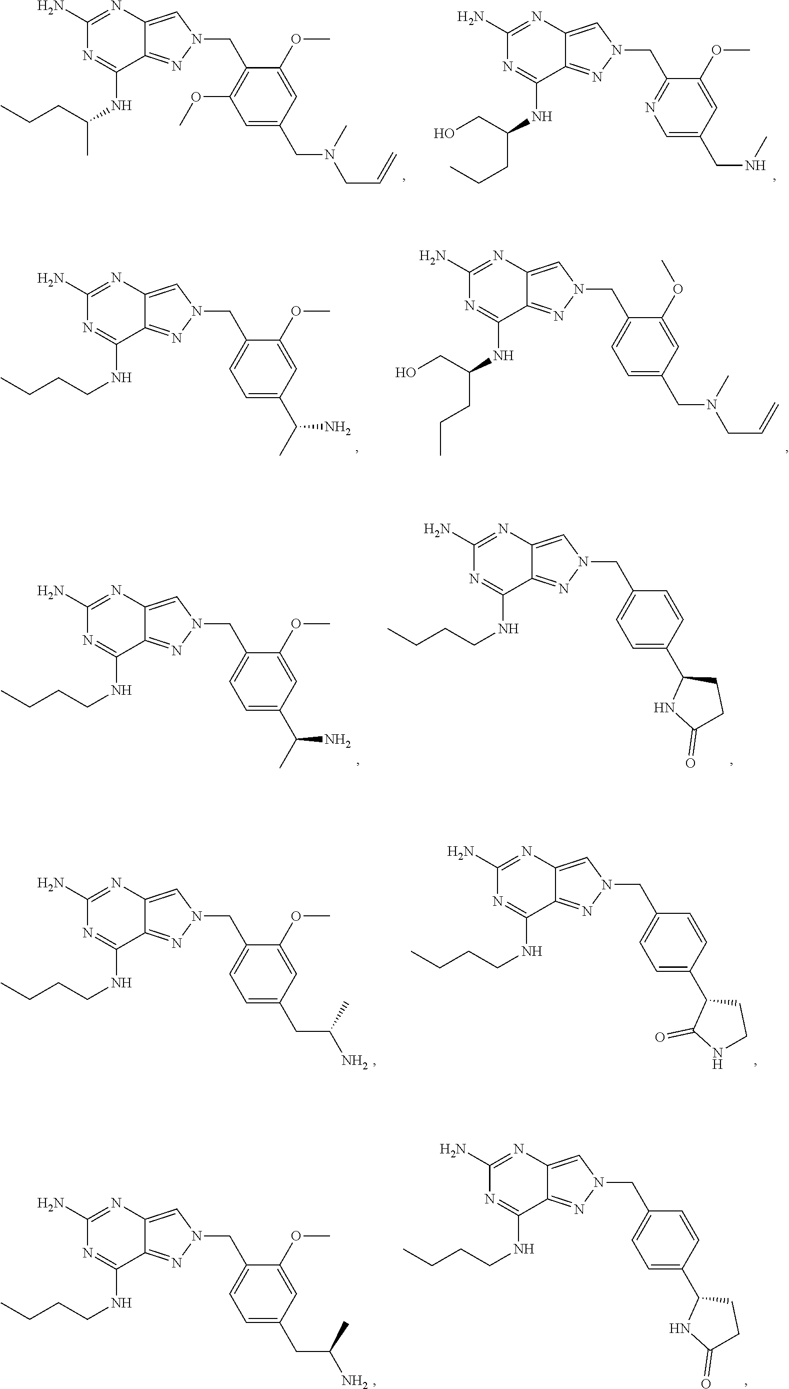 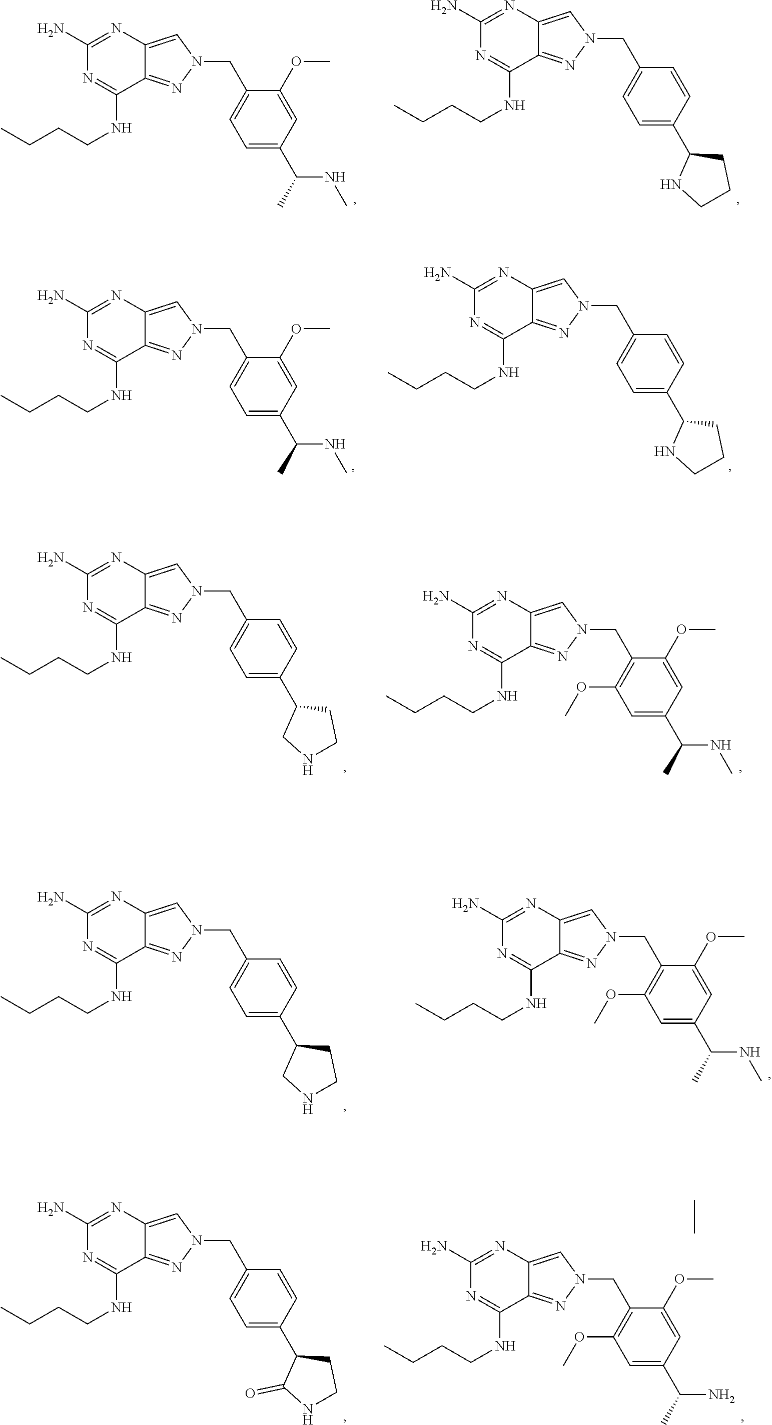 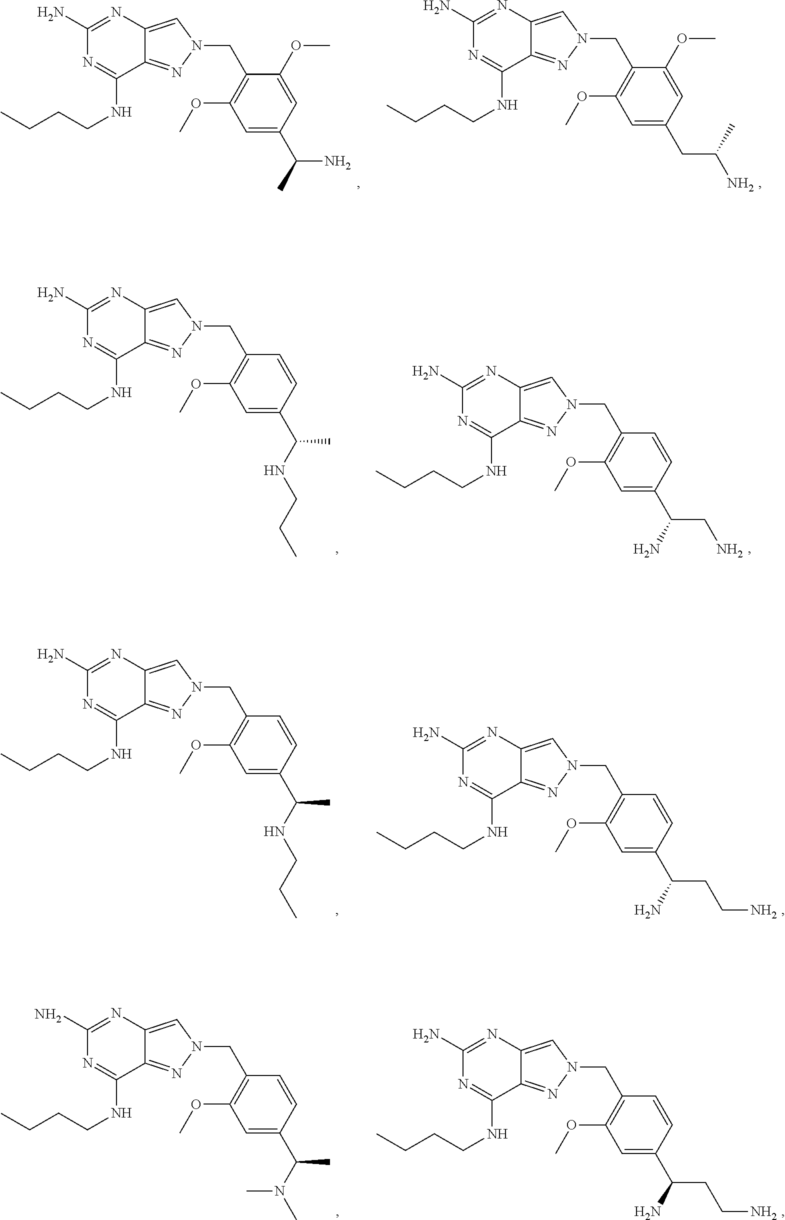 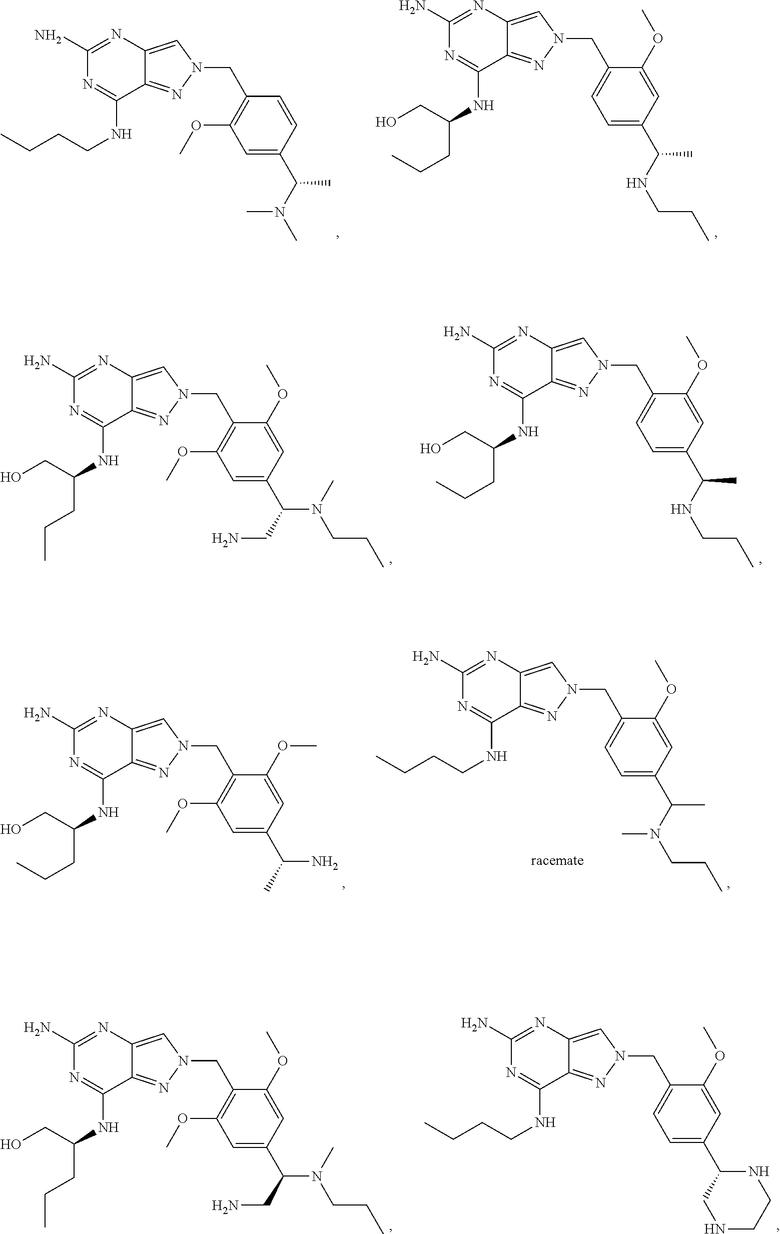 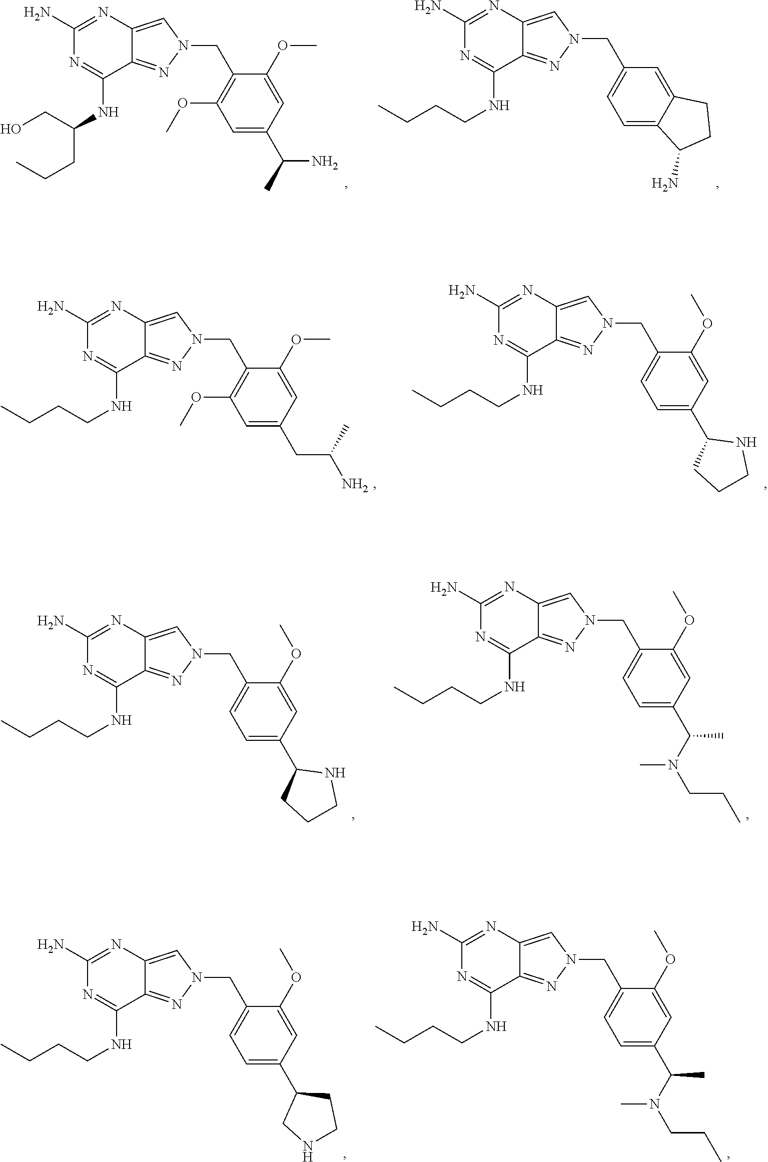 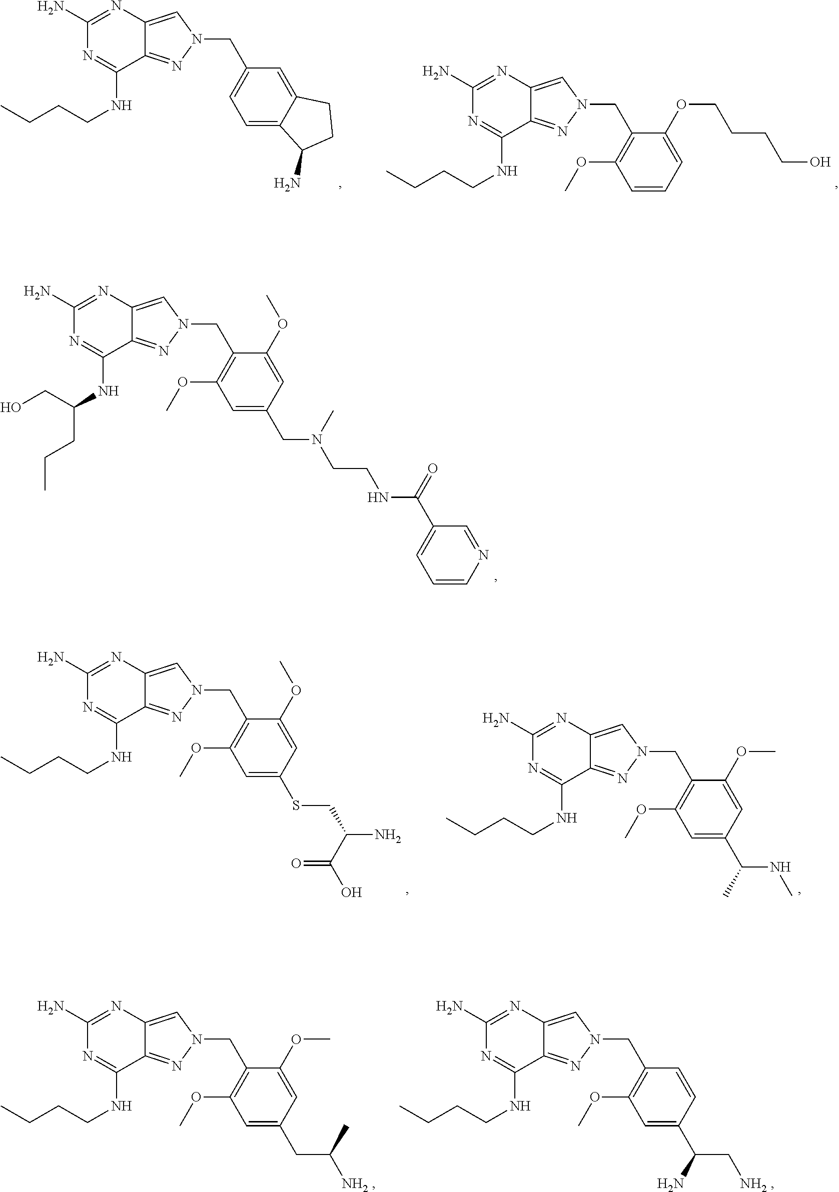 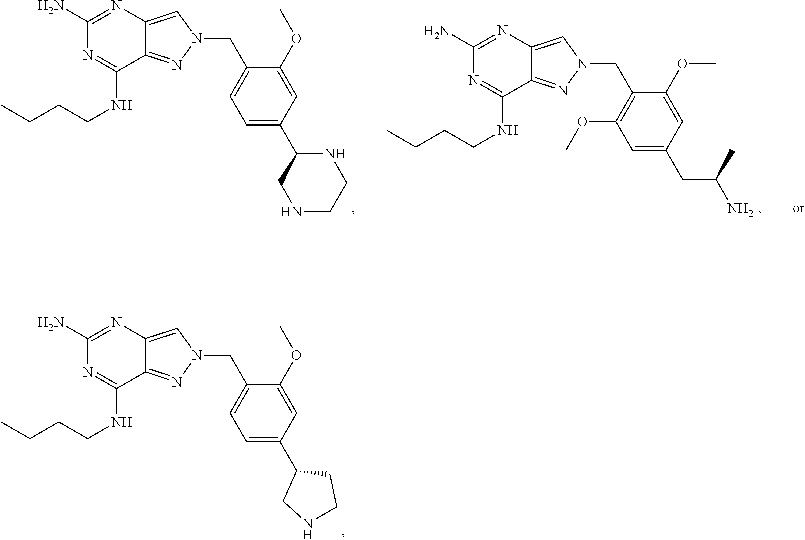 [SMILES: CCCCNc1nc(N)nc2cn(Cc3ccc([C@@H]4CNCCN4)cc3OC)nc12 ; CCCCNc1nc(N)nc2cn(Cc3c(OC)cc(C[C@@H](C)N)cc3OC)nc12 ; CCCCNc1nc(N)nc2cn(Cc3ccc([C@@H]4CCNC4)cc3OC)nc12]
or a pharmaceutically acceptable salt thereof.

13. A pharmaceutical composition comprising an effective amount of the compound of claim 1, or a pharmaceutically acceptable salt thereof, and a pharmaceutically acceptable carrier.

14. The pharmaceutical composition of claim 13, further comprising one or more additional therapeutic agents, wherein said additional therapeutic agents are anticancer agents.

15. The pharmaceutical composition of claim 14, wherein said additional therapeutic agents comprise an anti-human PD-1 antibody.

16. The pharmaceutical composition of claim 15, wherein said anti-human PD-1 antibody is pembrolizumab.

17. A method of treating cancer in a patient, said method comprising administering to said patient an effective amount of the compound according to claim 1, or a pharmaceutically acceptable salt thereof.

18. The method of claim 17, further comprising administering one or more additional therapeutic agents, wherein said additional therapeutic agents are anticancer agents.

19. The method of claim 18, wherein said additional therapeutic agents comprise an anti-human PD-1 antibody.

20. The method of claim 19, wherein said anti-human PD-1 antibody is pembrolizumab.
